(19) United States
(12) Patent Application Publication
(10) Pub. No.: US 2025/0171447 A1
(43) Pub. Date: May 29, 2025

(54) ADENOSINE RECEPTOR ANTAGONISTS, PHARMACEUTICAL COMPOSITIONS AND THEIR USE THEREOF

(71) Applicant: Merck Sharp & Dohme LLC, Rahway, NJ (US)

(72) Inventors: Amjad Ali, Freehold, NJ (US); Zachary G. Brill, San Francisco, CA (US); Alec Hiroshi Christian, Sharon, MA (US); Duane E. DeMong, Hanover, MA (US); Jared N. Cumming, Winchester, MA (US); Jenny Lorena Rico Duque, Canton, MA (US); Elisabeth T. Hennessy, Weston, MA (US); Derun Li, West Roxbury, MA (US); Yeon-Hee Lim, South San Francisco, CA (US); Christopher W. Plummer, Westfield, NJ (US); Jing Su, Scotch Plains, NJ (US)

(73) Assignee: Merck Sharp & Dohme LLC, Rahway, NJ (US)

(21) Appl. No.: 18/836,154

(22) PCT Filed: Feb. 14, 2023
(86) PCT No.: PCT/US2023/012982

§ 371 Date: Aug. 6, 2024

Related U.S. Application Data
(60) Provisional application No. 63/310,697, filed on Feb. 16, 2022.

(51) Int. Cl. C07D 487/04; A61K 31/519; A61K 31/538; A61K 45/06; C07D 519/00

(52) U.S. Cl. CPC C07D 487/04; A61K 31/519; A61K 31/538; A61K 45/06; C07D 519/00

(57) ABSTRACT

In its many embodiments, the invention provides compounds of structural Formula (I): (I), and pharmaceutically acceptable salts thereof, wherein, ring A, R¹, R² and R³ are as defined herein, and pharmaceutical compositions comprising one or more such compounds (alone and in combination with one or more other therapeutically active agents). The invention further provides methods for the preparation and use of the compounds of the invention, or a pharmaceutically acceptable salt thereof, alone and in combination with other therapeutic agents, as antagonists of A2a and/or A2b receptors, and in the treatment of a variety of diseases, conditions, or disorders that are mediated, at least in part, by the adenosine A2a receptor and/or the adenosine A2b receptor.

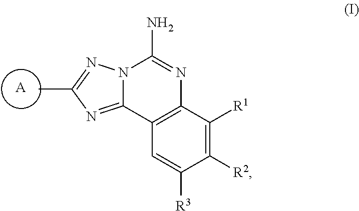

### FIELD OF THE INVENTION

The invention relates to novel compounds that inhibit at least one of the A2a and A2b adenosine receptors, and pharmaceutically acceptable salts thereof, and compositions comprising such compound(s) and salts. The invention further relates to methods for the synthesis of such compounds, and their use in the treatment of a variety of diseases, conditions, or disorders that are mediated, at least in part, by the adenosine A2a receptor and/or the adenosine A2b receptor. Such diseases, conditions, and disorders include but are not limited to cancer and immune-related disorders. The invention further relates to combination therapies, including but not limited to a combination comprising a compound of the invention and a PD-1 antagonist.

### BACKGROUND OF THE INVENTION

Adenosine is a purine nucleoside compound comprised of adenine and ribofuranose, a ribose sugar molecule. Adenosine occurs naturally in mammals and plays important roles in various biochemical processes, including energy transfer (as adenosine triphosphate and adenosine monophosphate) and signal transduction (as cyclic adenosine monophosphate). Adenosine also plays a causative role in processes associated with vasodilation, including cardiac vasodilation. It also acts as a neuromodulator (e.g., it is thought to be involved in promoting sleep). In addition to its involvement in these biochemical processes, adenosine is used as a therapeutic antiarrhythmic agent to treat supraventricular tachycardia and other indications.

The adenosine receptors are a class of purinergic G protein-coupled receptors with adenosine as the endogenous ligand. The four types of adenosine receptors in humans are referred to as A1, A2a, A2b, and A3. Modulation of A1 has been proposed for the management and treatment of neurological disorders, asthma, and heart and renal failure, among others. Modulation of A3 has been proposed for the management and treatment of asthma and chronic obstructive pulmonary diseases, glaucoma, cancer, stroke, and other indications. Modulation of the A2a and A2b receptors are also believed to be of potential therapeutic use.

In the central nervous system, A2a antagonists are believed to exhibit antidepressant properties and to stimulate cognitive functions. A2a receptors are present in high density in the basal ganglia, known to be important in the control of movement. Hence, A2a receptor antagonists are believed to be useful in the treatment of depression and to improve motor impairment due to neurodegenerative diseases such as Parkinson's disease, senile dementia (as in Alzheimer's disease), and in various psychoses of organic origin.

In the immune system, adenosine signaling through A2a receptors and A2b receptors, expressed on a variety of immune cells and endothelial cells, has been established as having an important role in protecting tissues during inflammatory responses. In this way (and others), tumors have been shown to evade host responses by inhibiting immune function and promoting tolerance. (See, e.g., Fishman, P., et al., Handb. Exp. Pharmacol. (2009) 193:399-441). Moreover, A2a and A2b cell surface adenosine receptors have been found to be upregulated in various tumor cells. Thus, antagonists of the A2a and/or A2b adenosine receptors represent a new class of promising oncology therapeutics. For example, activation of A2a adenosine receptors results in the inhibition of the immune response to tumors by a variety of cell types, including but not limited to the inhibition of natural killer cell cytotoxicity, the inhibition of tumor-specific CD4+/CD8+ activity, promoting the generation of LAG-3 and Foxp3+ regulatory T-cells, and mediating the inhibition of regulatory T-cells. Adenosine A2a receptor inhibition has also been shown to increase the efficacy of PD-1 inhibitors through enhanced anti-tumor T cell responses. As each of these immunosuppressive pathways has been identified as a mechanism by which tumors evade host responses, a cancer immunotherapeutic regimen that includes an antagonist of the A2a and/or A2b receptors, alone or together with one or more other therapeutic agents designed to mitigate immune suppression, may result in enhanced tumor immunotherapy. (See, e.g., P. Beavis, et al., Cancer Immunol. Res. DOI: 10.1158/2326-6066. CIR-14-0211, Feb. 11, 2015; Willingham, S B., et al., Cancer Immunol. Res., 6(10), 1136-49; and Leone R D, et al., Cancer Immunol. Immunother., August 2018, Vol. 67, Issue 8, 1271-1284).

Cancer cells release ATP into the tumor microenvironment when treated with chemotherapy and radiation therapy, which is subsequently converted to adenosine. (See Martins, I., et al., Cell Cycle, vol. 8, issue 22, pp. 3723 to 3728.) The adenosine can then bind to A2a receptors and blunt the anti-tumor immune response through mechanisms such as those described above. The administration of A2a receptor antagonists during chemotherapy or radiation therapy has been proposed to lead to the expansion of the tumor-specific T-cells while simultaneously preventing the induction of tumor-specific regulatory T-cells. (Young, A., et al., Cancer Discovery (2014) 4:879-888).

The combination of an A2a receptor antagonist with anti-tumor vaccines is believed to provide at least an additive therapeutic effect in view of their different mechanisms of action. Further, A2a receptor antagonists may be useful in combination with checkpoint blockers. By way of example, the combination of a PD-1 inhibitor and an adenosine A2a receptor inhibitor is thought to mitigate the ability of tumors to inhibit the activity of tumor-specific effector T-cells. (See, e.g., Willingham, S B., et al., Cancer Immunol. Res. (2018); 6(10), 1136-49; Leone, R D., et al., Cancer Immunol. Immunother., August 2018, Vol. 67, Issue 8, pp. 1271-1284; Fishman, P., et al., Handb. Exp. Pharmacol. (2009) 193:399-441; and Sitkovsky, M V., et al., (2014) Cancer Immunol. Res 2:598-605.)

The A2b receptor is a G protein-coupled receptor found in various cell types. A2b receptors require higher concentrations of adenosine for activation than the other adenosine receptor subtypes, including A2a. (Fredholm, B B., et al., Biochem. Pharmacol. (2001) 61:443-448). Conditions which activate A2b have been seen, for example, in tumors where hypoxia is observed. The A2b receptor may thus play an important role in pathophysiological conditions associated with massive adenosine release. While the pathway(s) associated with A2b receptor-mediated inhibition are not well understood, it is believed that the inhibition of A2b receptors (alone or together with A2a receptors) may block pro-tumorigenic functions of adenosine in the tumor microenvironment, including suppression of T-cell function and angiogenesis, and thus expand the types of cancers treatable by the inhibition of these receptors.

A2b receptors are expressed primarily on myeloid cells. The engagement of A2b receptors on myeloid derived suppressor cells (MDSCs) results in their expansion in vitro (Ryzhov, S. et al., J. Immunol. 2011, 187:6120-6129). MDSCs suppress T-cell proliferation and anti-tumor immune responses. Selective inhibitors of A2b receptors and A2b receptor knockouts have been shown to inhibit tumor growth in mouse models by increasing MDSCs in the tumor microenvironment (Iannone, R., et al., Neoplasia Vol. 13 No. 12, (2013) pp. 1400-1409; Ryzhov, S., et al., Neoplasia (2008) 10: 987-995). Thus, A2b receptor inhibition has become an attractive biological target for the treatment of a variety of cancers involving myeloid cells. Examples of cancers that express A2b receptors can be readily obtained through analysis of the publicly available TCGA database. Such cancers include lung, colorectal, head and neck, and cervical cancer, among others, and are discussed in further detail below.

Angiogenesis plays an important role in tumor growth. The angiogenesis process is highly regulated by a variety of factors and is triggered by adenosine under particular circumstances that are associated with hypoxia. The A2b receptor is expressed in human microvascular endothelial cells, where it plays an important role in the regulation of the expression of angiogenic factors such as the vascular endothelial growth factor (VEGF). In certain tumor types, hypoxia has been observed to cause an upregulation of the A2b receptors, suggesting that inhibition of A2b receptors may limit tumor growth by limiting the oxygen supply to the tumor cells. Furthermore, experiments involving adenylate cyclase activation indicate that A2b receptors are the sole adenosine receptor subtype in certain tumor cells, suggesting that A2b receptor antagonists may exhibit effects on particular tumor types. (See, e.g., Feoktistov, I., et al., (2003) Circ. Res. 92:485-492; and P. Fishman, P., et al., Handb. Exp. Pharmacol. (2009) 193:399-441).

In view of their promising and varied therapeutic potential, there remains a need in the art for potent and selective inhibitors of the A2a and/or A2b adenosine receptors, for use alone or in combination with other therapeutic agents. The invention addresses this and other needs.

### SUMMARY OF THE INVENTION

In one aspect, the invention provides compounds (hereinafter referred to as compounds of the invention) which have been found to be inhibitors of the adenosine A2a receptor and/or the adenosine A2b receptor. The compounds of the invention have a structure in accordance with the structural Formula (I):

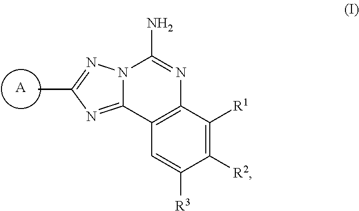

or a pharmaceutically acceptable salt thereof, wherein ring A, R¹, R² and R³ are as defined below.

In another aspect, the invention provides pharmaceutical compositions comprising at least one compound of the invention, or a pharmaceutically acceptable salt thereof, and a pharmaceutically acceptable carrier or diluent. Such compositions according to the invention may optionally further include one or more additional therapeutic agents as described herein.

In another aspect, the invention provides a method for treating or preventing a disease, condition, or disorder that is mediated, at least in part, by the adenosine A2a receptor and/or the adenosine A2b receptor in a subject (e.g., an animal or human) in need thereof, said method comprising administering to the subject a therapeutically effective amount of at least one compound of the invention, or a pharmaceutically acceptable salt thereof, alone or in combination with one or more additional therapeutic agents. These and other aspects and embodiments of the invention are described more fully below.

### DETAILED DESCRIPTION OF THE INVENTION

For each of the following embodiments, any variable not explicitly defined in the embodiment is as defined in Formula (I). In each of the embodiments described herein, each variable is selected independently of any other unless otherwise noted.

In certain embodiments described, the compounds of the invention have the structural formula of Formula (I):

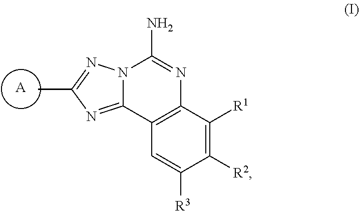

or a pharmaceutically acceptable salt thereof, wherein R¹, R² and R³ are independently selected from the group consisting of hydrogen, halogen, —CN, —OH, C₁-C₆alkyl, —OC₁-C₆alkyl, and —OC₁-C₆haloalkyl, wherein R¹, R² and R³ are not simultaneously hydrogen; ring A is a moiety selected from

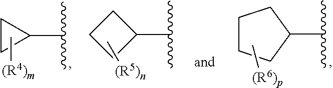

wherein m, n, and p are independently selected from the group consisting of 0, 1, 2 and 3; R⁴, R⁵ and R⁶ are independently selected from the group consisting of halogen, —OH, —CN, C₁-C₆alkyl, C₁-C₆alkyl-OH, C₁-C₆haloalkyl, C₃-C₆cycloalkyl, C₁-C₆alkylC₃-C₆cycloalkyl, aryl, C₁-C₆alkylaryl, heteroaryl, C₁-C₆alkylheteroaryl, heterocycloalkyl, C₁-C₆alkylheterocycloalkyl, —SO₂C₁-C₆alkyl, and —N(R⁷)₂; or m, n or p is 2, and the two R, R⁵ or R⁶, respectively, together with the carbon to which they are attached, form a C₃-C₆cycloalkyl or form a nitrogen containing ring, wherein the C₃-C₆cycloalkyl or nitrogen containing ring is unsubstituted or substituted with —COphenylC₁-C₆alkyl-OH, or —COOC₁-C₆alkylphenyl, wherein any of the above C₃-C₆cycloalkyl, aryl, C₁-C₆alkylaryl, heteroaryl, C₁-C₆alkylheteroaryl, or heterocycloalkyl is unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl; wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH; and
R⁷ is hydrogen, —CN, C₁-C₆alkyl, C₁-C₆alkylOH, C₁-C₆alkylCN, C₁-C₆alkylheterocycloalkyl, C₁-C₆alkylOC₁-C₆alkyl, —OC₁-C₆alkyl, C₁-C₆alkenyl, heterocycloalkyl, heteroaryl, aryl or C₃-C₆cycloalkyl, wherein the C₃-C₆cycloalkyl, aryl or heteroaryl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran.

Described herein are compounds wherein ring A is a moiety selected from

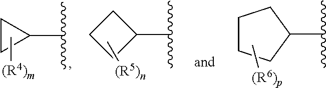

In certain embodiments, A is

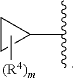

In certain embodiments, A is

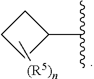

In certain embodiments, A is

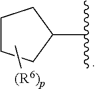

Described herein are compounds wherein m, n, and p are independently selected from the group consisting of 0, 1, 2 and 3. In certain embodiments, m is selected from the group consisting of 0, 1, 2 and 3. In certain embodiments, m is 1, 2 or 3. In certain embodiments, m is 1 or 2. In certain embodiments, m is 0. In certain embodiments, is m is 1. In certain embodiments, m is 2. In certain embodiments, m is 3.

In certain embodiments, n is selected from the group consisting of 0, 1, 2 and 3. In certain embodiments, n is 1, 2 or 3. In certain embodiments, n is 1 or 2. In certain embodiments, n is 0. In certain embodiments, is n is 1. In certain embodiments, n is 2. In certain embodiments, n is 3.

In certain embodiments, p is selected from the group consisting of 0, 1, 2 and 3. In certain embodiments, p is 1, 2 or 3. In certain embodiments, p is 1 or 2. In certain embodiments, p is 0. In certain embodiments, is p is 1. In certain embodiments, p is 2. In certain embodiments, p is 3.

Described herein are compounds, wherein R⁴, R⁵ and R⁶ are independently selected from the group consisting of halogen, —OH, —CN, C₁-C₆alkyl, C₁-C₆alkyl-OH, C₁-C₆haloalkyl, C₃-C₆cycloalkyl, C₁-C₆alkylC₃-C₆cycloalkyl, aryl, C₁-C₆alkylaryl, heteroaryl, C₁-C₆alkylheteroaryl, heterocycloalkyl, C₁-C₆alkylheterocycloalkyl, —SO₂C₁-C₆alkyl, and —N(R⁷)₂.

In certain embodiments, R⁴ is selected from the group consisting of halogen, —OH, —CN, C₁-C₆alkyl, C₁-C₆alkyl-OH, C₁-C₆haloalkyl, C₃-C₆cycloalkyl, C₁-C₆alkylC₃-C₆cycloalkyl, aryl, C₁-C₆alkylaryl, heteroaryl, C₁-C₆alkylheteroaryl, heterocycloalkyl, C₁-C₆alkylheterocycloalkyl, —SO₂C₁-C₆alkyl, and —N(R⁷)₂. In certain embodiments, R⁴ is —OH. In certain embodiments, R⁴ is —CN. In certain embodiments, R⁴ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁴ is methyl.

In certain embodiments, R⁴ is C₁-C₆alkyl-OH. Suitable alcohols include, but are not limited to, methanol, ethanol, propanol, butanol and iso-butanol. In certain embodiments, R⁴ is ethanol. In certain embodiments, R⁴ is C₁-C₆haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁴ is C₃-C₆cycloalkyl. Suitable examples of cycloalkyls include, but are not limited to, cyclopropyl, cyclobutyl, cyclopentyl and cyclohexyl. In certain embodiments, R⁴ is C₁-C₆alkylC₃-C₆cycloalkyl. Suitable examples of alkylcycloalkyls include, but are not limited to, CH₂cyclopropyl, CH₂cyclobutyl, CH₂cyclopentyl and CH₂cyclohexyl. In certain embodiments, R⁴ is aryl. Suitable aryls include phenyl. In certain embodiments, R⁴ is phenyl. In certain embodiments, R⁴ is C₁-C₆alkylaryl. Suitable alkylaryls include CH₂phenyl.

In certain embodiments, R⁴ is heteroaryl. Suitable heteroaryls include, but are not limited to, pyridyl (pyridinyl), oxazolyl, imidazolyl, triazolyl, furyl, triazinyl, thienyl, pyrimidyl, pyrazinyl, indolizinyl, cinnolinyl, phthalazinyl, quinazolinyl, naphthyridinyl, quinoxalinyl, purinyl, benzimidazolyl, quinolyl, benothiophenyl, isothiazolyl, isoquinolyl, triazolopyridinyl, and imidazolpyridinyl. In certain embodiments, R⁴ is pyrazolyl, pyridinyl, pyrazinyl, pyrimidinyl, benzimidazolyl, triazolopyridinyl, imidazolyl, or imidazolpyridinyl.

In certain embodiments, R⁴ is C₁-C₆alkylheteroaryl. Suitable alkylheteroaryls include the heteroaryls listed above attached to a hydrocarbon C₁-C₆alkyl chain.

In certain embodiments, R⁴ is heterocycloalkyl. Suitable heterocycloalkyls include, but are not limited to, azetidine, furan, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, isoindolinone, oxadiazolyl, dihydrobenzooxazine and dihydrocyclopentapyridinyl. In certain embodiments, R⁴ is isoindolinone, oxadiazolyl, dihydrobenzooxazine or tetrahydroquinoline.

In certain embodiments, R⁴ is C₁-C₆alkylheterocycloalkyl. Suitable alkylheterocycloalkyls include the heterocycloalkyls listed above attached to a hydrocarbon C₁-C₆alkyl chain.

In certain embodiments, R⁴ is —SO₂C₁-C₆alkyl. Suitable sulfoxides include, but are not limited to, —SO₂CH₃, —SO₂CH₂CH₂CH₃ and —SO₂CH₂CH₃.

In certain embodiments, R⁴ is —N(R⁷)₂. R⁷ is discussed in detail below.

In certain embodiments, R⁴ is

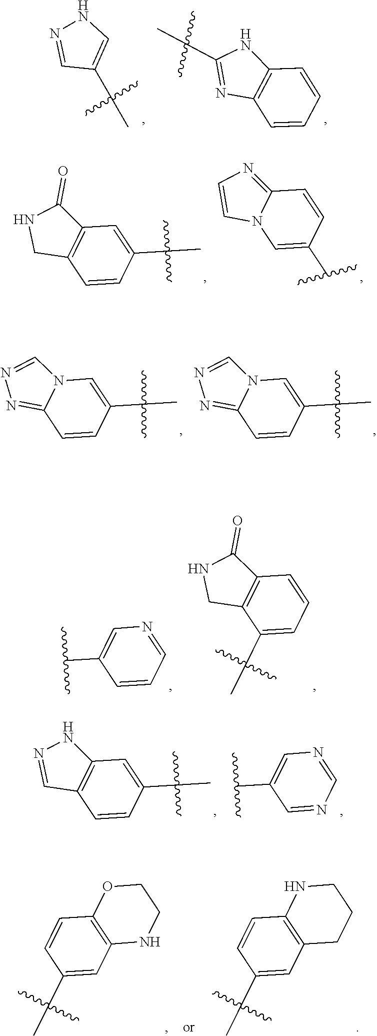

In certain embodiments, R⁵ is selected from the group consisting of halogen, —OH, —CN, C₁-C₆alkyl, C₁-C₆alkyl-OH, C₁-C₆haloalkyl, C₃-C₆cycloalkyl, C₁-C₆alkylC₃-C₆cycloalkyl, aryl, C₁-C₆alkylaryl, heteroaryl, C₁-C₆alkylheteroaryl, heterocycloalkyl, C₁-C₆alkylheterocycloalkyl, —SO₂C₁-C₆alkyl, and —N(R⁷)₂. In certain embodiments, R⁵ is —OH. In certain embodiments, R⁵ is —CN. In certain embodiments, R⁵ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁵ is methyl.

In certain embodiments, R⁵ is C₁-C₆alkyl-OH. Suitable alcohols include, but are not limited to, methanol, ethanol, propanol, butanol and iso-butanol. In certain embodiments, R⁵ is C₁-C₆haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁵ is C₃-C₆cycloalkyl. Suitable examples of cycloalkyls include, but are not limited to, cyclopropyl, cyclobutyl, cyclopentyl and cyclohexyl. In certain embodiments, R⁵ is C₁-C₆alkylC₃-C₆cycloalkyl. Suitable examples of alkylcycloalkyls include, but are not limited to, CH₂cyclopropyl, CH₂cyclobutyl, CH₂cyclopentyl and CH₂cyclohexyl. In certain embodiments, R⁵ is aryl. Suitable aryls include phenyl. In certain embodiments, R⁵ is phenyl. In certain embodiments, R⁵ is C₁-C₆alkylaryl. Suitable alkylaryls include CH₂phenyl.

In certain embodiments, R⁵ is heteroaryl. Suitable heteroaryls include, but are not limited to, pyridyl (pyridinyl), oxazolyl, imidazolyl, triazolyl, furyl, triazinyl, thienyl, pyrimidyl, pyrazinyl, indolizinyl, cinnolinyl, phthalazinyl, quinazolinyl, naphthyridinyl, quinoxalinyl, purinyl, benzimidazolyl, quinolyl, benothiophenyl, isothiazolyl, isoquinolyl, triazolopyridinyl, and imidazolpyridinyl. In certain embodiments, R⁵ is pyridinyl or pyrimidinyl.

In certain embodiments, R⁵ is C₁-C₆alkylheteroaryl. Suitable alkylheteroaryls include the heteroaryls listed above attached to a hydrocarbon C₁-C₆alkyl chain.

In certain embodiments, R⁵ is heterocycloalkyl. Suitable heterocycloalkyls include, but are not limited to, azetidine, furan, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, isoindolinone, oxadiazolyl, dihydrobenzooxazine and dihydrocyclopentapyridinyl. In certain embodiments, R⁵ is dihydropyrorroloprymidinyl or tetrahydropyridopyrimidinyl.

In certain embodiments, R⁵ is C₁-C₆alkylheterocycloalkyl. Suitable alkylheterocycloalkyls include the heterocycloalkyls listed above attached to a hydrocarbon C₁-C₆alkyl chain.

In certain embodiments, R⁵ is —SO₂C₁-C₆alkyl. Suitable sulfoxides include, but are not limited to, —SO₂CH₃, —SO₂CH₂CH₂CH₃ and —SO₂CH₂CH₃.

In certain embodiments, R⁵ is —N(R⁷)₂. R⁷ is discussed in detail below.

In certain embodiments, R⁵ is

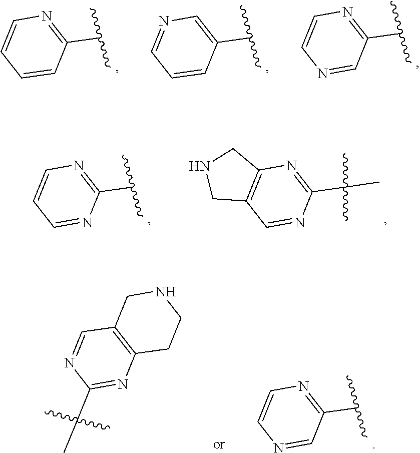

In certain embodiments, R⁶ is selected from the group consisting of halogen, —OH, —CN, C₁-C₆alkyl, C₁-C₆alkyl-OH, C₁-C₆haloalkyl, C₃-C₆cycloalkyl, C₁-C₆alkylC₃-C₆cycloalkyl, aryl, C₁-C₆alkylaryl, heteroaryl, C₁-C₆alkylheteroaryl, heterocycloalkyl, C₁-C₆alkylheterocycloalkyl, —SO₂C₁-C₆alkyl, and —N(R⁷)₂. In certain embodiments, R⁶ is —OH. In certain embodiments, R⁶ is —CN. In certain embodiments, R⁶ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁶ is methyl.

In certain embodiments, R⁶ is C₁-C₆alkyl-OH. Suitable alcohols include, but are not limited to, methanol, ethanol, propanol, butanol and iso-butanol. In certain embodiments, R⁶ is C₁-C₆haloalkyl. Suitable examples of haloalkyls include, but are not limited to, fluoromethyl, difluoromethyl, trifluoromethyl, 2-fluoroethyl, 1,2-difluoroethyl and 2,2-difluoroethyl. In certain embodiments, R⁶ is C₃-C₆cycloalkyl. Suitable examples of cycloalkyls include, but are not limited to, cyclopropyl, cyclobutyl, cyclopentyl and cyclohexyl. In certain embodiments, R⁶ is C₁-C₆alkylC₃-C₆cycloalkyl. Suitable examples of alkylcycloalkyls include, but are not limited to, CH₂cyclopropyl, CH₂cyclobutyl, CH₂cyclopentyl and CH₂cyclohexyl. In certain embodiments, R⁶ is aryl. Suitable aryls include phenyl. In certain embodiments, R⁶ is phenyl. In certain embodiments, R⁶ is C₁-C₆alkylaryl. Suitable alkylaryls include CH₂phenyl.

In certain embodiments, R⁶ is heteroaryl. Suitable heteroaryls include, but are not limited to, pyridyl (pyridinyl), oxazolyl, imidazolyl, triazolyl, furyl, triazinyl, thienyl, pyrimidyl, pyrazinyl, indolizinyl, cinnolinyl, phthalazinyl, quinazolinyl, naphthyridinyl, quinoxalinyl, purinyl, benzimidazolyl, quinolyl, benothiophenyl, isothiazolyl, isoquinolyl, triazolopyridinyl, and imidazolpyridinyl. In certain embodiments, R⁶ is pyridinyl.

In certain embodiments, R⁶ is C₁-C₆alkylheteroaryl. Suitable alkylheteroaryls include the heteroaryls listed above attached to a hydrocarbon C₁-C₆alkyl chain.

In certain embodiments, R⁶ is heterocycloalkyl. Suitable heterocycloalkyls include, but are not limited to, azetidine, furan, tetrahydropyranyl, tetrahydrofuranyl, pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydroquinolinyl, morpholinyl, tetrahydroisoquinolinyl, dihydroindolyl, isoindolinone, oxadiazolyl, dihydrobenzooxazine and dihydrocyclopentapyridinyl. In certain embodiments, R⁶ is isoindolinone, oxadiazolyl, dihydrobenzooxazine or tetrahydroquinoline.

In certain embodiments, R⁶ is C₁-C₆alkylheterocycloalkyl. Suitable alkylheterocycloalkyls include the heterocycloalkyls listed above attached to a hydrocarbon C₁-C₆alkyl chain.

In certain embodiments, R⁶ is —SO₂C₁-C₆alkyl. Suitable sulfoxides include, but are not limited to, —SO₂CH₃, —SO₂CH₂CH₂CH₃ and —SO₂CH₂CH₃.

In certain embodiments, R⁶ is —N(R⁷)₂. R⁷ is discussed in detail below.

In certain embodiments, R⁶ is

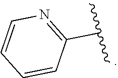 [SMILES: *c1ccccn1]

In certain embodiments of the compounds described herein, when m, n or p is 2, the two R⁴, R⁵ or R⁶, together with the carbon to which they are attached, form a C₃-C₆cycloalkyl or form a nitrogen containing ring, wherein the C₃-C₆cycloalkyl or nitrogen containing ring is unsubstituted or substituted with —COphenylC₁-C₆alkyl-OH or —COOC₁-C₆alkylphenyl.

In certain embodiments, of the compounds described herein, m is 2 and the two R⁴, together with the carbon to which they are attached, form a C₃-C₆cycloalkyl or form a nitrogen containing ring, wherein the C₃-C₆cycloalkyl or nitrogen containing ring is unsubstituted or substituted with —COphenylC₁-C₆alkyl-OH or —COOC₁-C₆alkylphenyl.

In certain embodiments, of the compounds described herein, m is 2 and the two R⁴, together with the carbon to which they are attached, form a C₃-C₆cycloalkyl, wherein the C₃-C₆cycloalkyl is unsubstituted or substituted with —COphenylC₁-C₆alkyl-OH or —COOC₁-C₆alkylphenyl.

In certain embodiments, m is 2 and the two R⁴ form a C₃-C₆cycloalkyl. In certain embodiments of the compounds described herein, m is 2 and the two R⁴, together with the carbon to which they are attached, form a C₃-C₆cycloalkyl, wherein the C₃-C₆cycloalkyl is unsubstituted.

In certain embodiments of the compounds described herein, m is 2 and the two R, together with the carbon to which they are attached, form a C₃-C₆cycloalkyl, wherein the C₃-C₆cycloalkyl is substituted with —COphenylC₁-C₆alkyl-OH or —COOC₁-C₆alkylphenyl. Suitable examples of cycloalkyls include, but are not limited to, cyclopropyl, cyclobutyl, cyclopentyl and cyclohexyl.

In certain embodiments of the compounds described herein, m is 2 and the two R⁴, together with the carbon to which they are attached, form a nitrogen containing ring, wherein the nitrogen containing ring is unsubstituted or substituted with —COphenylC₁-C₆alkyl-OH or —COOC₁-C₆alkylphenyl.

In certain embodiments of the compounds described herein, m is 2 and the two R⁴, together with the carbon to which they are attached, form a nitrogen containing ring, wherein the nitrogen containing ring is unsubstituted. Suitable examples of nitrogen-containing rings include, but are not limited to, aziridinyl, azirinyl, azetidinyl, azete, indoline, pyrrolidinyl, pyrrolyl, piperidinyl and pyridinyl.

In certain embodiments of the compounds described herein, m is 2 and the two R⁴, together with the carbon to which they are attached, form a nitrogen containing ring, wherein the nitrogen containing ring is substituted with —COphenylC₁-C₆alkyl-OH or —COOC₁-C₆alkylphenyl.

In certain embodiments of the compounds described herein, n is 2 and the two R⁵, together with the carbon to which they are attached, form a C₃-C₆cycloalkyl or form a nitrogen containing ring, wherein the C₃-C₆cycloalkyl or nitrogen containing ring is unsubstituted or substituted with —COphenylC₁-C₆alkyl-OH or —COOC₁-C₆alkylphenyl.

In certain embodiments of the compounds described herein, n is 2 and the two R⁵, together with the carbon to which they are attached, form a C₃-C₆cycloalkyl, wherein the C₃-C₆cycloalkyl is unsubstituted or substituted with —COphenylC₁-C₆alkyl-OH or —COOC₁-C₆alkylphenyl.

In certain embodiments of the compounds described herein, n is 2 and the two R⁵, together with the carbon to which they are attached, form a C₃-C₆cycloalkyl, wherein the C₃-C₆cycloalkyl is unsubstituted.

In certain embodiments of the compounds described herein, n is 2 and the two R⁵, together with the carbon to which they are attached, form a C₃-C₆cycloalkyl, wherein the C₃-C₆cycloalkyl is substituted with —COphenylC₁-C₆alkyl-OH or —COOC₁-C₆alkylphenyl. Suitable examples of cycloalkyls include, but are not limited to, cyclopropyl, cyclobutyl, cyclopentyl and cyclohexyl.

In certain embodiments, n is 2 and the two R⁵ form a nitrogen containing ring, wherein the nitrogen containing ring is unsubstituted or substituted with —COphenylC₁-C₆alkyl-OH, —COOC₁-C₆alkylphenyl.

In certain embodiments of the compounds described herein, n is 2 and the two R⁵, together with the carbon to which they are attached, form a nitrogen containing ring, wherein the nitrogen containing ring is unsubstituted or substituted with —COphenylC₁-C₆alkyl-OH or —COOC₁-C₆alkylphenyl.

In certain embodiments of the compounds described herein, n is 2 and the two R⁵, together with the carbon to which they are attached, form a nitrogen containing ring, wherein the nitrogen containing ring is unsubstituted. Suitable examples of nitrogen-containing rings include, but are not limited to, aziridinyl, azirinyl, azetidinyl, azete, indoline, pyrrolidinyl, pyrrolyl, piperidinyl and pyridinyl.

In certain embodiments of the compounds described herein, n is 2 and the two R⁵, together with the carbon to which they are attached, form a nitrogen containing ring, wherein the nitrogen containing ring is substituted with —COphenylC₁-C₆alkyl-OH or —COOC₁-C₆alkylphenyl.

In certain embodiments of the compounds described herein, p is 2 and the two R⁶, together with the carbon to which they are attached, form a C₃-C₆cycloalkyl or form a nitrogen containing ring, wherein the C₃-C₆cycloalkyl or nitrogen containing ring is unsubstituted or substituted with —COphenylC₁-C₆alkyl-OH or —COOC₁-C₆alkylphenyl.

In certain embodiments of the compounds described herein, p is 2 and the two R⁶, together with the carbon to which they are attached, form a C₃-C₆cycloalkyl, wherein the C₃-C₆cycloalkyl is unsubstituted or substituted with —COphenylC₁-C₆alkyl-OH or —COOC₁-C₆alkylphenyl.

In certain embodiments of the compounds described herein, p is 2 and the two R⁶, together with the carbon to which they are attached, form a C₃-C₆cycloalkyl, wherein the C₃-C₆cycloalkyl is unsubstituted.

In certain embodiments of the compounds described herein, p is 2 and the two R⁶, together with the carbon to which they are attached, form a C₃-C₆cycloalkyl, wherein the C₃-C₆cycloalkyl is substituted with —COphenylC₁-C₆alkyl-OH or —COOC₁-C₆alkylphenyl. Suitable examples of cycloalkyls include, but are not limited to, cyclopropyl, cyclobutyl, cyclopentyl and cyclohexyl.

In certain embodiments of the compounds described herein, p is 2 and the two R⁶, together with the carbon to which they are attached, form a nitrogen containing ring, wherein the nitrogen containing ring is unsubstituted or substituted with —COphenylC₁-C₆alkyl-OH or —COOC₁-C₆alkylphenyl.

In certain embodiments of the compounds described herein, p is 2 and the two R⁶, together with the carbon to which they are attached, form a nitrogen containing ring, wherein the nitrogen containing ring is unsubstituted. Suitable examples of nitrogen-containing rings include, but are not limited to, aziridinyl, azirinyl, azetidinyl, azete, indoline, pyrrolidinyl, pyrrolyl, piperidinyl and pyridinyl.

In certain embodiments of the compounds described herein, p is 2 and the two R⁶, together with the carbon to which they are attached, form a nitrogen containing ring, wherein the nitrogen containing ring is substituted with —COphenylC₁-C₆alkyl-OH or —COOC₁-C₆alkylphenyl.

Described herein are compounds wherein any of the above C₃-C₆cycloalkyl, aryl, C₁-C₆alkylaryl, heteroaryl, C₁-C₆alkylheteroaryl, or heterocycloalkyl is unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl; wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is unsubstituted.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with one substituent selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with two substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl.

In certain embodiments of the compounds described herein, any of the above C₃-C₆cycloalkyl groups are unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl.

In certain embodiments of the compounds described herein, any of the above aryl groups are unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl. In certain embodiments, described herein are compounds wherein any of the above C₁-C₆alkylaryl groups are unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl; wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH.

In certain embodiments, described herein are compounds wherein any of the above heteroaryl groups are unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl; wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH.

In certain embodiments, described herein are compounds wherein any of the above C₁-C₆alkylheteroaryl groups are unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl; wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH.

In certain embodiments described herein are compounds wherein any of the above heterocycloalkyl groups are unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl; wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with halogen.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —CN.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —OH.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with C₁-C₆alkyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with oxo.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —CON(R⁷)₂.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with C₁-C₆alkyl-OH.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with C₁-C₆haloalkyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with NHC₁-C₆alkyl-OH.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with NHCO(C₁-C₆alkyl).

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —SO₂NH₂.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with C₁-C₆alkenyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —OC₁-C₆alkyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —OC₁-C₆haloalkyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —N(R⁷)₂.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with C₁-C₆alkylN(R⁷)₂.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with C₁-C₆alkylheterocycloalkyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with heterocycloalkyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with heteroaryl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with C₃-C₆cycloalkyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with C₁-C₆alkylheteroaryl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —COOC₁-C₆alkyl.

In certain embodiments of the compounds described herein, any of the above C₃-C₆cycloalkyl, aryl, C₁-C₆alkylaryl, heteroaryl, C₁-C₆alkylheteroaryl, or heterocycloalkyl is substituted with —COC₁-C₆alkylaryl.

In certain embodiments, the compounds described herein have a structural formula of Formula (I):

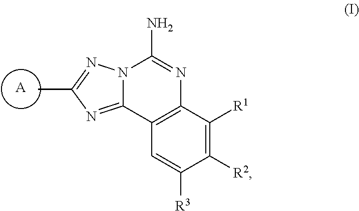

or a pharmaceutically acceptable salt thereof, wherein R¹, R² and R³ are independently selected from the group consisting of hydrogen, halogen, —CN, —OH, C₁-C₆alkyl, —OC₁-C₆alkyl, and —OC₁-C₆haloalkyl, wherein R¹, R² and R³ are not simultaneously hydrogen; ring A is a moiety selected from

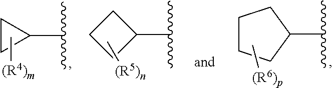

wherein m is 0, 1, 2 or 3; n is 0, 1, 2 or 3; p is 0, 1, 2 or 3;
R⁴ is selected from the group consisting of halogen, C₁-C₆alkyl, C₁-C₆alkyl-OH, C₁-C₆haloalkyl, C₃-C₆cycloalkyl, aryl, C₁-C₆alkylaryl, heteroaryl, and heterocycloalkyl, or m is 2 and the two R, together with the carbon to which they are attached, form a C₃-C₆cycloalkyl or form a nitrogen containing ring, wherein the nitrogen containing ring is unsubstituted or substituted with —COphenylC₁-C₆alkyl-OH, or —COOC₁-C₆alkylphenyl; wherein the C₃-C₆cycloalkyl, aryl, C₁-C₆alkylaryl, heteroaryl, or heterocycloalkyl is unsubstituted or substituted with one to three substituents independently selected from the group consisting of halogen, C₁-C₆alkyl, —CONH₂, oxo, —CONH(C₁-C₆alkyl), C₁-C₆alkyl-OH, NHCO(C₁-C₆alkyl), C₁-C₆haloalkyl, NHC₁-C₆alkyl-OH and —SO₂NH₂;
R⁵ is selected from the group consisting of halogen, —OH, —CN, C₁-C₆alkyl, C₁-C₆alkylOH, aryl, C₁-C₆alkylheteroaryl, heteroaryl, heterocycloalkyl, —SO₂C₁-C₆alkyl, and —N(R⁷)₂; wherein the aryl, C₁-C₆alkylheteroaryl, heteroaryl, or heterocycloalkyl is unsubstituted or substituted with one to three substituents independently selected from the group consisting of halogen, —CN, —OH, C₁-C₆alkylOH, C₁-C₆alkyl, oxo, C₁-C₆alkenyl, —OC₁-C₆alkyl, —OC₁-C₆haloalkyl, C₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl; wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH; or n is 2 and the two R⁵, together with the carbon to which they are attached, form a nitrogen containing ring, wherein the nitrogen containing ring is unsubstituted or substituted with —COphenylC₁-C₆alkyl-OH, or —COOC₁-C₆alkylphenyl;
R⁶ is selected from the group consisting of halogen, —OH, and heteroaryl, wherein the heteroaryl, is unsubstituted or substituted with C₁-C₆alkylNH₂; and
R⁷ is hydrogen, —CN, C₁-C₆alkyl, C₁-C₆alkylOH, C₁-C₆alkylCN, C₁-C₆alkylheterocycloalkyl, C₁-C₆alkylOC₁-C₆alkyl, —OC₁-C₆alkyl, C₁-C₆alkenyl, heterocycloalkyl, heteroaryl, aryl or C₃-C₆cycloalkyl, wherein the C₃-C₆cycloalkyl, aryl or heteroaryl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran.

In certain embodiments, ring A is

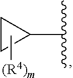

wherein
R⁴ is selected from the group consisting of:
halogen,
C₁-C₆alkyl,
C₁-C₆alkyl-OH,
C₁-C₆haloalkyl,
C₃-C₆cycloalkyl,
aryl,
C₁-C₆alkylaryl,
heteroaryl, and
heterocycloalkyl; and
m is 0, 1, 2 or 3, or
m is 2 and the two R, together with the carbon to which they are attached, form a C₃-C₆cycloalkyl,
wherein any of the above C₃-C₆cycloalkyl, aryl, C₁-C₆alkylaryl, heteroaryl, or heterocycloalkyl is unsubstituted or substituted with one to three substituents independently selected from the group consisting of halogen, C₁-C₆alkyl, oxo, —CONH₂, —CONH(C₁-C₆alkyl), C₁-C₆alkyl-OH, C₁-C₆haloalkyl, NHC₁-C₆alkyl-OH, NHCO(C₁-C₆alkyl) and —SO₂NH₂.

In certain embodiments, A is

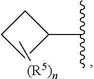

wherein
R⁵ is selected from the group consisting of:
halogen,
—OH,
CN,
C₁-C₆alkyl,
C₁-C₆alkyl-OH,
aryl,
C₁-C₆alkylheteroaryl;
heteroaryl,
heterocycloalkyl,
SO₂C₁-C₆alkyl, and
N(R⁷)₂; and
n is 0, 1, 2 or 3, or
n is 2 and the two R, together with the carbon to which they are attached, form a nitrogen containing ring, wherein the nitrogen containing ring is unsubstituted or substituted with —COphenylC₁-C₆alkyl-OH, or —COOC₁-C₆alkylphenyl,
   wherein any of the above aryl, C₁-C₆alkylheteroaryl, heteroaryl, or heterocycloalkyl is unsubstituted or substituted with one to three substituents independently selected from the group consisting of halogen, —CN, —OH, C₁-C₆alkylOH, C₁-C₆alkyl, C₁-C₆alkenyl, —OC₁-C₆alkyl, —OC₁-C₆haloalkyl, C₁-C₆haloalkyl, —N(R⁷)₂, C₁-C₆alkylN(R⁷)₂, C₁-C₆alkylpiperazinyl, oxetane, pyrrolidinyl, oxa-azabicycloheptane, C₁-C₆alkyloxa-azabicycloheptane, C₁-C₆alkylthiomorpholineoxide, thiomorpholineoxide, piperidinyl, C₃-C₆cycloalkyl, C₁-C₆alkylpyrrolidinyl, azetidine, C₁-C₆alkylazetidine, —COOC₁-C₆alkyl, morpholine, C₁-C₆alkylmorpholine, —CON(R⁷)₂, azabicyclooctane, C₁-C₆alkylazabicyclooctane, azabicycloheptane, C₁-C₆alkylazabicycloheptane, and —COC₁-C₆alkylphenyl; wherein the oxetane, C₃-C₆cycloalkyl, pyrrolidinyl, piperazinyl, C₁-C₆alkylpyrrolidinyl, azetidine, C₁-C₆alkylazetidine, piperidinyl, C₁-C₆alkylOH, C₁-C₆alkylN(R⁷)₂ and —COC₁-C₆alkylphenyl are unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH; and R⁷ is hydrogen, —CN, C₁-C₆alkylCN, C₁-C₆alkyl, tetrahydronaphthyridine, pyridinyl, phenyl, imidazolyl, pyrazinyl, bicyclopentane, C₁-C₆alkylOH, C₃-C₆cycloalkyl, oxetanyl, C₁-C₆alkenyl, C₁-C₆alkyltetrahydrofuran, —OC₁-C₆alkyl, or C₁-C₆alkylOC₁-C₆alkyl, wherein the C₃-C₆cycloalkyl, phenyl, pyrazinyl, or pyridinyl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran.

In certain embodiments, ring A is

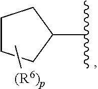

wherein
R⁶ is selected from the group consisting of:
halogen,
—OH, and
pyridinyl, wherein the pyridinyl is substituted with 1-3 C₁-C₆alkylNH₂ substituents; and
p is 0, 1, 2, or 3.

In certain embodiments, ring A is

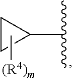

wherein:
R⁴ is selected from the group consisting of:
halogen,
C₁-C₆alkyl,
C₁-C₆alkyl-OH,
aryl,
C₁-C₆alkylaryl,
heteroaryl, and
heterocycloalkyl, wherein any of the above aryl, C₁-C₆alkylaryl, heteroaryl, or heterocycloalkyl is unsubstituted or substituted with one to three substituents independently selected from the group consisting of halogen, C₁-C₆alkyl, oxo, —CONH₂, —CONH(C₁-C₆alkyl), C₁-C₆alkyl-OH, C₁-C₆haloalkyl, NHC₁-C₆alkyl-OH, NHCO(C₁-C₆alkyl) and —SO₂NH₂; and
m is 1, 2 or 3.

In certain embodiments, A is

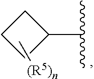

wherein R⁵ is selected from the group consisting of:
halogen,
—OH,
aryl,
heteroaryl,
heterocycloalkyl, and
—N(R⁷)₂;
wherein any of the above aryl, heteroaryl, or heterocycloalkyl is unsubstituted or substituted with one to three substituents independently selected from the group consisting of halogen, —CN, —OH, C₁-C₆alkylOH, C₁-C₆alkyl, C₁-C₆alkenyl, —OC₁-C₆alkyl, —OC₁-C₆haloalkyl, C₁-C₆haloalkyl, —N(R⁷)₂, C₁-C₆alkylN(R⁷)₂, C₁-C₆alkylpiperazinyl, oxetane, pyrrolidinyl, oxa-azabicycloheptane, C₁-C₆alkyloxa-azabicycloheptane, C₁-C₆alkylthiomorpholineoxide, thiomorpholineoxide, piperidinyl, C₃-C₆cycloalkyl, C₁-C₆alkylpyrrolidinyl, azetidine, C₁-C₆alkylazetidine, —COOC₁-C₆alkyl, morpholine, C₁-C₆alkylmorpholine, —CON(R⁷)₂, azabicyclooctane, C₁-C₆alkylazabicyclooctane, azabicycloheptane, C₁-C₆alkylazabicycloheptane, and —COC₁-C₆alkylphenyl,
wherein the oxetane, C₃-C₆cycloalkyl, pyrrolidinyl, piperazinyl, C₁-C₆alkylpyrrolidinyl, azetidine, C₁-C₆alkylazetidine, piperidinyl, C₁-C₆alkylOH, C₁-C₆alkylN(R⁷)₂ and —COC₁-C₆alkylphenyl are unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH; and
R⁷ is hydrogen, —CN, C₁-C₆alkylCN, C₁-C₆alkyl, tetrahydronaphthyridine, pyridinyl, phenyl, imidazolyl, pyrazinyl, bicyclopentane, C₁-C₆alkylOH, C₃-C₆cycloalkyl, oxetanyl, C₁-C₆alkenyl, C₁-C₆alkyltetrahydrofuran, —OC₁-C₆alkyl, or C₁-C₆alkylOC₁-C₆alkyl, wherein the C₃-C₆cycloalkyl, phenyl, pyrazinyl, or pyridinyl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran; and
n is 1, 2 or 3.

In certain embodiments, ring A is

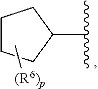

wherein R⁶ is selected from the group consisting of:
halogen,
—OH, and
pyridinyl, wherein the pyridinyl is substituted with 1-3 C₁-C₆alkylNH₂ substituents; and
p is 1, 2, or 3.

In certain embodiments, R⁴ is selected from the group consisting of halogen, C₁-C₆alkyl, C₁-C₆alkyl-OH, C₁-C₆haloalkyl, C₃-C₆cycloalkyl, C₁-C₆alkylphenyl, pyrazolyl, pyridinyl, pyrazinyl, phenyl, isoindolinone, oxadiazolyl, triazolyl, pyrimidinyl, pyridazinyl, benzimidazolyl, triazolopyridinyl, dihydrobenzooxazine, tetrahydroquinoline and imidazolyl; wherein the C₃-C₆cycloalkyl, C₁-C₆alkylphenyl, pyrazolyl, pyridinyl, pyrazinyl, phenyl, isoindolinone, oxadiazolyl, thiazolyl, triazolyl, pyrimidinyl, pyridazinyl, benzimidazolyl, triazolopyridinyl, dihydrobenzooxazine, tetrahydroquinoline and imidazolyl is unsubstituted or substituted with one to three substituents independently selected from the group consisting of halogen, C₁-C₆alkyl, —CONH₂, —CONH(C₁-C₆alkyl), C₁-C₆alkyl-OH, C₁-C₆haloalkyl, NHC₁-C₆alkyl-OH and —SO₂NH₂.

In certain embodiments, n is 1 and R⁴ is phenyl, wherein the phenyl is unsubstituted or substituted with propanol, —NHCOCH₃, —CONHCH₃, ethanol, fluorine, or —CONH₂.

In certain embodiments, n is 2 and R⁴ is independently selected from the group consisting of phenyl and fluorine.

In certain embodiments, n is 1 and R⁴ is selected from the group consisting of pyrazolyl, benzimidazolyl, dihydroisoindolone, imidazopyridinyl, triazolopyridinyl, pyridinyl, indazolyl, pyrimidinyl, benzoxazinyl and tetrahydroisoquinolinyl wherein the pyrazolyl, benzimidazolyl, dihydroisoindolone, imidazopyridinyl, triazolopyridinyl, pyridinyl, indazolyl, pyrimidinyl, benzoxazinyl or tetrahydroisoquinolinyl is unsubstituted or substituted with one or two substituents independently selected from the group consisting of methyl, ethyl, propanol, fluorine, NHCH₂CH₂OH, ethanol and trifluoromethyl.

In certain embodiments, n is 1 and R⁴ is selected from the group consisting of methyl, propanol and ethanol.

In certain embodiments, n is 1 and R⁴ is CH₂phenyl, wherein the CH₂phenyl is unsubstituted or substituted with fluorine.

In certain embodiments, n is 1 and R⁴ is selected from the group consisting of pyrazolyl, benzimidazolyl, dihydroisoindolone, imidazopyridinyl, triazolopyridinyl, pyridinyl, indazolyl, pyrimidinyl, benzoxazinyl and tetrahydroisoquinolinyl wherein the pyrazolyl, benzimidazolyl, dihydroisoindolone, imidazopyridinyl, triazolopyridinyl, pyridinyl, indazolyl, pyrimidinyl, benzoxazinyl or tetrahydroisoquinolinyl is unsubstituted or substituted with one or two substituents independently selected from the group consisting of methyl, ethyl, propanol, fluorine, NHCH₂CH₂OH, ethanol and trifluoromethyl.

In certain embodiments, R⁵ is selected from the group consisting of halogen, —OH, —CN, C₁-C₆alkyl, C₁-C₆alkyl-OH, phenyl, C₁-C₆alkylpyridinyl, pyridinyl, pyrimidinyl, triazolyl, pyrazinyl, imidazolyl, oxadiazolyl, dihydrocyclopentapyridinyl, dihydroimidazopyrazinyl, dihydrotriazolopyridinyl, dihydropyrrolopyrimidinyl, tetrahydroimidazopyrazinyl, tetrahydrotriazolopyridinyl, tetrahydropyridopyrimidinyl, oxidaneylpyridinyl, tetrahydronaphthyridinyl, pyridinone, —SO₂C₁-C₆alkyl, and NHR⁷; wherein the phenyl, C₁-C₆alkylpyridinyl, pyridinyl, pyrimidinyl, triazolyl, pyrazinyl, imidazolyl, oxadiazolyl, dihydrocyclopentapyridinyl, dihydroimidazopyrazinyl, dihydrotriazolopyridinyl, dihydropyrrolopyrimidinyl, tetrahydroimidazopyrazinyl, tetrahydrotriazolopyridinyl, tetrahydropyridopyrimidinyl, oxidaneylpyridinyl, tetrahydronaphthyridinyl and pyridinone are unsubstituted or substituted with one to three substituents independently selected from the group consisting of halogen, —CN, —OH, C₁-C₆alkylOH, C₁-C₆alkyl, C₁-C₆alkenyl, —OC₁-C₆alkyl, —OC₁-C₆haloalkyl, C₁-C₆haloalkyl, —N(R⁷)₂, C₁-C₆alkylN(R⁷)₂, C₁-C₆alkylpiperazinyl, oxetane, pyrrolidinyl, oxa-azabicycloheptane, C₁-C₆alkyloxa-azabicycloheptane, C₁-C₆alkylthiomorpholineoxide, thiomorpholineoxide, piperidinyl, C₃-C₆cycloalkyl, C₁-C₆alkylpyrrolidinyl, azetidine, C₁-C₆alkylazetidine, —COOC₁-C₆alkyl, morpholine, C₁-C₆alkylmorpholine, —CON(R⁷)₂, azabicyclooctane, C₁-C₆alkylazabicyclooctane, azabicycloheptane, C₁-C₆alkylazabicycloheptane, and —COC₁-C₆alkylphenyl; wherein the oxetane, C₃-C₆cycloalkyl, pyrrolidinyl, piperazinyl, C₁-C₆alkylpyrrolidinyl, azetidine, C₁-C₆alkylazetidine, piperidinyl, C₁-C₆alkylOH, C₁-C₆alkylN(R⁷)₂ and —COC₁-C₆alkylphenyl are unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH;
R⁷ is hydrogen, —CN, C₁-C₆alkylCN, C₁-C₆alkyl, tetrahydronaphthyridine, pyridinyl, phenyl, imidazolyl, pyrazinyl, bicyclopentane, C₁-C₆alkylOH, C₃-C₆cycloalkyl, oxetanyl, C₁-C₆alkenyl, C₁-C₆alkyltetrahydrofuran, —OC₁-C₆alkyl, or C₁-C₆alkylOC₁-C₆alkyl, wherein the C₃-C₆cycloalkyl, phenyl, pyrazinyl, or pyridinyl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran; and
n is 0, 1, 2 or 3.

In certain embodiments, n is 1 and R⁵ is phenyl, wherein the phenyl is unsubstituted or substituted with propanol, CHNH₂CF₃, CH₂piperazinyl, or piperidinyl, wherein the piperidinyl is further substituted with trifluoromethyl.

In certain embodiments, n is 1 or 2, and R⁵ is pyridinyl, —OH or NH₂, wherein the pyridinyl is unsubstituted or substituted with one or two substituents independently selected from the group consisting of methyl, fluorine, chlorine, cyclobutyl, propanol, butanol,

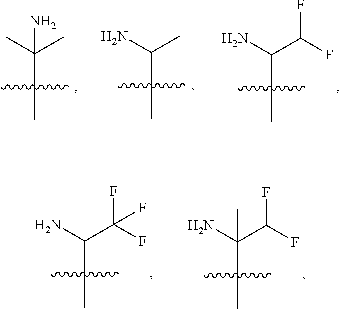

piperazinyl, azetidine, and oxetane, wherein the piperazinyl is further substituted with methyl, wherein the cyclobutyl is further substituted with two fluorine and —OH or two fluorine and NH₂, wherein the azetidine is further substituted with —CN, wherein the oxetane is further substituted with —OH or NH₂.

In certain embodiments, n is 1 and R⁴ is pyrazinyl, dihydropyrrolopyrimidinyl or dihydropyrrolopyrimidinyl, wherein the pyrazinyl is unsubstituted or substituted with 1-3 substituents independently selected from e group consisting of methyl, propanol, cyclopropyl, NH₂ trifluoromethyl, CH₂NH₂ and

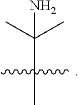 [SMILES: *C(C)(C)N]

In certain embodiments, R⁶ is selected from the group consisting of halogen, —OH, and pyridinyl, wherein the pyridinyl is unsubstituted or substituted with 1-3 C₁-C₆alkylNH₂ substituents; and p is 0, 1, 2 or 3.

In certain embodiments, p is 1 and R⁶ is pyridinyl, wherein the pyridinyl is substituted with

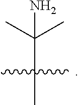 [SMILES: *C(C)(C)N]

In certain embodiments described herein, the compounds have a structural Formula (I):

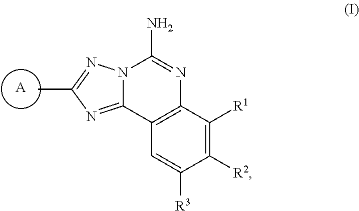

or a pharmaceutically acceptable salt thereof, wherein
R¹, R² and R³ are independently selected from the group consisting of hydrogen, halogen, —CN, —OH, C₁-C₆alkyl, —OC₁-C₆alkyl, and —OC₁-C₆haloalkyl, wherein R¹, R² and R³ are not simultaneously hydrogen;
ring A is a moiety selected from

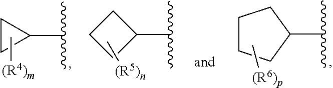

wherein
R, R⁵ and R⁶ are independently selected from the group consisting of:
halogen,
—OH,
CN,
C₁-C₆alkyl,
C₁-C₆alkyl-OH,
C₁-C₆haloalkyl,
C₃-C₆cycloalkyl,
C₁-C₆alkylC₃-C₆cycloalkyl,
aryl,
C₁-C₆alkylaryl,
heteroaryl,
C₁-C₆alkylheteroaryl,
heterocycloalkyl,
C₁-C₆alkylheterocycloalkyl,
SO₂C₁-C₆alkyl, and
—N(R⁷)₂; and
m, n, and p are independently selected from the group consisting of 0, 1, 2 and 3; or
m, n or p is 2, and the two R, R⁵ or R⁶, respectively, together with the carbon to which they are attached, form a C₃-C₆cycloalkyl or form a nitrogen containing ring, wherein the C₃-C₆cycloalkyl or nitrogen containing ring is unsubstituted or substituted with —COphenylC₁-C₆alkyl-OH, or —COOC₁-C₆alkylphenyl,
wherein any of the above aryl, C₁-C₆alkylaryl, or heteroaryl, is unsubstituted or substituted with one to three substituents independently selected from the group consisting of halogen, C₁-C₆alkyl, oxo, —CON(R⁷)₂, C₁-C₆alkyl-OH, C₁-C₆haloalkyl, NHCO(C₁-C₆alkyl), C₁-C₆alkylN(R⁷)₂, C₁-C₆alkylheterocycloalkyl, heterocycloalkyl, and C₃-C₆cycloalkyl; wherein the heterocycloalkyl, C₃-C₆cycloalkyl, or C₁-C₆alkylN(R⁷)₂ is unsubstituted or substituted with one to three substituents independently selected from the group consisting of —CN, C₁-C₆alkyl, halogen, —OH, C₁-C₆haloalkyl, and —N(R⁷)₂; and R⁷ is hydrogen, or C₁-C₆alkyl.

Described herein are compounds of Formula I wherein R¹, R² and R³ are independently selected from the group consisting of hydrogen, halogen, —CN, —OH, C₁-C₆alkyl, —OC₁-C₆alkyl, and —OC₁-C₆haloalkyl. In certain embodiments, R¹, R² and R³ are independently selected from the group consisting of hydrogen, halogen, —CN, —OH, C₁-C₆alkyl, —OC₁-C₆alkyl, and —OC₁-C₆haloalkyl, wherein R¹, R² and R³ are not simultaneously hydrogen.

In certain embodiments, R¹ is selected from the group consisting of hydrogen, halogen, —CN, —OH, C₁-C₆alkyl, —OC₁-C₆alkyl, and —OC₁-C₆haloalkyl. In certain embodiments, R¹ is hydrogen. In certain embodiments, R¹ is halogen. Examples of suitable halogens include chlorine, bromine, fluorine and iodine. In certain embodiments, R¹ is fluorine. In certain embodiments, R¹ is chlorine. In certain embodiments, R¹ is —CN. In certain embodiments, R¹ is —OH. In certain embodiments, R¹ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R¹ is methyl. In certain embodiments, R¹ is —OC₁-C₆alkyl. Suitable alkoxys include, but are not limited to, methoxy, ethoxy, n-propoxy, isopropoxy and n-butoxy. In certain embodiments, R¹ is methoxy. In certain embodiments, R¹ is —OC₁-C₆haloalkyl. Suitable haloalkoxys include, but are not limited to, fluoromethoxy, fluoroethoxy, difluoromethoxy, difluoroethoxy, trifluoromethoxy and trifluoroethoxy. In certain embodiments, R¹ is trifluoromethoxy.

In certain embodiments, R² is selected from the group consisting of hydrogen, halogen, —CN, —OH, C₁-C₆alkyl, —OC₁-C₆alkyl, and —OC₁-C₆haloalkyl. In certain embodiments, R² is hydrogen. In certain embodiments, R² is halogen. Examples of suitable halogens include chlorine, bromine, fluorine and iodine. In certain embodiments, R² is fluorine. In certain embodiments, R² is chlorine. In certain embodiments, R² is —CN. In certain embodiments, R² is —OH. In certain embodiments, R² is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R² is methyl. In certain embodiments, R² is —OC₁-C₆alkyl. Suitable alkoxys include, but are not limited to, methoxy, ethoxy, n-propoxy, isopropoxy and n-butoxy. In certain embodiments, R² is methoxy. In certain embodiments, R² is —OC₁-C₆haloalkyl. Suitable haloalkoxys include, but are not limited to, fluoromethoxy, fluoroethoxy, difluoromethoxy, difluoroethoxy, trifluoromethoxy and trifluoroethoxy. In certain embodiments, R² is trifluoromethoxy.

In certain embodiments, R³ is selected from the group consisting of hydrogen, halogen, —CN, —OH, C₁-C₆alkyl, —OC₁-C₆alkyl, and —OC₁-C₆haloalkyl. In certain embodiments, R³ is hydrogen. In certain embodiments, R³ is halogen. Examples of suitable halogens include chlorine, bromine, fluorine and iodine. In certain embodiments, R³ is fluorine. In certain embodiments, R³ is chlorine. In certain embodiments, R³ is —CN. In certain embodiments, R³ is —OH. In certain embodiments, R³ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R³ is methyl. In certain embodiments, R³ is —OC₁-C₆alkyl. Suitable alkoxys include, but are not limited to, methoxy, ethoxy, n-propoxy, isopropoxy and n-butoxy. In certain embodiments, R³ is methoxy. In certain embodiments, R³ is —OC₁-C₆haloalkyl. Suitable haloalkoxys include, but are not limited to, fluoromethoxy, fluoroethoxy, difluoromethoxy, difluoroethoxy, trifluoromethoxy and trifluoroethoxy. In certain embodiments, R³ is trifluoromethoxy.

In certain embodiments R¹, R² and R³ are not simultaneously hydrogen. In certain embodiments R¹, R² and R³ are independently selected from the group consisting of hydrogen, halogen, and —OC₁-C₆alkyl. In certain embodiments R¹, R² and R³ are independently selected from the group consisting of hydrogen, halogen, and —OC₁-C₆alkyl, wherein R¹, R² and R³ are not simultaneously hydrogen.

In certain embodiments, R¹ is hydrogen, R² is methoxy and R³ is fluorine or hydrogen. In certain embodiments, R¹ is fluorine, R² is hydrogen and R³ is fluorine or hydrogen. In certain embodiments, R¹ is methoxy, R² is hydrogen and R³ is fluorine or hydrogen. In certain embodiments, R¹ is fluorine or chlorine, R² is hydrogen and R³ is fluorine.

Described herein are compounds wherein R⁷ is hydrogen, —CN, C₁-C₆alkyl, C₁-C₆alkylOH, C₁-C₆alkylCN, C₁-C₆alkylheterocycloalkyl, C₁-C₆alkylOC₁-C₆alkyl, —OC₁-C₆alkyl, C₁-C₆alkenyl, heterocycloalkyl, heteroaryl, aryl or C₃-C₆cycloalkyl, wherein the C₃-C₆cycloalkyl, aryl or heteroaryl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran. In certain embodiments, R⁷ is hydrogen. In certain embodiments, R⁷ is —CN. In certain embodiments, R⁷ is C₁-C₆alkyl. Examples of C₁-C₆alkyl groups include but are not limited to, methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1,2-dimethylpropyl, 1-ethylpropyl, n-hexyl, isohexyl, 1-methylpentyl, 2-methylpentyl, 3-methylpentyl, 1,1-dimethylbutyl, 1,2-dimethylbutyl, 2,2-dimethylbutyl, 1-ethylbutyl, 1,1,2-trimethylpropyl, 1,2,2-trimethylpropyl, 1-ethyl-2-methylpropyl and 1-ethyl-1-methylpropyl. In certain embodiments, R⁷ is haloC₁-C₆alkyl. Suitable examples of haloalkyls include, but are not limited to, fluoromethyl, difluoromethyl, trifluoromethyl, 2-fluoroethyl, 1,2-difluoroethyl and 2,2-difluoroethyl.

In certain embodiments, R⁷ is C₁-C₆alkylOH. Examples of suitable alcohols include, but are not limited to, methanol, ethanol, propanol, butanol and iso-butanol. In certain embodiments, R⁷ is C₁-C₆alkylCN. In certain embodiments, R⁷ is C₁-C₆alkylheterocycloalkyl. In certain embodiments, R⁷ is C₁-C₆alkylOC₁-C₆alkyl. In certain embodiments, R⁷ is —OC₁-C₆alkyl.

Suitable alkoxys include, but are not limited to, methoxy, ethoxy, n-propoxy, isopropoxy and n-butoxy.

In certain embodiments, R⁷ is C₁-C₆alkenyl. In certain embodiments, R⁷ is heterocycloalkyl. Non-limiting examples of heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. In certain embodiments, R⁷ is heteroaryl. In certain embodiments, R⁷ is aryl. Suitable examples of aryls include, but are not limited to, monocyclic aryl groups such as phenyl and bicyclic aryl groups such as naphthyl. In certain embodiments, R⁷ is C₃-C₆cycloalkyl. Suitable examples of cycloalkyls include, but are not limited to, cyclopropyl, cyclobutyl, cyclopentyl and cyclohexyl.

In another embodiment, the compounds of the invention comprise those compounds identified herein as examples, including the compounds below, and pharmaceutically acceptable salts thereof.

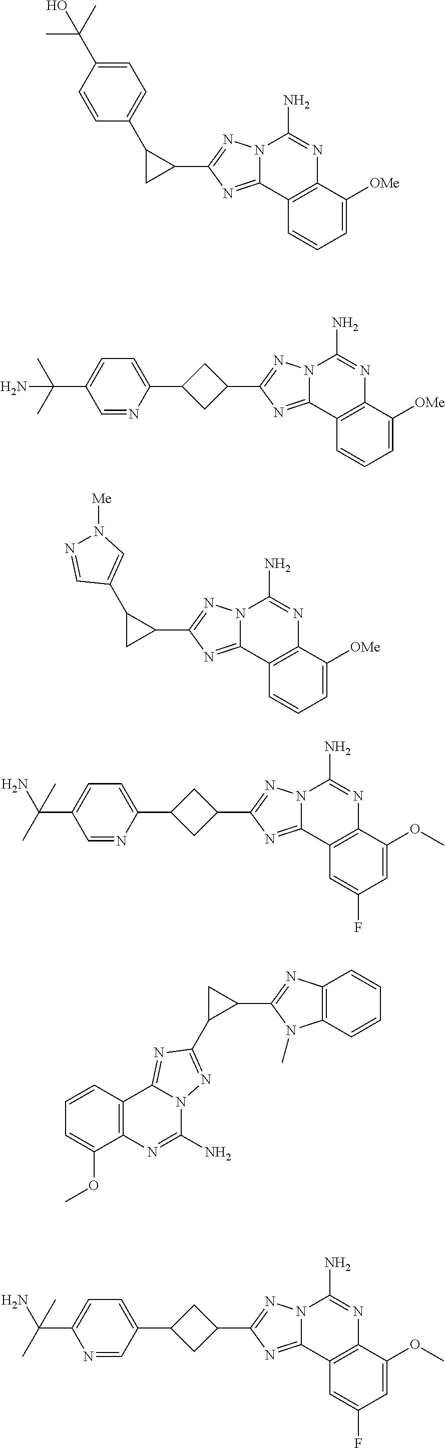

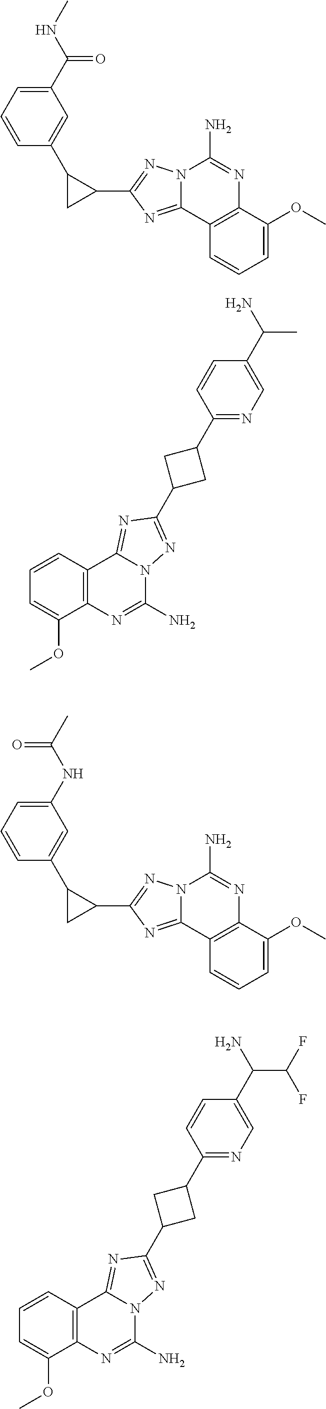

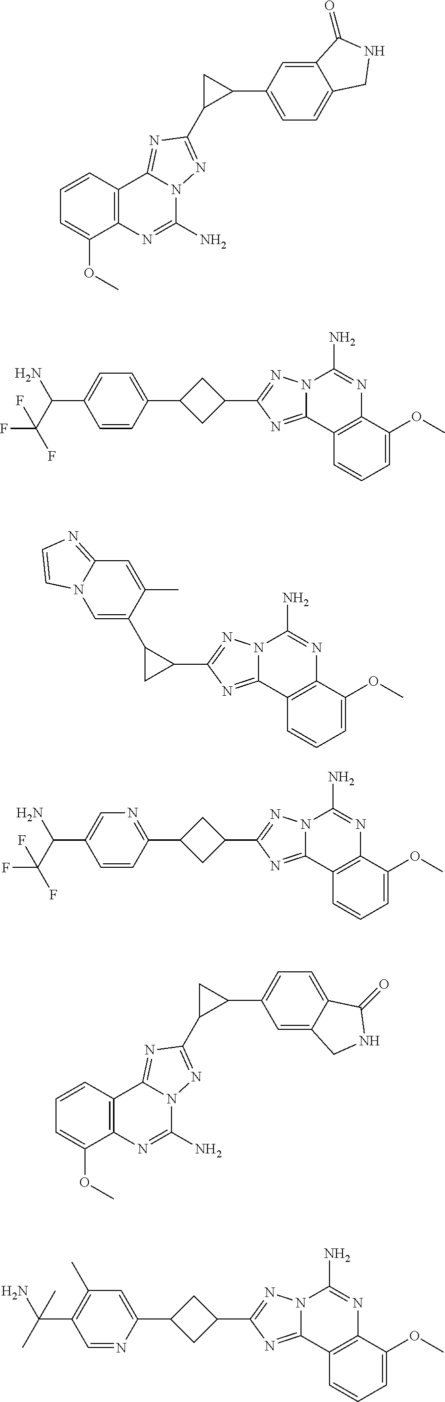

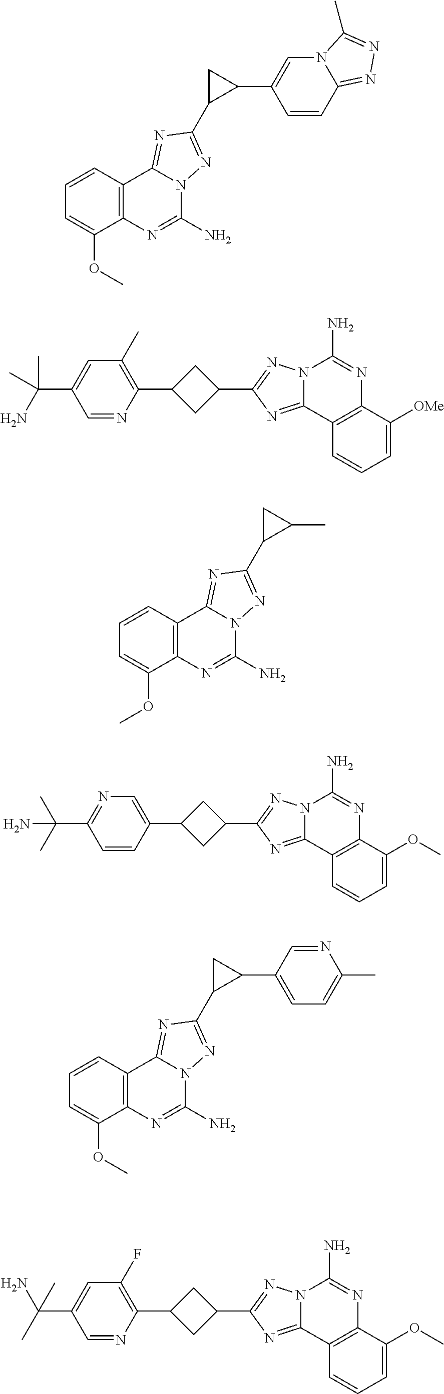

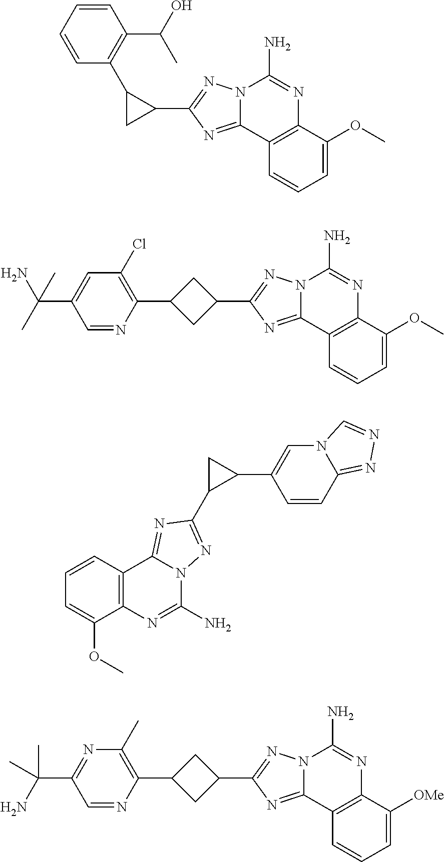

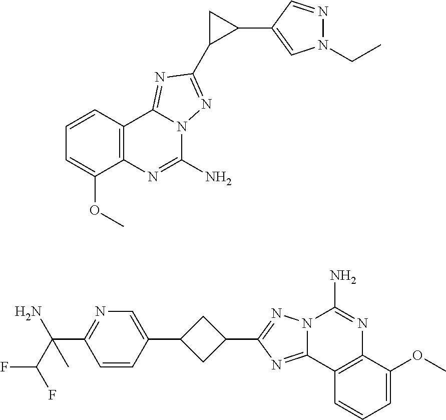 [SMILES: CCn1cc(C2CC2c2nc3c4cccc(OC)c4nc(N)n3n2)cn1 ; COc1cccc2c1nc(N)n1nc(C3CC(c4ccc(C(C)(N)C(F)F)nc4)C3)nc21]

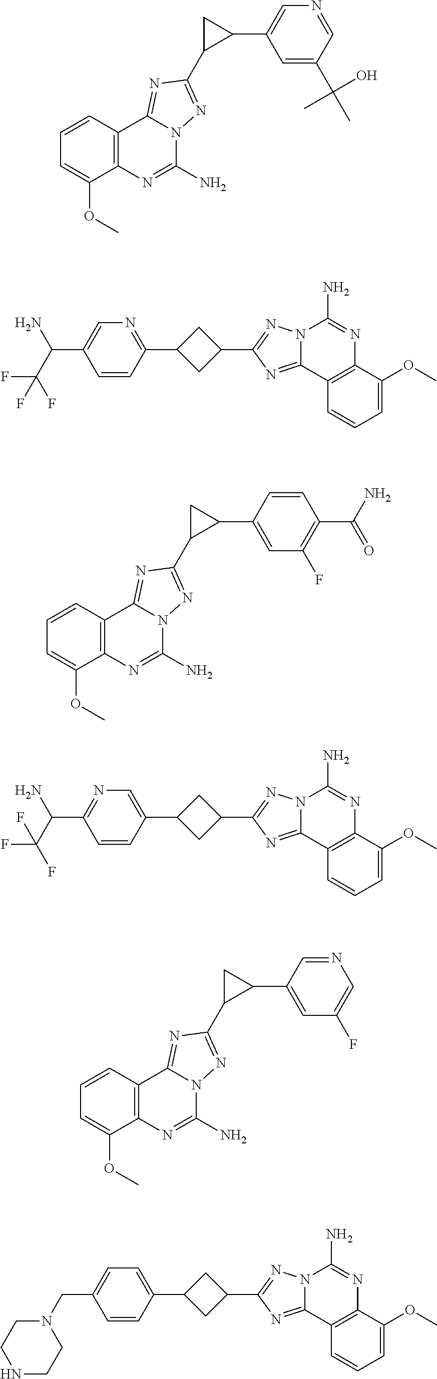 [SMILES: COc1cccc2c1nc(N)n1nc(C3CC3c3cncc(C(C)(C)O)c3)nc21 ; COc1cccc2c1nc(N)n1nc(C3CC(c4ccc(C(N)C(F)(F)F)cn4)C3)nc21 ; COc1cccc2c1nc(N)n1nc(C3CC3c3ccc(C(N)=O)c(F)c3)nc21 ; COc1cccc2c1nc(N)n1nc(C3CC(c4ccc(C(N)C(F)(F)F)nc4)C3)nc21 ; COc1cccc2c1nc(N)n1nc(C3CC3c3cncc(F)c3)nc21 ; COc1cccc2c1nc(N)n1nc(C3CC(c4ccc(CN5CCNCC5)cc4)C3)nc21]

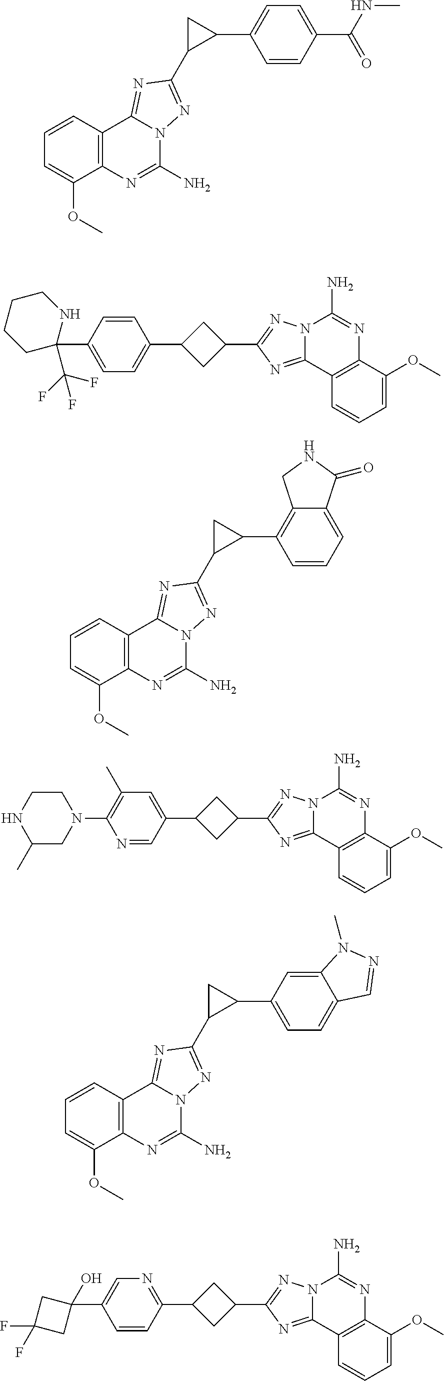

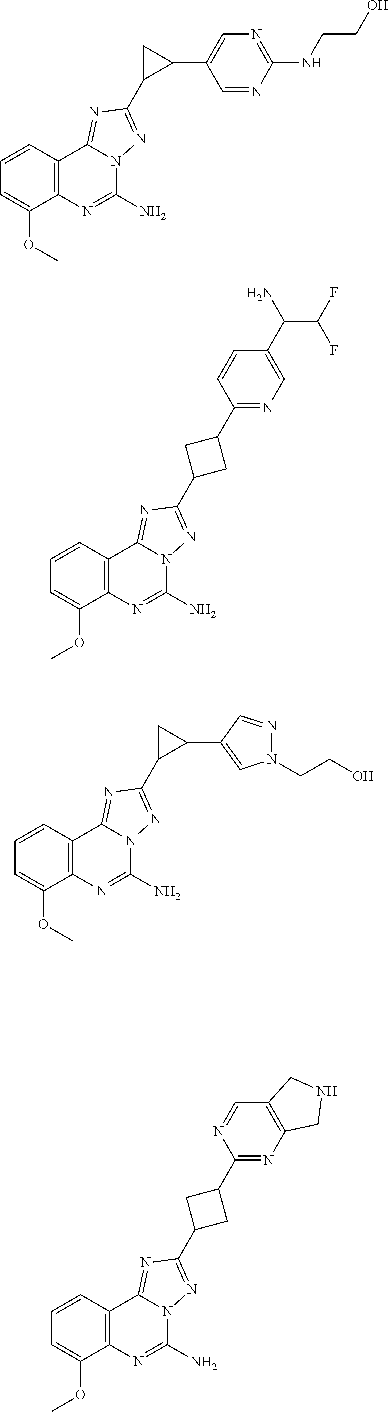 [SMILES: COc1cccc2c1nc(N)n1nc(C3CC3c3cnc(NCCO)nc3)nc21 ; COc1cccc2c1nc(N)n1nc(C3CC(c4ccc(C(N)C(F)F)cn4)C3)nc21 ; COc1cccc2c1nc(N)n1nc(C3CC3c3cnn(CCO)c3)nc21 ; COc1cccc2c1nc(N)n1nc(C3CC(c4ncc5c(n4)CNC5)C3)nc21]

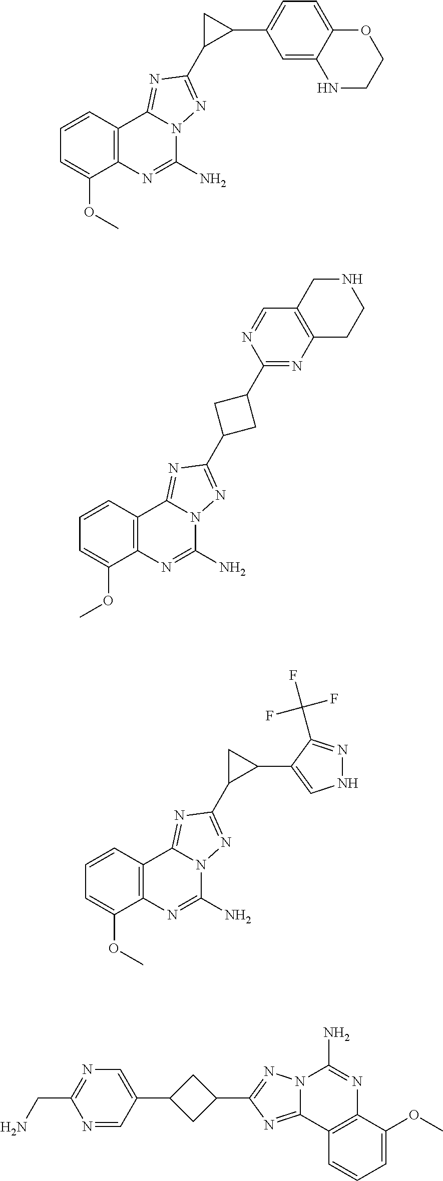 [SMILES: COc1cccc2c1nc(N)n1nc(C3CC3c3ccc4c(c3)NCCO4)nc21 ; COc1cccc2c1nc(N)n1nc(C3CC(c4ncc5c(n4)CCNC5)C3)nc21 ; COc1cccc2c1nc(N)n1nc(C3CC3c3c[nH]nc3C(F)(F)F)nc21 ; COc1cccc2c1nc(N)n1nc(C3CC(c4cnc(CN)nc4)C3)nc21]

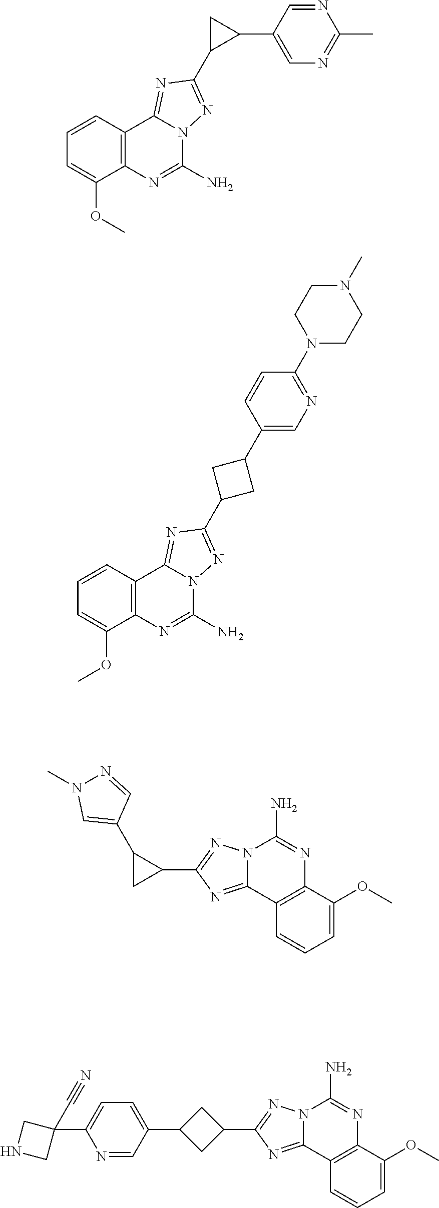

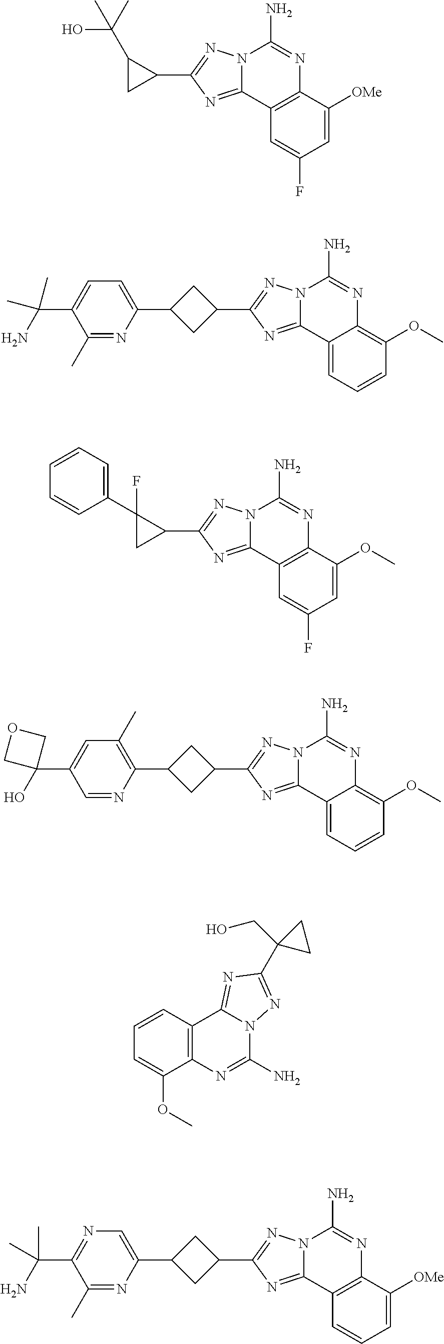

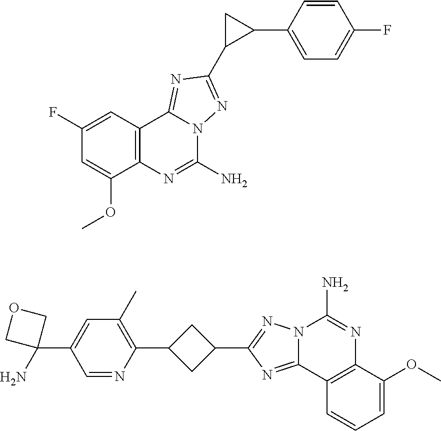 [SMILES: COc1cc(F)cc2c1nc(N)n1nc(C3CC3c3ccc(F)cc3)nc21 ; COc1cccc2c1nc(N)n1nc(C3CC(c4ncc(C5(N)COC5)cc4C)C3)nc21]

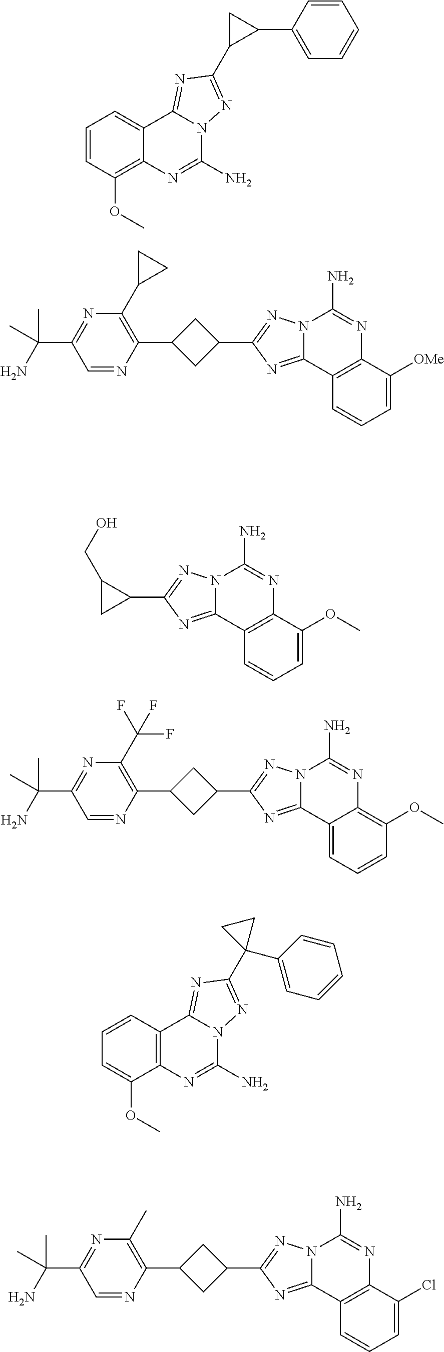

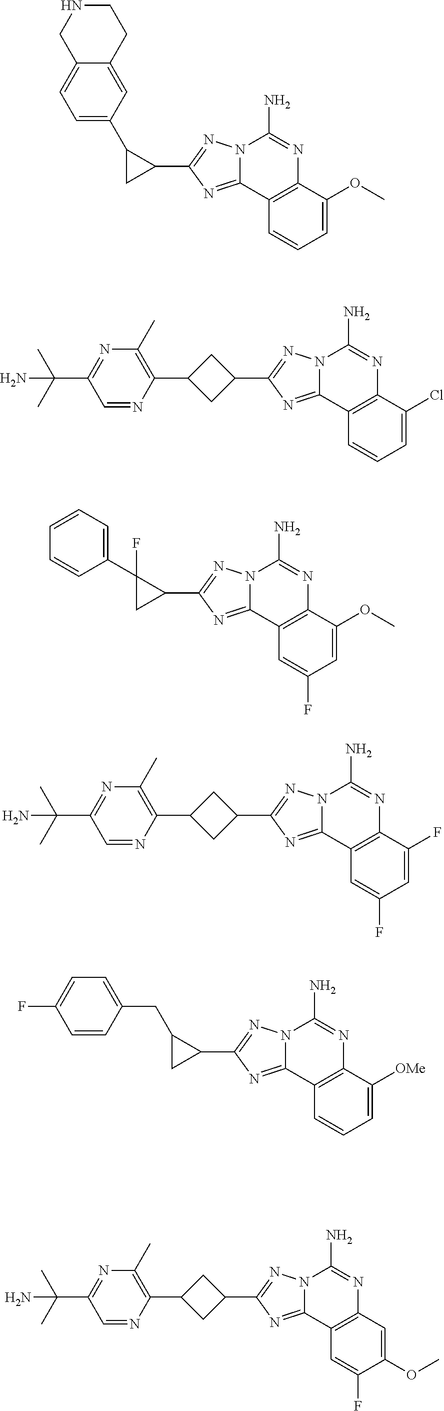 [SMILES: COc1cccc2c1nc(N)n1nc(C3CC3c3ccc4c(c3)CCNC4)nc21 ; Cc1nc(C(C)(C)N)cnc1C1CC(c2nc3c4cccc(Cl)c4nc(N)n3n2)C1 ; COc1cc(F)cc2c1nc(N)n1nc(C3CC3(F)c3ccccc3)nc21 ; Cc1nc(C(C)(C)N)cnc1C1CC(c2nc3c4cc(F)cc(F)c4nc(N)n3n2)C1 ; COc1cccc2c1nc(N)n1nc(C3CC3Cc3ccc(F)cc3)nc21 ; COc1cc2nc(N)n3nc(C4CC(c5ncc(C(C)(C)N)nc5C)C4)nc3c2cc1F]

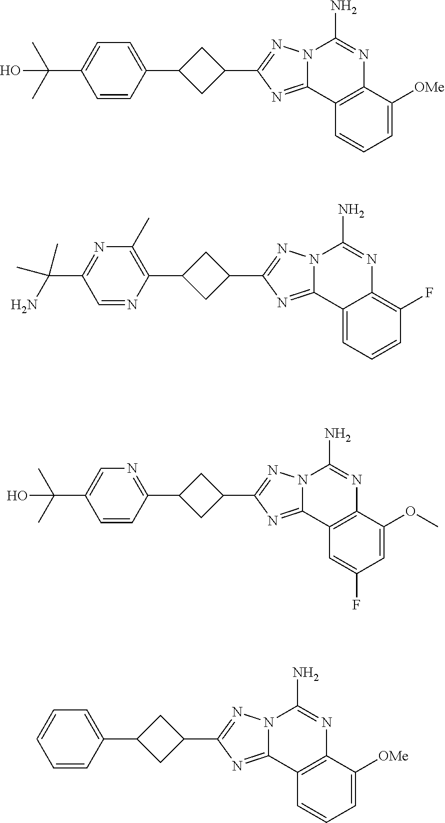 [SMILES: COc1cccc2c1nc(N)n1nc(C3CC(c4ccc(C(C)(C)O)cc4)C3)nc21 ; Cc1nc(C(C)(C)N)cnc1C1CC(c2nc3c4cccc(F)c4nc(N)n3n2)C1 ; COc1cc(F)cc2c1nc(N)n1nc(C3CC(c4ccc(C(C)(C)O)cn4)C3)nc21 ; COc1cccc2c1nc(N)n1nc(C3CC(c4ccccc4)C3)nc21]

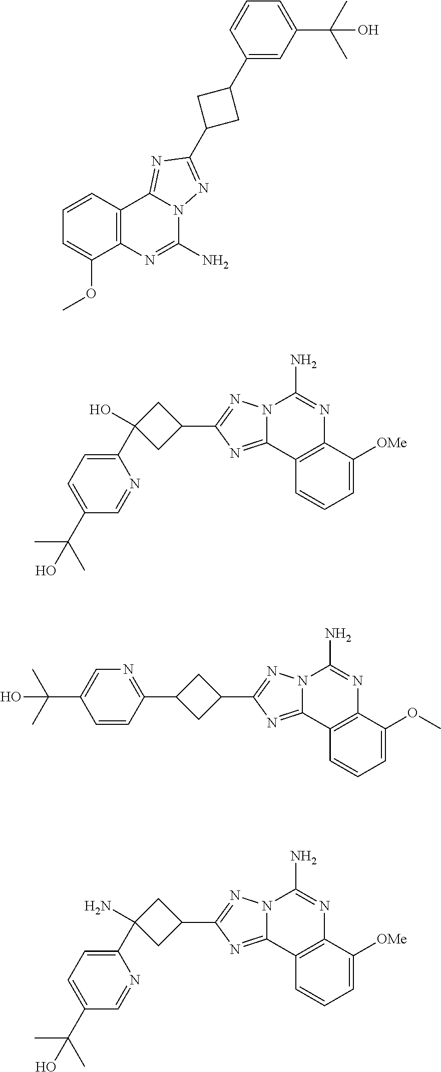

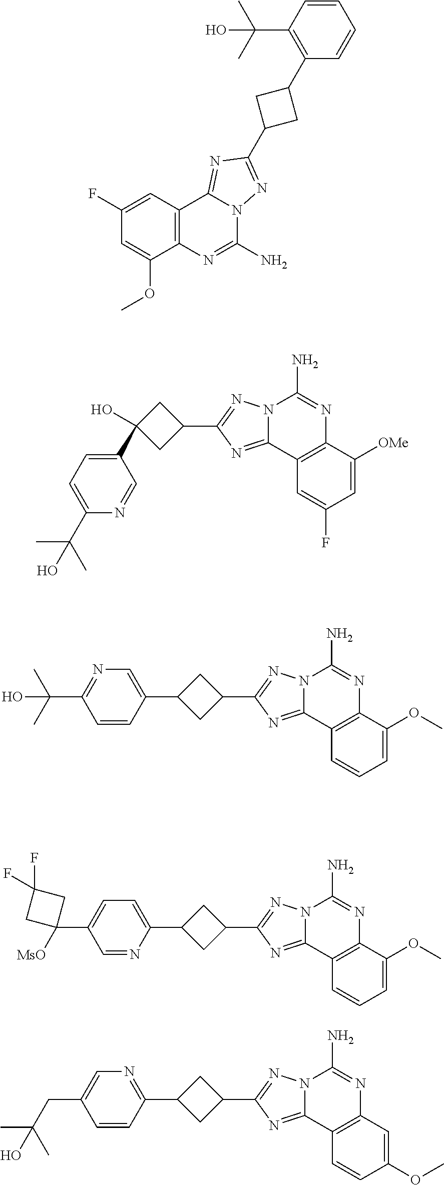

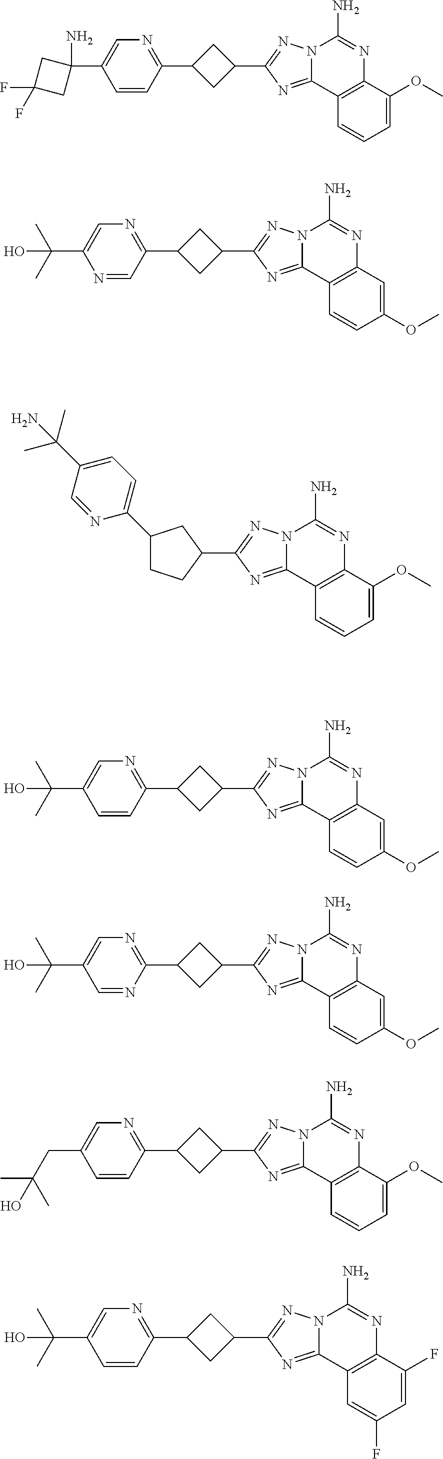

Incertain embodiments, described herein are the compounds below, and pharmaceutically acceptable salts thereof.

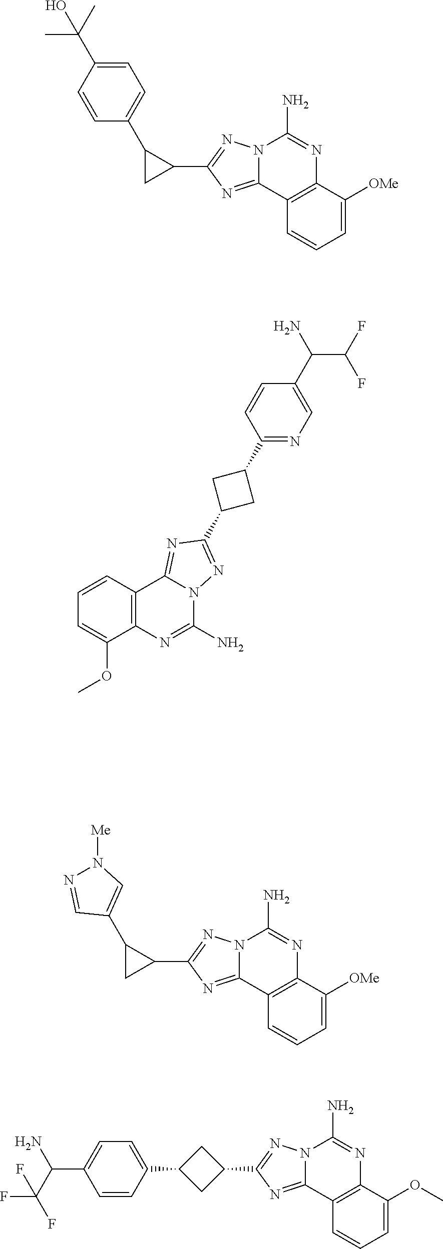 [SMILES: COc1cccc2c1nc(N)n1nc(C3CC3c3ccc(C(C)(C)O)cc3)nc21 ; COc1cccc2c1nc(N)n1nc([C@H]3C[C@@H](c4ccc(C(N)C(F)F)cn4)C3)nc21 ; COc1cccc2c1nc(N)n1nc(C3CC3c3cnn(C)c3)nc21 ; COc1cccc2c1nc(N)n1nc([C@H]3C[C@@H](c4ccc(C(N)C(F)(F)F)cc4)C3)nc21]

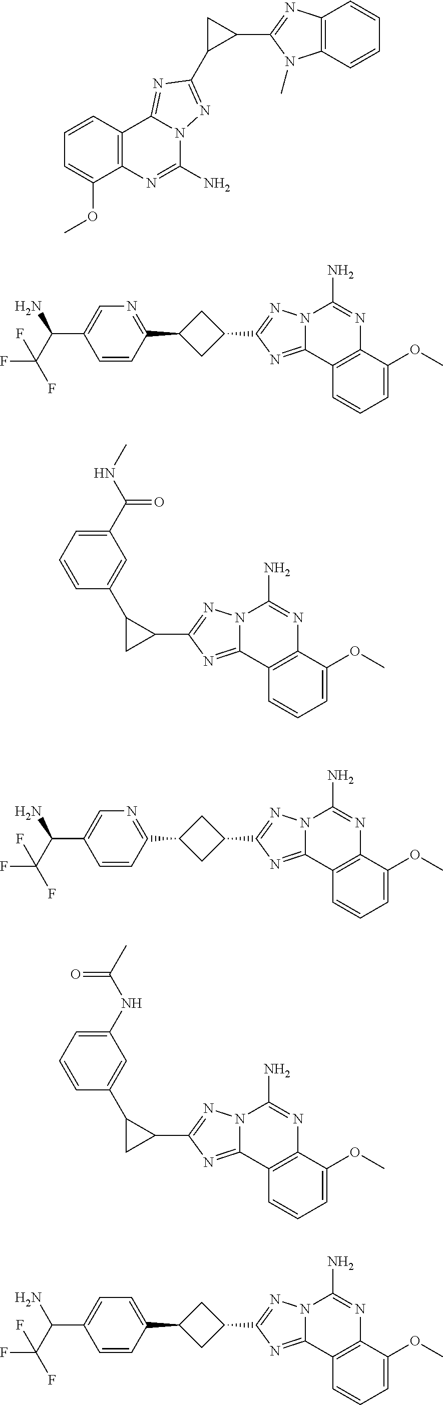 [SMILES: COc1cccc2c1nc(N)n1nc(C3CC3c3nc4ccccc4n3C)nc21 ; COc1cccc2c1nc(N)n1nc([C@H]3C[C@H](c4ccc([C@H](N)C(F)(F)F)cn4)C3)nc21 ; CNC(=O)c1cccc(C2CC2c2nc3c4cccc(OC)c4nc(N)n3n2)c1 ; COc1cccc2c1nc(N)n1nc([C@H]3C[C@@H](c4ccc([C@H](N)C(F)(F)F)cn4)C3)nc21 ; COc1cccc2c1nc(N)n1nc(C3CC3c3cccc(NC(C)=O)c3)nc21 ; COc1cccc2c1nc(N)n1nc([C@H]3C[C@H](c4ccc(C(N)C(F)(F)F)cc4)C3)nc21]

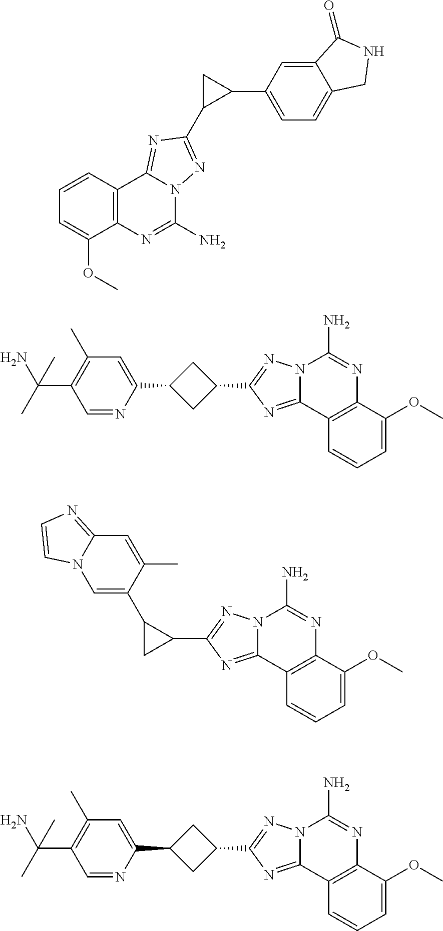

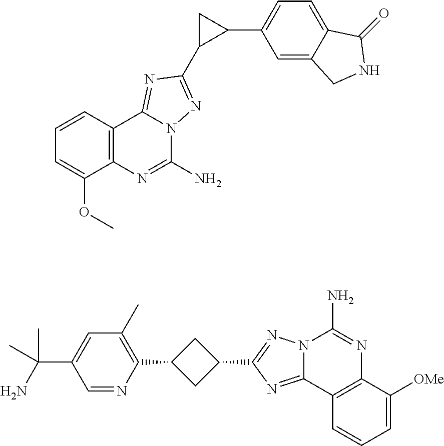 [SMILES: COc1cccc2c1nc(N)n1nc(C3CC3c3ccc4c(c3)CNC4=O)nc21 ; COc1cccc2c1nc(N)n1nc([C@H]3C[C@@H](c4ncc(C(C)(C)N)cc4C)C3)nc21]

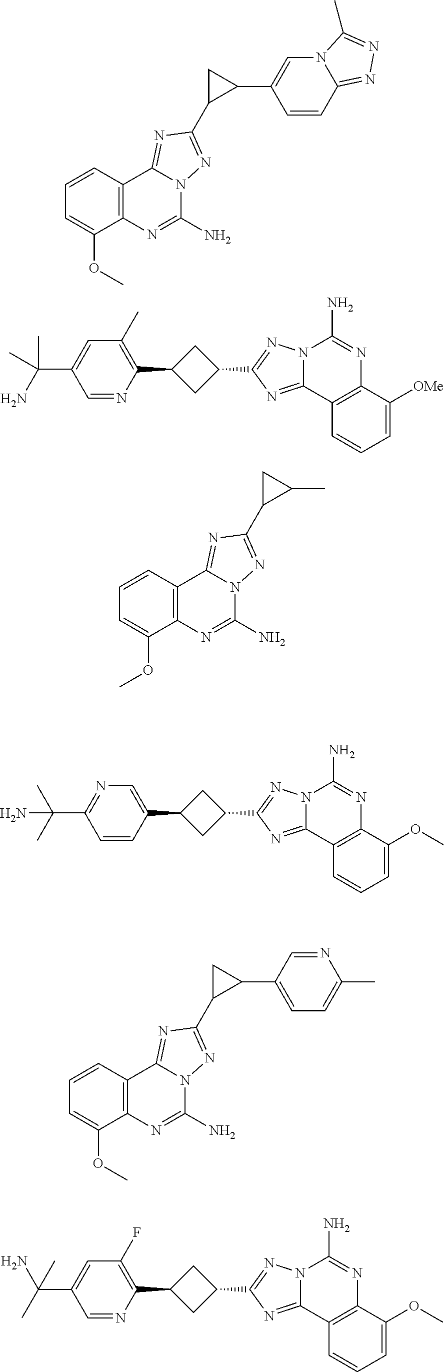

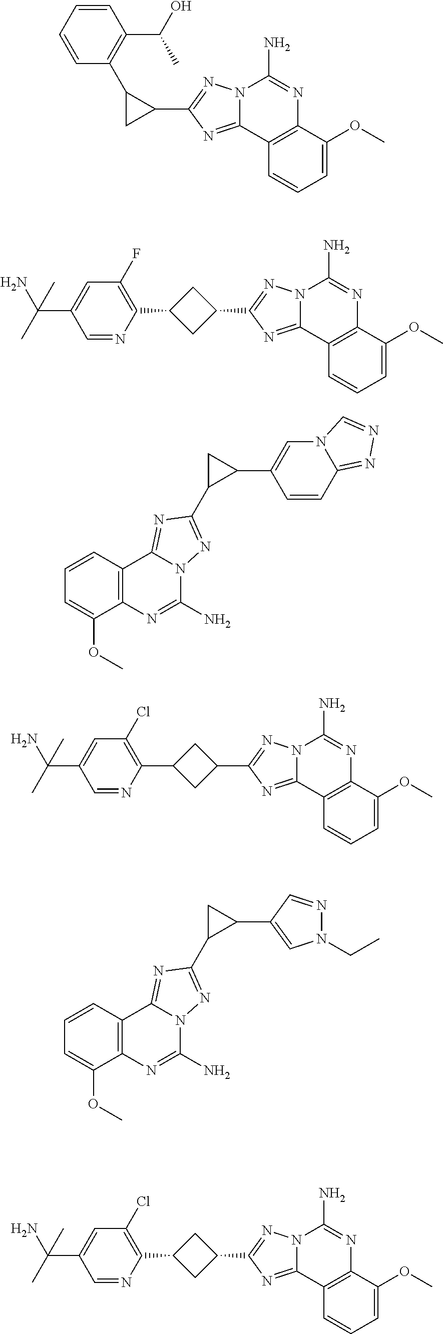

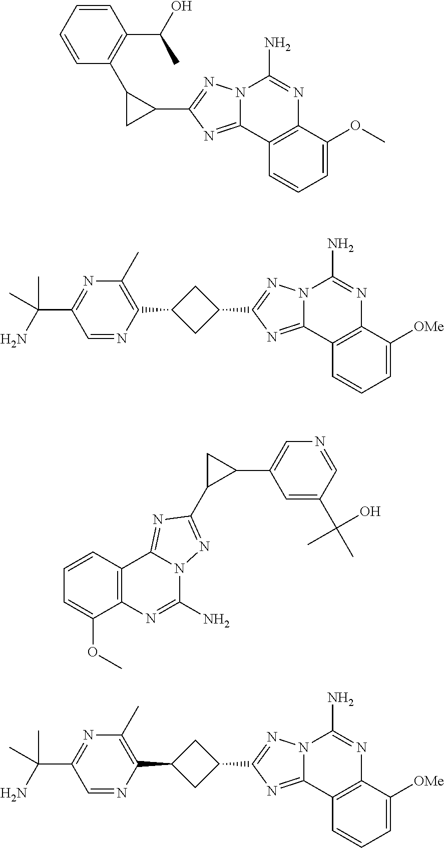

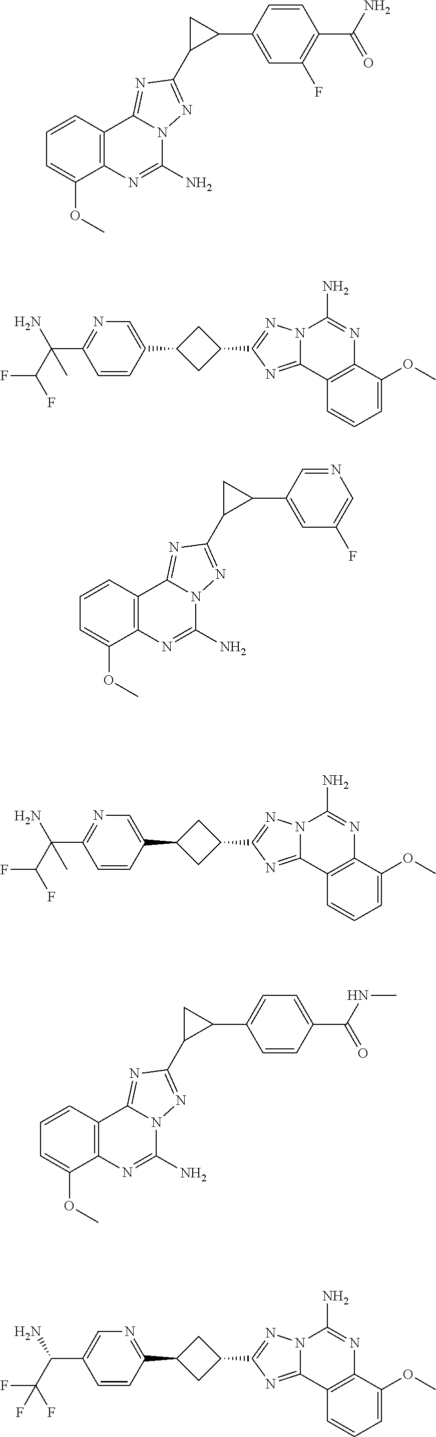 [SMILES: COc1cccc2c1nc(N)n1nc(C3CC3c3ccc(C(N)=O)c(F)c3)nc21 ; COc1cccc2c1nc(N)n1nc([C@H]3C[C@@H](c4ccc(C(C)(N)C(F)F)nc4)C3)nc21 ; COc1cccc2c1nc(N)n1nc(C3CC3c3cncc(F)c3)nc21 ; COc1cccc2c1nc(N)n1nc([C@H]3C[C@H](c4ccc(C(C)(N)C(F)F)nc4)C3)nc21 ; CNC(=O)c1ccc(C2CC2c2nc3c4cccc(OC)c4nc(N)n3n2)cc1 ; COc1cccc2c1nc(N)n1nc([C@H]3C[C@H](c4ccc([C@@H](N)C(F)(F)F)cn4)C3)nc21]

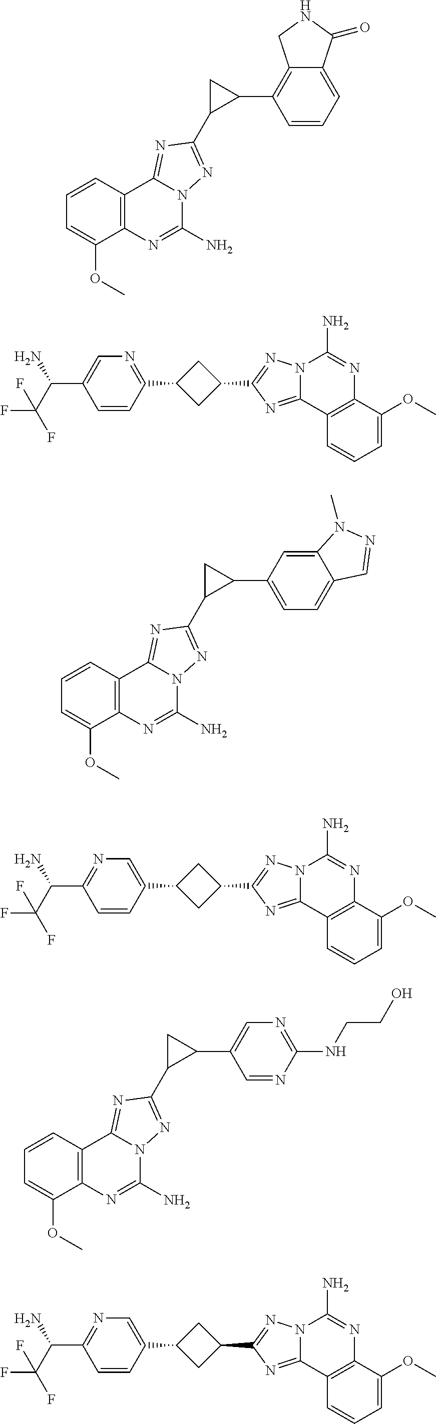

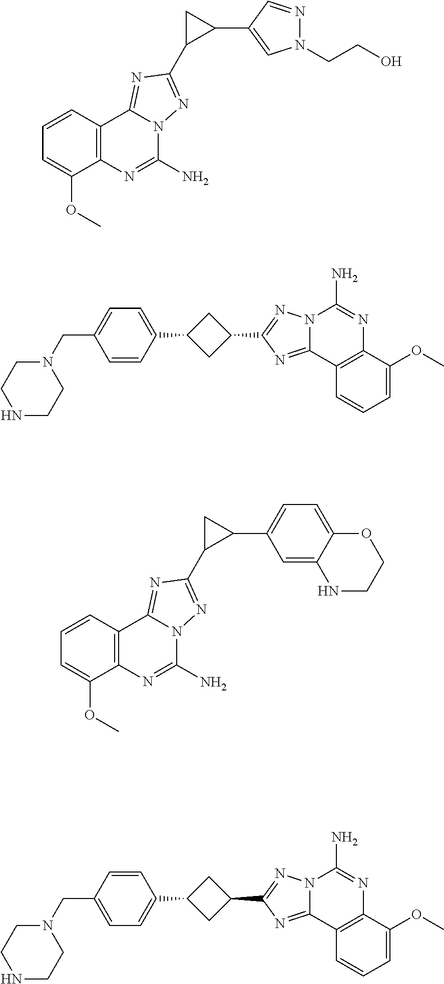 [SMILES: COc1cccc2c1nc(N)n1nc(C3CC3c3cnn(CCO)c3)nc21 ; COc1cccc2c1nc(N)n1nc([C@H]3C[C@@H](c4ccc(CN5CCNCC5)cc4)C3)nc21 ; COc1cccc2c1nc(N)n1nc(C3CC3c3ccc4c(c3)NCCO4)nc21 ; COc1cccc2c1nc(N)n1nc([C@H]3C[C@H](c4ccc(CN5CCNCC5)cc4)C3)nc21]

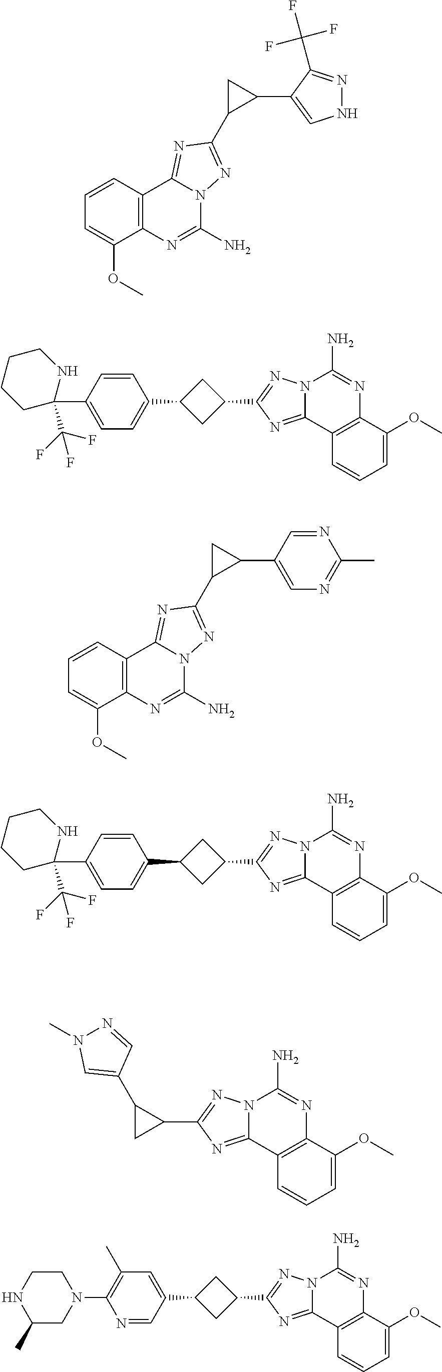

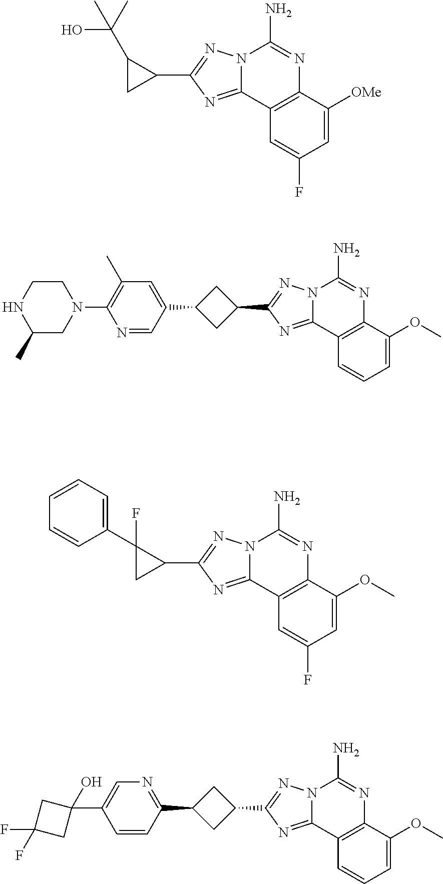 [SMILES: COc1cc(F)cc2c1nc(N)n1nc(C3CC3C(C)(C)O)nc21 ; COc1cccc2c1nc(N)n1nc([C@H]3C[C@H](c4cnc(N5CCN[C@H](C)C5)c(C)c4)C3)nc21 ; COc1cc(F)cc2c1nc(N)n1nc(C3CC3(F)c3ccccc3)nc21 ; COc1cccc2c1nc(N)n1nc([C@H]3C[C@H](c4ccc(C5(O)CC(F)(F)C5)cn4)C3)nc21]

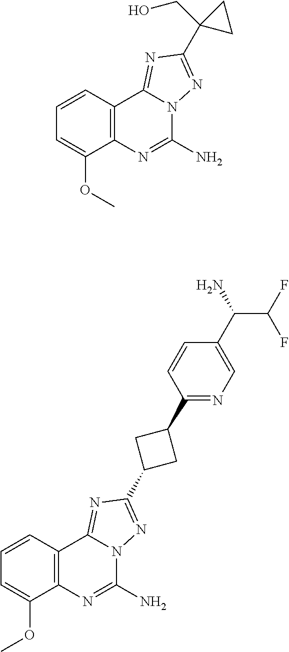 [SMILES: COc1cccc2c1nc(N)n1nc(C3(CO)CC3)nc21 ; COc1cccc2c1nc(N)n1nc([C@H]3C[C@H](c4ccc([C@H](N)C(F)F)cn4)C3)nc21]

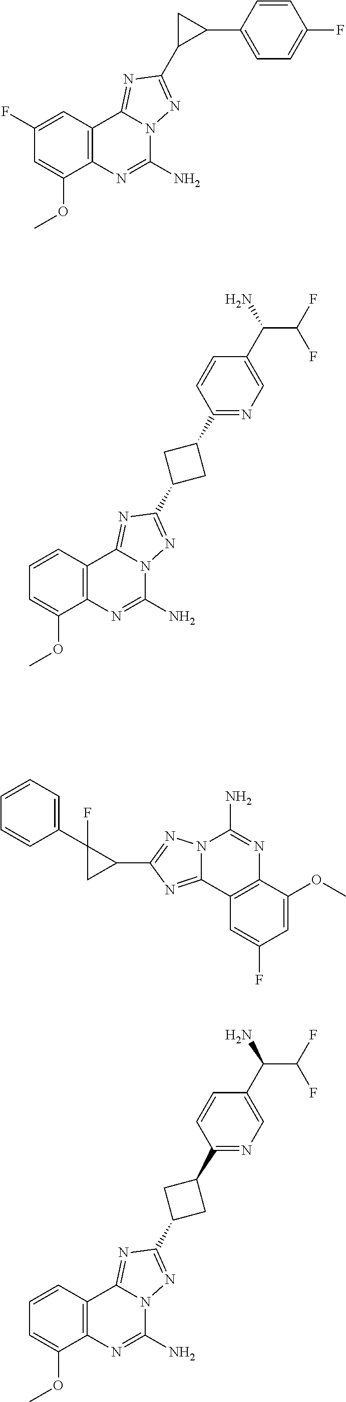 [SMILES: COc1cc(F)cc2c1nc(N)n1nc(C3CC3c3ccc(F)cc3)nc21 ; COc1cccc2c1nc(N)n1nc([C@H]3C[C@@H](c4ccc([C@H](N)C(F)F)cn4)C3)nc21 ; COc1cc(F)cc2c1nc(N)n1nc(C3CC3(F)c3ccccc3)nc21 ; COc1cccc2c1nc(N)n1nc([C@H]3C[C@H](c4ccc([C@@H](N)C(F)F)cn4)C3)nc21]

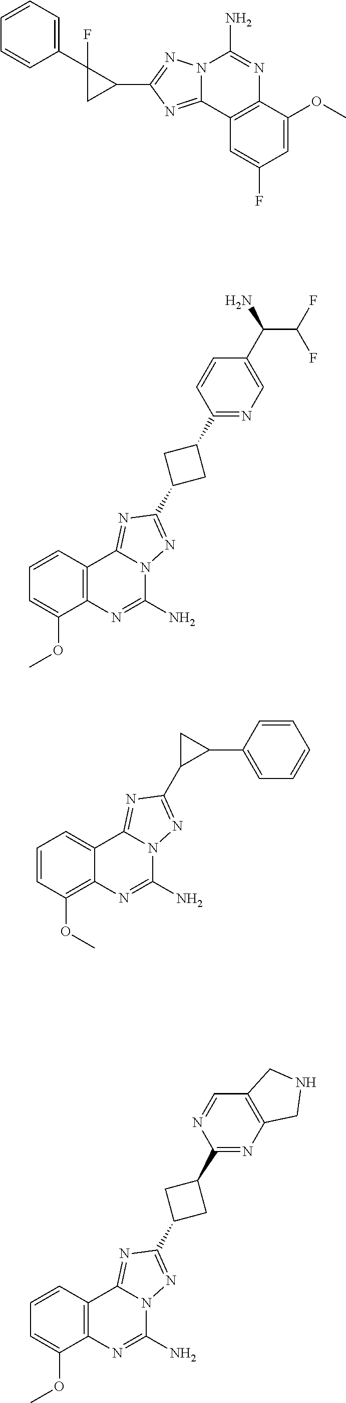 [SMILES: COc1cc(F)cc2c1nc(N)n1nc(C3CC3(F)c3ccccc3)nc21 ; COc1cccc2c1nc(N)n1nc([C@H]3C[C@@H](c4ccc([C@@H](N)C(F)F)cn4)C3)nc21 ; COc1cccc2c1nc(N)n1nc(C3CC3c3ccccc3)nc21 ; COc1cccc2c1nc(N)n1nc([C@H]3C[C@H](c4ncc5c(n4)CNC5)C3)nc21]

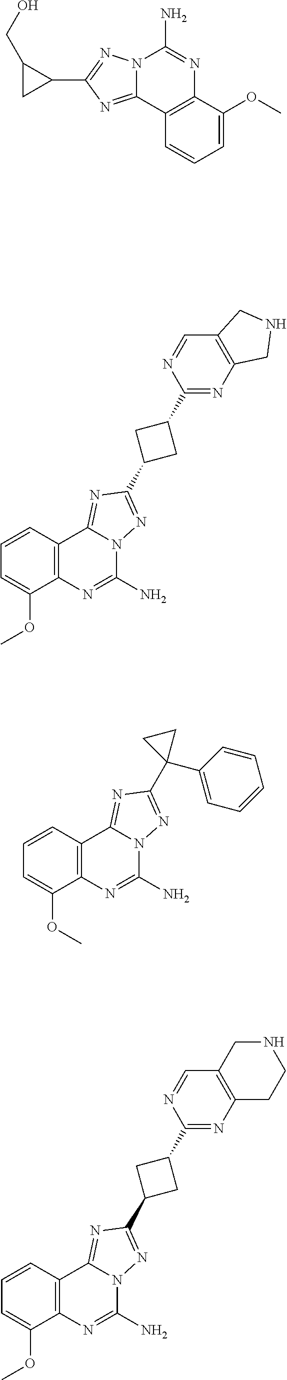 [SMILES: COc1cccc2c1nc(N)n1nc(C3CC3CO)nc21 ; COc1cccc2c1nc(N)n1nc([C@H]3C[C@@H](c4ncc5c(n4)CNC5)C3)nc21 ; COc1cccc2c1nc(N)n1nc(C3(c4ccccc4)CC3)nc21 ; COc1cccc2c1nc(N)n1nc([C@H]3C[C@H](c4ncc5c(n4)CCNC5)C3)nc21]

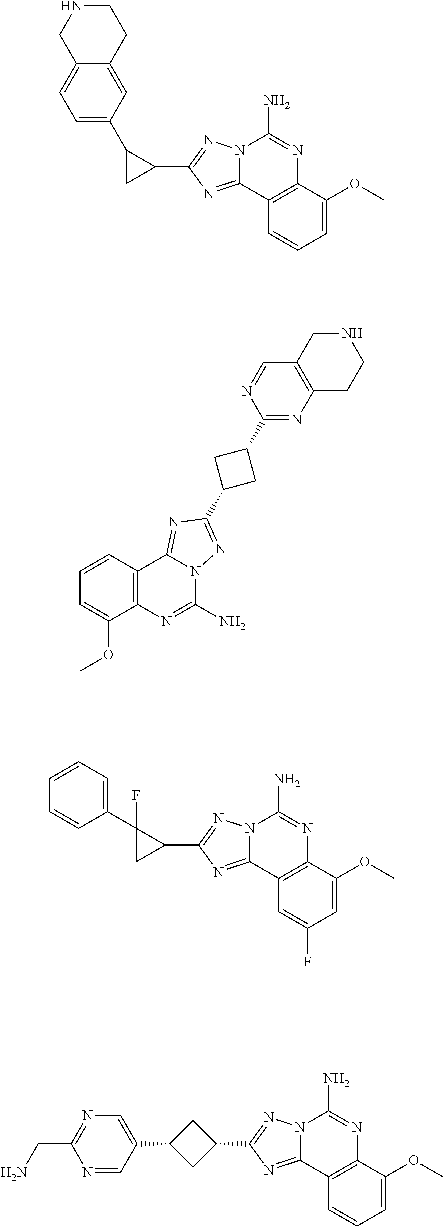 [SMILES: COc1cccc2c1nc(N)n1nc(C3CC3c3ccc4c(c3)CCNC4)nc21 ; COc1cccc2c1nc(N)n1nc([C@H]3C[C@@H](c4ncc5c(n4)CCNC5)C3)nc21 ; COc1cc(F)cc2c1nc(N)n1nc(C3CC3(F)c3ccccc3)nc21 ; COc1cccc2c1nc(N)n1nc([C@H]3C[C@@H](c4cnc(CN)nc4)C3)nc21]

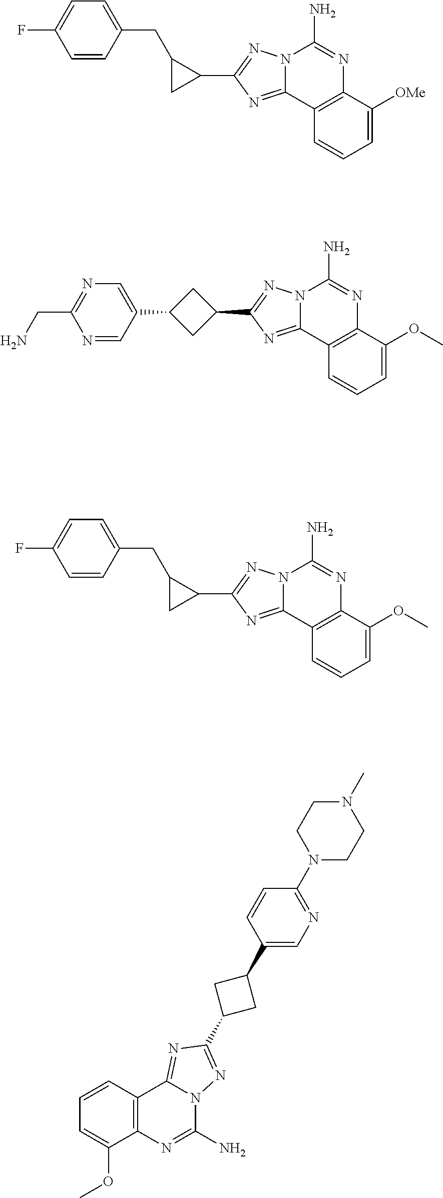 [SMILES: COc1cccc2c1nc(N)n1nc(C3CC3Cc3ccc(F)cc3)nc21 ; COc1cccc2c1nc(N)n1nc([C@H]3C[C@H](c4cnc(CN)nc4)C3)nc21 ; COc1cccc2c1nc(N)n1nc(C3CC3Cc3ccc(F)cc3)nc21 ; COc1cccc2c1nc(N)n1nc([C@H]3C[C@H](c4ccc(N5CCN(C)CC5)nc4)C3)nc21]

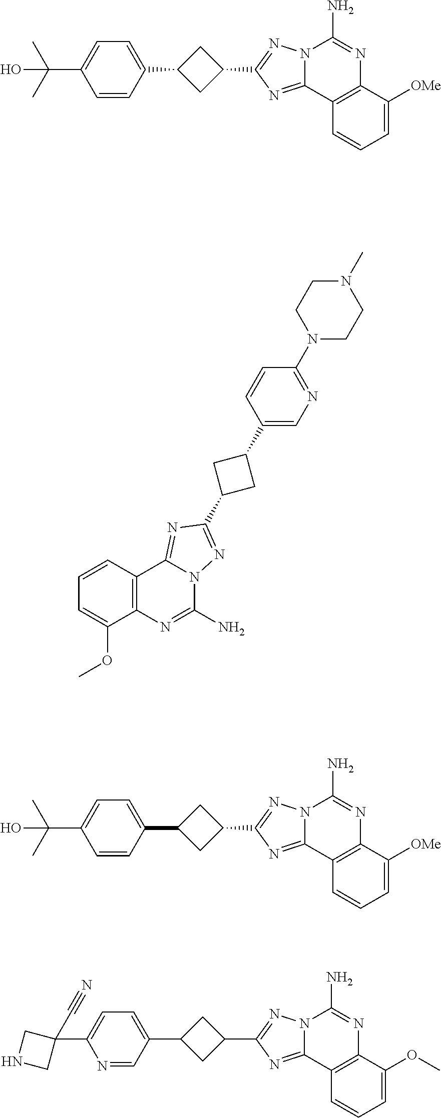 [SMILES: COc1cccc2c1nc(N)n1nc([C@H]3C[C@@H](c4ccc(C(C)(C)O)cc4)C3)nc21 ; COc1cccc2c1nc(N)n1nc([C@H]3C[C@@H](c4ccc(N5CCN(C)CC5)nc4)C3)nc21 ; COc1cccc2c1nc(N)n1nc([C@H]3C[C@H](c4ccc(C(C)(C)O)cc4)C3)nc21 ; COc1cccc2c1nc(N)n1nc(C3CC(c4ccc(C5(C#N)CNC5)nc4)C3)nc21]

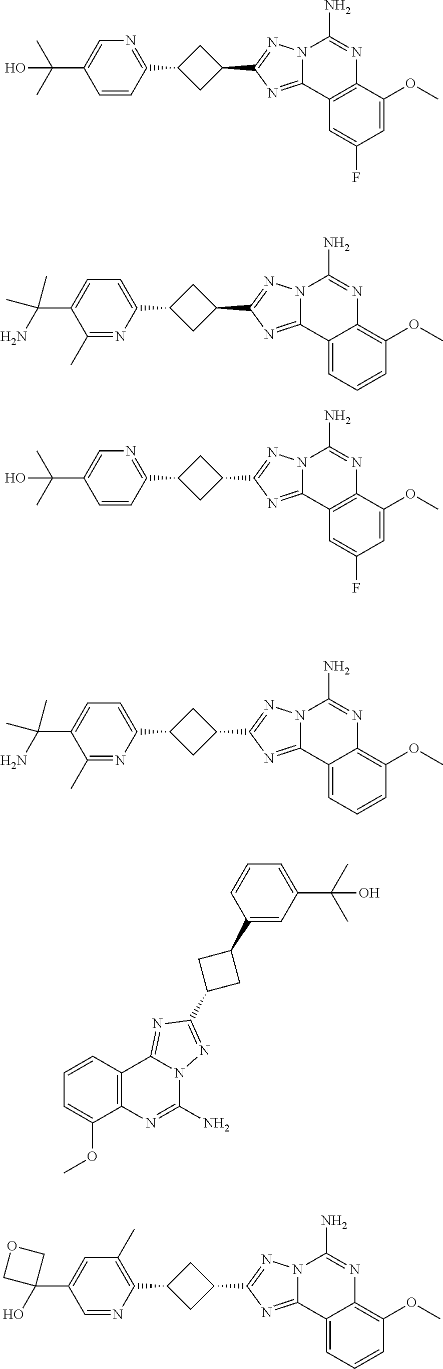

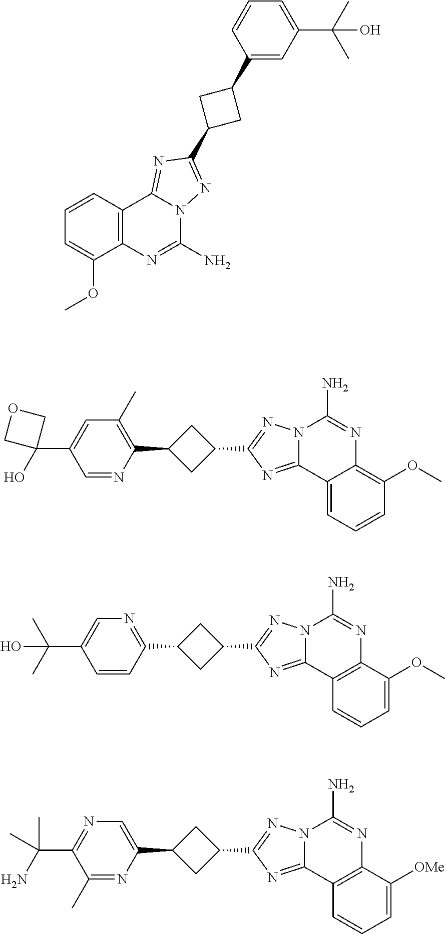

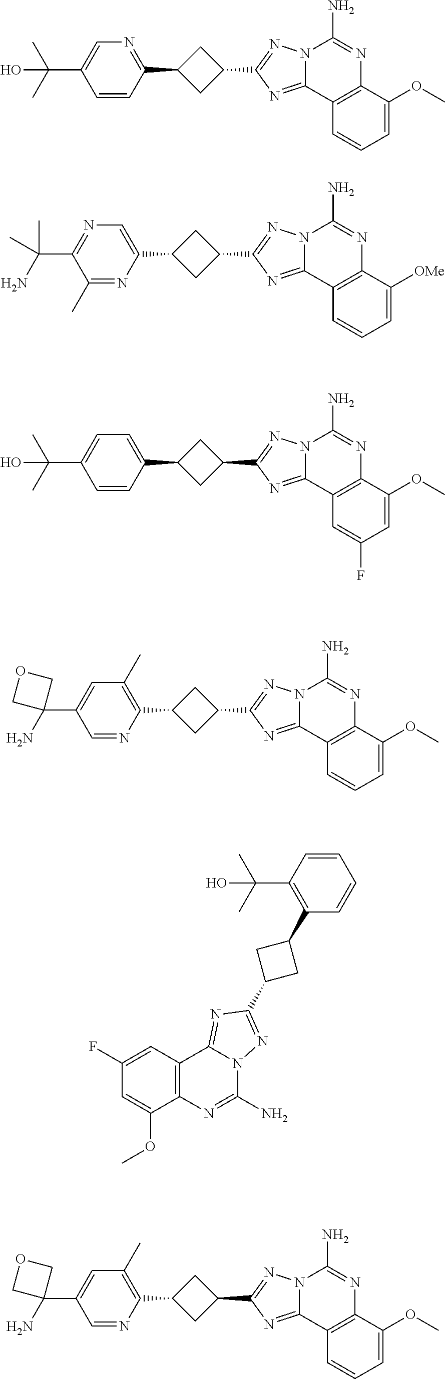

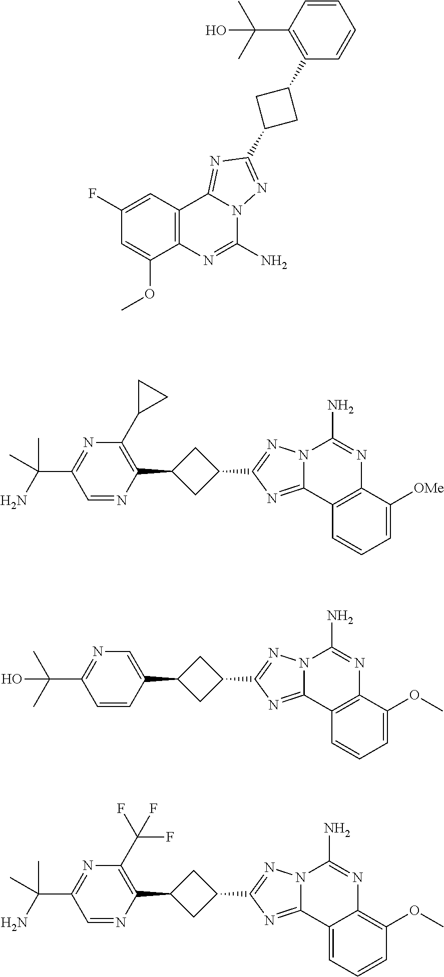

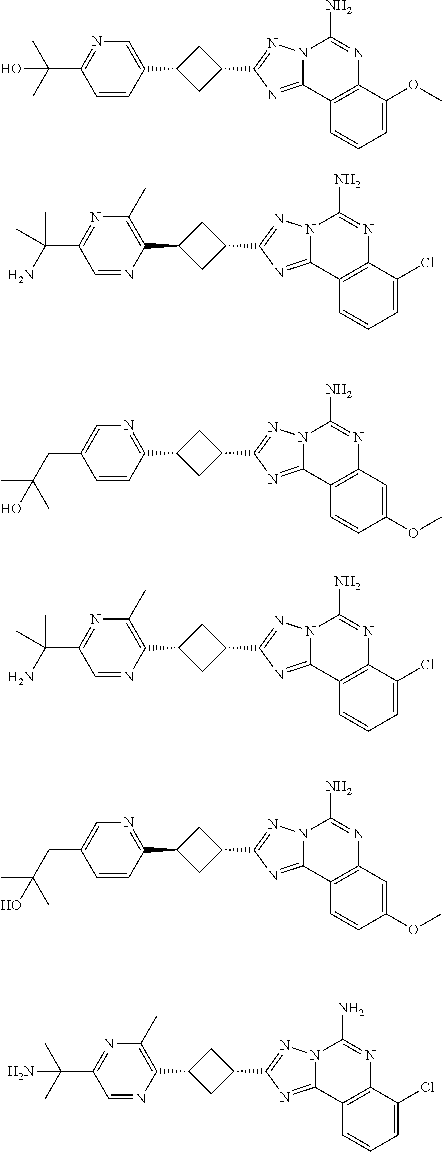

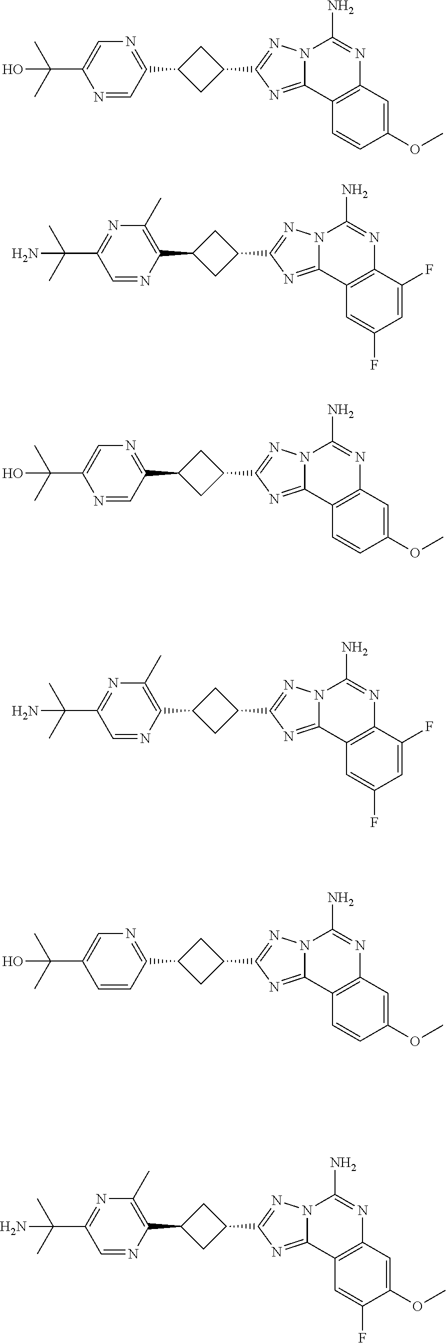

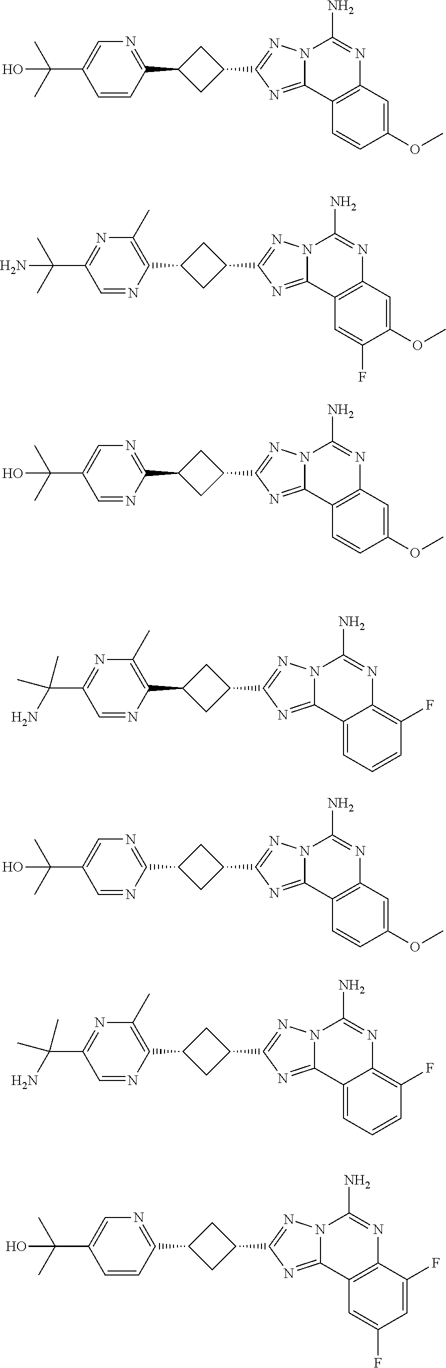

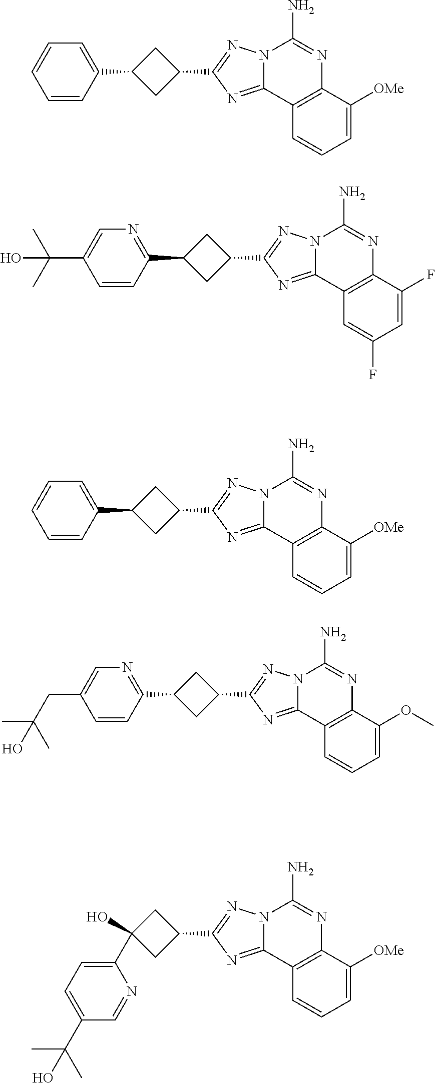

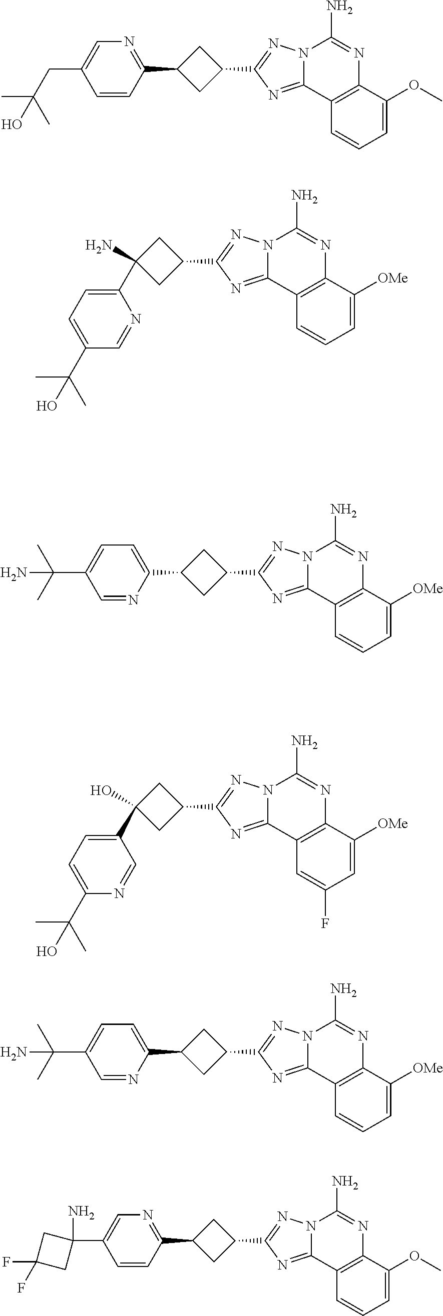

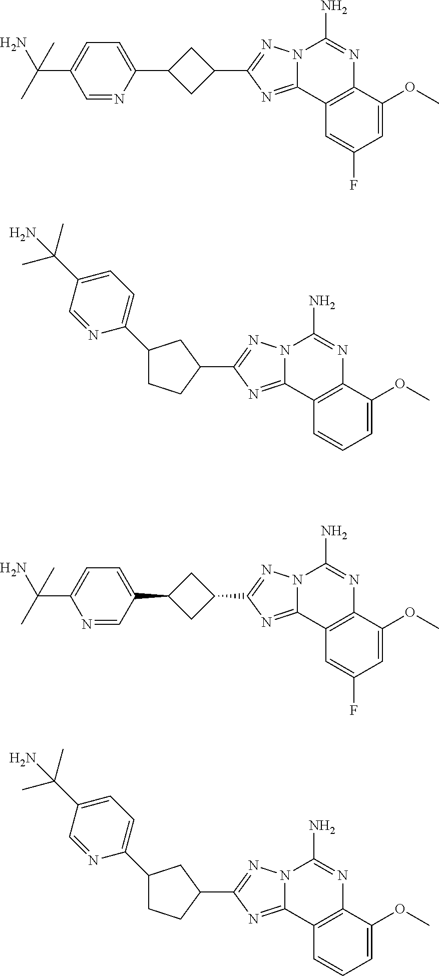

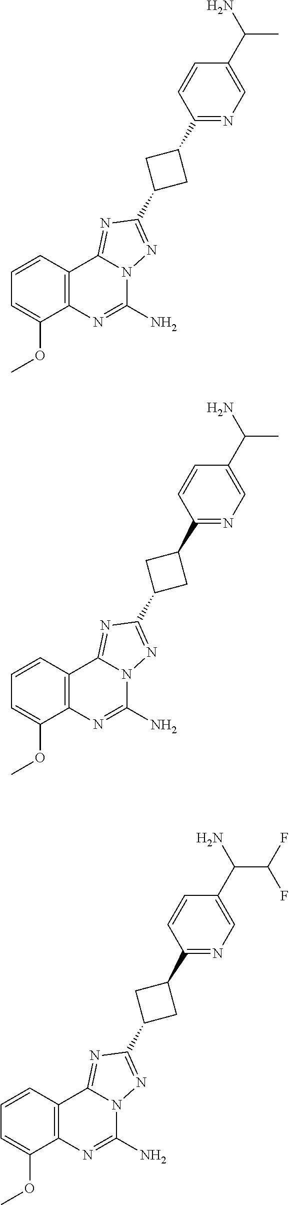 [SMILES: COc1cccc2c1nc(N)n1nc([C@H]3C[C@@H](c4ccc(C(C)N)cn4)C3)nc21 ; COc1cccc2c1nc(N)n1nc([C@H]3C[C@H](c4ccc(C(C)N)cn4)C3)nc21 ; COc1cccc2c1nc(N)n1nc([C@H]3C[C@H](c4ccc(C(N)C(F)F)cn4)C3)nc21]

### Oncology

In some embodiments, the disease, condition or disorder is a cancer. Any cancer for which a PD-1 antagonist and/or an A2a and/or A2b inhibitor are thought to be useful by those of ordinary skill in the art are contemplated as cancers treatable by this embodiment, either as a monotherapy or in combination with other therapeutic agents discussed below. Cancers that express high levels of A2a receptors or A2b receptors are among those cancers contemplated as treatable by the compounds of the invention. Examples of cancers that express high levels of A2a and/or A2b receptors may be discerned by those of ordinary skill in the art by reference to the Cancer Genome Atlas (TCGA) database. Non-limiting examples of cancers that express high levels of A2a receptors include cancers of the kidney, breast, lung, and liver. Non-limiting examples of cancers that express high levels of the A2b receptor include lung, colorectal, head & neck cancer, and cervical cancer.

Thus, one embodiment provides a method of treating cancer comprising administering an effective amount of a compound of the invention, or a pharmaceutically acceptable salt thereof, to a subject in need of such treatment, wherein said cancer is a cancer that expresses a high level of A2a receptor. A related embodiment provides a method of treating cancer comprising administering an effective amount of a compound of the invention, or a pharmaceutically acceptable salt thereof, to a subject in need of such treatment, wherein said cancer is selected from kidney (or renal) cancer, breast cancer, lung cancer, and liver cancer.

Another embodiment provides a method of treating cancer comprising administering an effective amount of a compound of the invention, or a pharmaceutically acceptable salt thereof, to a subject in need of such treatment, wherein said cancer is a cancer that expresses a high level of A2b receptor. A related embodiment provides a method of treating cancer comprising administering an effective amount of a compound of the invention, or a pharmaceutically acceptable salt thereof, to a subject in need of such treatment, wherein said cancer is selected from lung cancer, colorectal cancer, head & neck cancer, and cervical cancer.

Additional non-limiting examples of cancers which may be treatable by administration of a compound of the invention (alone or in combination with one or more additional agents described below) include cancers of the prostate (including but not limited to metastatic castration resistant prostate cancer), colon, rectum, pancreas, cervix, stomach, endometrium, brain, liver, bladder, ovary, testis, head, neck, skin (including melanoma and basal carcinoma), mesothelial lining, white blood cell (including lymphoma and leukemia) esophagus, breast, muscle, connective tissue, lung (including but not limited to small cell lung cancer, non-small cell lung cancer, and lung adenocarcinoma), adrenal gland, thyroid, kidney, or bone. Additional cancers treatable by a compound of the invention include glioblastoma, mesothelioma, renal cell carcinoma, gastric carcinoma, sarcoma, choriocarcinoma, cutaneous basocellular carcinoma, testicular seminoma, and Kaposi's sarcoma.

### CNS and Neurological Disorders

In other embodiments, the disease, condition or disorder is a central nervous system or a neurological disorder. Non-limiting examples of such diseases, conditions or disorders include movement disorders such as tremors, bradykinesias, gait disorders, dystonias, dyskinesias, tardive dyskinesias, other extrapyramidal syndromes, Parkinson's disease, and disorders associated with Parkinson's disease. The compounds of the invention also have the potential, or are believed to have the potential, for use in preventing or reducing the effect of drugs that cause or worsen such movement disorders.

### Infections

In other embodiments, the disease, condition or disorder is an infective disorder. Non-limiting examples of such diseases, conditions or disorders include an acute or chronic viral infection, a bacterial infection, a fungal infection, or a parasitic infection. In one embodiment, the viral infection is human immunodeficiency virus. In another embodiment, the viral infection is cytomegalovirus.

### Immune Disease

In other embodiments, the disease, condition or disorder is an immune-related disease, condition or disorder. Non-limiting examples of immune-related diseases, conditions, or disorders include multiple sclerosis and bacterial infections. (See, e.g., Safarzadeh, E. et al., Inflamm Res 2016 65(7): 511-20; and Antonioli, L., et al., Immunol Lett S0165-2478(18)30172-X 2018).

### Additional Indications

Other diseases, conditions, and disorders that have the potential to be treated or prevented, in whole or in part, by the inhibition of the A2a and/or A2b adenosine receptor(s) are also candidate indications for the compounds of the invention and salts thereof. Non-limiting examples of other diseases, conditions or disorders in which a compound of the invention, or a pharmaceutically acceptable salt thereof, may be useful include the treatment of hypersensitivity reaction to a tumor antigen and the amelioration of one or more complications related to bone marrow transplant or to a peripheral blood stem cell transplant. Thus, in another embodiment, the invention provides a method for treating a subject receiving a bone marrow transplant or a peripheral blood stem cell transplant by administering to said subject a therapeutically effective amount of a compound of the invention, or a pharmaceutically acceptable salt thereof, sufficient to increase the delayed-type hypersensitivity reaction to tumor antigen, to delay the time-to-relapse of post-transplant malignancy, to increase relapse-free survival time post-transplant, and/or to increase long-term post-transplant survival.

### Combination Therapy

In another aspect, the invention provides methods for the use of a compound of the invention, or a pharmaceutically acceptable salt thereof, (or a pharmaceutically acceptable composition comprising a compound of the invention or pharmaceutically acceptable salt thereof) in combination with one or more additional agents. Such additional agents may have some adenosine A2a and/or A2b receptor activity, or, alternatively, they may function through distinct mechanisms of action. The compounds of the invention may be used in combination with one or more other drugs in the treatment, prevention, suppression or amelioration of diseases or conditions for which the compounds of the invention or the other drugs described herein may have utility, where the combination of the drugs together are safer or more effective than either drug alone. The combination therapy may have an additive or synergistic effect. Such other drug(s) may be administered in an amount commonly used therefore, contemporaneously or sequentially with a compound of the invention or a pharmaceutically acceptable salt thereof. When a compound of the invention is used contemporaneously with one or more other drugs, the pharmaceutical composition may in specific embodiments contain such other drugs and the compound of the invention or its pharmaceutically acceptable salt in separate doses or in unit dosage form. However, the combination therapy may also include therapies in which the compound of the invention or its pharmaceutically acceptable salt and one or more other drugs are administered sequentially, on different or overlapping schedules. It is also contemplated that when used in combination with one or more other active ingredients, the compounds of the invention and the other active ingredients may be used in lower doses than when each is used singly. Accordingly, the pharmaceutical compositions comprising the compounds of the invention include those that contain one or more other active ingredients, in addition to a compound of the invention or a pharmaceutically acceptable salt thereof.

The weight ratio of the compound of the invention to the second active ingredient may be varied and will depend upon the effective dose of each ingredient. Generally, an effective dose of each will be used. Thus, for example, when a compound of the invention is used in combination with another agent, the weight ratio of the compound of the invention to the other agent may generally range from about 1000:1 to about 1:1000, in particular embodiments from about 200:1 to about 1:200. Combinations of a compound of the invention and other active ingredients will generally also be within the aforementioned range, but in each case, an effective dose of each active ingredient should generally be used.

Given the immunosuppressive role of adenosine, the administration of an A2a receptor antagonist, an A2b receptor antagonist, and/or an A2a/A2b receptor dual antagonist according to the invention may enhance the efficacy of immunotherapies such as PD-1 antagonists. Thus, in one embodiment, the additional therapeutic agent comprises an anti-PD-1 antibody. In another embodiment, the additional therapeutic agent is an anti-PD-L 1 antibody.

As noted above, PD-1 is recognized as having an important role in immune regulation and the maintenance of peripheral tolerance. PD-1 is moderately expressed on naive T-cells, B-cells and NKT-cells and up-regulated by T-cell and B-cell receptor signaling on lymphocytes, monocytes and myeloid cells (Sharpe et al., Nature Immunology (2007); 8:239-245).

Two known ligands for PD-1, PD-L1 (B7-H1) and PD-L2 (B7-DC) are expressed in human cancers arising in various tissues. In large sample sets of, for example, ovarian, renal, colorectal, pancreatic, and liver cancers, and in melanoma, it was shown that PD-L1 expression correlated with poor prognosis and reduced overall survival irrespective of subsequent treatment. (Dong et al., Nat Med. 8(8):793-800 (2002); Yang et al., Invest Ophthamol Vis Sci. 49: 2518-2525 (2008); Ghebeh et al., Neoplasia 8:190-198 (2006); Hamanishi et al., Proc. Natl. Acad. Sci. USA 104: 3360-3365 (2007); Thompson et al., Cancer 5: 206-211 (2006); Nomi et al., Clin. Cancer Research 13:2151-2157 (2007); Ohigashi et al., Clin. Cancer Research 11: 2947-2953; Inman et al., Cancer 109: 1499-1505 (2007); Shimauchi et al., Int. J. Cancer 121:2585-2590 (2007); Gao et al., Clin. Cancer Research 15: 971-979 (2009); Nakanishi J., Cancer Immunol Immunother. 56: 1173-1182 (2007); and Hino et al., Cancer 00: 1-9 (2010)).

Similarly, PD-1 expression on tumor infiltrating lymphocytes was found to mark dysfunctional T-cells in breast cancer and melanoma (Ghebeh et al., BMC Cancer. 2008 8:5714-(2008); and Ahmadzadeh et al., Blood 114: 1537-1544 (2009)) and to correlate with poor prognosis in renal cancer (Thompson et al., Clinical Cancer Research 15: 1757-1761(2007)). Thus, it has been proposed that PD-L1 expressing tumor cells interact with PD-1 expressing T-cells to attenuate T-cell activation and to evade immune surveillance, thereby contributing to an impaired immune response against the tumor.

Immune checkpoint therapies targeting the PD-1 axis have resulted in groundbreaking improvements in clinical response in multiple human cancers (Brahmer, et al., N Engl J Med 2012, 366: 2455-65; Garon et al., N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; and Wolchok et al., N Engl J Med 2013, 369: 122-33).

“PD-1 antagonist” means any chemical compound or biological molecule that blocks binding of PD-L 1 expressed on a cancer cell to PD-1 expressed on an immune cell (T-cell, B-cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment methods, medicaments and uses of the invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP 005009. Human PD-L1 and PD-L2 amino acid sequences can be found in NCBI Locus No.: NP_054862 and NP_079515, respectively.

PD-1 antagonists useful in any of the treatment methods, medicaments and uses of the invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in some embodiments, the human constant region is an IgG1 or IgG4 constant region. In some embodiments, the antigen binding fragment is selected from the group consisting of Fab, Fab′-SH, F(ab′)2, scFv and Fv fragments. Examples of PD-1 antagonists include, but are not limited to, pembrolizumab (KEYTRUDA®, Merck and Co., Inc., Rahway, NJ, USA). “Pembrolizumab” (formerly known as MK-3475, SCH 900475 and lambrolizumab and sometimes referred to as “pembro”) is a humanized IgG4 mAb with the structure described in WHO Drug Information, Vol. 27, No. 2, pages 161-162 (2013). Additional examples of PD-1 antagonists include nivolumab (OPDIVO®, Bristol-Myers Squibb Company, Princeton, NJ, USA), atezolizumab (MPDL3280A; TECENTRIQ®, Genentech, San Francisco, CA, USA), durvalumab (IMFINZI®, Astra Zeneca Pharmaceuticals, LP, Wilmington, DE, avelumab (BAVENCIO®, Merck KGaA, Darmstadt, Germany and Pfizer, Inc., New York, NY), cemiplimab (LIBTAYO®, Regeneron Pharmaceuticals, Inc., Tarrytown, NY and Sanofi-Aventis U.S. LLC., Bridgewater, NJ) and dostarlimab (JEMPERLI®, GlaxoSmithKline LLC, Philadelphia, PA).

Examples of monoclonal antibodies (mAbs) that bind to human PD-1, and useful in the treatment methods, medicaments and uses of the invention, are described in U.S. Pat. Nos. 7,488,802, 7,521,051, 8,008,449, 8,354,509, 8,168,757, WO2004/004771, WO2004/072286, WO2004/056875, and US2011/0271358.

Examples of mAbs that bind to human PD-L1, and useful in the treatment methods, medicaments and uses of the invention, are described in WO2013/019906, WO2010/077634 A1 and U.S. Pat. No. 8,383,796. Specific anti-human PD-L1 mAbs useful as the PD-1 antagonist in the treatment method, medicaments and uses of the invention include MPDL3280A, BMS-936559, MEDI4736, MSB0010718C and an antibody which comprises the heavy chain and light chain variable regions disclosed in WO2013/019906.

Other PD-1 antagonists useful in any of the treatment methods, medicaments and uses of the invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesin molecules that specifically bind to PD-1 are described in WO2010/027827 and WO2011/066342. Specific fusion proteins useful as the PD-1 antagonist in the treatment methods, medicaments and uses of the invention include AMP-224 (also known as B7-DCIg), which is a PD-L2-FC fusion protein that binds to human PD-1.

Thus, one embodiment provides a method of treating cancer comprising administering an effective amount of a compound of the invention, or a pharmaceutically acceptable salt thereof, in combination with a PD-1 antagonist to a subject in need thereof. In such embodiments, the compounds of the invention, or a pharmaceutically acceptable salt thereof, and PD-1 antagonist are administered concurrently or sequentially.

Specific non-limiting examples of such cancers in accordance with this embodiment include melanoma (including unresectable or metastatic melanoma), head & neck cancer (including recurrent or metastatic head and neck squamous cell cancer (HNSCC)), classical Hodgkin lymphoma (cHL), urothelial carcinoma, gastric cancer, cervical cancer, primary mediastinal large-B-cell lymphoma, microsatellite instability-high (MSI-H) cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, and salivary cancer.

In one embodiment, there is provided a method of treating cancer comprising administering an effective amount of a compound of the invention, or a pharmaceutically acceptable salt thereof, to a person in need thereof, in combination with a PD-1 antagonist, wherein said cancer is selected from unresectable or metastatic melanoma, recurrent or metastatic head and neck squamous cell cancer (HNSCC), classical Hodgkin lymphoma (cHL), urothelial carcinoma, gastric cancer, cervical cancer, primary mediastinal large-B-cell lymphoma, microsatellite instability-high cancer (MSI-H), non-small cell lung cancer, and hepatocellular carcinoma. In one such embodiment, the agent is a PD-1 antagonist. In one such embodiment, the agent is pembrolizumab. In another such embodiment, the agent is nivolumab. In another such embodiment, the agent is atezolizumab.

Pembrolizumab is approved by the U.S. FDA for the treatment of patients with unresectable or metastatic melanoma, Stage IIB, IIC, or III melanoma following complete resection, non-small cell lung cancer (NSCLC), head and neck squamous cell cancer (HNSCC), classical Hodgkin Lymphoma (cHL), primary mediastinal large B-cell lymphoma, urothelial carcinoma, microsatellite instability-high or mismatch repair deficient cancer, microsatellite instability-high or mismatch repair deficient colorectal cancer (CRC), gastric cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, cutaneous squamous cell carcinoma (cSCC), triple-negative breast cancer (TNBC), as described in the Prescribing Information for KEYTRUDA™ (Merck & Co., Inc., Rahway, NJ USA; initial U.S. approval 2014, updated January 2023). In another embodiment, there is provided a method of treating cancer comprising administering an effective amount of a compound of the invention, or a pharmaceutically acceptable salt thereof, in combination with pembrolizumab to a person in need thereof, wherein said cancer is selected from unresectable or metastatic melanoma, Stage IIB, IIC, or III melanoma following complete resection, non-small cell lung cancer (NSCLC), head and neck squamous cell cancer (HNSCC), classical Hodgkin Lymphoma (cHL), microsatellite instability-high or mismatch repair deficient cancer, microsatellite instability-high or mismatch repair deficient colorectal cancer (CRC), gastric cancer, esophageal cancer, cervical cancer, hepatocellular carcinoma (HCC), Merkel cell carcinoma (MCC), renal cell carcinoma (RCC), endometrial carcinoma, tumor mutational burden-high (TMB-H) cancer, cutaneous squamous cell carcinoma (cSCC), triple-negative breast cancer (TNBC).

In another embodiment, there is provided a method of treating cancer comprising administering an effective amount of a compound of the invention, or a pharmaceutically acceptable salt thereof in combination with a PD-1 antagonist, to a person in need thereof, wherein said cancer is selected from unresectable or metastatic melanoma, Stage IIB, IIC, or III melanoma following complete resection, non-small cell lung cancer, head and neck squamous cell cancer, classical Hodgkin Lymphoma, primary mediastinal large B cell lymphoma, microsatellite instability-high or mismatch repair deficient cancer, microsatellite instability-high or mismatch repair deficient colorectal cancer, urothelial carcinoma, gastric cancer, esophageal cancer, cervical cancer, hepatocellular carcinoma, Merkel cell carcinoma, renal cell carcinoma, endometrial carcinoma, tumor mutational burden-high cancer, cutaneous squamous cell carcinoma, triple-negative breast cancer. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab. In another such embodiment, the agent is cemiplimab. In another such embodiment, the agent is dostarlimab.

In another embodiment, there is provided a method of treating cancer comprising administering an effective amount of a compound of the invention, or a pharmaceutically acceptable salt thereof, in combination with a PD-1 antagonist, to a person in need thereof, wherein said cancer is selected from melanoma, non-small cell lung cancer, small cell lung cancer, head and neck cancer, bladder cancer, breast cancer, gastrointestinal cancer, multiple myeloma, hepatocellular cancer, lymphoma, renal cancer, mesothelioma, ovarian cancer, esophageal cancer, anal cancer, biliary tract cancer, colorectal cancer, cervical cancer, thyroid cancer, and salivary cancer. In one such embodiment, the agent is pembrolizumab. In another such embodiment, the agent is nivolumab. In another such embodiment, the agent is atezolizumab. In another such embodiment, the agent is durvalumab. In another such embodiment, the agent is avelumab. In another such embodiment, the agent is cemiplimab. In another such embodiment, the agent is dostarlimab

In one embodiment, there is provided a method of treating unresectable or metastatic melanoma comprising administering an effective amount of a compound of the invention, or a pharmaceutically acceptable salt thereof, in combination with a PD-1 antagonist to a person in need thereof. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating recurrent or metastatic HNSCC comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating classical Hodgkin lymphoma (cHL) comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another such embodiment, the agent is cemiplimab. In another such embodiment, the agent is dostarlimab.

In one embodiment, there is provided a method of treating triple-negative breast cancer comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another embodiment, the agent is avelumab. In another such embodiment, the agent is atezolizumab. In another such embodiment, the agent is cemiplimab. In another such embodiment, the agent is dostarlimab.

In one embodiment, there is provided a method of treating urothelial carcinoma comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating gastric cancer comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another such embodiment, the agent is cemiplimab. In another embodiment, the agent is avelumab. In another such embodiment, the agent is dostarlimab.

In one embodiment, there is provided a method of treating cervical cancer comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating primary mediastinal large-B-cell lymphoma comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating microsatellite instability-high (MSI-H) cancer comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating non-small cell lung cancer comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating hepatocellular carcinoma comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating Merkel cell carcinoma comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating renal cell carcinoma comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating endometrial cell carcinoma comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In one embodiment, there is provided a method of treating cutaneous squamous cell carcinoma comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another such embodiment, the agent is cemiplimab. In another embodiment, the agent is avelumab. In another such embodiment, the agent is dostarlimab.

In one embodiment, there is provided a method of treating tumor mutational burden-high cancer comprising administering an effective amount of a compound of the invention, or a pharmaceutically acceptable salt thereof, in combination with a PD-1 antagonist, to a person in need thereof. In one such embodiment, the agent is pembrolizumab. In another such embodiment, the agent is nivolumab. In another such embodiment, the agent is atezolizumab. In another embodiment, the agent is avelumab. In another such embodiment, the agent is cemiplimab. In another such embodiment, the agent is dostarlimab.

In another embodiment, the additional therapeutic agent is at least one immunomodulator other than an A2a or A2b receptor inhibitor. Non-limiting examples of immunomodulators include CD40L, B7, B7RP1, anti-CD40, anti-CD38, anti-ICOS, 4-IBB ligand, dendritic cell cancer vaccine, IL2, IL12, ELC/CCL19, SLC/CCL21, MCP-1, IL-4, IL-18, TNF, IL-15, MDC, IFN-a/-13, M-CSF, IL-3, GM-CSF, IL-13, anti-IL-10 and indolamine 2,3-dioxygenase 1 (IDO1) inhibitors.

In another embodiment, the additional therapeutic agent comprises radiation. Such radiation includes localized radiation therapy and total body radiation therapy.

In another embodiment, the additional therapeutic agent is at least one chemotherapeutic agent. Non-limiting examples of chemotherapeutic agents contemplated for use in combination with the compounds of the invention include: pemetrexed, alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nucleoside analogs (e.g., gemcitabine); nitroso ureas such as carmustine, lomustine, and streptozocin; topoisomerase 1 inhibitors (e.g., irinotecan); platinum complexes such as cisplatin, carboplatin and oxaliplatin; bioreductive alkylators such as mitomycin, procarbazine, dacarbazine and altretamine); anthracycline-based therapies (e.g., doxorubicin, daunorubicin, epirubicin and idarubicin); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, estramustine, vinblastine, docetaxol, epothilone derivatives, and paclitaxel); hormonal agents (e.g., estrogens; conjugated estrogens; ethynyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); luteinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide).

In another embodiment, the additional therapeutic agent is at least one signal transduction inhibitor (STI). Non-limiting examples of signal transduction inhibitors include BCR/ABL kinase inhibitors, epidermal growth factor (EGF) receptor inhibitors, HER-2/neu receptor inhibitors, and famesyl transferase inhibitors (FTIs).

In another embodiment, the additional therapeutic agent is at least one anti-infective agent. Non-limiting examples of anti-infective agents include cytokines, non-limiting examples of which include granulocyte-macrophage colony stimulating factor (GM-CSF) and an flt3-ligand.

In another embodiment, the invention provides a method for treating or preventing a viral infection (e.g., a chronic viral infection) including, but not limited to, hepatitis C virus (HCV), human papilloma virus (HPV), cytomegalovirus (CMV), Epstein-Barr virus (EBV), varicella zoster virus, coxsackievirus, and human immunodeficiency virus (HIV).

In another embodiment, the invention provides a method for the treatment of an infective disorder, said method comprising administering to a subject in need thereof an effective amount of a compound of the invention, or a pharmaceutically acceptable salt thereof, in combination with a vaccine. In some embodiments, the vaccine is an anti-viral vaccine, including, for example, an anti-HIV vaccine. Other antiviral agents contemplated for use include an anti-HIV, anti-HPV, anti HCV, anti HSV agents and the like. In other embodiments, the vaccine is effective against tuberculosis or malaria. In still other embodiments, the vaccine is a tumor vaccine (e.g., a vaccine effective against melanoma); the tumor vaccine may comprise genetically modified tumor cells or a genetically modified cell line, including genetically modified tumor cells or a genetically modified cell line that has been transfected to express granulocyte-macrophage stimulating factor (GM-CSF). In another embodiment, the vaccine includes one or more immunogenic peptides and/or dendritic cells.

In another embodiment, the invention provides for the treatment of an infection by administering a compound of the invention, or a pharmaceutically acceptable salt thereof, and at least one additional therapeutic agent, wherein a symptom of the infection observed after administering both the compound of the invention (or a pharmaceutically acceptable salt thereof) and the additional therapeutic agent is improved over the same symptom of infection observed after administering either alone. In some embodiments, the symptom of infection observed is reduction in viral load, increase in CD4+ T cell count, decrease in opportunistic infections, increased survival time, eradication of chronic infection, or a combination thereof.

### Definitions

As used herein, unless otherwise specified, the following terms have the following meanings.

Unsatisfied valences in the text, schemes, examples, structural formulae, and any Tables herein are assumed to have a hydrogen atom or atoms of sufficient number to satisfy the valences.

When a variable appears more than once in any moiety or in any compound of the invention (e.g., aryl, heterocycle, —N(R)₂), the selection of moieties defining that variable for each occurrence is independent of its definition at every other occurrence unless specified otherwise in the local variable definition.

As used herein, unless otherwise specified, the term “A2a receptor antagonist” (equivalently, A2a antagonist) and/or “A2b receptor antagonist” (equivalently, A2b antagonist) means a compound exhibiting a potency (IC₅₀) of less than about 1 μM with respect to the A2a and/or A2b receptors, respectively, when assayed in accordance with the procedures described herein. Certain compounds of the invention exhibit at least 10-fold selectivity for antagonizing the A2a receptor and/or the A2b receptor over any other adenosine receptor (e.g., A1 or A3).

As described herein, unless otherwise indicated, the use of a compound in treatment means that an amount of the compound, generally presented as a component of a formulation that comprises other excipients, is administered in aliquots of an amount, and at time intervals, which provides and maintains at least a therapeutic serum level of at least one pharmaceutically active form of the compound over the time interval between dose administrations.

The phrase “at least one” used in reference to the number of components comprising a composition, for example, “at least one pharmaceutical excipient” means that one member of the specified group is present in the composition, and more than one may additionally be present.

Components of a composition are typically aliquots of isolated pure material added to the composition, where the purity level of the isolated material added into the composition is the normally accepted purity level for a reagent of the type.

Whether used in reference to a substituent on a compound or a component of a pharmaceutical composition the phrase “one or more”, means the same as “at least one”.

“Concurrently” and “contemporaneously” both include in their meaning (1) simultaneously in time (e.g., at the same time); and (2) at different times but within the course of a common treatment schedule.

“Consecutively” means one following the other.

“Sequentially” refers to a series administration of therapeutic agents that awaits a period of efficacy to transpire between administering each additional agent; this is to say that after administration of one component, the next component is administered after an effective time period after the first component; the effective time period is the amount of time given for realization of a benefit from the administration of the first component.

“Effective amount” or “therapeutically effective amount” is meant to describe the provision of an amount of at least one compound of the invention or of a composition comprising at least one compound of the invention which is effective in treating or inhibiting a disease or condition described herein, and thus produce the desired therapeutic, ameliorative, inhibitory or preventative effect. For example, in treating a cancer as described herein with one or more of the compounds of the invention optionally in combination with one or more additional agents, “effective amount” (or “therapeutically effective amount”) means, for example, providing the amount of at least one compound of the invention that results in a therapeutic response in a patient afflicted with the disease, condition, or disorder, including a response suitable to manage, alleviate, ameliorate, or treat the condition or alleviate, ameliorate, reduce, or eradicate one or more symptoms attributed to the condition and/or long-term stabilization of the condition, for example, as may be determined by the analysis of pharmacodynamic markers or clinical evaluation of patients afflicted with the condition.

“Patient” and “subject” means an animal, such as a mammal (e.g., a human being) and is preferably a human being.

“Treat” or “treatment” means to administer an agent, such as a composition containing any of the compounds described herein, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting, delaying or slowing the progression of such symptom(s) by any clinically measurable degree. The amount of an agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. The term further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

“Prodrug” means compounds that are rapidly transformed, for example, by hydrolysis in blood, in vivo to the parent compound, e.g., conversion of a prodrug of a compound of the invention to a compound of the invention, or to a salt thereof. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference; the scope of this invention includes prodrugs of the novel compounds of this invention.

The term “substituted” means that one or more of the moieties enumerated as substituents (or, where a list of substituents are not specifically enumerated, the substituents specified elsewhere in this application) for the particular type of substrate to which said substituent is appended, provided that such substitution does not exceed the normal valence rules for the atom in the bonding configuration presented in the substrate, and that the substitution ultimate provides a stable compound, which is to say that such substitution does not provide compounds with mutually reactive substituents located geminal or vicinal to each other; and wherein the substitution provides a compound sufficiently robust to survive isolation to a useful degree of purity from a reaction mixture.

Where optional substitution by a moiety is described (e.g. “optionally substituted”) the term means that if substituents are present, one or more of the enumerated (or default) moieties listed as optional substituents for the specified substrate can be present on the substrate in a bonding position normally occupied by the default substituent, for example, a hydrogen atom on an alkyl chain can be substituted by one of the optional substituents, in accordance with the definition of “substituted” presented herein.

“Alkyl” means an aliphatic hydrocarbon group, which may be straight or branched, comprising 1 to 10 carbon atoms. “(C₁-C₆)alkyl” means an aliphatic hydrocarbon group, which may be straight or branched, comprising 1 to 6 carbon atoms. Branched means that one or more lower alkyl groups such as methyl, ethyl or propyl, are attached to a linear alkyl chain. Non-limiting examples of alkyl groups include methyl, ethyl, n-propyl, isopropyl, n-butyl, i-butyl, and t-butyl.

“Haloalkyl” means an alkyl as defined above wherein one or more hydrogen atoms on the alkyl (up to and including each available hydrogen group) is replaced by a halogen atom. As appreciated by those of skill in the art, “halo” or “halogen” as used herein is intended to include chloro (Cl), fluoro (F), bromo (Br) and iodo (I). In some embodiments, halogen is chloro (Cl) or fluoro (F).

“Aryl” means an aromatic monocyclic or multicyclic ring system comprising 6 to 14 carbon atoms, preferably 6 to 10 carbon atoms. The aryl group can be optionally substituted with one or more “ring system substituents” which may be the same or different and are as defined herein. Non-limiting examples of suitable aryl groups include phenyl and naphthyl. “Monocyclic aryl” means phenyl.

“Heteroaryl” means an aromatic monocyclic or multicyclic ring system comprising 5 to 14 ring atoms, preferably 5 to 10 ring atoms, in which one or more of the ring atoms is an element other than carbon, for example nitrogen, oxygen or sulfur, alone or in combination. In some embodiments, heteroaryls contain 5 to 6 ring atoms. The “heteroaryl” can be optionally substituted by one or more substituents, which may be the same or different, as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl (which alternatively may be referred to as thiophenyl), pyrimidinyl, pyridone (including N-substituted pyridones), isoxazolyl, isothiazolyl, oxazolyl, oxadiazolyl, thiazolyl, thiadiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like. The term “monocyclic heteroaryl” refers to monocyclic versions of heteroaryl as described above and includes 4- to 7-membered monocyclic heteroaryl groups comprising from 1 to 4 ring heteroatoms, said ring heteroatoms being independently selected from the group consisting of N, O, and S, and oxides thereof. The point of attachment to the parent moiety is to any available ring carbon or ring heteroatom. Non-limiting examples of monocyclic heteroaryl moieties include pyridyl, pyrazinyl, furanyl, thienyl, pyrimidinyl, pyridazinyl, pyridinyl, thiazolyl, isothiazolyl, oxazolyl, oxadiazolyl, isoxazolyl, pyrazolyl, furazanyl, pyrrolyl, pyrazolyl, triazolyl, thiadiazolyl (e.g., 1,2,4-thiadiazolyl), imidazolyl, and triazinyl (e.g., 1,2,4-triazinyl), and oxides thereof.

“Cycloalkyl” means a non-aromatic fully saturated monocyclic or multicyclic ring system comprising 3 to 10 carbon atoms, preferably 3 to 6 carbon atoms. The cycloalkyl can be optionally substituted with one or more substituents, which may be the same or different, as described herein. Monocyclic cycloalkyl refers to monocyclic versions of the cycloalkyl moieties described herein. Non-limiting examples of suitable monocyclic cycloalkyls include cyclopropyl, cyclopentyl, cyclohexyl, cycloheptyl and the like. Non-limiting examples of multicyclic cycloalkyls include [1.1.1]-bicyclopentane, 1-decalinyl, norbornyl, adamantyl and the like.

“Heterocycloalkyl” (or “heterocyclyl”) means a non-aromatic saturated monocyclic or multicyclic ring system comprising 3 to 10 ring atoms, preferably 5 to 10 ring atoms, in which one or more of the atoms in the ring system is an element other than carbon, for example nitrogen, oxygen or sulfur, alone or in combination. There are no adjacent oxygen and/or sulfur atoms present in the ring system. In some embodiments, heterocycloalkyl groups contain 4, 5 or 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more substituents, which may be the same or different, as described herein. The nitrogen or sulfur atom of the heterocyclyl can be optionally oxidized to the corresponding N-oxide, S-oxide or S,S-dioxide. Thus, the term “oxide,” when it appears in a definition of a variable in a general structure described herein, refers to the corresponding N-oxide, S-oxide, or S,S-dioxide. “Heterocyclyl” also includes rings wherein ═O replaces two available hydrogens on the same carbon atom (i.e., heterocyclyl includes rings having a carbonyl group in the ring). Such ═O groups may be referred to herein as “oxo.” An example of such a moiety is pyrrolidinone (or pyrrolidone):

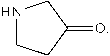

As used herein, the term “monocyclic heterocycloalkyl” refers to monocyclic versions of the heterocycloalkyl moieties described herein and include a 4- to 7-membered monocyclic heterocycloalkyl groups comprising from 1 to 4 ring heteroatoms, said ring heteroatoms being independently selected from the group consisting of N, N-oxide, O, S, S-oxide, S(O), and S(O)₂. The point of attachment to the parent moiety is to any available ring carbon or ring heteroatom. Non-limiting examples of monocyclic heterocycloalkyl groups include piperidyl, oxetanyl, pyrrolyl, piperazinyl, morpholinyl, thiomorpholinyl, thiazolidinyl, 1,4-dioxanyl, tetrahydrofuranyl, tetrahydrothiophenyl, beta lactam, gamma lactam, delta lactam, beta lactone, gamma lactone, delta lactone, and pyrrolidinone, and oxides thereof. Non-limiting examples of lower alkyl-substituted oxetanyl include the moiety:

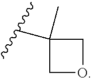 [SMILES: *C1(C)COC1]

It is noted that in hetero-atom containing ring systems of this invention, there are no hydroxyl groups on carbon atoms adjacent to a N, O or S, and there are no N or S groups on carbon adjacent to another heteroatom.

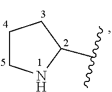 [SMILES: *C1CCCN1]

there is no —OH attached directly to carbons marked 2 and 5.

The linen [symbol] as a bond generally indicates a mixture of, or either of, the possible isomers, e.g., containing (R)- and (S)-stereochemistry. For example:

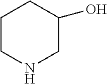

means containing both

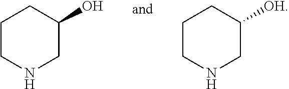

The wavy line [symbol] as used herein, indicates a point of attachment to the rest of the compound. Lines drawn into the ring systems, such as, for example:

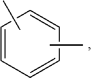

indicate that the indicated line (bond) may be attached to any of the substitutable ring atoms.

“Oxo” is defined as an oxygen atom that is double bonded to a ring carbon in a cycloalkyl, cycloalkenyl, heterocyclyl, heterocyclenyl, or other ring described herein, e.g.,

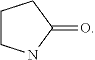

As well known in the art, a bond drawn from a particular atom wherein no moiety is depicted at the terminal end of the bond indicates a methyl group bound through that bond to the atom, unless stated otherwise. For example:

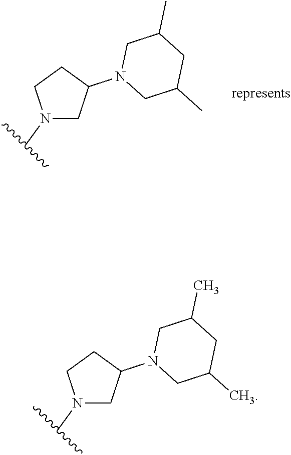 [SMILES: *N1CCC(N2CC(C)CC(C)C2)C1 ; *N1CCC(N2CC(C)CC(C)C2)C1]

One or more compounds of the invention may also exist as, or optionally be converted to, a solvate. Preparation of solvates is generally known. Thus, for example, M. Caira et al., J. Pharmaceutical Sci., 93(3), 601-611 (2004) describe the preparation of the solvates of the antifungal fluconazole in ethyl acetate as well as from water. Similar preparations of solvates, and hemisolvate, including hydrates (where the solvent is water or aqueous based) and the like are described by E. C. van Tonder et al., AAPS PharmSciTech., 5(1), article 12 (2004); and A. L. Bingham et al., Chem. Commun., 603-604 (2001). A typical, non-limiting, process involves dissolving the inventive compound in desired amounts of the desired solvent (for example, an organic solvent, an aqueous solvent, water or mixtures of two or more thereof) at a higher than ambient temperature, and cooling the solution, with or without an antisolvent present, at a rate sufficient to form crystals which are then isolated by standard methods. Analytical techniques such as, for example I.R. spectroscopy, show the presence of the solvent (including water) in the crystals as a solvate (or hydrate in the case where water is incorporated into the crystalline form).

The term “purified”, “in purified form” or “in isolated and purified form” for a compound refers to the physical state of said compound after being isolated from a synthetic process or natural source or combination thereof. Thus, the term “purified”, “in purified form” or “in isolated and purified form” for a compound refers to the physical state of said compound after being obtained from a purification process or processes described herein or well known to the skilled artisan, and in sufficient purity to be characterized by standard analytical techniques described herein or well known to the skilled artisan.

This invention also includes the compounds of the invention in isolated and purified form obtained by routine techniques. Polymorphic forms of the compounds of the invention, and of the salts, solvates and prodrugs of the thereof, are intended to be included in the invention. Certain compounds of the invention may exist in different isomeric forms (e.g., enantiomers, diastereoisomers, atropisomers). The inventive compounds include all isomeric forms thereof, both in pure form and admixtures of two or more, including racemic mixtures.

In similar manner, unless indicated otherwise, presenting a structural representation of any tautomeric form of a compound which exhibits tautomerism is meant to include all such tautomeric forms of the compound. Accordingly, where compounds of the invention, their salts, and solvates and prodrugs thereof, may exist in different tautomeric forms or in equilibrium among such forms, all such forms of the compound are embraced by, and included within the scope of the invention. Examples of such tautomers include, but are not limited to, ketone/enol tautomeric forms, imine-enamine tautomeric forms, and for example heteroaromatic forms such as the following moieties:

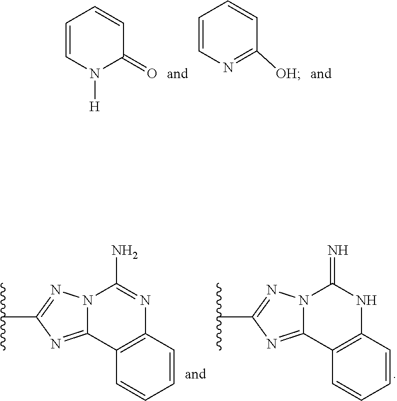 [SMILES: O=c1cccc[nH]1 ; Oc1ccccn1 ; *c1nc2c3ccccc3nc(N)n2n1 ; *c1nc2c3ccccc3[nH]c(=N)n2n1]

Where a reaction scheme appearing in an example employs a compound having one or more stereocenters, the stereocenters are indicated with an asterisk, as shown below:

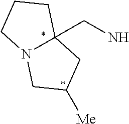 [SMILES: CC1CN2CCCC2(CN)C1]

Accordingly, the above depiction consists of the following pairs of isomers: (i) Trans-isomers ((2R,7aS)-2-methylhexahydro-1H-pyrrolizin-7a-yl)methanamine (Compound ABC-1) and ((2S,7aR)-2-methylhexahydro-1H-pyrrolizin-7a-yl)methanamine (Compound ABC-2); and (ii) Cis-isomers ((2R,7aR)-2-methylhexahydro-1H-pyrrolizin-7a-yl)methanamine (Compound ABC-3) and ((2S,7aS)-2-methylhexahydro-1H-pyrrolizin-7a-yl)methanamine (Compound ABC-4).

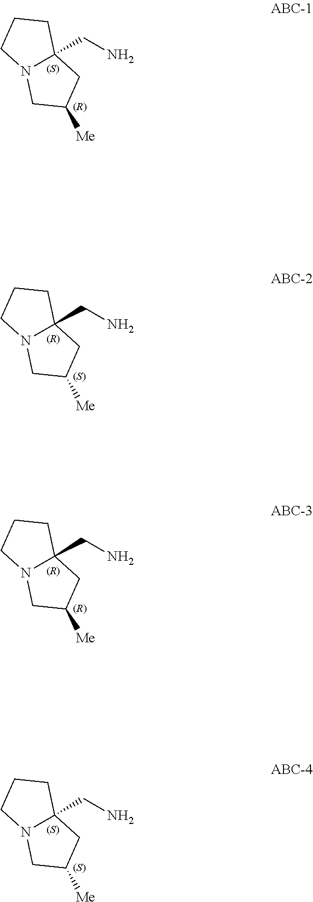

All stereoisomers of the compounds of the invention (including salts and solvates of the inventive compounds and their prodrugs), such as those which may exist due to asymmetric carbons present in a compound of the invention and including enantiomeric forms (which may exist even in the absence of asymmetric carbons), rotameric forms, atropisomers, and diastereomeric forms, are contemplated within the scope of this invention. Individual stereoisomers of the compounds of the invention may be isolated in a pure form, for example, substantially free of other isomers, or may be isolated as an admixture of two or more stereoisomers or as a racemate. The chiral centers of the invention can have the S or R configuration as defined by the IUPAC 1974 Recommendations. The use of the terms “salt”, “solvate” “prodrug” and the like, is intended to equally apply to salts, solvates and prodrugs of isolated enantiomers, stereoisomer pairs or groups, rotamers, tautomers, or racemates of the inventive compounds.

Where diastereomeric mixtures can be separated into their individual diastereomers on the basis of their physical chemical differences by known methods, for example, by chiral chromatography and/or fractional crystallization, simple structural representation of the compound contemplates all diastereomers of the compound. As is known, enantiomers may also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individually isolated diastereomers to the corresponding purified enantiomers.

As the term is employed herein, salts of the inventive compounds, whether acidic salts formed with inorganic and/or organic acids, basic salts formed with inorganic and/or organic bases, salts formed which include zwitterionic character, for example, where a compound contains both a basic moiety, for example, but not limited to, a nitrogen atom, for example, an amine, pyridine or imidazole, and an acidic moiety, for example, but not limited to a carboxylic acid, are included in the scope of the inventive compounds described herein. The formation of pharmaceutically useful salts from basic (or acidic) pharmaceutical compounds are discussed, for example, by S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference.

The invention contemplates all available salts, including salts which are generally recognized as safe for use in preparing pharmaceutical formulations and those which may be formed presently within the ordinary skill in the art and are later classified as being “generally recognized as safe” for use in the preparation of pharmaceutical formulations, termed herein as “pharmaceutically acceptable salts”. Examples of pharmaceutically acceptable acid addition salts include, but are not limited to, acetates, including trifluoroacetate salts, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates) undecanoates, and the like.

Examples of pharmaceutically acceptable basic salts include, but are not limited to, ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexyl-amine, choline, tromethamine, and salts with amino acids such as arginine, lysine and the like. Basic nitrogen-containing groups may be converted to an ammonium ion or quarternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), arylalkyl halides (e.g., benzyl and phenethyl bromides), and others.

All such acid and base salts are intended to be pharmaceutically acceptable salts within the scope of the invention and all acid and base salts are considered equivalent to the free forms of the corresponding compounds for purposes of the scope of the invention.

A functional group in a compound termed “protected” means that the group is in modified form to preclude undesired side reactions at the protected site when the protected compound is subjected to particular reaction conditions aimed at modifying another region of the molecule. Suitable protecting groups are known, for example, as by reference to standard textbooks, for example, T. W. Greene et al., Protective Groups in organic Synthesis (1991), Wiley, New York.

In the compounds of the invention, the atoms may exhibit their natural isotopic abundances, or one or more of the atoms may be artificially enriched in a particular isotope having the same atomic number, but an atomic mass or mass number different from the atomic mass or mass number predominantly found in nature. The invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds of the invention can be prepared without undue experimentation by conventional techniques well known to those skilled in the art or by processes analogous to those described in the Schemes and Examples herein using appropriate isotopically-enriched reagents and/or intermediates.

The invention also embraces isotopically-labeled compounds of the invention which are structurally identical to those recited herein, but for the fact that a statistically significant percentage of one or more atoms in that form of the compound are replaced by an atom having an atomic mass or mass number different from the atomic mass or mass number of the most abundant isotope usually found in nature, thus altering the naturally occurring abundance of that isotope present in a compound of the invention. Examples of isotopes that can be preferentially incorporated into compounds of the invention include isotopes of hydrogen, carbon, nitrogen, oxygen, phosphorus, iodine, fluorine and chlorine, for example, but not limited to: ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, and ³⁶Cl, ¹²³I and ¹²⁵I. It will be appreciated that other isotopes also may be incorporated by known means.

Certain isotopically-labeled compounds of the invention (e.g., those labeled with ³H, ¹¹C and ¹⁴C) are recognized as being particularly useful in compound and/or substrate tissue distribution assays using a variety of known techniques. In some embodiments, tritiated (i.e., ³H) and carbon-14 (i.e., ¹⁴C) isotopes are used for their ease of preparation and detection. Further, substitution of a naturally abundant isotope with a heavier isotope, for example, substitution of protium with deuterium (i.e., ²H) may afford certain therapeutic advantages resulting from greater metabolic stability (e.g., increased in vivo half-life or reduced dosage requirements) and hence may be useful in some circumstances. Isotopically labeled compounds of the invention can generally be prepared by following procedures analogous to those disclosed in the reaction Schemes and/or in the Examples herein below, by substituting an appropriate isotopically labeled reagent for a non-isotopically labeled reagent, or by well-known reactions of an appropriately prepared precursor to the compound of the invention which is specifically prepared for such a “labeling” reaction. Such compounds are included also in the invention.

The term “composition” is intended to encompass a product comprising the specified ingredients in the specified amounts, and any product which results, directly or indirectly, from combination of the specified ingredients in the specified amounts.

The term “pharmaceutical composition” as used herein encompasses both the bulk composition and individual dosage units comprised of one, or more than one (e.g., two), pharmaceutically active agents such as, for example, a compound of the invention (optionally together with an additional agent as described herein), along with any pharmaceutically inactive excipients. As will be appreciated by those of ordinary skill in the art, excipients are any constituent that adapts the composition to a particular route of administration or aids the processing of a composition into a dosage form without itself exerting an active pharmaceutical effect. The bulk composition and each individual dosage unit can contain fixed amounts of the aforesaid one, or more than one, pharmaceutically active agents. The bulk composition is material that has not yet been formed into individual dosage units.

It will be appreciated that pharmaceutical formulations of the invention may comprise more than one compound of the invention (or a pharmaceutically acceptable salt thereof), for example, the combination of two or three compounds of the invention, each present in such a composition by adding to the formulation the desired amount of the compound in a pharmaceutically acceptably pure form. It will be appreciated also that in formulating compositions of the invention, a composition may comprise, in addition to one or more of compounds of the invention, one or more other agents which also have pharmacological activity, as described herein.

While formulations of the invention may be employed in bulk form, it will be appreciated that for most applications the inventive formulations will be incorporated into a dosage form suitable for administration to a patient, each dosage form comprising an amount of the selected formulation which contains an effective amount of one or more compounds of the invention.

Examples of suitable dosage forms include, but are not limited to, dosage forms adapted for: (i) oral administration, e.g., a liquid, gel, powder, solid or semi-solid pharmaceutical composition which is loaded into a capsule or pressed into a tablet and may comprise additionally one or more coatings which modify its release properties, for example, coatings which impart delayed release or formulations which have extended release properties; (ii) a dosage form adapted for intramuscular administration (IM), for example, an injectable solution or suspension, and which may be adapted to form a depot having extended release properties; (iii) a dosage form adapted for intravenous administration (IV), for example, a solution or suspension, for example, as an IV solution or a concentrate to be injected into a saline IV bag; (iv) a dosage form adapted for administration through tissues of the oral cavity, for example, a rapidly dissolving tablet, a lozenge, a solution, a gel, a sachets or a needle array suitable for providing intramucosal administration; (v) a dosage form adapted for administration via the mucosa of the nasal or upper respiratory cavity, for example a solution, suspension or emulsion formulation for dispersion in the nose or airway; (vi) a dosage form adapted for transdermal administration, for example, a patch, cream or gel; (vii) a dosage form adapted for intradermal administration, for example, a microneedle array; and (viii) a dosage form adapted for delivery via rectal or vaginal mucosa, for example, a suppository.

For preparing pharmaceutical compositions comprising compounds of the invention, generally the compounds of the invention will be combined with one or more pharmaceutically acceptable excipients. These excipients impart to the composition properties which make it easier to handle or process, for example, lubricants or pressing aids in powdered medicaments intended to be tableted, or adapt the formulation to a desired route of administration, for example, excipients which provide a formulation for oral administration, for example, via absorption from the gastrointestinal tract, transdermal or transmucosal administration, for example, via adhesive skin “patch” or buccal administration, or injection, for example, intramuscular or intravenous, routes of administration. These excipients are collectively termed herein “a carrier”. Typically, formulations may comprise up to about 95 percent active ingredient, although formulations with greater amounts may be prepared.

Pharmaceutical compositions can be solid, semi-solid or liquid. Solid form preparations can be adapted to a variety of modes of administration, examples of which include, but are not limited to, powders, dispersible granules, mini-tablets, beads, which can be used, for example, for tableting, encapsulation, or direct administration. Liquid form preparations include, but are not limited to, solutions, suspensions and emulsions which for example, but not exclusively, can be employed in the preparation of formulations intended for parenteral injection, for intranasal administration, or for administration to some other mucosal membrane. Formulations prepared for administration to various mucosal membranes may also include additional components adapting them for such administration, for example, viscosity modifiers.

Aerosol preparations, for example, suitable for administration via inhalation or via nasal mucosa, may include solutions and solids in powder form, which may be in combination with a pharmaceutically acceptable propellant, for example, an inert compressed gas, e.g., nitrogen.

Also included are solid form preparations which are intended to be converted, shortly before use, to a suspension or a solution, for example, for oral or parenteral administration. Examples of such solid forms include, but are not limited to, freeze dried formulations and liquid formulations adsorbed into a solid absorbent medium.

The compounds of the invention may also be deliverable transdermally or transmucosally, for example, from a liquid, suppository, cream, foam, gel, or rapidly dissolving solid form. It will be appreciated that transdermal compositions can also take the form of creams, lotions, aerosols and/or emulsions and can be provided in a unit dosage form which includes a transdermal patch of any know in the art, for example, a patch which incorporates either a matrix comprising the pharmaceutically active compound or a reservoir which comprises a solid or liquid form of the pharmaceutically active compound.

Examples of pharmaceutically acceptable carriers and methods of manufacture for various compositions mentioned above may be found in A. Gennaro (ed.), Remington: The Science and Practice of Pharmacy, 20^{th} Edition, (2000), Lippincott Williams & Wilkins, Baltimore, MD.

Preferably, the pharmaceutical preparation is in a unit dosage form. In such form, the preparations subdivided into suitably sized unit doses containing appropriate quantities of the active component, e.g., an effective amount to achieve the desired purpose.

The actual dosage employed may be varied depending upon the requirements of the patient and the severity of the condition being treated. Determination of the proper dosage regimen for a particular situation is within the skill in the art. For convenience, the total daily dosage may be divided and administered in portions during the day as required.

In accordance with the invention, antagonism of adenosine A2a and/or A2b receptors is accomplished by administering to a patient in need of such therapy an effective amount of one or more compounds of the invention, or a pharmaceutically acceptable salt thereof.

In some embodiments the compound to be administered is in the form of a pharmaceutical composition comprising the compound of the invention, or a pharmaceutically acceptable salt thereof, and at least one pharmaceutically acceptable carrier (described herein). It will be appreciated that pharmaceutically formulations of the invention may comprise more than one compound of the invention, or a salt thereof, for example, the combination of two or three compounds of the invention, or, additionally or alternatively, another active agent such as those described herein, each present by adding to the formulation the desired amount of the compound or a salt thereof (or agent, where applicable) which has been isolated in a pharmaceutically acceptably pure form.

As mentioned above, administration of a compound of the invention to effect antagonism of A2a and/or A2b receptors is preferably accomplished by incorporating the compound into a pharmaceutical formulation incorporated into a dosage form, for example, one of the above-described dosage forms comprising an effective amount of at least one compound of the invention (e.g., 1, 2 or 3, or 1 or 2, or 1, and usually 1 compound of the invention), or a pharmaceutically acceptable salt thereof. Methods for determining safe and effective administration of compounds which are pharmaceutically active, for example, a compound of the invention, are known to those skilled in the art, for example, as described in the standard literature, for example, as described in the “Physicians' Desk Reference” (PDR), e.g., 1996 edition (Medical Economics Company, Montvale, NJ 07645-1742, USA), the Physician's Desk Reference, 56^{th} Edition, 2002 (published by Medical Economics company, Inc. Montvale, NJ 07645-1742), or the Physician's Desk Reference, 57^{th} Edition, 2003 (published by Thompson PDR, Montvale, NJ 07645-1742); the disclosures of which is incorporated herein by reference thereto. The amount and frequency of administration of the compounds of the invention and/or the pharmaceutically acceptable salts thereof will be regulated according to the judgment of the attending clinician considering such factors as age, condition and size of the patient as well as severity of the symptoms being treated. Compounds of the invention can be administered at a total daily dosage of up to 1,000 mg, which can be administered in one daily dose or can be divided into multiple doses per 24-hour period, for example, two to four doses per day.

As those of ordinary skill in the art will appreciate, an appropriate dosage level for a compound (or compounds) of the invention will generally be about 0.01 to 500 mg per kg patient body weight per day which can be administered in single or multiple doses. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day or may be administered once or twice per day.

Those skilled in the art will appreciate that treatment protocols utilizing at least one compound of the invention can be varied according to the needs of the patient. Thus, compounds of the invention used in the methods of the invention can be administered in variations of the protocols described above. For example, compounds of the invention can be administered discontinuously rather than continuously during a treatment cycle.

In general, in whatever form administered, the dosage form administered will contain an amount of at least one compound of the invention, or a salt thereof, which will provide a therapeutically effective serum level of the compound in some form for a suitable period of time such as at least 2 hours, more preferably at least four hours or longer. In general, as is known in the art, dosages of a pharmaceutical composition providing a therapeutically effective serum level of a compound of the invention can be spaced in time to provide serum level meeting or exceeding the minimum therapeutically effective serum level on a continuous basis throughout the period during which treatment is administered. As will be appreciated the dosage form administered may also be in a form providing an extended-release period for the pharmaceutically active compound which will provide a therapeutic serum level for a longer period, necessitating less frequent dosage intervals. As mentioned above, a composition of the invention can incorporate additional pharmaceutically active components or be administered simultaneously, contemporaneously, or sequentially with other pharmaceutically active agents as may be additionally needed or desired in the course of providing treatment. As will be appreciated, the dosage form administered may also be in a form providing an extended-release period for the pharmaceutically active compound which will provide a therapeutic serum level for a longer period, necessitating less frequent dosage intervals.

### EXAMPLES

The compounds of the invention can be prepared readily according to the following schemes and specific examples, or modifications thereof, using readily available starting materials, reagents, and conventional synthetic procedures. In these reactions, it is also possible to make use of variants which are themselves known to those of ordinary skill in this art but are not mentioned in detail. The general procedures for making the compounds claimed in this invention can be readily understood and appreciated by one skilled in the art from viewing the following Schemes and descriptions.

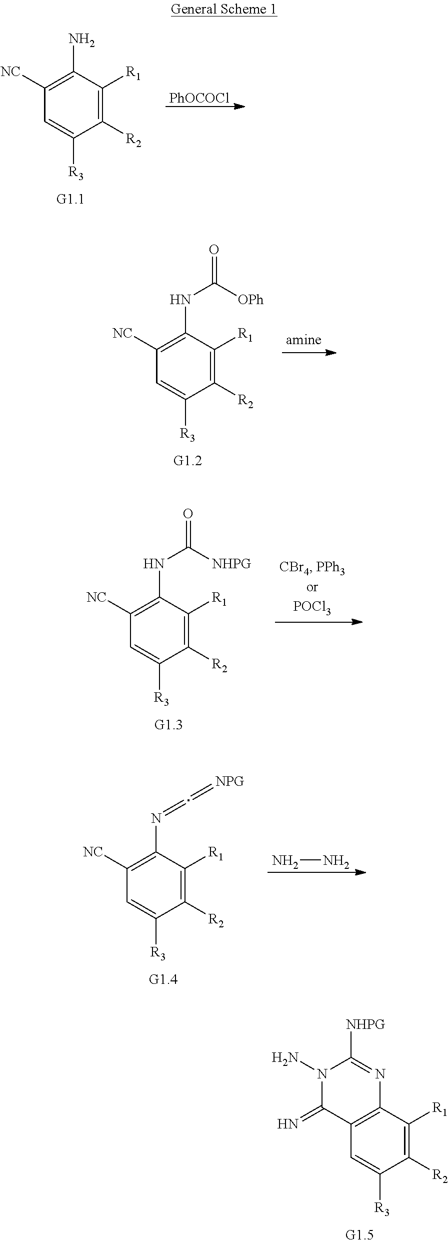

One general strategy for the synthesis of compounds of type G1.5 is via the four-step procedure shown in General Scheme 1, wherein R₁, R₂, and R₃ are as defined in Formula (I) and PG is defined as a protecting group. In the first step, amino benzonitriles G1.1 can be treated with phenyl chloroformate to form carbamate G1.2. In the second step, these carbamates can be reacted with amines to form protected ureas G1.3. In the third step, the protected ureas can be dehydrated using either a combination of tetrabromomethane and triphenylphosphine or phosphorus (V) oxychloride to form carbodiimide G1.4. In the fourth step, the carbodiimide can be reacted with hydrazine to afford products of type G1.5, which can be subsequently purified by silica gel column chromatography, preparative reverse-phase HPLC, and/or SFC.

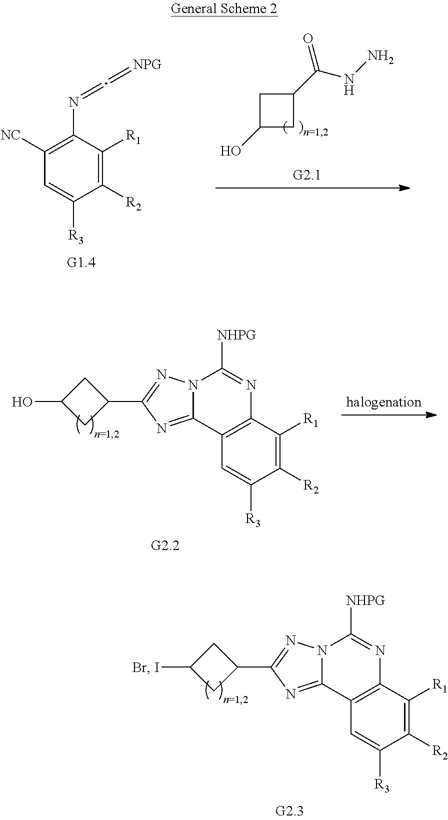

One general strategy for the synthesis of compounds of type G2.3 is via the two-step procedure shown in General Scheme 2, wherein R₁, R₂, R₃, R₅, and R₆ are as defined in Formula (I) and PG is defined as a protecting group. In the first step, carbodiimide G1.4 can react with hydrazide G2.1 using an acid such as acetic acid to form the cyclic alcohol G2.2. In the second step, the cyclic alcohol can be converted to the halogenated cycloalkanes G2.3 using different halogenation conditions such as tetrabromomethane/triphenylphosphine or imidazole/triphenylphosphine/iodide, which can be subsequently purified by silica gel column chromatography, preparative reverse-phase HPLC, and/or SFC.

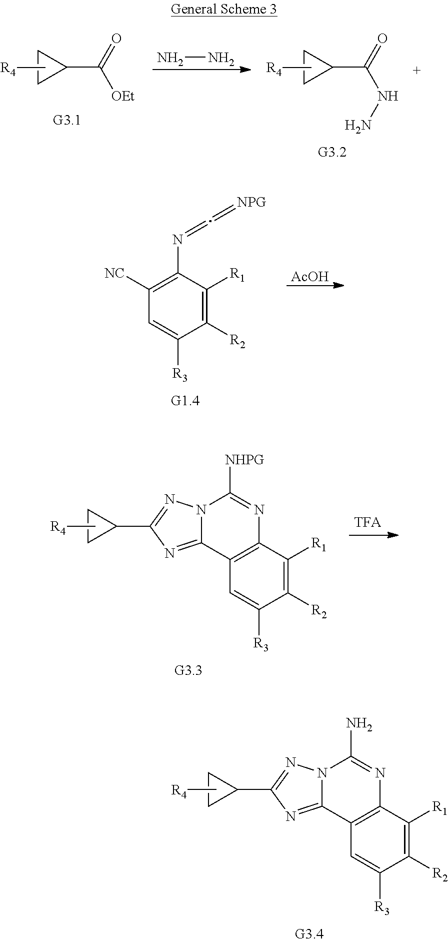

One general strategy for the synthesis of compounds of type G3.4 is via the three-step procedure shown in General Scheme 3, wherein R₁, R₂, R₃, and R₄, are as defined in Formula (I) and PG is defined as a protecting group. In the first step, cyclopropyl ester G3.1 is converted to hydrazide G3.2 using hydrazine. In the second step, hydrazide G3.2 is reacted with carbodiimide G1.4 in acetic acid to form tricycle G3.3. In the third step, G3.3 is deprotected using TFA to afford products of type G3.4, which can be subsequently purified by silica gel column chromatography, preparative reverse-phase HPLC, and/or chiral SFC.

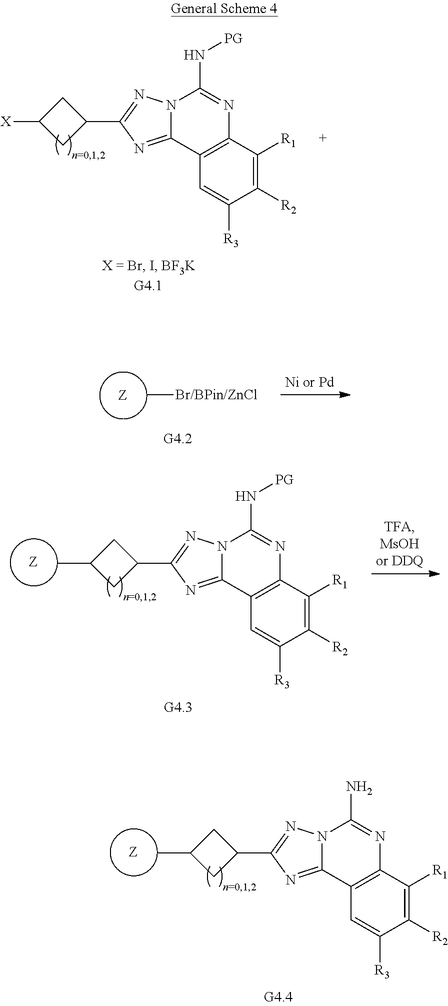

One general strategy for the synthesis of compounds of type G4.4 is via the two-step procedure shown in General Scheme 4, wherein R₁, R₂, and R₃ are defined in Formula (I), and wherein X is defined as either a BF₃K salt, I, or Br, and wherein Z is defined is alkyl, aryl, or heteroaryl, and PG is defined as a protecting group. In the first step, cyclobutane G4.1 is reacted with G4.2 under cross-coupling conditions using either nickel or palladium catalysis to form G4.3. In the second step, G4.3 is deprotected using TFA, MsOH, or DDQ to afford products of type G4.4, which can be subsequently purified by silica gel column chromatography, preparative reverse-phase HPLC, and/or chiral SFC.

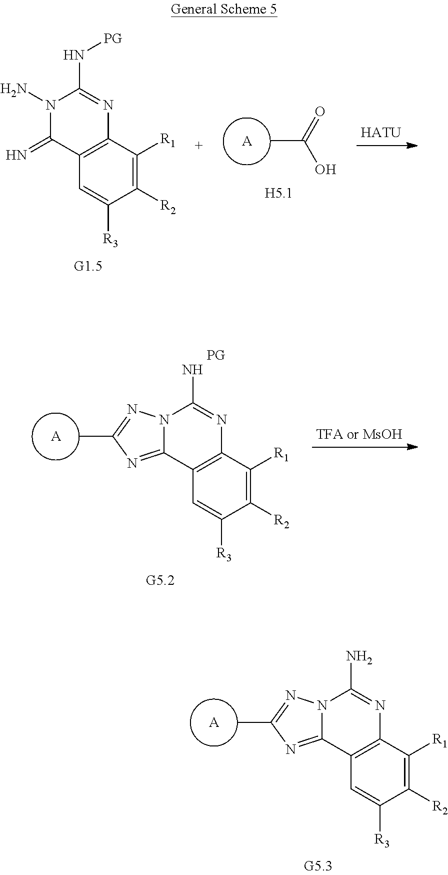

One general strategy for the synthesis of compounds of type G5.3 is via the two-step procedure shown in General Scheme 5, wherein ring A, R₁, R₂, and R₃ are defined in Formula (I), and PG is defined as a protecting group. In the first step, G1.5 is condensed with G5.1 using HATU to form G5.2. In the second step, G5.2 is deprotected using TFA or MsOH to afford products of type G5.3, which can be subsequently purified by silica gel column chromatography, preparative reverse-phase HPLC, and/or chiral SFC.

### EXPERIMENTALS

The following abbreviations may be used in the following experimentals:

| ° C | Degrees Celsius |
|---|---|
| AcOH | Acetic acid |
| aq. | Aqueous |
| BF₃K | Potassium, trifluoroborate |
| Boc-anhydride | Di-tert-butyl dicarbonate |
| CDI | 1,1′-Carbonyldiimidazole |
| COMU | (1-Cyano-2-ethoxy-2- |
| | oxoethylidenaminooxy)dimethylamino- |
| | morpholino-carbenium hexafluorophosphate |
| CPME | Cyclopentyl methyl ether |
| DCE | 1,2-Dichloroethane |
| DCM | Dichloromethane |
| DDQ | 2,3-Dichloro-5,6-dicyano-p-benzoquinone |
| DIEA | N,N-Diisopropylethylamine |
| DIPA | Diisopropylamine |
| DMA | Dimethylacetamide |
| DMAc | Dimethylacetamide |
| DMF | Dimethylformamide |
| DMSO | Dimethyl sulfoxide |
| DMSO-d₆ | Deuterated dimethyl sulfoxide |
| Dppf | Bis(diphenylphosphino)ferrocene |
| ESI | Electrospray Ionization |
| Et₃N | Triethylamine |
| Et₂O | Diethylether |
| EtOAc | Ethyl acetate |
| EtOH | Ethanol |
| H | Hours |
| HATU | 1-[Bis(dimethylamino)methylene]-1H-1,2,3- |
| | triazolo[4,5-b]pyridinium 3-oxid |
| | hexafluorophosphate |
| HCl | Hydrochloric Acid |
| Hex | Hexanes |
| HPLC | High Performance Liquid Chromatography |
| IPA | Isopropyl alcohol |
| KHMDS | Potassium bis(trimethylsilyl)amide |
| KOH | Potassium hydroxide |
| LiOMe | Lithium methoxide |
| M | Molar |
| MeCN | Acetonitrile |
| MeOD-d4 | Deuterated methanol |
| MeOH | Methanol |
| MHz | Megahertz |
| Min | Minutes |
| mL | Milliliters |
| MS | Mass Spectroscopy |
| MsCl | p-Toluenesulfonyl chloride |
| MsOH | Methanesulfonic acid |
| MTBE | Methyl tert-butyl ether |
| NBS | N-Bromosuccinimide |
| Nm | Nanometers |
| NMR | Nuclear Magnetic Resonance |
| Pet. Ether | Petroleum ether |
| PG | Protecting group |
| Prep-HPLC | Preperatory HPLC |
| RBF | Round-bottom flask |
| RT | Room Temperature |
| sat. | Saturated |
| SEM-Cl | 2-(Trimethylsilyl)ethoxymethyl chloride |
| TFA | Trifluoroacetic acid |
| THF | Tetrahydrofuran |
| TMSBr | Bromotrimethylsilane |
| TMSCF₃ | Trimethyl(trifluoromethyl)silane |
| XPhos | 2-dicyclohexylphosphino-2′,4′,6′-triisopropyl- |
| | 1,1′-biphenyl |
| XPhos Pd G2 | 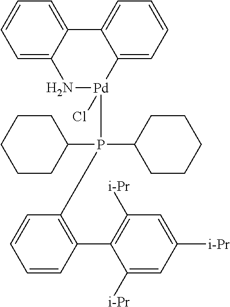 Chloro(2-dicyclohexylphosphino-2′,4′,6′- triisopropyl-1,1′-bipheny1)[2-(2′-amino-1,1′- biphenyl)]palladium(II) CAS# 1310584-14-5 |

### General Experimental Information

Unless otherwise noted, all reactions were magnetically stirred and performed under an inert atmosphere such as nitrogen or argon.

Unless otherwise noted, diethyl ether used in the experiments described below was Fisher ACS certified material and stabilized with BHT.

Phase separator refers to ISOLUTE® Phase Separators from Biotage which are used to separate aqueous and chlorinated solvents during liquid-liquid extraction.

Unless otherwise noted, “degassed” refers to a solvent from which oxygen has been removed, generally by bubbling an inert gas such as nitrogen or argon through the solution for 10 to 15 minutes with an outlet needle to normalize pressure.

Unless otherwise noted, “concentrated” means evaporating the solvent from a solution or mixture using a rotary evaporator or vacuum pump.

Unless otherwise noted, silica gel chromatography was carried out on an ISCO®, Analogix®, or Biotage® automated chromatography system using a commercially available cartridge as the column. Columns were usually filled with silica gel as the stationary phase. Reverse phase preparative HPLC conditions can be found below. Aqueous solutions were concentrated on a Genevac® evaporator or were lyophilized.

Unless otherwise noted, proton nuclear magnetic resonance (¹H NMR) spectra and proton-decoupled carbon nuclear magnetic resonance (¹³C{¹H} NMR) spectra were recorded on 400, 500, or 600 MHz Bruker or Varian NMR spectrometers at ambient temperature. All chemical shifts (8) were reported in parts per million (ppm). Proton resonances were referenced to residual protium in the NMR solvent, which can include, but is not limited to, CDCl₃, DMSO-d₆, and MeOD-d₄. Carbon resonances are referenced to the carbon resonances of the NMR solvent. Data are represented as follows: chemical shift, multiplicity (br=broad, br s=broad singlet, s=singlet, d=doublet, dd=doublet of doublets, ddd=doublet of doublet of doublets, t=triplet, q=quartet, m=multiplet), coupling constants (J) in Hertz (Hz), integration.

### Preparative Reversed-Phase HPLC-MSD Conditions: TFA (Acidic):

### Chromatography and Mass Spectrometry:

Isolation of compound from the reaction mixture was carried out under reverse-phase purification using an Agilent 1200 HPLC-MSD system consisting of a 6130B single quadrupole mass-selective detector (MSD), G1315B diode array detector (DAD), G2258A autosampler, two G1361A preparative pumps, one G1379A quaternary pump with degasser, one G1312A binary pump, and three G1364B fraction collectors from Agilent Technologies (Agilent Technologies, Palo Alto, CA). System control and data analysis were performed using Agilent's ChemStation software, revision B.04.03. A Waters SunFire C18 OBD Prep Column, 100A, 5 μm, 19 mm×150 mm column was used as the stationary phase (Waters Corporation, Milford, MA, USA). Gradient elution was carried out using water (solvent A) and acetonitrile (solvent B) as a mobile phase. A 10% trifluoroacetic acid solution was added into the mobile phase as a modifier using a static mixer prior to the column, pumped at 1% of the total mobile phase flowrate. Electrospray (ESI) Mass-triggered fraction collected was employed using positive ion polarity scanning to monitor for the target mass.

### Preparative Reversed Phase HPLC-MSD Conditions: NH₄OH (Basic):

### Chromatography and Mass Spectrometry:

Isolation of compound from the reaction mixture was carried out under reverse-phase purification using an Agilent 1200 HPLC-MSD system consisting of a 6130B single quadrupole mass-selective detector (MSD), G1315B diode array detector (DAD), G2258A autosampler, two G1361A preparative pumps, one G1379A quaternary pump with degasser, one G1312A binary pump, and three G1364B fraction collectors from Agilent Technologies (Agilent Technologies, Palo Alto, CA). System control and data analysis were performed using Agilent's ChemStation software, revision B.04.03. A Waters XBridge C18 OBD Prep Column, 100A, 5 μm, 19 mm×150 mm column was used as the stationary phase (Waters Corporation, Milford, MA, USA). Gradient elution was carried out using water (solvent A) and acetonitrile (solvent B) as a mobile phase. A 10% ammonium hydroxide solution was added into the mobile phase as a modifier using a static mixer prior to the column, pumped at 1% of the total mobile phase flowrate. Electrospray (ESI) Mass-triggered fraction collected was employed using positive ion polarity scanning to monitor for the target mass.

### Intermediates

### 2-(3-Bromocyclobutyl)-N-(2,4-dimethoxybenzyl)-7-methoxy-[1,2,4]triazolo[1,5-c]quinazolin-5-amine (Int-1)

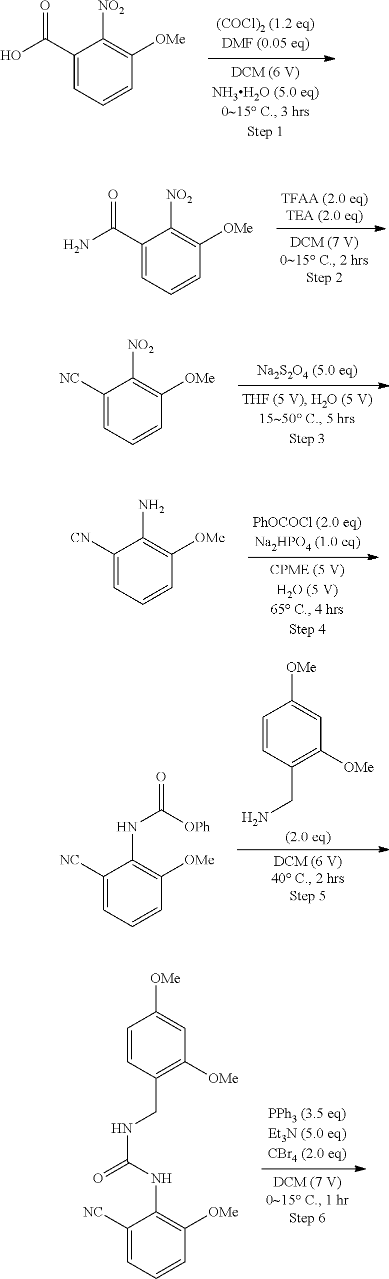

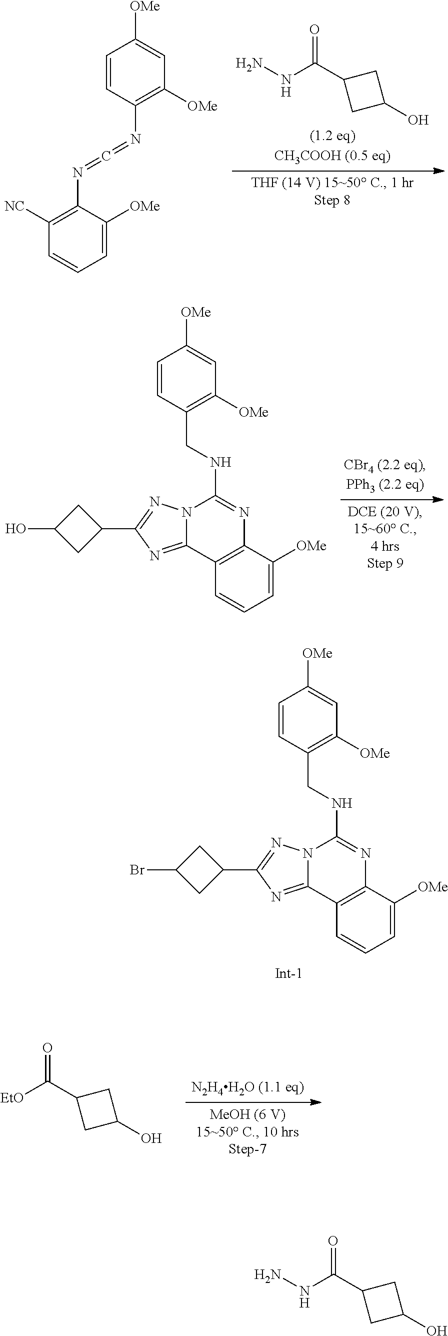 [SMILES: COc1ccc(N=C=Nc2c(C#N)cccc2OC)c(OC)c1 ; NNC(=O)C1CC(O)C1 ; COc1ccc(CNc2nc3c(OC)cccc3c3nc(C4CC(O)C4)nn23)c(OC)c1 ; COc1ccc(CNc2nc3c(OC)cccc3c3nc(C4CC(Br)C4)nn23)c(OC)c1 ; CCOC(=O)C1CC(O)C1 ; NNC(=O)C1CC(O)C1]

Step 1: Two reactions were run in parallel. 3-Methoxy-2-nitrobenzoic acid (375 g, 1.90 mol, 1.0 equiv) was added to DCM (2.0 L) at 15° C. and stirred. Oxalyl chloride (289 g, 2.28 mmol, 1.2 equiv) and DMF (6.95 g, 0.095 mol, 0.05 equiv) were added and the reaction was stirred at 15° C. for 3 hours. NH₃·H₂O (1.33 kg, 9.51 mol, 25% purity, 5.0 equiv.) was added dropwise over 12 minutes at 0° C. The reaction was stirred at 15° C. for 1 hour. The two reactions were combined and filtered. The filter cake was washed with water (500 mL×3) and triturated with MTBE (3.0 L) at 20° C. for 30 minutes. The solid was filtered and dried under vacuum to afford 3-methoxy-2-nitrobenzamide.

Step 2: Two reactions were run in parallel. 3-Methoxy-2-nitrobenzamide (350 g, 1.12 mol, 1.0 equiv) and triethylamine (4% mL, 3.57 mol, 2.0 equiv) were added to DCM (2.3 L) at 15° C. Trifluoroacetic anhydride (4% mL, 3.57 mol, 2.0 equiv) was added and the reaction stirred at 15° C. for 2 hours. The two reactions were combined and quenched with saturated NaHCO₃ (5.0 L) at 15° C., followed by extraction with ethyl acetate (2.0 L×2). The organic layer was washed with brine (1.0 L), dried over anhydrous Na₂SO₄, filtered, and concentrated under reduced pressure, to afford 3-methoxy-2-nitrobenzonitrile.

Step 3: Two reactions were performed in parallel. 3-Methoxy-2-nitrobenzonitrile (300 g, 1.68 mol, 1.0 equiv) was taken up in THF (1.5 L) and water (1.5 L) at 15° C. Na₂S₂O₄ (1.5 kg, 8.62 mol, 5.0 equiv) was added to the reaction in portions over 4 hours at 40° C. and the reaction was stirred at 50° C. for 2 hours. The two reactions were combined, quenched with water (2.0 L), and extracted with ethyl acetate (2.0 L×3). The organic layer was washed with brine (2.0 L), dried over anhydrous Na₂SO₄, filtered, and concentrated under reduced pressure to afford 2-amino-3-methoxybenzonitrile.

Step 4: Two reactions were performed in parallel. 2-Amino-3-methoxybenzonitrile (200 g, 1.35 mol, 1.0 equiv), water (1.0 L), and sodium phosphate dibasic (191 g, 1.35 mol, 1.0 equiv) were added to CPME (1.0 L) at 15° C. Phenyl chloroformate (338 mL, 2.7 mol, 2.0 equiv) was added to the mixture at 65° C. over 1.5 hours and the reaction stirred at 60° C. for 0.5 hours. The reaction was cooled to 15° C. and the two reactions were combined. The reaction mixture was filtered and solid was collected. The filtrate was concentrated under reduced pressure and was combined with the solid to afford phenyl (2-cyano-6-methoxyphenyl)carbamate which was used in the next step without further purification.

Step 5: Two reactions were performed in parallel. (2-Cyano-6-methoxyphenyl)carbamate (250 g, 931 mmol, 1.0 equiv) was added to DCM (1.7 L) at 15° C. 2,4-Dimethoxylbenzylamine (280 mL, 1860 mmol, 2.0 equiv) was added to the reaction mixture at 45° C. and the reaction was stirred at 45° C. for 3 hours. The two reactions were combined at 15° C. and were filtered. The filtrate was concentrated under reduced pressure and was combined with the solid to afford 1-(2-cyano-6-methoxyphenyl)-3-(2,4-dimethoxybenzyl)urea.

Step 6: 1-(2-Cyano-6-methoxyphenyl)-3-(2,4-dimethoxybenzyl)urea (500 g, 1.46 mol, 1.0 equiv), triphenylphosphine (1.34 kg, 5.13 mol, 3.5 equiv), and triethylamine (1.02 L, 7.32 mol, 5.0 equiv) were added to DCM (3.3 L) at 20° C. Carbon tetrabromide (971 g, 2.93 mol, 2.0 equiv) was added to the reaction at 0° C. in portions over 1 hour. The reaction was stirred at 0° C. for 1 hour. The reaction was filtered, and the filtrate was concentrated under reduced pressure to give a residue. The residue was purified by silica gel column chromatography (100:1 to 1:1 pet. ether:EtOAc) to afford 2-((((2,4-dimethoxybenzyl)imino)methylene)amino)-3-methoxybenzonitrile.

Step 7: Ethyl 3-hydroxycyclobutane-1-carboxylate (160 g, 1.11 mol, 1.0 equiv) was added to MeOH (950 mL) at 15° C. Hydrazine hydrate (83.4 mL, 1.68 mol, 1.5 equiv) was added and reaction stirred at 50° C. for 12 hours. The reaction was concentrated under reduced pressure to afford 3-hydroxycyclobutane-1-carbohydrazide.

Step 8: 3-Hydroxycyclobutane-1-carbohydrazide (74.3 g, 571 mmol, 1.2 equiv) and acetic acid (13.6 mL, 238 mmol, 0.5 equiv) were taken up in THF (1.2 L). 2-((((2,4-dimethoxybenzyl)imino)methylene)amino)-3-methoxybenzonitrile (200 g, 476 mmol, 77% purity, 1.0 equiv) in THF (1.2 L) was added dropwise to the solution over 30 minutes. The reaction was stirred at 50° C. for 2 hours. Saturated NaHCO₃ (800 mL) was added to the reaction at 15° C. and extracted with ethyl acetate (500 mL×3). The organic layer was washed with brine (500 mL), dried over anhydrous Na₂SO₄, filtered, and concentrated under reduced pressure to afford a residue. The residue was purified by silica gel column chromatography (100:1 to 1:1 pet. ether:EtOAc) to afford 3-(5-((2,4-dimethoxybenzyl)amino)-7-methoxy-[1,2,4]triazolo[1,5-c]quinazolin-2-yl)cyclobutan-1-ol.

Step 9: 3-(5-((2,4-Dimethoxybenzyl)amino)-7-methoxy-[1,2,4]triazolo[1,5-c]quinazolin-2-yl)cyclobutan-1-ol (100 g, 229 mmol, 1.0 equiv) was taken up in DCE (2.0 L). Carbon tetrabromide (167 g, 505 mmol, 2.2 equiv) and triphenylphosphine (132 g, 505 mmol, 2.2 equiv) were added and the reaction mixture was heated at 60° C. for 4 hours. The reaction was then cooled to 25° C., was poured into a saturated solution of Na₂SO₃ (1.5 L), and the organic layer was separated. The organic layer was washed with brine and concentrated under reduced pressure to give a residue. The residue was purified by prep-HPLC (silica gel column Phenomenex luna C18 (250*70 mm, 15 um); mobile phase: [water (0.1% TFA)—MeCN]; B %: 50%-78%, 20 minutes). The solution was adjusted to pH 7-8 with aqueous NaHCO₃ solution and concentrated under reduced pressure to remove the MeCN. The aqueous layer was extracted with DCM (1.0 L then 500 mL) and concentrated under reduced pressure to afford 2-(3-bromocyclobutyl)-N-(2,4-dimethoxybenzyl)-7-methoxy-[1,2,4]triazolo[1,5-c]quinazolin-5-amine (Int-111111111). ESI MS m/z=498/500 [M+H]⁺

### N-(2,4-Dimethoxybenzyl)-2-(3-iodocyclobutyl)-7-methoxy-[1,2,4]triazolo[1,5-c]quinazolin-5-amine (Int-2)

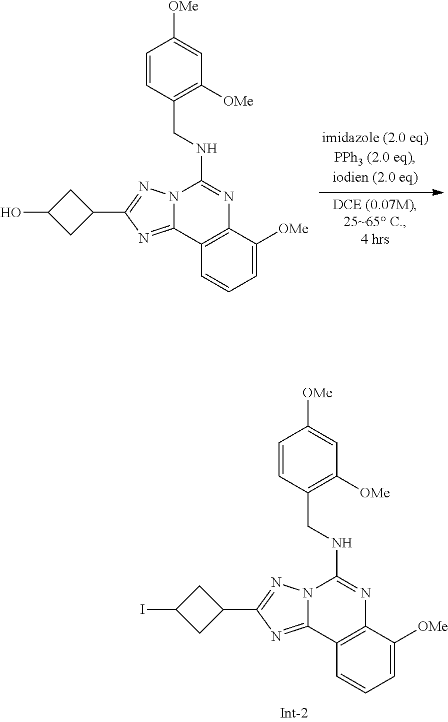 [SMILES: COc1ccc(CNc2nc3c(OC)cccc3c3nc(C4CC(O)C4)nn23)c(OC)c1 ; COc1ccc(CNc2nc3c(OC)cccc3c3nc(C4CC(I)C4)nn23)c(OC)c1]

A stirring suspension of 3-(5-((2,4-dimethoxybenzyl)amino)-7-methoxy-[1,2,4]triazolo[1,5-c]quinazolin-2-yl)cyclobutan-1-ol (12.0 g, 27.6 mmol, 1.0 equiv), triphenylphosphine (14.5 g, 55.1 mmol, 2.0 equiv) and imidazole (3.75 g, 55.1 mmol, 2.0 equiv) in DCE (410 mL) was treated with iodine (14.0 g, 55.1 mmol, 2.0 equiv). The resulting mixture was heated at 65° C. and stirred for 16 hours. After cooling, the reaction was quenched with sat. aq. Na₂S₂O₃ (200 mL), and the biphasic mixture was stirred for 20 min at 25° C. Water (200 mL) was added, the layers were separated, and the aqueous layer was extracted with DCM (150 mL×2). The combined organic layers were dried over anhydrous MgSO₄, filtered and concentrated to a crude solid. This crude material was taken up in a mixture of MeOH (100 mL), EtOAc (100 mL) and acetone (200 mL), heated to reflux and then slowly cooled to 4° C. and allowed to stand at 4° C. for 16 hours until significant precipitate was observed. The mixture was then filtered, rinsing with acetone (50 mL), and the filter cake was dried to afford N-(2,4-dimethoxybenzyl)-2-(3-iodocyclobutyl)-7-methoxy-[1,2,4]triazolo[1,5-c]quinazolin-5-amine (Int-2). ESI MS m/z=546 [M+H]⁺

### 3-(5-((2,4-Dimethoxybenzyl)amino)-7-methoxy-[1,2,4]triazolo[1,5-c]quinazolin-2-yl)cyclobutan-1-one (Int-3)

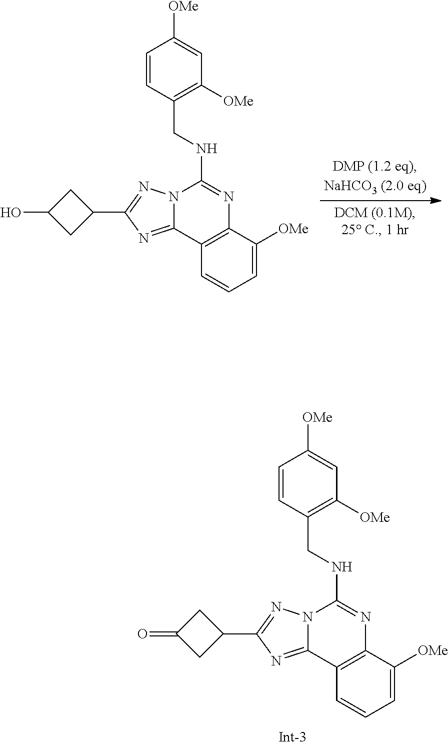 [SMILES: COc1ccc(CNc2nc3c(OC)cccc3c3nc(C4CC(O)C4)nn23)c(OC)c1 ; COc1ccc(CNc2nc3c(OC)cccc3c3nc(C4CC(=O)C4)nn23)c(OC)c1]

A 50 mL round bottom flask was charged with 3-(5-((2,4-dimethoxybenzyl)amino)-7-methoxy-[1,2,4]triazolo[1,5-c]quinazolin-2-yl)cyclobutan-1-ol (400 mg, 0.919 mmol, 1.0 equiv). DCM (9.2 mL) was added, followed by NaHCO₃ (154 mg, 1.84 mmol, 2.0 equiv) and Dess-Martin periodinane (468 mg, 1.10 mmol, 1.2 equiv). The resulting mixture was stirred for 1 hour at 25° C. under an atmosphere of air. The reaction was then quenched with sat. aq. NaHCO₃ (7 mL) and sat. aq. Na₂S₂O₃ (7 mL), and the biphasic mixture was stirred for 15 min at 25° C. The layers were then separated, and the aq. layer was extracted with DCM (3×15 mL). The combined organic layers were dried over anhydrous MgSO₄, filtered and concentrated under reduced pressure. The crude residue was purified by silica gel column chromatography (0-100% EtOAc in hex), to provide 3-(5-((2,4-dimethoxybenzyl)amino)-7-methoxy-[1,2,4]triazolo[1,5-c]quinazolin-2-yl)cyclobutan-1-one (Int-3). ESI MS m/z=434 [M+H]⁺

### N-(2,4-dimethoxybenzyl)-2-(3-iodocyclobutyl)-8-methoxy-[1,2,4]triazolo[1,5-c]quinazolin-5-amine (Int-4)

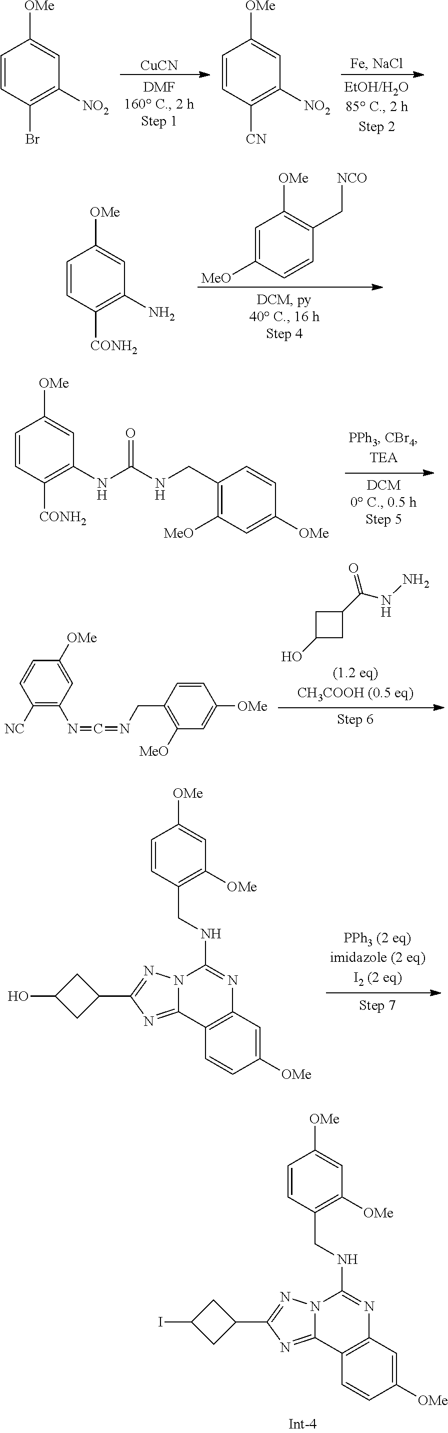

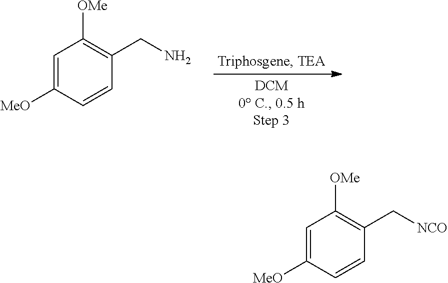

Step 1: Two reactions were carried out in parallel. 1-bromo-4-methoxy-2-nitrobenzene (450 g, 1.94 mol, 1.0 equiv) was taken up in DMF (2200 mL) and CuCN (261 g, 2.91 mol, 1.5 equiv) was added into the mixture. The mixture was stirred at 160° C. for 2 hours. The two batches were combined and poured into 800 mL of water, then filtered to give a residue. The residue was washed with DCM (5000 mL×5) and concentrated under reduced pressure to afford 4-methoxy-2-nitrobenzonitrile and was used in the next step without further purification.

Step 2: Two reactions were performed in parallel. 4-methoxy-2-nitrobenzonitrile (235 g, 1.32 mol, 1.0 equiv), NaCl (145 g, 2.48 mol, 1.88 equiv), and water (1100 mL) were added to EtOH (1100 mL). Iron (332 g, 5.94 mol, 4.5 equiv) was added to the mixture at 70° C. and the mixture was stirred at 85° C. for 2 hours. The two batches were combined, filtered, and the filtrate was concentrated. The remaining aqueous mixture was extracted with EtOAc (1000 mL×2) and the organic phase was concentrated under reduced pressure to afford 2-amino-4-methoxybenzamide and was used in the next step without further purification.

Step 3: Three reactions were performed in parallel. To a mixture of triphosgene (474 g, 160 mol, 1.0 equiv) in DCM (2600 mL) was added 2,4-dimethoxybenzylamine (267 g, 1.6 mol, 1.0 equiv) in DCM (800 mL) at 0° C. Triethylamine (582 g, 5.75 mol, 3.6 equiv) was then added at 0° C. and the mixture was stirred at 0° C. for 30 minutes. The mixture was filtered, and the filtrate was concentrated under reduced pressure. The residue was purified by silica gel column chromatography (0-10% EtOAc in pet. ether) to afford 1-(isocyanatomethyl)-2,4-dimethoxybenzene.

Step 4: Two reactions were performed in parallel. 2-amino-4-methoxybenzamide (130 g, 782 mmol, 1.0 equiv) was added to DCM (900 mL) followed by pyridine (127 g, 1.61 mol, 2.06 equiv) then 1-(isocyanatomethyl)-2,4-dimethoxybenzene (272 g, 1.41 mol, 1.8 equiv). The mixture was stirred at 40° C. for 16 hours. The reactions were combined and filtered. The filter cake was collected to afford 2-(3-(2,4-dimethoxybenzyl)ureido)-4-methoxybenzamide.

Step 5: Three reactions were run in parallel. 2-(3-(2,4-dimethoxybenzyl)ureido)-4-methoxybenzamide (100 g, 293 mmol, 1 equiv), triphenylphosphine (231 g, 879 mmol, 3.0 equiv), and triethylamine (178 g, 1.76 mol, 6.0 equiv) were taken up in DCM (3000 mL). Tetrabromomethane (291 g, 879 mmol, 3.0 equiv) in DCM (500 mL) was then added dropwise at 0° C. over 30 minutes. The reaction was concentrated under reduced pressure and purified by silica gel column chromatography (pet. ether:EtOAc:DCM=5:1:1) followed by a second purification (pet. ether:EtOAc 20:1 to 5:1) to afford 2-((((2,4-dimethoxybenzyl)imino)methylene)amino)-4-methoxybenzonitrile.

Step 6: 2-((((2,4-dimethoxybenzyl)imino)methylene)amino)-4-methoxybenzonitrile (1.5 g, 4.8 mmol, 1.0 equiv) and 3-hydroxycyclobutane-1-carbohydrazide (750 mg, 5.76 mmol, 1.2 equiv) were taken up in 1,4-dioxane (16 mL) and acetic acid (137 μL, 2.4 mmol, 0.5 equiv) was added. The reaction mixture was stirred at 70° C. for 16 hours. The reaction mixture was concentrated under reduced pressure and purified by silica gel column chromatography (0-100% EtOAc in hex) to afford 3-(5-((2,4-dimethoxybenzyl)amino)-8-methoxy-[1,2,4]triazolo[1,5-c]quinazolin-2-yl)cyclobutan-1-ol.

Step 7: Iodine (1.03 g, 4.07 mmol, 2.0 equiv) was added to a suspension of 3-(5-((2,4-dimethoxybenzyl)amino)-8-methoxy-[1,2,4]triazolo[1,5-c]quinazolin-2-yl)cyclobutan-1-ol (886 mg, 2.04 mmol, 1.0 equiv), triphenylphosphine (1.07 g, 4.07 mmol, 2.0 equiv), and imidazole (277 mg, 4.07 mmol, 2.0 equiv) in DCM (30 mL). The mixture was stirred at 65° C. for 16 hours. The reaction was quenched with sodium thiosulfate (40 mL), followed by the addition of DCM (20 mL) and biphasic mixture was stirred at room temperature for 10 minutes. The layers were separated and the aqueous layer was extracted with DCM (50 mL×2). The combined organic layers were dried over anhydrous MgSO₄ and concentrated under reduced pressure. The crude residue was purified by silica gel column chromatography (0-100% EtOAc in hex) to afford N-(2,4-dimethoxybenzyl)-2-(3-iodocyclobutyl)-8-methoxy-[1,2,4]triazolo[1,5-c]quinazolin-5-amine (Int-4). ESI MS m/z=546 [M+H]⁺

### 3-(5-((2,4-dimethoxybenzyl)amino)-9-fluoro-7-methoxy-[1,2,4]triazolo[1,5-c]quinazolin-2-yl)cyclobutan-1-ol (Int-5)

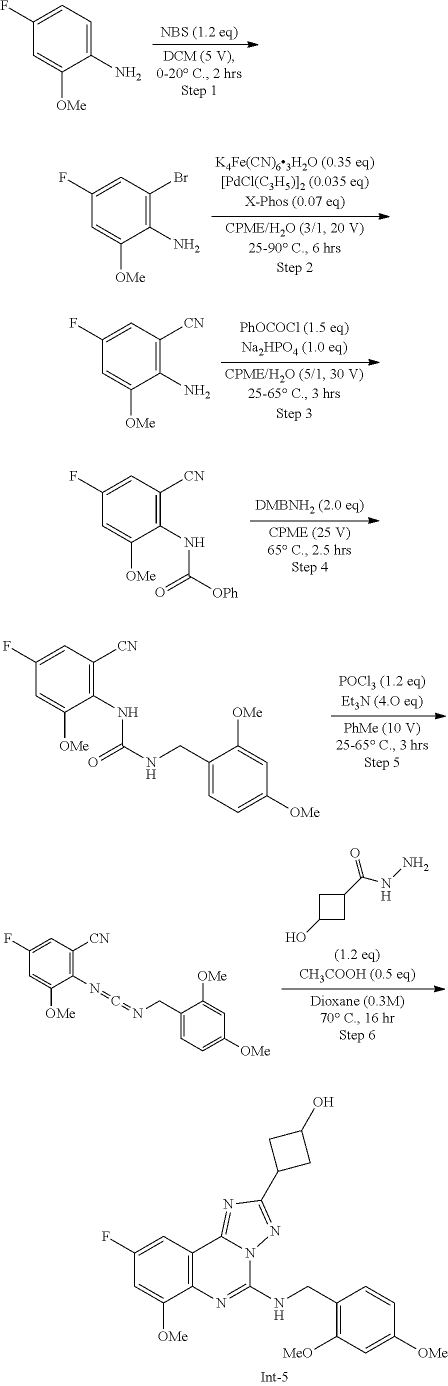

Step 1: Two reactions were run in parallel. 4-Fluoro-2-methoxyaniline (200 g, 1.4 mol, 1.0 equiv) was taken up in DCM (1.0 L). NBS (302 g, 1.7 mol, 1.2 equiv) was added to the reaction mixture at 0° C. and the reaction was stirred at 20° C. for 2 hours. The two reactions were combined, filtered, and concentrated under reduced pressure to afford 2-bromo-4-fluoro-6-methoxyaniline.

Step 2: Two reactions were run in parallel. XPhos (8.34 g, 17.5 mmol, 0.07 equiv) and [PdCl(C₃H₅)]₂ (3.2 g, 8.7 mmol, 0.035 equiv) were added to CPME (825 mL) and the mixture was stirred at 25° C. for 0.5 hours. 2-bromo-4-fluoro-6-methoxyaniline (55 g, 249 mmol, 1.0 equiv) was added to the reaction and was stirred at 25° C. for 0.5 hours. K₄Fe(CN)₆·3H₂O (36.9 g, 87.4 mmol, 0.35 equiv) in water (275 mL) was added to the reaction mixture and was stirred at 90° C. for 5 hours. The reaction mixture was cooled to 25° C. and the two reactions were combined. The reaction mixture was filtered through Celite™ and washed with CPME (300 mL). The organic layer was separated to obtain 2-amino-5-fluoro-3-methoxybenzonitrile as a solution in CPME which was used in the next step without further purification.

Step 3: 2-Amino-5-fluoro-3-methoxybenzonitrile (83 g, 499 mmol, 1.0 equiv) in CPME (2075 mL) was added to a reactor, followed by the addition of water (415 mL). Sodium phosphate dibasic (70.9 g, 499 mmol, 1.0 equiv) was added and the reaction mixture was stirred at 65° C. Phenyl chloroformate (93.8 mL, 740 mmol, 1.5 equiv) was added over 0.5 hours and stirred at 65° C. for 2.5 hours. The reaction mixture was cooled to 25° C., filtered with washing with CPME (200 mL), and the organic layer was separated to afford phenyl (2-cyano-4-fluoro-6-methoxyphenyl)carbamate as a solution in CPME and was used in the next step without further purification.

Step 4: Phenyl (2-cyano-4-fluoro-6-methoxyphenyl)carbamate (143 g, 499 mmol, 1.0 equiv) in CPME (3575 mL) was added to a reactor under N₂. 2,4-Dimethoxylbenzylamine (150 mL, 999 mmol, 2.0 equiv) was added to the reactor at 65° C. and was stirred at 65° C. for 2.5 hours. The reaction was filtered and was washed with EtOAc (1.0 L). The solution was concentrated to afford 1-(2-cyano-4-fluoro-6-methoxyphenyl)-3-(2,4-dimethoxybenzyl)urea.

Step 5: Triethylamine (154 mL, 1.11 mol, 4.0 equiv) was added to 1-(2-Cyano-4-fluoro-6-methoxyphenyl)-3-(2,4-dimethoxybenzyl)urea (100 g, 278 mmol, 1 equiv) in toluene (1.0 L), followed by phosphorus (V) oxychloride (31 mL, 333 mmol, 1.2 equiv). The reaction mixture was stirred and heated at 65° C. for 3 hours, then cooled to 0° C. The reaction mixture was filtered through Celite™ and was washed with toluene (200 mL). The organic layer was isolated and concentrated under reduced pressure to afford 2-((((2,4-dimethoxybenzyl)imino)methylene)amino)-5-fluoro-3-methoxybenzonitrile.

Step 6: 3-Hydroxycyclobutane-1-carbohydrazide (750 mg, 5.76 mmol, 1.2 equiv) and 2-((((2,4-dimethoxybenzyl)imino)methylene)amino)-5-fluoro-3-methoxybenzonitrile (1.64 g, 4.80 mmol, 1.0 equiv) were taken up in 1,4-dioxane (16 mL) and acetic acid (137 μL, 2.40 mmol) and the reaction mixture was heated at 70° C. and stirred for 16 hours. After cooling, the reaction mixture was directly purified by silica gel chromatography (0-100% EtOAc in hexanes) to 3-(5-((2,4-dimethoxybenzyl)amino)-9-fluoro-7-methoxy-[1,2,4]triazolo[1,5-c]quinazolin-2-yl)cyclobutan-1-ol (Int-S). ESI MS m/z=454 [M+H]⁺

### N-(2,4-dimethoxybenzyl)-9-fluoro-2-(3-iodocyclobutyl)-7-methoxy-[1,2,4]triazolo[1,5-c]quinazolin-5-amine (Int-6)

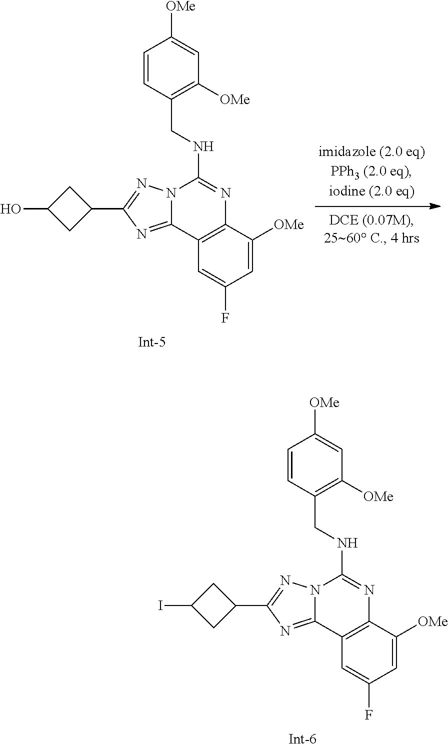 [SMILES: COc1ccc(CNc2nc3c(OC)cc(F)cc3c3nc(C4CC(O)C4)nn23)c(OC)c1 ; COc1ccc(CNc2nc3c(OC)cc(F)cc3c3nc(C4CC(I)C4)nn23)c(OC)c1]

A stirring suspension of 3-(5-((2,4-dimethoxybenzyl)amino)-9-fluoro-7-methoxy-[1,2,4]triazolo[1,5-c]quinazolin-2-yl)cyclobutan-1-ol (Int-5) (658 mg g, 1.45 mmol, 1.0 equiv), triphenylphosphine (761 mg, 2.90 mmol, 2.0 equiv) and imidazole (198 mg, 2.90 mmol, 2.0 equiv) in DCE (29 mL) was treated with iodine (737 g, 2.90 mmol, 2.0 equiv). The resulting mixture was heated at 65° C. and stirred for 16 hours. After cooling, the reaction was quenched with sat. aq. Na₂S₂O₃ (40 mL), and the biphasic mixture was stirred for 20 min at 25° C. The aqueous layer was extracted with DCM (50 mL×2). The combined organic layers were dried over anhydrous MgSO₄, filtered and concentrated to a crude residue. This crude residue was purified by silica gel column chromatography (0-100% EtOAc in hexanes) to afford N-(2,4-dimethoxybenzyl)-9-fluoro-2-(3-iodocyclobutyl)-7-methoxy-[1,2,4]triazolo[1,5-c]quinazolin-5-amine (Int-6). ESI MS m/z=564 [M+H]⁺

### N-(tert-butyl)-7-methoxy-2-((1r,3r)-3-(trifluoro-l4-boranyl)cyclobutyl)-[1,2,4]triazolo[1,5-c]quinazolin-5-amine, potassium salt (Int-7)

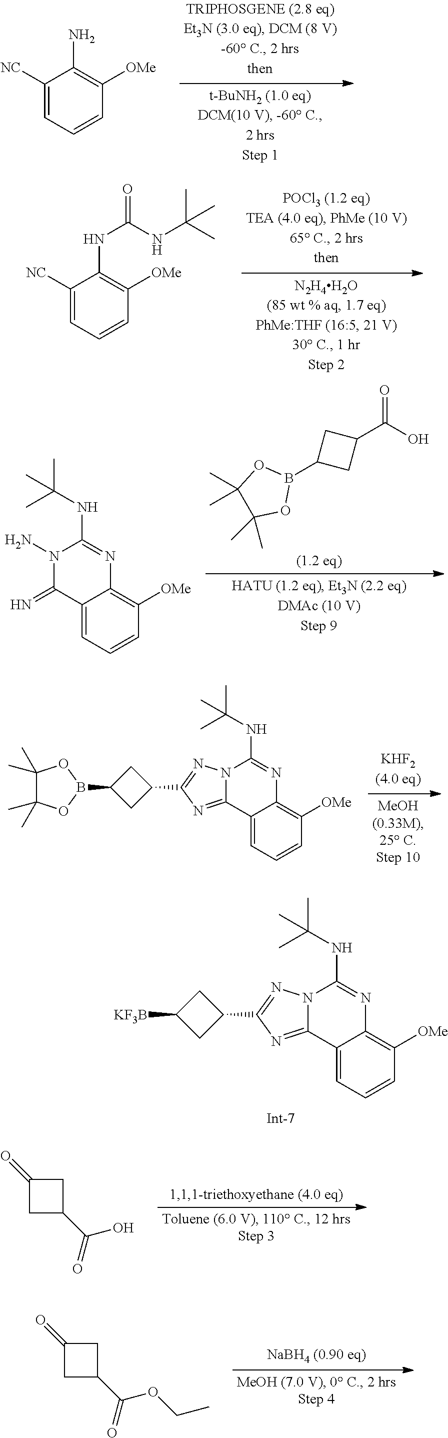

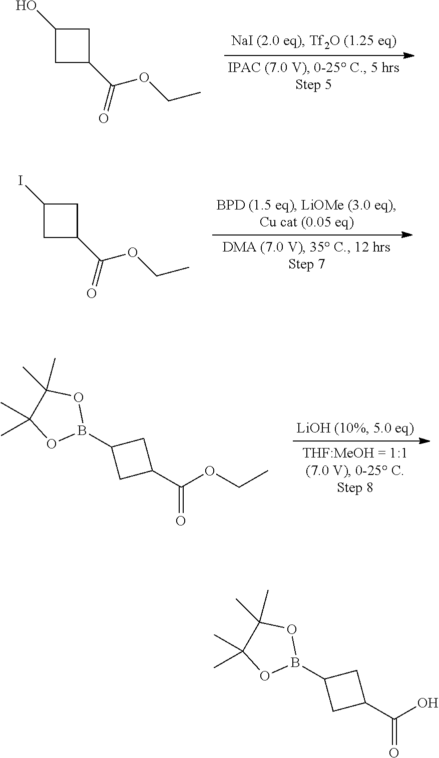

Step 1: Triphosgene (105 g, 0.35 mol, 0.35 equiv) was taken up in DCM (600 mL) at 15 TC. The solution was purged and bubbled with N₂ 3 times. 2-amino-3-methoxybenzonitrile (150 g, 1.01 mol, 1.0 equiv) in DCM (600 mL) at −60° C. was added to the solution and the reaction was stirred at that temperature for 2 hours. tert-Butylamine (74.1 g, 1.01 mol, 1.0 equiv) was added dropwise at −60° C. and reaction was stirred at that temperature for 2 hours. The reaction mixture was concentrated under reduced pressure and diluted with water (600 mL), then extracted with DCM (500 mL×2). The organic layer was collected and concentrated under reduced pressure to afford 1-(tert-butyl)-3-(2-cyano-6-methoxyphenyl)urea and was used in the next step without further purification.

Step 2: Triethylamine (98.2 g, 485 mmol, 4.0 equiv) was added to a solution of 1-(tert-butyl)-3-(2-cyano-6-methoxyphenyl)urea (60.0 g, 261 mmol, 1.0 equiv) in toluene (600 mL) followed by phosphorus (V) oxychloride (44.6 g, 291 mmol, 1.2 equiv) and the reaction was stirred at 65° C. for 2 hours. The reaction was filtered and the filtrate was washed with brine (600 mL). The organic layer was dried over anhydrous Na₂SO₄ and was diluted with THF (300 mL). Hydrazine hydrate (15 mL, 313 mmol, 98% purity, 1.2 equiv) was added dropwise to this mixture at 25° C. and stirred at 25° C. for 1 hour. The reaction mixture was washed with brine (300 mL×2) and the organic layer was concentrated under reduced pressure to afford N²-(tert-butyl)-4-imino-8-methoxyquinazoline-2,3(4H)-diamine.

Step 3: Two reactions were performed in parallel. 1,1,1-Triethoxyethane (642 mL, 3.51 mol, 4.0 equiv) and 3-oxocyclobutane-1-carboxylic acid (100 g, 876 mmol, 1.0 equiv) were taken up in toluene (600 mL) and purged with N₂ 3 times. The reaction mixture was heated at 110° C. for 2 hours and then cooled to 25° C. The two reactions were combined and the organic layer washed with 1M HCl (500 mL), then saturated NaHCO₃ (500 mL), then brine (500 mL). The organic layer was concentrated under reduced pressure to afford ethyl 3-oxocyclobutane-1-carboxylate and was used in the next step without further purification.

Step 4: Two reactions were performed in parallel. Ethyl 3-oxocyclobutane-1-carboxylate (100 g, 703 mmol, 1.0 equiv) was taken up in MeOH (700 mL), cooled to 0° C., and was purged with N₂ 3 times. Sodium borohydride (23.9 g, 633 mmol, 0.9 equiv) was added slowly to the mixture and was stirred at 0° C. for 2 hours. The two reactions were combined and 1M HCl (300 mL) was added slowly to the reaction mixture. The mixture was diluted with water (1.0 L) and extracted with EtOAc (500 mL×2). The organic layer was washed with brine (500 mL) and concentrated under reduced pressure to afford ethyl 3-hydroxycyclobutane-1-carboxylate, which was used in the next step without further purification.

Step 5: Ethyl 3-hydroxycyclobutane-1-carboxylate (190 g, 1.32 mol, 1.0 equiv) was taken up in isopropyl acetate (1.3 L) and bubbled with N₂ 3 times. The mixture was cooled to ° C., then pyridine (159 mL, 1.98 mol, 1.5 equiv) followed by triflic anhydride (272 mL, 1.56 mol, 1.25 equiv) were added slowly at 0° C. and was stirred for 2 hours at 0° C. The mixture was warmed to 25° C. and 1M HCl (200 mL) was slowly added to the solution. The solution was washed with 1M HCl (250 mL), followed by water (300 mL), and brine (300 mL). To the organic layer was added NaI (394 g, 2.63 mol, 2.0 equiv) and the mixture was heated and stirred at 50° C. for 10 hours. Water (250 mL) was added to the reaction mixture and was stirred for 15 minutes. Aqueous saturated Na₂S₂O₃ (250 mL) was added and mixed for 30 minutes. The organic layer was washed with brine (250 mL), dried over anhydrous Na₂SO₄, filtered, and concentrated under reduced pressure. The residue was purified by silica gel column chromatography (10:1 to 1:1 pet. ether:EtOAc) to afford ethyl 3-iodocyclobutane-1-carboxylate.

Step 7: Two reactions were run in parallel. Ethyl 3-hydroxycyclobutane-1-carboxylate (100 g, 390 mmol, 1.0 equiv) was taken up in DMA (500 mL), followed by the addition of bis(pinacolato)diboron (149 g, 590 mmol, 1.0 equiv) and LiOMe (44.7 g, 1.18 mol, 3.0 equiv). The solution mixture was purged with N₂ 3 times. (9,9-dimethyl-9H-xanthene-4,5-diyl)bis(diphenylphosphane) copper (I) iodide (14.7 g, 190 mmol, 0.05 equiv) was added to the mixture and heated and stirred at 35° C. for 12 hours. Isopropyl acetate (150 mL) was added and aged for 10 minutes with stirring. The mixtures were combined, filtered through Celite™, and washed with isopropyl acetate (200 mL). Brine (400 mL) was added and the organic layer was washed with brine (600 mL), dried over anhydrous Na₂SO₄, filtered, and concentrated under reduced pressure. The residue was purified by silica gel column chromatography (50:1 to 1:1 pet. ether:EtOAc) to afford ethyl 3-(4,4,5,5-tetramethyl-1,3,2-dioxaborolan-2-yl)cyclobutane-1-carboxylate.

Step 8: LiOH·H₂O (41.6 g, 992 mmol, 2.1 equiv) was added to ethyl 3-(4,4,5,5-tetramethyl-1,3,2-dioxaborolan-2-yl)cyclobutane-1-carboxylate (120 g, 472 mmol, 1.0 equiv) in THF (600 mL) and water (100 mL). The mixture was stirred at 15° C. for 4 hours. 1M HCl (500 mL) was added to bring the pH to ˜2, then extracted with MTBE (200 mL×5). The organic layers were washed with brine (300 mL), dried over anhydrous Na₂SO₄, filtered, and concentrated under reduced pressure to afford 3-(4,4,5,5-tetramethyl-1,3,2-dioxaborolan-2-yl)cyclobutane-1-carboxylic acid.

Step 9: Three reactions were run in parallel. 3-(4,4,5,5-tetramethyl-1,3,2-dioxaborolan-2-yl)cyclobutane-1-carboxylic acid (10.3 g, 45.9 mmol, 1.2 equiv) and triethylamine (11.6 g, 114 mmol, 3.0 equiv) were added to a solution of N-(tert-butyl)-4-imino-8-methoxyquinazoline-2,3(4H)-diamine in DMAc (100 mL) followed by HATU (29.1 g, 76.5 mmol, 2.0 equiv). The mixture was heated and stirred at 65° C. for 12 hours. The three reactions were combined and water (300 mL) was added dropwise, followed by stirring for 0.5 hours. The mixture was filtered and the solid was collected. The solid was purified by silica gel column chromatography (35:1 pet. ether:EtOAc) to afford N-(tert-butyl)-7-methoxy-2-((1r,3r)-3-(4,4,5,5-tetramethyl-1,3,2-dioxaborolan-2-yl)cyclobutyl)-[1,2,4]triazolo[1,5-c]quinazolin-5-amine.

Step 10: Potassium hydrogen fluoride (1.5 mL, 4.4 mmol, 4 equiv) was added to a mixture of N-(tert-butyl)-7-methoxy-2-((1r,3r)-3-(4,4,5,5-tetramethyl-1,3,2-dioxaborolan-2-yl)cyclobutyl)-[1,2,4]triazolo[1,5-c]quinazolin-5-amine (500 mg, 1.1 mmol, 1.0 equiv) in MeOH (3 mL) and was stirred at 25° C. for 12 hours. Water (1 mL) was added and was aged for 1 hour. Solids were collected by filtration and washed with water (2 mL) and dried over N₂ sweep to afford N-(tert-butyl)-7-methoxy-2-((1r,3r)-3-(trifluoro-14-boranyl)cyclobutyl)-[1,2,4]triazolo[1,5-c]quinazolin-5-amine, potassium salt (Int-7). ESI MS m/z=370 [M+H]⁺ (hydrolysis during LCMS)

### N²-(2,4-dimethoxybenzyl)-8-fluoro-4-iminoquinazoline-2,3(4H)-diamine (Int-8)

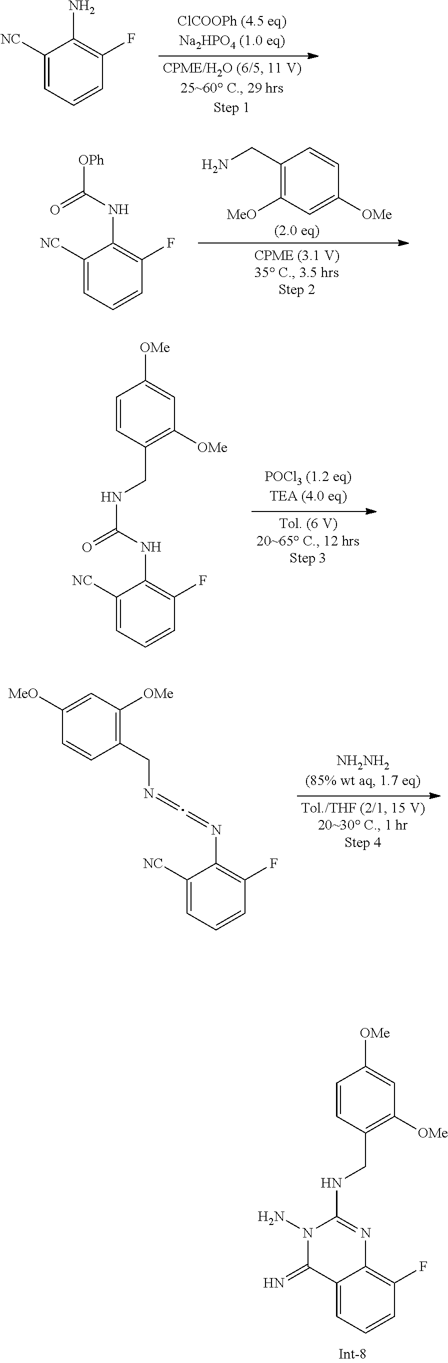

Step 1: 2-Amino-3-fluorobenzonitrile (85 g, 624 mmol, 1.0 equiv) was dissolved in CPME (510 mL), followed by the addition of aqueous Na₂HPO₄ (1.5 M, 416 mL, 1.0 equiv) at 25° C. The mixture was heated at 65° C. Phenyl chloroformate (117 mL, 936 mmol, 1.5 equiv) was added to the mixture over 3 hours at 65° C. and stirred at 65° C. for 12 hours. A second portion of phenyl chloroformate (117 mL, 936 mmol, 1.5 equiv) was added to the mixture over 3 hours at 65° C. and stirred at 65° C. for 4 hours. A third portion of phenyl chloroformate (117 mL, 936 mmol, 1.5 equiv) was added to the mixture over 3 hours at 65° C. and stirred at 65° C. for 4 hours. The mixture was phase cut at 65° C. and the organic layer was cooled to 30° C. Phenyl (2-cyano-6-fluorophenyl)carbamate was isolated as a solution in CPME and used in the next step without further purification.

Step 2: To a solution of (2-cyano-6-fluorophenyl)carbamate in CPME (510 mL) was added 2,4-dimethoxybenzylamine (188 mL, 1250 mmol, 2.0 equiv) dropwise over 30 minutes at 35° C. The reaction was stirred at 35° C. for 3 hours. The solid was collected to afford 1-(2-cyano-6-fluorophenyl)-3-(2,4-dimethoxybenzyl)urea and was used in the next step without further purification

Step 3: Five identical reactions were set up in parallel. 1-(2-Cyano-6-fluorophenyl)-3-(2,4-dimethoxybenzyl)urea (42.6 g, 129 mmol, 1.0 equiv) was taken up in toluene (255 mL) and triethylamine (72.0 mL, 517 mmol, 4.0 equiv) was added. Phosphorus (V) oxychloride (14.4 mL, 155 mmol, 1.2 equiv) was added at 25° C. and was then heated to 65° C., where it was stirred at that temperature for 12 hours. The five reactions were then combined after the 12 hours. The combined reaction mixture was cooled to 0° C. and was added to a solution of 2M KOH (1.5 L) and toluene (400 mL) at 0° C. The reaction mixture was filtered through Celite™ and washed with toluene (400 mL). The filtrate was phase cut and the organic layer was washed with 10% aqueous NaCl (1.0 L) to afford 2-((((2,4-dimethoxybenzyl)imino)methylene)amino)-3-fluorobenzonitrile as a solution in toluene which was used in the next step without further purification.

Step 4: Five reactions were set up in parallel. 2-((((2,4-Dimethoxybenzyl)imino)methylene)amino)-3-fluorobenzonitrile (40.2 g, 129 mmol, 1.0 equiv) in toluene (415 mL) was added into a solution of hydrazine hydrate (12.5 mL, 219 mmol, 85.0% purity, 1.7 equiv) and THF (200 mL) at 25° C. and was stirred at that temperature for 1 hour. The five reactions were combined after the 1 hour. 10% Aqueous NaCl (1.0 L) was added and the phases were cut. The organic layer was concentrated to near dryness, then toluene (1.0 L) was added. This was repeated once more, then concentrated to near dryness. The vessel was cooled to 20° C. and heptane (1.0 L) was added. The solution was stirred for 12 hours, then was filtered and the solid was collected. Acetonitrile was added to the filtrate, the solution was stirred for 1 h and the resulting solids were collected. The solids were combined to afford N²-(2,4-dimethoxybenzyl)-8-fluoro-4-iminoquinazoline-2,3(4H)-diamine (Int-1). ESI MS m/z=344 [M+H]⁺

### N²-(tert-butyl)-8-chloro-4-iminoquinazoline-2,3(4H)-diamine (Int-9)

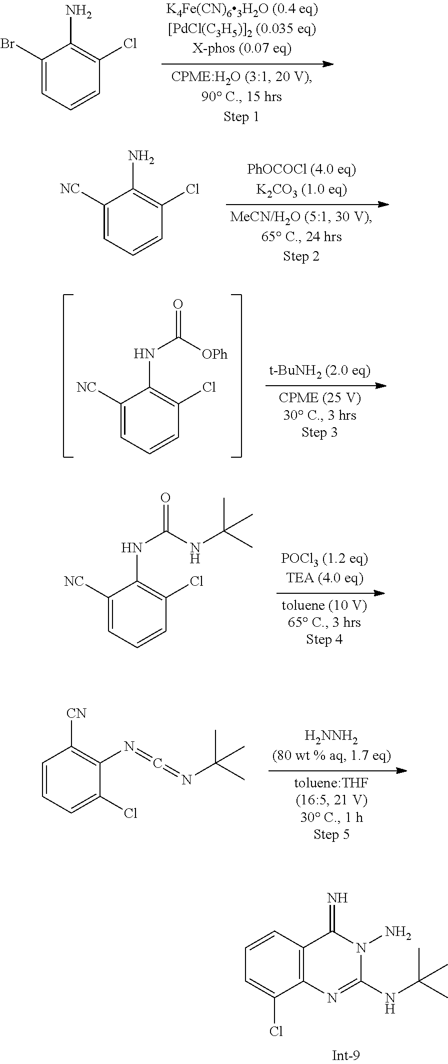

Step 1: Two reactions were performed in parallel. CPME (1.75 L) was added to a reactor with an overhead stirrer followed by [PdCl(C₃H₅)]₂ (10.9 g, 28.7 mmol, 0.035 equiv) and X-Phos (28.3 g, 59.3 mmol, 0.07 equiv). The mixture was stirred at 25° C. for 2 hours. 2-Bromo-6-chloroaniline (175 g, 848 mmol, 1.0 equiv) in CPME (875 mL) was added to the reactor and was stirred at 25 5° C. for 1 hour. K₄Fe(CN)₆·3H₂O (97.5 g, 231 mmol, 0.4 equiv) in water (600 mL) was added to the reactor and was stirred at 90° C. for 12 hours. The reactor was cooled to 20° C. and the batches were filtered through Celite™ and was washed with CPME (400 mL). The filtrate was phase separated and the organic layer was concentrated under reduced pressure to give a residue. The residue was purified by silica gel column chromatography (100:1 to 10:1 pet. ether:EtOAc) to afford 2-amino-3-chlorobenzonitrile.

Step 2: Three reactions were run in parallel. 2-Amino-3-chlorobenzonitrile (62.0 g, 406 mmol, 1.0 equiv) in MeCN (310 mL) was charged to a reactor with an overhead stirrer, followed by water (62.0 mL) and potassium carbonate (56.2 g, 406 mmol, 1 equiv). The mixture was stirred at 65° C. Phenyl chloroformate (50.9 mL, 206 mmol, 1 equiv) was added and was stirred at 65 15° C. for 4 hours. A second equivalent of phenyl chloroformate (50.9 mL, 206 mmol, 1 equiv) was added and was stirred at 65° C. for 4 hours. A third equivalent of phenyl chloroformate (50.9 mL, 206 mmol, 1 equiv) was added and was stirred at 65° C. for 12 hours. A fourth equivalent of phenyl chloroformate (50.9 mL, 206 mmol, 1 equiv) was added and was stirred at 65° C. for 4 hours. The phases were separated at 65° C. and the organic phase was cooled to 30° C. Phenyl (2-chloro-6-cyanophenyl)carbamate was isolated as a solution in CPME and was used in the next step without further purification.

Step 3: Three reactions were run in parallel. Phenyl (2-chloro-6-cyanophenyl)carbamate (110 g, 403 mmol, 1 equiv) and CPME (1.0 L) were added to a reactor with an overhead stirrer. tert-Butylamine (84.8 mL, 810 mmol, 2.0 equiv) was added to the reactor at 30° C. and was stirred for 2 hours. The three batches were combined and filtered. The filtrate was concentrated under reduced pressure to give 1-(tert-butyl)-3-(2-chloro-6-cyanophenyl)urea and was used in the next step without further purification.

Step 4: 1-(Tert-butyl)-3-(2-chloro-6-cyanophenyl)urea (190 g, 754 mmol, 1.0 equiv) was added to reactor with an overhead stirrer and toluene (1.9 L) and triethylamine (420 mL, 3020 mmol, 4.0 equiv) was added. Phosphorus (V) oxychloride (84.1 mL, 905 mmol, 1.2 equiv) was added at 25° C. and was then heated to 65° C., where it was stirred at that temperature for 2 hours. The reaction mixture was cooled to 0° C. and was added to a solution of 2M KOH (1.0 L) and toluene (400 mL) at 0° C. The reaction mixture was filtered through Celite™ and washed with toluene (500 mL). The filtrate was phase cut and the organic layer was washed with 10% NaCl (500 mL). The organic layer was collected to afford 2-(((tert-butylimino)methylene)amino)-3-chlorobenzonitrile as a solution in toluene and was used in the next step without further purification.

Step 5: 2-(((Tert-butylimino)methylene)amino)-3-chlorobenzonitrile (166 g, 710 mmol, 1.0 equiv) in toluene (2.66 L) was added into a solution of hydrazine hydrate (69.0 mL, 1210 mmol, 85.0% purity, 1.7 equiv) and THF (830 mL) in a reactor with an overhead stirrer at 25° C. and was stirred at that temperature for 1 hour. 10% NaCl (1.0 L) was added and the phases were cut. The organic layer was concentrated to near dryness, then toluene (1.0 L) was added. This was repeated once more, then concentrated to near dryness. The vessel was cooled to 20° C. and heptane (1.5 L) was added. The solution was stirred for 12 hours. The mixture was filtered and concentrated under reduced pressure to afford N²-(tert-butyl)-8-chloro-4-iminoquinazoline-2,3(4H)-diamine (Int-9 as a solid). ESI MS m/z=266 [M+H]⁺

### N²-(tert-butyl)-6-fluoro-4-imino-7-methoxyquinazoline-2,3(4H)-diamine (Int-10)

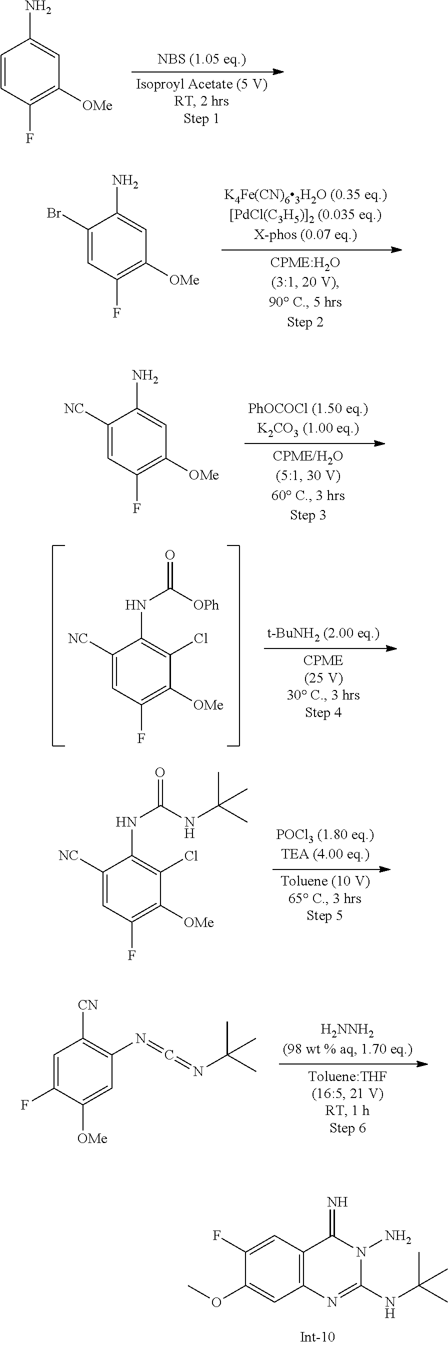

Step 1: Two reactions were run in parallel. 4-Fluoro-3-methoxyaniline (135 g, 940 mmol, 1 equiv) was added to isopropyl acetate (670 mL) at 25° C. The reactor was cooled to 0° C. and NBS (177 g, 980 mmol, 1.05 equiv) was added. The mixture was stirred at 25° C. for 2 hours. The two reactions were combined and quenched by addition of aqueous sodium carbonate (500 mL), then was diluted with CPME (500 mL), and extracted with CPME (500 mL×3). The combined organic layers were concentrated under reduced pressure and the resulting residue of 2-bromo-4-fluoro-5-methoxyaniline was used in the next step without further purification.

Step 2: Two reactions were run in parallel. CPME (2700 mL) and water (900 mL) was added to a reactor. 2-Bromo-4-fluoro-5-methoxyaniline (180 g, 818 mmol, 1 equiv) was added and mixture was stirred at 25° C. under N₂ for 30 minutes. K₄Fe(CN)₆·3H₂O (120 g, 285 mmol, 0.35 equiv) was added followed by [PdCl(C₃H₅)]₂ (10.4 g, 28.6 mmol, 0.035 equiv) and XPhos (27.3 g, 57.2 mmol, 0.07 equiv). The mixture was stirred at 90° C. for 3 hours. The two reactions were combined, cooled to 25° C., and filtered through Celite™. The organic layer was collected to give 2-amino-5-fluoro-4-methoxybenzonitrile in CPME and was used in the next step directly.

Step 3: Water (975 mL) was added to a reactor, followed by 2-amino-5-fluoro-4-methoxybenzonitrile (390 g, 1170 mmol) in CPME (4785 mL) at 25° C. Sodium phosphate dibasic (450 g, 1170 mmol, 1 equiv) was added and the mixture was warmed to 60° C. Phenyl chloroformate (275 g, 1740 mmol, 1.5 equiv) was added and the mixture was stirred at 60° C. for 3 hours. The mixture was separated and the organic layer was collected to give phenyl (2-cyano-4-fluoro-5-methoxyphenyl)carbamate in CPME and was used in the next step directly.

Step 4: Phenyl (2-cyano-4-fluoro-5-methoxyphenyl)carbamate (335 g, 1170 mmol) in CPME (4785 mL) was added to a reactor, followed by tert-butylamine (171 g, 2340 mmol, 2.0 equiv) and the mixture was stirred at 25° C. for 3 hours. The reaction mixture was washed with 2M KOH (500 mL×2) and the organic layer was washed with brine (500 mL×3), dried over anhydrous Na₂SO₄, and concentrated under reduced pressure to give a residue. The residue was triturated with MTBE (1500 mL) at 25° C. for 10 hours and the solid was collected to afford 1-(tert-butyl)-3-(2-cyano-4-fluoro-5-methoxyphenyl)urea.

Step 5: 1-(Tert-butyl)-3-(2-cyano-4-fluoro-5-methoxyphenyl)urea (150 g, 560 mmol, 1.0 equiv) was added to reactor with an overhead stirrer and toluene (1.5 L) and triethylamine (78.8 mL, 560 mmol, 4.0 equiv) was added. Phosphorus (V) oxychloride (95.1 mL, 990 mmol, 1.8 equiv) was added at 25° C. and was then heated to 65° C., where it was stirred at that temperature for 3 hours. The reaction mixture was cooled to 0° C. and was added to a solution of 2M KOH (900 mL) and toluene (300 mL) at 0° C. The reaction mixture was filtered through Celite™ and the filtrate was phase cut and the organic layer was washed with brine (1.0 L×3), dried over anhydrous Na₂SO₄, filtered and concentrated under reduced pressure to give 2-(((tert-butylimino)methylene)amino)-5-fluoro-4-methoxybenzonitrile as a residue that was taken up as a solution in toluene to be used in the next step directly.

Step 6: 2-(((Tert-butylimino)methylene)amino)-5-fluoro-4-methoxybenzonitrile (139 g, 565 mmol, 1.0 equiv) in toluene (2.2 L) was added into a solution of hydrazine hydrate (384 mL, 960 mmol, 98.0% purity, 1.7 equiv) and THF (699 mL) in a reactor with an overhead stirrer at 25° C. and was stirred at that temperature for 1 hour. The organic layer was washed with brine (1.0 L×3), dried over anhydrous Na₂SO₄, filtered and concentrated under reduced pressure to give N-(tert-butyl)-6-fluoro-4-imino-7-methoxyquinazoline-2,3(4H)-diamine (Int-10). ESI MS m/z=280 [M+H]⁺

### N²-(tert-butyl)-6,8-difluoro-4-iminoquinazoline-2,3(4H)-diamine (Int-11)

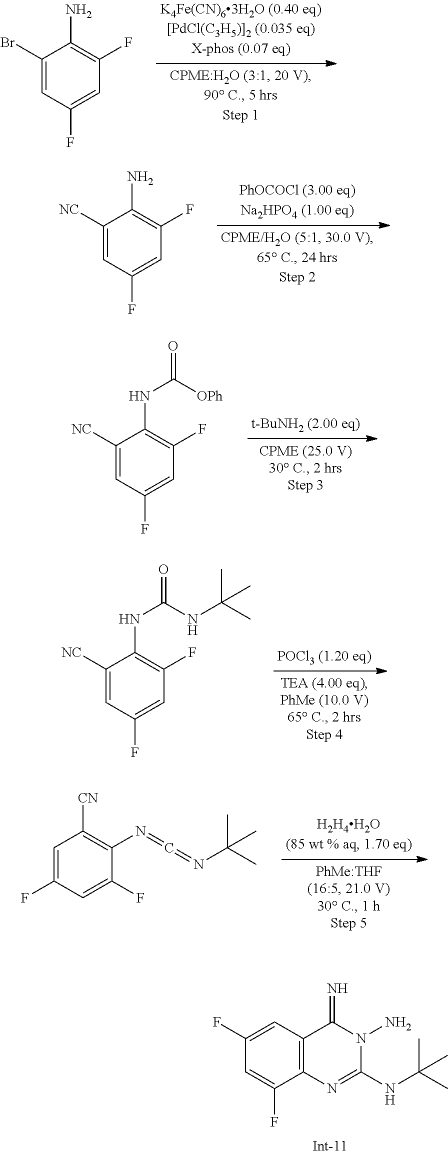

Step 1: Two reactions were run in parallel. CPME was added to a reactor with an overhead stirrer (1.45 L) followed by [PdCl(C₃H₅)]₂ (8.9 g, 24.4 mmol, 0.035 equiv) and X-Phos (23.3 g, 48.8 mmol, 0.07 equiv). The mixture was stirred at 25° C. for 2 hours. 2-bromo-4,6-difluoroaniline (145 g, 697 mmol, 1.0 equiv) in CPME (725 mL) was added to the reactor and the reaction was stirred at 25° C. for 1 hour. K₄Fe(CN)₆·3H₂O (118 g, 279 mmol, 0.4 equiv) in water (725 mL) was added to the reactor and was stirred at 90° C. for 12 hours. The reactor was cooled to 20° C. and the batches were combined and worked up together. The mixture was filtered through Celite™ and was washed with CPME (580 mL). The filtrate was phase separated and the organic layer was concentrated under reduced pressure to afford 2-amino-3,5-difluorobenzonitrile as a residue that was then taken up in CPME and used in the next step without further purification.

Step 2: Two reactions were run in parallel. 2-Amino-3,5-difluorobenzonitrile (80 g, 519 mmol, 1.0 equiv) was dissolved in CPME (2.0 L), followed by the addition of Na₂HPO₄ (73.7 g, 519 mmol, 1.0 equiv) and water (400 mL) at 25° C. The mixture was heated and stirred at 65° C. Phenyl chloroformate (97.5 mL, 779 mmol, 1.5 equiv) was added to the mixture and stirred at 65° C. for 12 hours. A second portion of phenyl chloroformate (97.5 mL, 779 mmol, 1.5 equiv) was added to the mixture and stirred at 65° C. for 12 hours. The mixture was phase cut at 65° C. and the organic layer was cooled to 30° C. Phenyl (2-cyano-4,6-difluorophenyl)carbamate was isolated as a residue that was then taken up in CPME and used in the next step without further purification.

Step 3: Two reactions were run in parallel. Phenyl (2-cyano-4,6-difluorophenyl)carbamate (80.0 g, 292 mmol, 1 equiv) in CPME (2.0 L) was added to a reactor with an overhead stirrer. tert-Butylamine (61.3 mL, 583 mmol, 2.0 equiv) was added to the reactor at 30° C. and was stirred for 2 hours. The two batches were combined and filtered. The filtrate was concentrated under reduced pressure to give 1-(tert-butyl)-3-(2-cyano-4,6-difluorophenyl)urea and was used in the next step without further purification.

Step 4: 1-(Tert-butyl)-3-(2-cyano-4,6-difluorophenyl)urea (150 g, 592 mmol, 1.0 equiv) was taken up in toluene (1.5 L) and triethylamine (330 mL, 2370 mmol, 4.0 equiv) was added. Phosphorus (V) oxychloride (66.1 mL, 711 mmol, 1.2 equiv) was added at 25° C. and was then heated to 65° C., where it was stirred at that temperature for 2 hours. The reaction mixture was cooled to 0° C. and was added to a solution of 2M KOH (900 mL) and toluene (300 mL) at 0° C. The reaction mixture was filtered through Celite™ and washed with toluene (900 mL). The filtrate was phase cut and the organic layer was washed with 10% wt NaCl (750 mL). The organic layer was collected to afford 2-(((tert-butylimino)methylene)amino)-3,5-difluorobenzonitrile as a solution in toluene and was used in the next step without further purification.

Step 5: 2-(((Tert-butylimino)methylene)amino)-3,5-difluorobenzonitrile (150 g, 638 mmol, 1.0 equiv) in toluene (2.4 L) was added into a solution of hydrazine hydrate (62.0 mL, 1080 mmol, 85.0% purity, 1.7 equiv) and THF (750 mL) in a reactor with an overhead stirrer at 25° C. and was stirred at that temperature for 1 hour. 10% NaCl (750 mL) was added and the phases were cut. The organic layer was concentrated to near dryness, then toluene (1.0 L) was added. This was repeated once more, then concentrated to near dryness. The vessel was cooled to 20° C. and heptane (1.5 L) was added. The solution was stirred for 12 hours, then filtered and concentrated under reduced pressure to afford N¹-(tert-butyl)-6,8-difluoro-4-iminoquinazoline-2,3(4H)-diamine (Int-11 as a solid). ESI MS m/z=268 [M+H]⁺

### N-(2,4-dimethoxybenzyl)-2-(2-iodocyclopropyl)-7-methoxy-[1,2,4]triazolo[1,5-c]quinazolin-5-amine (Int-12)

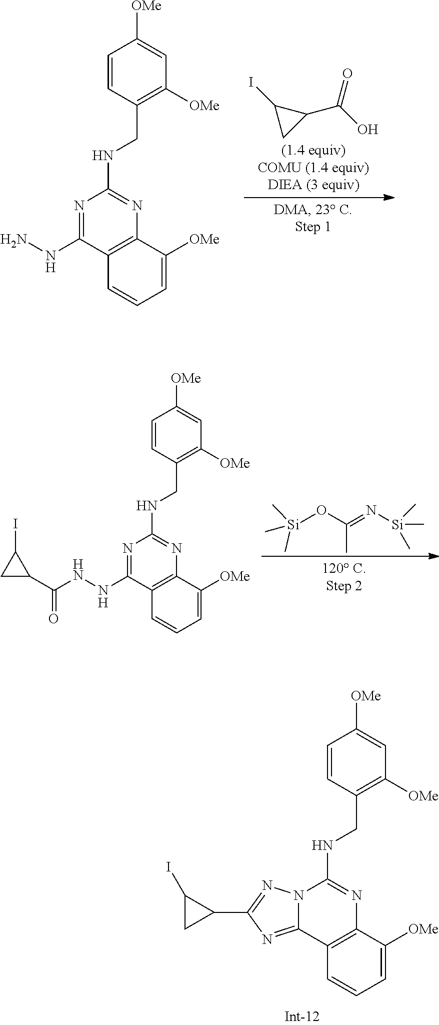

Step 1: COMU (1012 mg, 2.36 mmol, 1.4 equiv) and DMA (11.7 mL) were added to a 50 mL round-bottomed flask charged with 2-iodocyclopropane-1-carboxylic acid (501 mg, 2.364 mmol, 1.4 equiv) followed by DIEA (885 μl, 5.06 mmol, 3.0 equiv). The flask was capped and the contents were stirred at room temperature for 10 minutes. N-(2,4-dimethoxy benzyl)-4-hydrazineyl-8-methoxyquinazolin-2-amine (600 mg, 1.69 mmol, 1.0 equiv) was added and the resulting reaction mixture stirred at 25° C. for 18 hours. The mixture was concentrated, diluted with chloroform/isopropanol—3:1 (5 mL) and washed with aqueous sodium hydrogen carbonate (saturated, 5 mL). The organic layer was collected using a phase separator column (25 mL) and concentrated to afford crude N-(2-((2,4-dimethoxybenzyl)amino)-8-methoxyquinazolin-4-yl)-2-iodocyclopropane-1-carbohydrazide which was used in the next step without further purification. MS (ESI): m/z (M+H)+550.

Step 2: N,O-bis(trimethylsilyl)acetamide (6 mL, 24.48 mmol, 14.5 equiv) was added to N′-(2-((2,4-dimethoxybenzyl)amino)-8-methoxyquinazolin-4-yl)-2-iodocyclopropane-1-carbohydrazide (927 mg, 1.687 mmol, 1.0 equiv) and the mixture was stirred at 120° C. for 2 hours. The mixture was concentrated, diluted with ethyl acetate (20 mL) and washed with aqueous sodium hydrogen carbonate (saturated, 20 mL). The organic layer was collected using a separatory funnel and concentrated. The crude mixture was purified via silica gel column chromatography eluting from 0-50% hexanes in ethyl acetate to afford N-(2,4-dimethoxybenzyl)-2-(2-iodocyclopropyl)-7-methoxy-[1,2,4]triazolo[1,5-c]quinazolin-5-amine (Int-12). MS (ESI): m/z (M+H)+532.

### 5-(2-azidopropan-2-yl)-2-bromopyridine (Int-13)

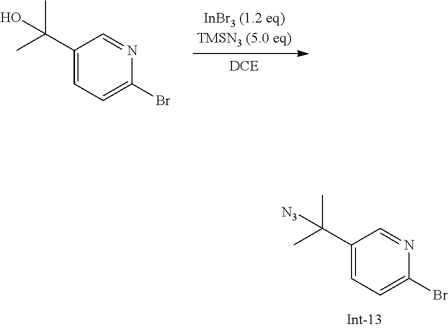

A 250 mL flask was charged with 2-(6-bromopyridin-3-yl)propan-2-ol (2.5 g, 11.57 mmol, 1.0 equiv) and indium(III) bromide (4.92 g, 13.88 mmol, 1.2 equiv), then evacuated and backfilled with nitrogen. 1,2-Dichloroethane (100 mL) was then added, followed by trimethylsilyl azide (7.68 mL, 57.8 mmol, 5.0 equiv), and the resulting mixture was heated to 60° C. and stirred for 18 hours. The mixture was then heated to 80° C. and stirred for 6 hours. The reaction mixture was quenched with saturated aqueous NaHCO₃ (50 mL) and water (25 mL). The layers were then separated. The aqueous layer was then extracted with 25% IPA in chloroform (60 mL×3). All the organic layers were combined, dried over anhydrous MgSO₄ and concentrated under reduced pressure. The crude material was used directly in the subsequent step. ESI MS m/z=241/243 [M+H]⁺

### tert-butyl (2-(6-bromo-2-methylpyridin-3-yl)propan-2-yl)carbamate (Int-14)

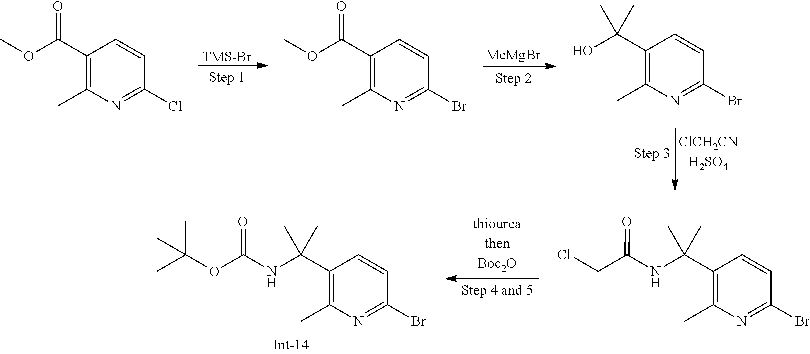

Step 1: Methyl 6-chloro-2-methylnicotinate (5.12 g, 27.6 mmol, 1.0 equiv) was dissolved in acetonitrile (55.2 ml) and purged with argon for 10 min. The solution was cooled to 0° C. Bromotrimethylsilane (9.10 ml, 69.0 mmol, 2.5 equiv) was added dropwise and reaction stirred 18 hours at 80° C. The solution was slowly added to ice water while mixing and extracted with EtOAc (3×). The combined organic layers were washed with brine, dried over anhydrous Na₂SO₄, filtered, and concentrated under reduced pressure. The crude residue was purified by silica gel column chromatography (0-100% EtOAc in hex) to afford methyl 6-bromo-2-methylnicotinate. ESI MS m/z=230/232 [M+H]⁺

Step 2: Methyl 6-bromo-2-methylnicotinate (5.96 g, 25.9 mmol, 1.0 equiv) was dissolved in THF (86 ml) and cooled to 0° C. Methylmagnesium bromide in THF (30.5 ml, 104 mmol, 4.0 equiv) was added dropwise and the reaction was stirred at 0° C. for 2 hours. The reaction was quenched slowly with sat. NH₄Cl then extracted with EtOAc (3×). The resulting organic layers were combined, washed with brine, dried over anhydrous Na₂SO₄, filtered, and concentrated under reduced pressure. The crude residue was purified by silica gel column chromatography (0-100% EtOAc in hex) to afford 2-(6-bromo-2-methylpyridin-3-yl)propan-2-ol. ESI MS m/z=230/232 [M+H]⁺

Step 3: 2-(6-bromo-2-methylpyridin-3-yl)propan-2-ol (5.38 g, 23.4 mmol, 1.0 equiv) was taken up in chloroacetonitrile (80 ml) and cooled to 0° C. Sulfuric acid (6.23 ml, 117 mmol, 5 equiv) was added dropwise and reaction was warmed to 30° C. and was stirred for 16 hours. The reaction was quenched via addition to ice cold sat. NaHCO₃ and extracted with EtOAc (3×). The resulting organic layers were combined, washed with brine, and concentrated under reduced pressure. The crude residue was purified by silica gel column chromatography (0-100% EtOAc in hex) to afford N-(2-(6-bromo-2-methylpyridin-3-yl)propan-2-yl)-2-chloroacetamide. ESI MS m/z=305/307 [M+H]⁺

Step 4: N-(2-(6-bromo-2-methylpyridin-3-yl)propan-2-yl)-2-chloroacetamide (3.16 g, 10.3 mmol, 1.0 equiv) was taken up in ethanol (11.2 ml) and acetic acid (2.24 ml). Thiourea (1.181 g, 15.5 mmol, 1.5 equiv) was added at 25° C. and the reaction heated at 80° C. for 18 hours. The reaction was concentrated under reduced pressure to afford 2-(6-bromo-2-methylpyridin-3-yl)propan-2-amine which was used without further purification in the next step.

Step 5: 2-(6-Bromo-2-methylpyridin-3-yl)propan-2-amine (2.36 g, 10.3 mmol, 1.0 equiv) was taken up in water (51 ml) and THF (51 ml). Sodium bicarbonate (3.46 g, 41.2 mmol, 4.0 equiv) then Boc-anhydride (6.75 g, 30.9 mmol, 3.0 equiv) were added and reaction was stirred at 25° C. for 2 hours. The reaction was diluted with water and extracted with EtOAc (3×). The resulting organic layers were combined, washed with brine, dried over anhydrous Na₂SO₄, filtered, and concentrated under reduced pressure. The crude residue was purified by silica gel column chromatography (0-100% EtOAc in hex) to afford tert-butyl (2-(6-bromo-2-methylpyridin-3-yl)propan-2-yl)carbamate (Int-1414). ESI MS m/z=329/331 [M+H]⁺

### 3-(6-Bromo-5-methylpyridin-3-yl)oxetan-3-ol (Int-15)

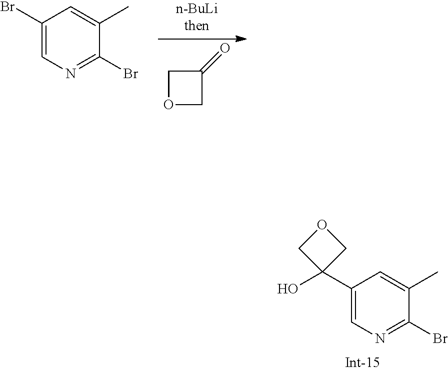

2,5-Dibromo-3-methylpyridine (389 mg, 1.55 mmol, 3.1 equiv) was dissolved in Et₂O (20 ml) and degassed for 10 min with argon. The solution was cooled to −78° C. and n-butyllithium (620 μl, 1.55 mmol, 3.1 equiv) was added dropwise. The reaction was stirred for 30 min. at −78° C. 3-oxetanone (32.1 μl, 0.5 mmol, 1.0 equiv) was added dropwise and the reaction was stirred at −78° C. for 1 hour. Saturated aqueous NH₄Cl was added and reaction warmed to 25° C. The mixture was extracted with Et₂O (3×). The organic layers were combined, washed with brine, dried over anhydrous Na₂SO₄, filtered, and concentrated under reduced pressure. The crude residue was purified by silica gel column chromatography (0-100% 3:1 EtOAc:EtOH in hex) to afford 3-(6-bromo-5-methylpyridin-3-yl)oxetan-3-ol (Int-15). ESI MS m/z=244/246 [M+H]⁺

### N-(3-(6-bromo-5-methylpyridin-3-yl)oxetan-3-yl)-2-methylpropane-2-sulfinamide (Int-16)

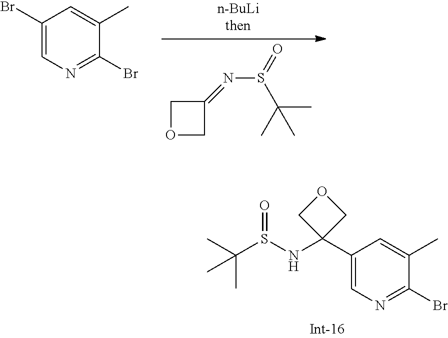

2,5-Dibromo-3-methylpyridine (529 mg, 2.11 mmol, 3.1 equiv) was dissolved in Et₂O (13.6 ml) and bubble degassed for 10 min with argon. The solution was cooled to −78° C. and n-butyllithium (843 μl, 2.108 mmol, 3.1 equiv) was added dropwise. The reaction was stirred for 30 min. at −78° C. 2-methyl-N-(oxetan-3-ylidene)propane-2-sulfinamide (119 mg, 0.68 mmol, 1.0 equiv) was added dropwise and reaction was stirred at −78° C. for 1 hour. Saturated aqueous NH₄Cl was added and the reaction was warmed to 25° C. The mixture was extracted with Et₂O (3×), the organic layers were washed with brine, and dried over anhydrous Na₂SO₄. The combined organic layers were concentrated under reduced pressure. The crude residue was purified by silica gel column chromatography (0-100% 3:1 EtOAc:EtOH in hex) to afford N-(3-(6-bromo-5-methylpyridin-3-yl)oxetan-3-yl)-2-methylpropane-2-sulfinamide (Int-16). ESI MS m/z=347/349 [M+H]⁺

### tert-butyl (2-(5-bromo-6-methylpyrazin-2-yl)propan-2-yl)carbamate (Int-17)

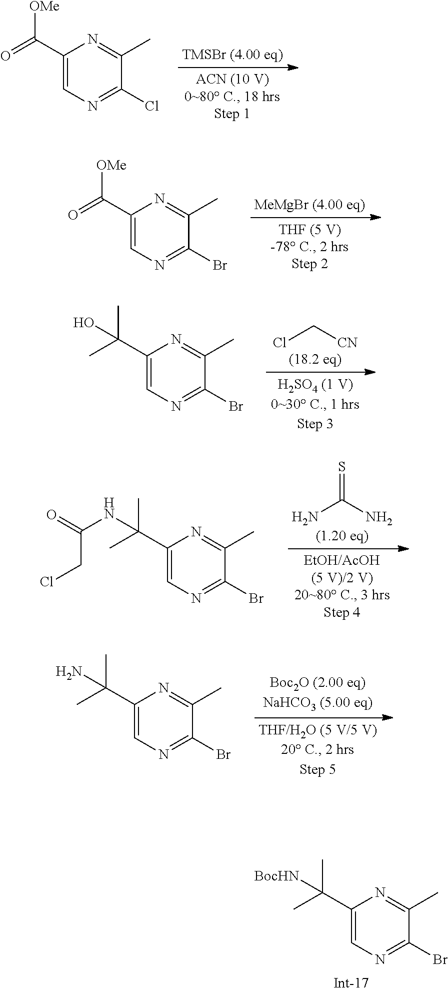

Step 1: Three reactions were performed in parallel. TMSBr (1.11 L, 8.57 mol, 4.0 equiv) was added to a solution of methyl 5-chloro-6-methylpyrazine-2-carboxylate (400 g, 2.14 mol, 1.0 equiv) in MeCN (2.0 L) at 0° C. The reaction mixture was heated and stirred at 80° C. for 18 hours. The three reactions were combined and concentrated under reduced pressure. The mixture was poured into aqueous sodium carbonate (5.0 L) and the mixture was filtered. The filter cake was washed with water (3.0 L) and was purified by silica gel column chromatography (1:0 to 0:1 pet. ether:EtOAc) to afford methyl 5-bromo-6-methylpyrazine-2-carboxylate. ESI MS m/z=231/233 [M+H]⁺

Step 2: Four reactions were run in parallel. Methylmagnesium bromide in THF (1.15 L at 3.0 M, 3.46 mol, 4.0 equiv) was added to a solution of methyl 5-bromo-6-methylpyrazine-2-carboxylate (200 g, 0.865 mol, 1.0 equiv) in THF (2.0 L) at −78° C. and the mixture was stirred at −78° C. for 2 hours. The mixture was poured into aqueous ammonium chloride (10.0 L) and extracted with EtOAc (3.0 L×3). The organic layer was washed with brine (3.0 L×2), dried over anhydrous Na₂SO₄, and concentrated under reduced pressure. The residue was purified by silica gel column chromatography (1:0 to 0:1 pet. ether:EtOAc) to afford 2-(5-bromo-6-methylpyrazin-2-yl)propan-2-ol. ESI MS m/z=231/233 [M+H]⁺

Step 3: 2-(5-Bromo-6-methylpyrazin-2-yl)propan-2-ol (230 g, 0.995 mol, 1.0 equiv) and chloroacetonitrile (1.15 L, 18.1 mol, 18.2 equiv) were cooled to ° C. Sulfuric acid (230 mL) was added dropwise at 0° C. and reaction was stirred at 30° C. for 1 hour. The mixture was poured into aqueous sodium carbonate (300 mL) and extracted with EtOAc (300 mL×3). The organic phases were combined, washed with brine (300 mL×2), dried over anhydrous Na₂SO₄, filtered, and concentrated under reduced pressure. The residue was purified by silica gel column chromatography (1:0 to 0:1 pet. ether:EtOAc) to afford N-(2-(5-bromo-6-methylpyrazin-2-yl)propan-2-yl)-2-chloroacetamide.

Step 4: Thiourea (44.7 g, 587 mmol, 1.2 equiv) was added to a solution of N-(2-(5-bromo-6-methylpyrazin-2-yl)propan-2-yl)-2-chloroacetamide (150 g, 487 mmol, 1.0 equiv) in EtOH (950 mL) followed by acetic acid (280 mL, 4.89 mol, 10.0 equiv). The reaction mixture was stirred at 80° C. for 3 hours. The reaction mixture was filtered and the filtrate was concentrated under reduced pressure to afford crude 2-(5-bromo-6-methylpyrazin-2-yl)propan-2-amine and was used in the next step without further purification.

Step 5: Sodium bicarbonate (205 g, 2.44 mol, 5.0 equiv) at 20° C. was added to a solution of 2-(5-bromo-6-methylpyrazin-2-yl)propan-2-amine (112 g, 489 mmol, 1.0 equiv) in THF (594 mL) and water (710 mL), followed by di-tert-butyl dicarbonate (213 g, 978 mmol, 2.0 equiv) portion wise at 0° C. The reaction mixture was stirred at 20° C. for 2 hours. The mixture was poured into water (3.0 L) and extracted with EtOAc (1.0 L×3). The organic layers were combined, washed with brine (1.0 L×2), dried over anhydrous Na₂SO₄, filtered, and concentrated under reduced pressure. The residue was purified by silica gel column chromatography (1:0 to 0:1 pet. ether:EtOAc) to afford a crude residue that was triturated with pet. ether (300 mL) for 2 hours to afford tert-butyl (2-(5-bromo-6-methylpyrazin-2-yl)propan-2-yl)carbamate (Int-17). ESI MS m/z=330/332 [M+H]⁺

### N-(2-(5-bromo-3-methylpyrazin-2-yl)propan-2-yl)-2-methylpropane-2-sulfinamide (Int-18)

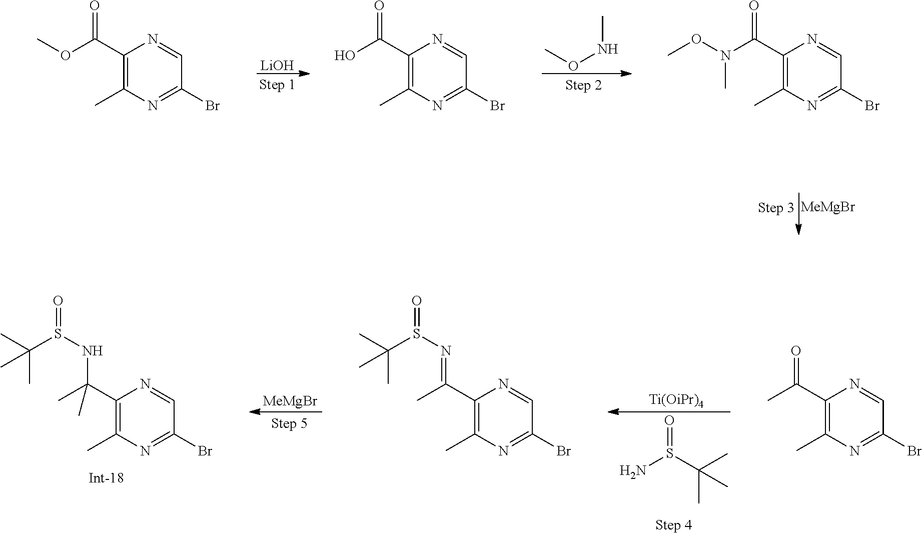

Step 1: Lithium hydroxide (0.495 g, 20.7 mmol, 1.0 equiv) in water (17 ml) was added to a solution of methyl 5-bromo-3-methylpyrazine-2-carboxylate (4.78 g, 20.7 mmol, 1.0 equiv) in THF (86 ml) and allowed to stir at 25° C. The mixture was diluted with EtOAc and water and the layers separated. The aqueous layer was adjusted to pH˜2 with 1N HCl (aq.) and extracted with EtOAc (3×). These combined organic layers were washed with brine, dried over anhydrous Na₂SO₄, filtered, and concentrated to afford 5-bromo-3-methylpyrazine-2-carboxylic acid, which was used directly in next reaction. ESI MS m/z=217/219 [M+H]⁺

Step 2: 5-Bromo-3-methylpyrazine-2-carboxylic acid (4.74 g, 21.8 mmol, 1.0 equiv), N,O-dimethylhydroxylamine hydrochloride (2.56 g, 26.2 mmol, 1.2 equiv), and HATU (41.5 g, 109 mmol, 5 equiv) were taken up in DCM (218 mL). Triethylamine (12.18 ml 87 mmol, 4.0 equiv) was added and reaction stirred at 25° C. for 48 hours. The reaction was quenched with sat. aq. NaHCO₃ and extracted with DCM (3×). The resulting organic layers were combined, washed with brine, dried over anhydrous Na₂SO₄, filtered, and concentrated under reduced pressure to afford a residue which was purified by silica gel column chromatography (0-100% EtOAc in hex) to afford 5-bromo-N-methoxy-N,3-dimethylpyrazine-2-carboxamide. ESI MS m/z=260/262 [M+H]⁺

Step 3: 5-Bromo-N-methoxy-N,3-dimethylpyrazine-2-carboxamide (2 g, 7.7 mmol, 1.0 equiv) was dissolved in THF (38.4 ml) and bubble degassed with Ar for 10 min. The solution was cooled to 0° C. Methylmagnesium bromide in THF (3.60 ml, 11.5 mmol, 1.5 equiv) was added dropwise with stirring. The reaction was stirred at 0° C. for 1 hour. The reaction was stirred at 25° C. for 1 hour. The reaction was quenched with sat. aq. NH₄Cl at 0° C. and the mixture extracted with EtOAc (3×). The resulting organic layers were combined, washed with brine, dried over anhydrous Na₂SO₄, filtered, and concentrated under reduced pressure. The resulting residue was purified by silica gel column chromatography (0-100% EtOAc in hex) to afford 1-(5-bromo-3-methylpyrazin-2-yl)ethan-1-one.

Step 4: 2-Methylpropane-2-sulfinamide (0.631 g, 5.21 mmol, 1.0 equiv) and 1-(5-bromo-3-methylpyrazin-2-yl)ethan-1-one (1.12 g, 5.21 mmol, 1.0 equiv) were dissolved in THF (5.21 ml). Titanium (IV) isopropoxide (3.08 ml, 10.42 mmol, 2.0 equiv) was added and reaction stirred at 50° C. for 12 hours. The reaction was diluted with EtOAc and quenched with sat. aq. sodium potassium tartrate solution. The reaction was extracted with EtOAc. The organic layers were combined, washed with brine, dried over anhydrous Na₂SO₄, filtered, and concentrated under reduced pressure. The resulting residue was purified by silica gel column chromatography (0-100% EtOAc in hex) to afford (Z)—N-(1-(5-bromo-3-methylpyrazin-2-yl)ethylidene)-2-methylpropane-2-sulfinamide. ESI MS m/z=318/320 [M+H]⁺

Step 5: (Z)—N-(1-(5-bromo-3-methylpyrazin-2-yl)ethylidene)-2-methylpropane-2-sulfinamide (935 mg, 2.94 mmol, 1.0 equiv) was dissolved in THF (14.700 mL) and bubble degassed with Ar for 5 min. The solution was cooled to 0° C. and methylmagnesium bromide in THF (1.728 mL, 5.88 mmol, 2.0 equiv) was added dropwise. The reaction was warmed to 25° C. and stirred for 1 hour. Methylmagnesium bromide in THF (1.728 mL, 5.88 mmol, 2.0 equiv) was added at 25° C. and the reaction was stirred for 1 hour. The reaction was cooled to 0° C. and methylmagnesium bromide in THF (1.728 mL, 5.88 mmol, 2.0 equiv) was added and the reaction was stirred at 0° C. for 1 hour. The reaction was quenched slowly with sat. aq. NH₄Cl and extracted with EtOAc (3×). The resulting organic layers were combined, washed with brine, dried over anhydrous Na₂SO₄, filtered, and concentrated under reduced pressure. The resulting residue was purified by silica gel column chromatography (0-100% EtOAc in hex) to afford N-(2-(5-bromo-3-methylpyrazin-2-yl)propan-2-yl)-2-methylpropane-2-sulfinamide (Int-18). ESI MS m/z=334/336 [M+H]⁺

### Tert-butyl (2-(5-bromo-6-(trifluoromethyl)pyrazin-2-yl)propan-2-yl)carbamate (Int-19)

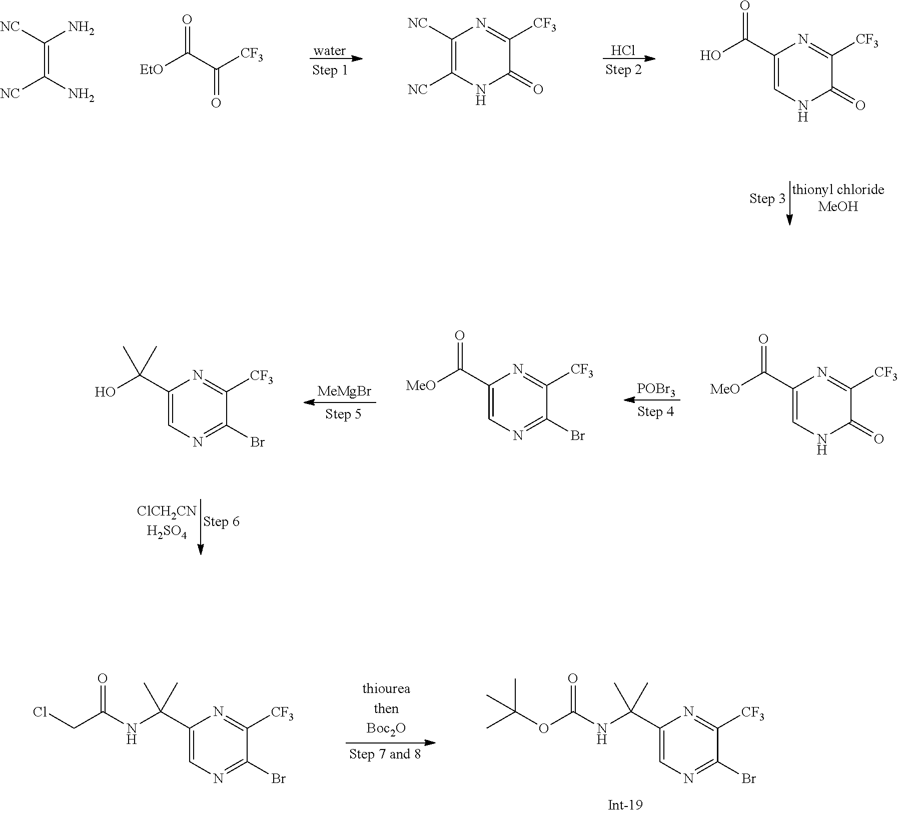

Step 1: Diaminomaleonitrile (6.36 g, 58.8 mmol, 1.0 equiv) was taken up in water (118 ml) and heated at 50° C. Ethyl 3,3,3-trifluoro-2-oxopropanoate (7.79 ml, 58.8 mmol, 1.0 equiv) was added dropwise and the reaction mixture stirred at 50° C. for 4 hours. The reaction was cooled to 25° C. and stirred for 18 hours. The reaction was cooled to 0° C. and filtered. The filtrate was extracted with 3:1 CHCl₃:iPrOH (3×). The resulting organic layers were combined, dried over anhydrous Na₂SO₄, filtered, and concentrated under reduced pressure to afford 6-oxo-5-(trifluoromethyl)-1,6-dihydropyrazine-2,3-dicarbonitrile which was used directly in next step. ESI MS m/z=213 [M−H]⁻

Step 2: 6-Oxo-5-(trifluoromethyl)-1,6-dihydropyrazine-2,3-dicarbonitrile (12.0 g, 56.2 mmol, 1.0 equiv) was taken up in concentrated hydrochloric acid (56.2 ml) and heated at 100° C. for 18 hours. The reaction was cooled to 0° C. The reaction was filtered, and the filtrate was concentrated. The resulting residue was washed with Et₂O and dried under vacuum to afford 5-oxo-6-(trifluoromethyl)-4,5-dihydropyrazine-2-carboxylic acid. ESI MS m/z=231 [M+Na]⁺

Step 3: Methanol (290 ml) was cooled to 0° C. and thionyl chloride (19.01 ml, 261 mmol, 4.5 equiv) was added dropwise. The reaction was stirred at 0° C. for 30 min. 5-Oxo-6-(trifluoromethyl)-4,5-dihydropyrazine-2-carboxylic acid (12.08 g, 58.1 mmol, 1.0 equiv) was added portionwise and the reaction heated at 70° C. for 2 hours. The reaction was concentrated, basified with sat. aq. NaHCO₃, and concentrated. MeOH was added, followed by anhydrous Na₂SO₄. The mixture was filtered. The resulting filtrate was concentrated under reduced pressure and purified by silica gel column chromatography (0-10% MeOH in DCM) to give methyl 5-oxo-6-(trifluoromethyl)-4,5-dihydropyrazine-2-carboxylate. ESI MS m/z=223 [M+H]⁺

Step 4: Methyl 5-oxo-6-(trifluoromethyl)-4,5-dihydropyrazine-2-carboxylate (3 g, 13.5 mmol, 1.0 equiv) and phosphorus oxybromide (7.74 g, 27.0 mmol, 2.0 equiv) was taken up in 1,4-dioxane (67.5 ml) and bubble degassed for 5 min with Ar. The mixture was heated at 100° C. for hours. The reaction was cooled to 25° C. then cooled to 0° C. Ice water was added slowly and the mixture was then extracted with EtOAc (3×). The organic layers were combined, dried over anhydrous Na₂SO₄, filtered, and concentrated under reduced pressure. The resulting residue was purified by silica gel column chromatography (0-50% EtOAc in hex) to afford methyl 5-bromo-6-(trifluoromethyl)pyrazine-2-carboxylate. ESI MS m/z=285/287 [M+H]⁺

Step 5: Methyl 5-bromo-6-(trifluoromethyl)pyrazine-2-carboxylate (3.27 g, 11.47 mmol, 1.0 equiv) was dissolved in THF (57.4 ml) and the resulting solution cooled to −78° C. Methylmagnesium bromide in THF (10.12 ml, 34.4 mmol, 3.0 equiv) was added dropwise and the reaction stirred at 0° C. for 2 hours. The reaction was cooled back down to −78° C., slowly quenched with sat. aq. NH₄Cl and extracted with EtOAc (3×). The resulting organic layers were combined, dried over anhydrous Na₂SO₄, and concentrated under reduced pressure. The resulting residue was purified by silica gel column chromatography (0-100% EtOAc in hex) to afford 2-(5-bromo-6-(trifluoromethyl)pyrazin-2-yl)propan-2-ol. ESI MS m/z=285/287 [M+H]⁺

Step 6: 2-(5-Bromo-6-(trifluoromethyl)pyrazin-2-yl)propan-2-ol (1.43 g, 5.02 mmol, 1.0 equiv) was taken up in chloroacetonitrile (9.52 ml, 150 mmol, 30 equiv.) and cooled to 0° C. Sulfuric acid (1.337 ml, 25.08 mmol, 5.0 equiv) was added dropwise and reaction was warmed to 30° C. and was stirred for 18 hours. Reaction was quenched by adding into ice cold sat. NaHCO₃, extracted with EtOAc (3×), the organic layer was washed with brine, dried over anhydrous Na₂SO₄, and concentrated under reduced pressure. Purified by silica gel column chromatography to afford N-(2-(5-bromo-6-(trifluoromethyl)pyrazin-2-yl)propan-2-yl)-2-chloroacetamide. ESI MS m/z=360/362 [M+H]⁺

Step 7: N-(2-(5-bromo-6-(trifluoromethyl)pyrazin-2-yl)propan-2-yl)-2-chloroacetamide (1.42 g, 3.94 mmol, 1.0 equiv) was taken up in ethanol (16.41 ml) and acetic acid (3.28 ml). Thiourea (0.450 g, 5.91 mmol, 1.5 equiv) was added at 25° C. and the reaction heated at 80° C. for 4 hours. The reaction was concentrated under reduced pressure and used directly in the next step.

Step 8: The residue from previous reaction (1.119 g, 3.9 mmol, 1.0 equiv) was taken up in water (9.85 ml) and THF (9.85 ml). Sodium bicarbonate (1.324 g, 15.8 mmol, 4.0 equiv) then Boc-anhydride (1.830 ml, 7.9 mmol, 2.0 equiv) were added and reaction stirred at 25° C. for 18 hours. The reaction was diluted with water and extracted with EtOAc (3×). The organic layers were combined, washed with brine, dried over anhydrous Na₂SO₄, filtered, and concentrated under reduced pressure. The resulting residue was purified by silica gel column chromatography (0-100% EtOAc in hex) to afford a solid that was taken up in MTBE (50 mL). Ecosorb C-941 (20 wt %) was added and the mixture stirred at 25° C. for 1 hour. The mixture was filtered through Celite™ and concentrated under reduced pressure to afford tert-butyl (2-(5-bromo-6-(trifluoromethyl)pyrazin-2-yl)propan-2-yl)carbamate (Int-19). ESI MS m/z=384/386 [M+H]⁺

### Tert-butyl (2-(5-bromo-6-cyclopropylpyrazin-2-yl)propan-2-yl)carbamate (Int-20)

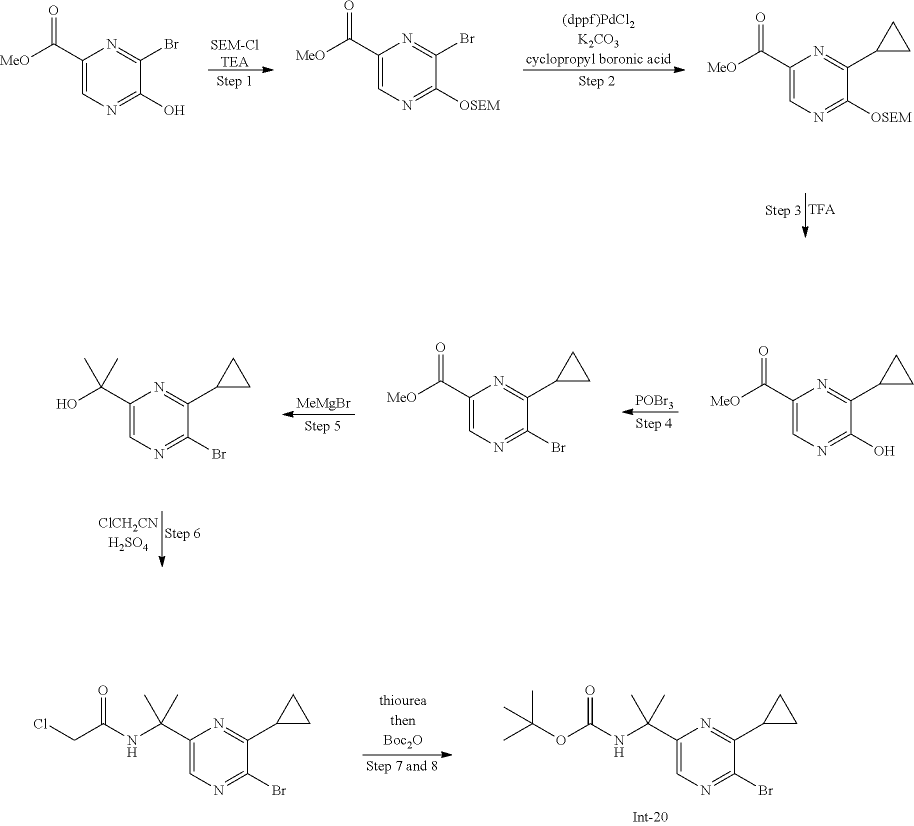

Step 1: Methyl 6-bromo-5-hydroxypyrazine-2-carboxylate (5 g, 21.5 mmol, 1.0 equiv) was taken up in DCM (107 ml) and the resulting solution cooled to 0° C. Triethylamine (5.98 ml, 42.9 mmol, 2.0 equiv) and SEM-Cl (7.61 ml, 42.9 mmol, 2.0 equiv) were added and the reaction stirred at 25° C. for 18 hours. The reaction was diluted with water and extracted with DCM (2×). The organic layers were combined, washed with brine, dried over anhydrous Na₂SO₄, filtered, and concentrated under reduced pressure. The resulting residue was purified by silica gel column chromatography (0-50% EtOAc in hex) to afford methyl 6-bromo-5-((2-(trimethylsilyl)ethoxy)methoxy)pyrazine-2-carboxylate. ESI MS m/z=363/365 [M+H]⁺

Step 2: Methyl 6-bromo-5-((2-(trimethylsilyl)ethoxy)methoxy)pyrazine-2-carboxylate (5.81 g, 16.0 mmol, 1.0 equiv), cyclopropylboronic acid (2.06 g, 24.0 mmol, 1.5 equiv), 1,1′-bis(diphenylphosphino)ferrocene-palladium(II)dichloride dichloromethane complex (1.307 g, 1.6 mmol, 0.1 equiv), and potassium carbonate (6.63 g, 48.0 mmol, 3.0 equiv) were added to 40 mL vial. 1,4-Dioxane (65.5 mL) and water (14.6 ml) were added, and reaction was bubbled with argon for 5 minutes (divided into 4 reaction vials). The reaction was sealed and stirred at 100° C. for 3 hours. The reaction was cooled to 25° C. and filtered through Celite™ while washing with EtOAc. The filtrate was diluted with brine and extracted with EtOAc (3×). The combined organic layers were dried over anhydrous Na₂SO₄, filtered and concentrated under reduced pressure. The resulting crude residue was purified by silica gel column chromatography (0-50% EtOAc in hex) to afford methyl 6-cyclopropyl-5-((2-(trimethylsilyl)ethoxy)methoxy)pyrazine-2-carboxylate. ESI MS m/z=325 [M+H]⁺

Step 3: Methyl 6-cyclopropyl-5-((2-(trimethylsilyl)ethoxy)methoxy)pyrazine-2-carboxylate (2.57 g, 7.92 mmol, 1.0 equiv) was taken up in TFA (39.6 ml) and stirred at 25° C. for 1 hour. The reaction was cooled to 0° C. and slowly quenched with sat. aq. NaHCO₃ and extracted with DCM (3×). The combined organic layers were dried over anhydrous Na₂SO₄, filtered, and concentrated under reduced pressure. The resulting crude residue was purified by silica gel column chromatography (0-100% 3:1 EtOAc:EtOH in hex) to afford methyl 6-cyclopropyl-5-hydroxypyrazine-2-carboxylate. ESI MS m/z=195 [M+H]⁺

Step 4: Methyl 6-cyclopropyl-5-oxo-4,5-dihydropyrazine-2-carboxylate (1.36 g, 7.0 mmol, 1.0 equiv) and phosphorus oxybromide (4.02 g, 14.0 mmol, 2.0 equiv) were taken up in 1,4-dioxane (17.5 ml) and bubble degassed for 5 minutes with Ar. The reaction mixture was heated at 100° C. for 1.5 hours, cooled to 25° C. then cooled to 0° C. Saturated aqueous NaHCO₃ was added slowly and the mixture extracted with EtOAc (3×). The combined organic layers were dried over anhydrous Na₂SO₄, filtered, and concentrated under reduced pressure. The resulting crude residue was purified by silica gel column chromatography (0-100% EtOAc in hex) to afford methyl 5-bromo-6-cyclopropylpyrazine-2-carboxylate. ESI MS m/z=257/259 [M+H]⁺

Step 5: Methyl 5-bromo-6-cyclopropylpyrazine-2-carboxylate (257 mg, 1.0 mmol, 1.0 equiv) dissolved in THF (4998 μl) and cooled to −78° C. Methylmagnesium bromide in THF (882 μl, 3.0 mmol, 3.0 equiv) added dropwise and reaction stirred at 0° C. for 2 hours. Cooled back down to −78° C. and slowly quenched with sat. NH₄Cl, extracted with EtOAc (3×), dried over anhydrous Na₂SO₄, and concentrated under reduced pressure. Purified by silica gel column chromatography (0-100% EtOAc in hex) to afford 2-(5-bromo-6-cyclopropylpyrazin-2-yl)propan-2-ol. ESI MS m/z=257/259 [M+H]⁺

Step 6: 2-(5-Bromo-6-cyclopropylpyrazin-2-yl)propan-2-ol (159 mg, 0.62 mmol, 1.0 equiv) was taken up in chloroacetonitrile (1174 μl, 18.6 mmol, 18.6 equiv) and cooled to 0° C. Sulfuric acid (165 μl, 3.1 mmol, 3.0 equiv) was added dropwise and reaction was warmed to 30° C. and was stirred for 18 hours. Reaction was cooled to 0° C. and diluted with water. Solid NaHCO₃ was added until pH ˜10 and extracted with EtOAc (3×), the organic layer was washed with brine, dried over anhydrous Na₂SO₄, and concentrated under reduced pressure. the solid was filtered off and filtrate purified by silica gel column chromatography (0-100% EtOAc in hex) to afford N-(2-(5-bromo-6-cyclopropylpyrazin-2-yl)propan-2-yl)-2-chloroacetamide. ESI MS m/z=332/334 [M+H]⁺

Step 7: N-(2-(5-Bromo-6-cyclopropylpyrazin-2-yl)propan-2-yl)-2-chloroacetamide (167 mg, 0.50 mmol, 1.0 equiv) taken up in ethanol (2092 μl) and acetic acid (418 μl). Thiourea (57.3 mg, 0.75 mmol, 1.5 equiv) added at 25° C. and reaction heated at 80° C. for 3 hours. Concentrated under reduced pressure and used directly in next step.

Step 8: Residue from previous reaction (129 mg, 0.50 mmol) was taken up in water (1255 μl) and THF (1255 μl). Sodium bicarbonate (169 mg, 2.0 mmol, 4.0 equiv) then Boc-anhydride (233 μl, 1.0 mmol, 2.0 equiv) added and reaction stirred at 25° C. for 2 hours. Reaction diluted with water, extracted with EtOAc (3×), wash with brine, dried over anhydrous Na₂SO₄, and concentrated under reduced pressure. Purified by silica gel column chromatography (0-100% EtOAc in hex). Solid taken up in MTBE (50 mL) and Ecosorb C-941 (20 wt %) was added. Stirred at 25° C. for 1 hour. Filtered through Celite™ and concentrated under reduced pressure to afford tert-butyl (2-(5-bromo-6-cyclopropylpyrazin-2-yl)propan-2-yl)carbamate (Int-20). ESI MS m/z=356/358 [M+H]⁺

### 3-Phenylcyclobutane-1-carbohydrazide (Int-21)

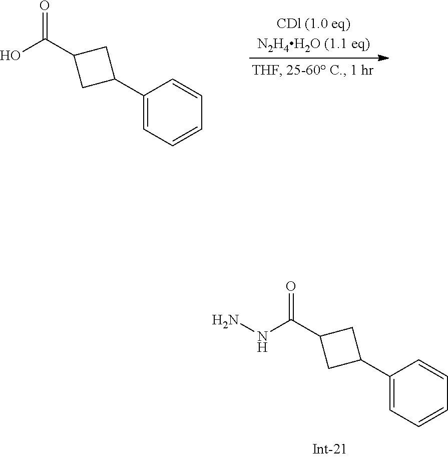 [SMILES: O=C(O)C1CC(c2ccccc2)C1 ; NNC(=O)C1CC(c2ccccc2)C1]

A 20 mL scintillation vial was charged with CDI (60 mg, 2.84 mmol, 1.0 equiv) and THF (3.5 mL), and the resulting mixture was stirred at 25° C. 3-phenylcyclobutane-1-carboxylic acid (500 mg, 2.84 mmol, 1.0 equiv) was added, and the reaction was heated at 60° C. for 1 h. A second 20 mL scintillation vial was charged with hydrazine hydrate (276 μL, 5.67 mmol, 2.0 equiv) and THF (3.5 mL). The reaction mixture containing the intermediate acylimidazole was added in portions to the hydrazine hydrate solution over 1 hour. After the addition was complete, the reaction was concentrated. The residue was taken up in EtOAc (50 mL), washed with water (20 mL), dried over anhydrous Na₂SO₄, filtered and concentrated under reduced pressure to provide 3-phenylcyclobutane-1-carbohydrazide (Int-21). ESI MS m/z=191 [M+H]⁺

### 3-(5-(2-((Tert-butoxycarbonyl)amino)propan-2-yl)-3-methylpyrazin-2-yl)cyclobutane-1-carboxylic acid (Int-22)

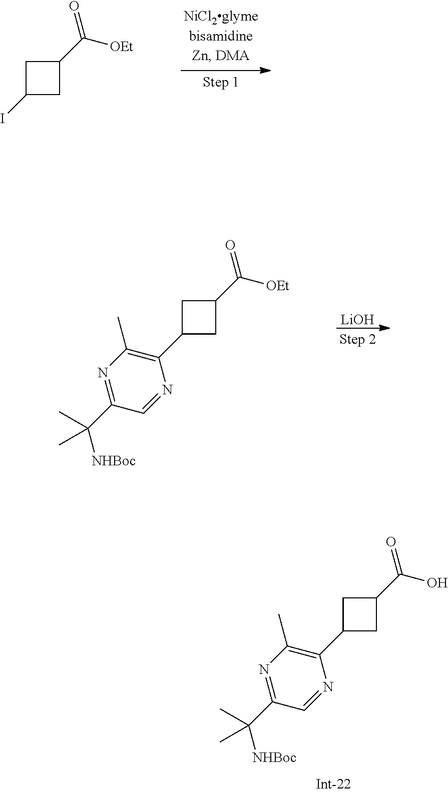 [SMILES: CCOC(=O)C1CC(I)C1 ; CCOC(=O)C1CC(c2ncc(C(C)(C)NC(=O)OC(C)(C)C)nc2C)C1 ; "LiOH" ; Cc1nc(C(C)(C)NC(=O)OC(C)(C)C)cnc1C1CC(C(=O)O)C1]

Step 1: Tert-butyl (2-(5-bromo-6-methylpyrazin-2-yl)propan-2-yl)carbamate (3.38 g, 10.23 mmol), pyridine-2,6-bis(carboximidamide) dihydrochloride (0.829 g, 3.54 mmol), ethyl 3-iodocyclobutane-1-carboxylate (2 g, 7.87 mmol), nickel(II) chloride ethylene glycol dimethyl ether complex (0.778 g, 3.54 mmol), and zinc (2.059 g, 31.5 mmol) added. DMA (79 ml) added and bubbled with Ar for 5 min. Heated at 50° C. for 18 hours. Diluted with EtOAc and passed through Celite™. The organic layer was washed with brine (2×) and dried over anhydrous Na₂SO₄. Concentrated under reduced pressure and purified by silica gel column chromatography (0-100% EtOAc in hex) to afford ethyl 3-(5-(2-((tert-butoxycarbonyl)amino)propan-2-yl)-3-methylpyrazin-2-yl)cyclobutane-1-carboxylate. ESI MS m/z=378 [M+H]⁺

Step 2: Ethyl 3-(5-(2-((tert-butoxycarbonyl)amino)propan-2-yl)-3-methylpyrazin-2-yl)cyclobutane-1-carboxylate (429 mg, 1.136 mmol) taken up in THF (4546 μl) and water (1136 μl). Lithium hydroxide (54.4 mg, 2.273 mmol) added and stirred at 25° C. for 18 hours. Diluted with EtOAc and organic layer collected. Aqueous layer acidified to pH 1 and extracted with EtOAc (3×), dried over anhydrous Na₂SO₄, and concentrated under reduced pressure to afford 3-(5-(2-((tert-butoxycarbonyl)amino)propan-2-yl)-3-methylpyrazin-2-yl)cyclobutane-1-carboxylic acid (Int-22). ESI MS m/z=350 [M+H]⁺

### N-(1-(6-bromopyridin-3-yl)-2,2,2-trifluoroethyl)-2-methylpropane-2-sulfinamide (Int-23)

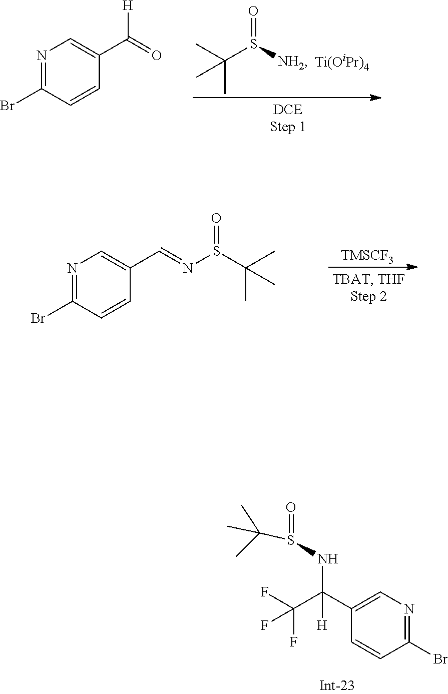

Step 1: In a 5 mL microwave vial 6-bromonicotinaldehyde (2 g, 10.75 mmol) and (R)-2-methylpropane-2-sulfinamide (1.955 g, 16.13 mmol) were added, and the mixture was backfilled with Ar for 2 min. DCE (10 mL) and titanium isopropoxide (IV) (4.7 mL, 16.13 mmol) were added and the resulting solution was placed in the microwave and stirred at 100° C. for 15 minutes. The mixture was diluted with DCM (50 mL) and water (10 mL), resulting in an emulsion, allowed it to stir for 5 minutes, and then filtered under vacuum. The resulting solution was transferred to a separatory funnel, the layers were separated, the aqueous layer was extracted with DCM (20 mL). The combined organic layers were dried over anhydrous Na₂SO₄ filter and concentrated under reduced pressure to afford (R,E)-N-((6-bromopyridin-3-yl)methylene)-2-methylpropane-2-sulfinamide.

Step 2: Tetrabutylammonium difluorotriphenylsilicate(IV) (4.52 g, 8.37 mmol) was added to a mixture of (R,E)-N-((6-bromopyridin-3-yl)methylene)-2-methylpropane-2-sulfinamide (2.2 g, 7.61 mmol) in THF (127 mL). The mixture was cooled to −60° C. In a separate flask a solution of trimethyl(trifluoromethyl)silane (2.7 mL, 18.26 mmol) in THF (0.3 M) was prepared. The resulting solution was transferred via cannula to the reaction mixture. The reaction was quenched in cold with NH₄Cl (sat aq solution), allowed it to warm to room temperature, DCM (30 mL) was added, and the mixture was stirred for 5 min. Then transferred to a separatory funnel, the layers were separated and the aqueous layer was extracted with DCM (20 mL×2). The combined organic layers were dried over anhydrous Na₂SO₄, filter and concentrated under reduced pressure. The crude material was purified by silica gel column chromatography (0-70% EtOAc in hex) to afford (R)—N-(1-(6-bromopyridin-3-yl)-2,2,2-trifluoroethyl)-2-methylpropane-2-sulfinamide (Int-23). ESI MS m/z=359/361 [M+H]⁺

TABLE 1
Int-24 was prepared using the same procedure as Int-23 starting with (S)-2-
methylpropane-2-sulfinamide.

| Compound | Structure | Name | Exact mass [M + H]+ |
|---|---|---|---|
| Int-24 | 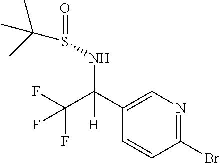 [SMILES: CC(C)(C)[S@](=O)NC(c1ccc(Br)nc1)C(F)(F)F] | (S)-N-(1-(6- bromopyridin-3-yl)- 2,2,2-trifluoroethyl)- 2-methylpropane-2- sulfinamide | 359/361 |

### N-(1-(6-bromopyridin-3-yl)-2,2-difluoroethyl)-2-methylpropane-2-sulfinamide (Int-25)

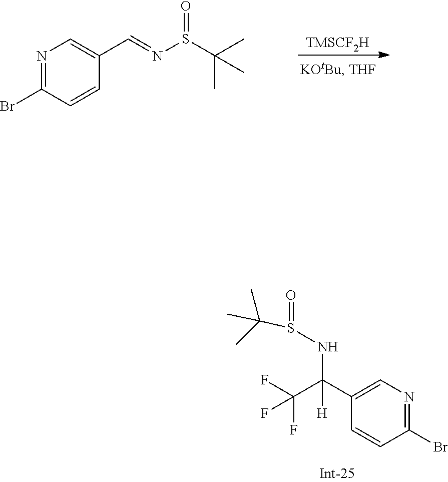 [SMILES: "TMSCF2H" ; CC(C)(C)S(=O)/N=C/c1ccc(Br)nc1 ; CC(C)(C)S(=O)NC(c1ccc(Br)nc1)C(F)(F)F]

Potassium tert-butoxide in THF (1M, 2075 μl, 2.075 mmol) was added dropwise to a solution of (E)-N-((6-bromopyridin-3-yl)methylene)-2-methylpropane-2-sulfinamide (200 mg, 0.692 mmol) in THF (6916 μl), cooled to −78° C. under argon. The resulting solution was slowly warmed to room temperature over 2 hours. The reaction was cooled back down to −78° C. and quenched with sat. aq. NH₄Cl (8 mL). After removing the dry ice bath, water (10 mL) and DCM (25 mL) were added, and the layers were separated. The aqueous layer was extracted with DCM (2×25 mL) and the combined organic layers were dried over anhydrous MgSO₄ and concentrated. The crude material was purified by silica gel column chromatography (0-100% 3:1 EtOAc:EtOH in hex) to provide N-(1-(6-bromopyridin-3-yl)-2,2-difluoroethyl)-2-methylpropane-2-sulfinamide (Int-25). ESI MS m/z=341/343 [M+H]⁺

### (S)—N-(2-(5-bromopyridin-2-yl)-1,1-difluoropropan-2-yl)-2-methylpropane-2-sulfinamide (Int-26)

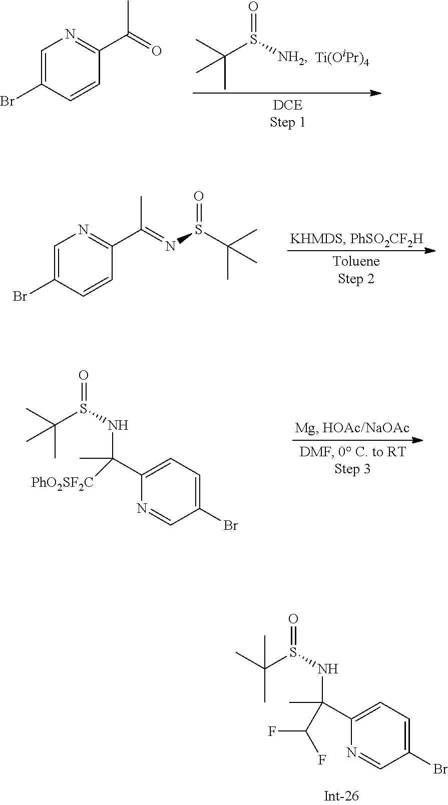

Step 1: In a 20 mL microwave vial 1-(5-bromopyridin-2-yl)ethan-1-one (1 g, 5.0 mmol) and (S)-2-methylpropane-2-sulfinamide (0.909 g, 7.50 mmol) were added, the mixture was purged with Ar for 2 min. DCE (10.00 mL) and titanium (IV) isopropoxide (2.2 mL, 7.50 mmol) were added and the resulting solution was placed in the microwave and stirred at 100° C. for 30 min. The mixture was diluted with DCM (30 mL) and water (10 mL), resulting an emulsion. The reaction mixture stirred for 5 min, then filtered under vacuum. The resulting biphasic solution was transferred to a separatory funnel, the layers were separated, the aqueous layer was extracted with DCM (20 mL) and then with IPA:CHCl₃ (1:3). The combined organic layers were dried over anhydrous Na₂SO₄, filtered, and concentrated under reduced pressure. The crude mixture was purified by silica gel column chromatography (0-50% EtOAc in hex) to afford (S,E)-N-(1-(5-bromopyridin-2-yl)ethylidene)-2-methylpropane-2-sulfinamide. ESI MS m/z=303/305 [M+H]⁺

Step 2: In a 20 mL vial containing (S,E)-N-(1-(5-bromopyridin-2-yl)ethylidene)-2-methylpropane-2-sulfinamide (50 mg, 0.17 mmol) and ((difluoromethyl)sulfonyl)benzene (38.0 mg, 0.198 mmol) in toluene (1.4 mL) at −78° C., KHMDS in toluene (429 μl, 0.214 mmol) was added dropwise. The reaction was stirred for 1 hour, then the mixture was quenched cold by addition of cold brine, then the bath was removed. The mixture was diluted with DCM (10 mL) and allowed to warm to room temperature and transferred to a phase separator cartridge. The aqueous layer was extracted with DCM (5 mL). The combined organic layers were concentrated under reduced pressure. The crude material was purified by silica gel column chromatography (0-70% EtOAc in hex) to afford (S)—N-(2-(5-bromopyridin-2-yl)-1,1-difluoro-1-(phenylsulfonyl)propan-2-yl)-2-methylpropane-2-sulfinamide. ESI MS m/z=495/497 [M+H]⁺

Step 3: 7.0 mL of AcOH/NaOAc (1:1) buffer solution (8 mol/L) was added to a solution of (S)—N-(2-(5-bromopyridin-2-yl)-1,1-difluoro-1-(phenylsulfonyl)propan-2-yl)-2-methylpropane-2-sulfinamide (380 mg, 0.77 mmol) in DMF (7.7 mL) at room temperature. Magnesium (373 mg, 15.34 mmol) was added portionwise at 0° C. The ice bath was removed after 15 min, and the reaction was stirred at room temperature for 3 hours. The mixture was quenched by adding NaHCO₃ (sat. aqueous solution) and the mixture was diluted with DCM. The layers were separated and the crude mixture was purified by silica gel column chromatography (0-70% 3:1 EtOAc:EtOH in hex) to afford (S)—N-(2-(5-bromopyridin-2-yl)-1,1-difluoropropan-2-yl)-2-methylpropane-2-sulfinamide (Int-W). ESI MS m/z=355/357 [M+H]⁺

### N-(1-(5-bromopyridin-2-yl)-2,2,2-trifluoroethyl)-2-methylpropane-2-sulfinamide (Int-27

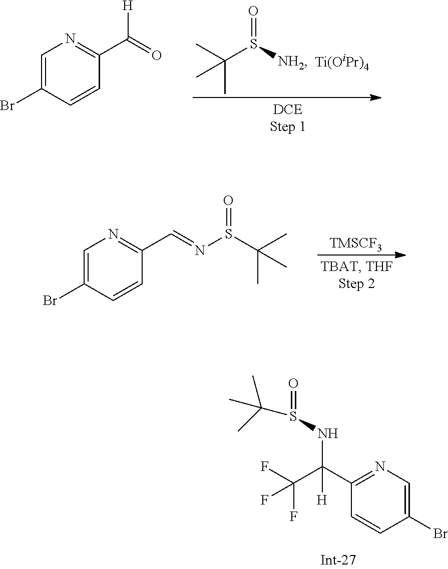

Step 1: In a 20 mL microwave vial 5-bromopicolinaldehyde (1.5 g, 8.06 mmol) and (R)-2-methylpropane-2-sulfinamide (1.466 g, 12.10 mmol) were added. The mixture was purged with Ar for 2 min. DCE (8.06 mL) and titanium (IV) isopropoxide (3.54 mL, 12.10 mmol) were added. The resulting solution was placed in the microwave and stirred at 100° C. for 15 min. The mixture was diluted with DCM (70 mL) and water (10 mL), resulting an emulsion which was stirred for 5 min, and then filtered under vacuum to break the emulsion. The resulting solution was transferred to a separatory funnel, the layers were separated, the aqueous layer was extracted with DCM (20 mL), then the aqueous layer was further extracted with IPA:CHCl₃ 1:3 (30 mL×2). The combined organic layers were dried over anhydrous Na₂SO₄, filtered, and concentrated under reduced pressure. The crude mixture was purified by silica gel column chromatography (0-50% EtOAc in hex) to afford (E)-N-((5-bromopyridin-2-yl)methylene)-2-methylpropane-2-sulfinamide.

Step 2: In a 500 mL round bottom flask (E)-N-((5-bromopyridin-2-yl)methylene)-2-methylpropane-2-sulfinamide (0.7 g, 2.421 mmol), tetrabutylammonium difluorotriphenylsilicate (IV) (1.437 g, 2.66 mmol) were added, followed by addition of THF (48.4 ml). The resulting solution was cooled to −60° C. and stirred for 10 min. The solution became a slurry. In a second flask a 0.3 M solution in THF (12 mL) of trimethyl(trifluoromethyl)silane (0.537 ml, 3.63 mmol) was prepared and the resulting solution was transferred via cannula to the original reaction mixture. Upon total addition of the TMSCF₃ solution, the reaction mixture was stirred for 20 min. After 40 min of stirring, the reaction was cooled to 0° C. and quenched with NH₄Cl (sat aq solution) (15 mL), then DCM was added (30 mL). The cold bath was removed, and the mixture was allowed to stir at 25° C. for 10 min., then transferred to a phase separator. The layers were separated, and the aqueous layer was extracted with DCM (15 mL). The combined organic layers were dried over anhydrous Na₂SO₄, filtered and concentrated under reduced pressure. The resulting crude material was purified by silica gel column chromatography (0-70% EtOAc in hex) to afford (R)—N-(1-(5-bromopyridin-2-yl)-2,2,2-trifluoroethyl)-2-methylpropane-2-sulfinamide (Int-27). ESI MS m/z=359/361 [M+H]⁺

### 1-(6-bromopyridin-3-yl)-2-methylpropan-2-ol (Int-28)

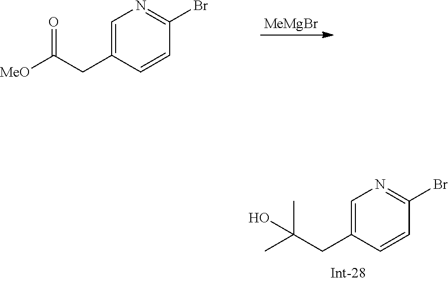

Methyl 2-(6-bromopyridin-3-yl)acetate (1 g, 4.35 mmol) and THF (9.66 ml) were added to a 50 mL round bottom flask. The mixture was stirred and cooled at −30° C. Methylmagnesium bromide in THF (3M, 3.33 ml, 10.00 mmol) was added to the mixture over 5 minutes. The mixture was warmed to room temperature over 15 minutes and stirred at RT for 30 minutes. To the mixture was added saturated aqueous ammonium chloride (10 mL) and DCM (15 mL) and stirred for 20 minutes. The mixture was diluted with water (5 mL) and extracted 3× with DCM (15 mL) using a phase separator cartridge. The combined organic layers were dried over anhydrous MgSO₄, filtered and concentrated. The crude material was purified by silica gel column chromatography (0-100% 3:1 EtOAc:EtOH in hex) to afford 1-(6-bromopyridin-3-yl)-2-methylpropan-2-ol (Int-28). ESI MS m/z=330/332 [M+H]⁺

### 2-(5-bromopyrazin-2-yl)propan-2-ol (Int-29)

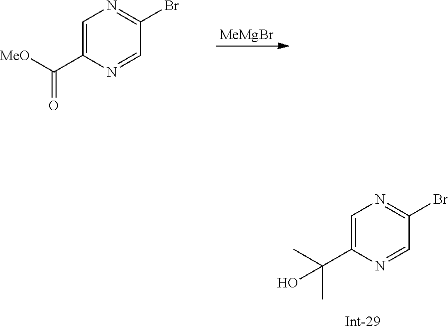

Methyl 5-bromopyrazine-2-carboxylate (1 g, 4.61 mmol) and THF (10.24 ml) was added to a 50 mL round bottom flask. The mixture was stirred and cooled at −30° C. Methylmagnesium bromide in THF (3M, 3.53 mL, 10.60 mmol) was added to the mixture over 5 minutes. The mixture was warmed to room temperature over 15 minutes and stirred at room temperature for 30 minutes. Saturated aqueous ammonium chloride (10 mL) and DCM (15 mL) was added, and the mixture was stirred for 20 minutes. The mixture was diluted with water (10 mL) and extracted 3× with DCM (20 mL) using a phase separator cartridge. The combined organic layers were dried over anhydrous MgSO₄, filtered and concentrated. The crude material was purified by silica gel column chromatography (0-100% 3:1 EtOAc:EtOH in hex) to afford 2-(5-bromopyrazin-2-yl)propan-2-ol (Int-29). ESI MS m/z=217/219 [M+H]⁺

### 2-(2-bromopyrimidin-5-yl)propan-2-ol (Int-30)

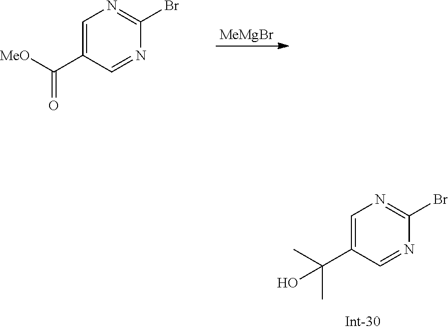

Methyl 2-bromopyrimidine-5-carboxylate (1 g, 4.61 mmol) and THF (10.24 ml) was added to a 50 mL round bottom flask. The mixture was stirred and cooled at −30° C. Methylmagnesium bromide in THF (3M, 3.53 ml, 10.60 mmol) was added to the mixture over 5 minutes. The mixture was warmed to room temperature over 15 minutes and stirred at room temperature for 30 minutes. Saturated aqueous ammonium chloride (10 mL) and DCM (15 mL) was added and the mixture was stirred for 20 minutes. The mixture was diluted with water (10 mL) and extracted 3× with DCM (20 mL) using a phase separator cartridge. The combined organic layers were dried over anhydrous MgSO₄, filtered and concentrated. The crude material was purified by silica gel column chromatography (0-100% 3:1 EtOAc:EtOH in hex) to afford 2-(2-bromopyrimidin-5-yl)propan-2-ol (Int-&). ESI MS m/z=217/219 [M+H]⁺

### 1-(6-bromopyridin-3-yl)-3,3-difluorocyclobutan-1-ol (Int-31)

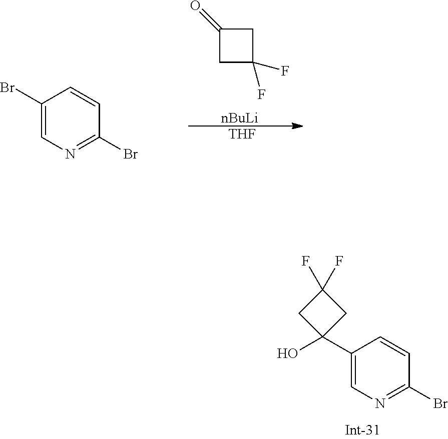

A 250 mL round bottom flask was charged with 2,5-dibromopyridine (4.47 g, 18.86 mmol), then backfilled and evacuated 3× with nitrogen. Diethyl ether (94 mL) was then added, and the resulting solution was then cooled to −78° C. N-butyllithium (7.54 mL, 18.86 mmol) was added dropwise, and the resulting suspension was stirred at −78° C. for 30 min. 3,3-Difluorocyclobutan-1-one (1 g, 9.43 mmol) was dissolved in THF (12 mL) and this solution was slowly added to the reaction flask, with the bottle and syringe rinsed with THF (8 mL). The resulting reaction mixture was then stirred for 2 hours at −78° C. The reaction was quenched with saturated aqueous ammonium chloride (50 mL) and treated with DCM (200 mL) and water (50 mL). The layers were separated, and the aqueous layer was extracted with DCM (3×80 mL). The combined organic layers were dried over anhydrous MgSO₄ and concentrated. The crude material was purified by silica gel column chromatography (0-100% EtOAc in hex) to provide 1-(6-bromopyridin-3-yl)-3,3-difluorocyclobutan-1-ol (Int-31). ESI MS m/z=264/266 [M+H]⁺

### EXAMPLES

### Example 1: 2-(4-((1r,2r)-2-(5-amino-7-methoxy-[1,2,4]triazolo[1,5-c]quinazolin-2-yl)cyclopropyl)phenyl)propan-2-ol (Ex-1)

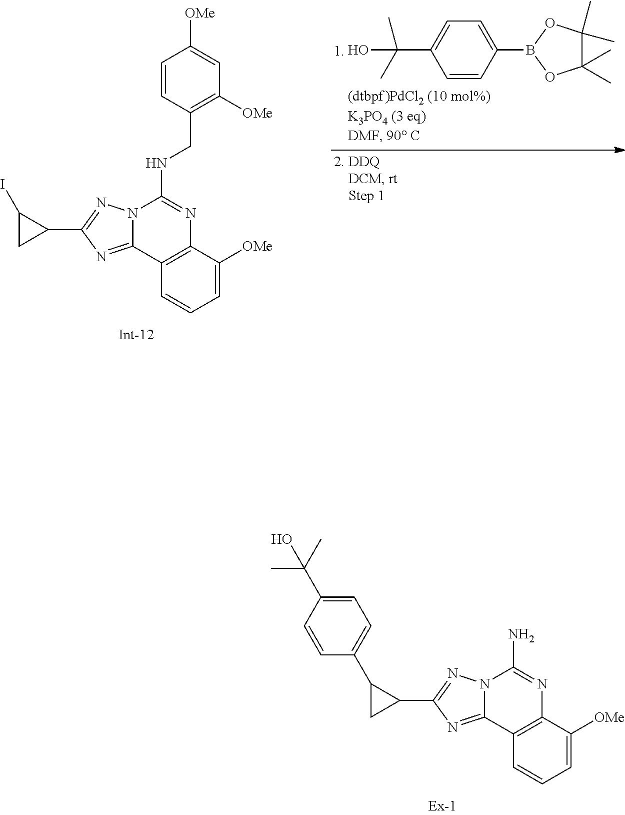 [SMILES: COc1ccc(CNc2nc3c(OC)cccc3c3nc(C4CC4I)nn23)c(OC)c1 ; CC(C)(O)c1ccc(B2OC(C)(C)C(C)(C)O2)cc1 ; COc1cccc2c1nc(N)n1nc(C3CC3c3ccc(C(C)(C)O)cc3)nc21]

N-(2,4-dimethoxybenzyl)-2-(2-iodocyclopropyl)-7-methoxy-[1,2,4]triazolo[1,5-c]quinazolin-5-amine (Int-12) (75 mg, 0.141 mmol, 1.0 equiv), 2-(4-(4,4,5,5-tetramethyl-1,3,2-dioxaborolan-2-yl)phenyl)propan-2-ol (55.5 mg, 0.212 mmol, 1.5 equiv), and 1,1′-bis(di-tert-butylphosphino)ferrocene palladium dichloride (9.20 mg, 0.014 mmol, 0.1 equiv) were dissolved in DMF (235 μl) and potassium phosphate (180 μl, 0.423 mmol, 3.0 equiv) (50% water) was added. The mixture was purged with N₂ for 5 min, then heated at 90° C. for 18 hours. The reaction mixture was diluted with DCM and water and the layers were separated using a phase separator. The organic layer was concentrated, and the residual oil was redissolved in DCM (1.41 mL) and DDQ (32.0 mg, 0.141 mmol, 1.0 equiv) was added. The reaction mixture was stirred at 25° C. for 3 hours and then loaded directly onto a silica gel column eluting from 0-10% MeOH in DCM to yield 2-(4-(2-(5-amino-7-methoxy-[1,2,4]triazolo[1,5-c]quinazolin-2-yl)cyclopropyl)phenyl)propan-2-ol (Ex-145). ESI MS m/z=390 [M+H]⁺. ¹H NMR (500 MHz, DMSO-d₆) δ 7.76 (s, 2H), 7.72 (d, J=8.0 Hz, 1H), 7.39 (d, J=8.1 Hz, 2H), 7.30 (t, J=7.9 Hz, 1H), 7.22 (d, J=8.0 Hz, 1H), 7.18 (d, J=8.2 Hz, 2H), 3.92 (s, 3H), 2.58 (m, 1H), 2.47 (dd, J=8.9, 4.4 Hz, 1H), 1.76 (dt, J=9.3, 4.8 Hz, 1H), 1.58 (dt, J=9.0, 5.4 Hz, 1H), 1.08 (s, 6H).

### Example 2: 7-methoxy-2-((1r,2r)-2-(1-methyl-1H-pyrazol-4-yl)cyclopropyl)-[1,2,4]triazolo[1,5-c]quinazolin-5-amine (Ex-2)

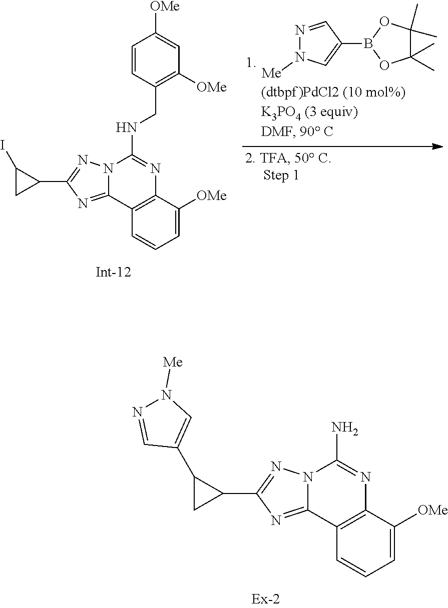 [SMILES: COc1ccc(CNc2nc3c(OC)cccc3c3nc(C4CC4I)nn23)c(OC)c1 ; Cn1cc(B2OC(C)(C)C(C)(C)O2)cn1 ; COc1cccc2c1nc(N)n1nc(C3CC3c3cnn(C)c3)nc21]

N-(2,4-dimethoxybenzyl)-2-(2-iodocyclopropyl)-7-methoxy-[1,2,4]triazolo[1,5-c]quinazolin-5-amine (Int-12) (50 mg, 0.094 mmol), 1-methyl-4-(4,4,5,5-tetramethyl-1,3,2-dioxaborolan-2-yl)-1H-pyrazole (27.4 mg, 0.141 mmol), and 1,1′-bis(di-tert-butylphosphino)ferrocene palladium dichloride (6.13 mg, 9.41 μmol) were dissolved in DMF (157 μl) and potassium phosphate (120 μl, 0.282 mmol) (50% water) was added. The mixture was purged with N₂ for 5 min, heated at 90° C. for 2 h. The mixture was quenched with sat. aqueous NH₄Cl (200 μL) and diluted with ethyl acetate. A small amount of Celite™ was added and the reaction was vigorously stirred for five minutes. The heterogeneous mixture was poured over a Celite™ filter that had a layer of anhydrous MgSO₄ on top and rinsed with ethyl acetate. The filtrate was concentrated, re-dissolved in trifluoroacetic acid (725 μl, 9.41 mmol) and heated to 50° C. for 16 hours. The reaction mixture was cooled to room temperature, concentrated, diluted with 2 mL DCM and quenched with 2 mL sat'd aq. NaHCO₃. The DCM layer was collected using a phase separator and was concentrated. The residual oil was dissolved in 3 mL DMSO, filtered, and submitted for RP-HPLC purification using the acidic method to yield 7-methoxy-2-(2-(1-methyl-1H-pyrazol-4-yl)cyclopropyl)-[1,2,4]triazolo[1,5-c]quinazolin-5-amine, TFA (Ex-2). ESI MS m/z=336 [M+H]⁺. ¹H NMR (500 MHz, DMSO-d₆) δ 8.01 (s, 2H), 7.72 (dd, J=7.9, 1.1 Hz, 1H), 7.62 (s, 1H), 7.36 (s, 1H), 7.33 (t, J=7.9 Hz, 1H), 7.26 (d, J=7.1 Hz, 1H), 3.93 (s, 3H), 3.78 (s, 3H), 2.47-2.39 (m, 1H), 2.32 (dt, J=9.4, 5.1 Hz, 1H), 1.64 (dt, J=9.2, 4.5 Hz, 1H), 1.42 (ddd, J=8.6, 6.1, 4.3 Hz, 1H).

TABLE 2
Using the appropriate starting materials and a method similar to that described above, the
following examples were prepared:

| | | | Exact Mass |
|---|---|---|---|
| Example | Structure | Name | [M + H]+ |
| Ex-3 | 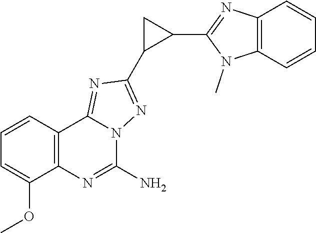 [SMILES: COc1cccc2c1nc(N)n1nc(C3CC3c3nc4ccccc4n3C)nc21] | 7-methoxy-2-[2-(1- methyl-1H- benzimidazol-2- yl)cyclopropyl][1,2,4] triazolo[1,5-c] quinazolin-5-amine | 386 |
| Ex-4 | 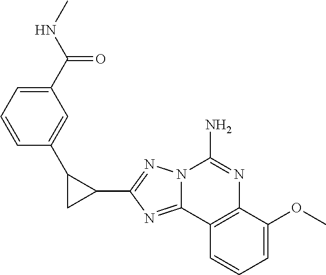 [SMILES: CNC(=O)c1cccc(C2CC2c2nc3c4cccc(OC)c4nc(N)n3n2)c1] | 3-[2-(5-amino-7- methoxy[1,2,4]triazolo [1,5-c]quinazolin-2-yl)cyclopropyl]-N-methylbenzamide | 389 |
| Ex-5 | 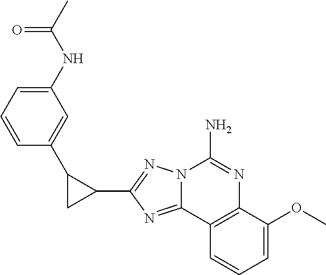 [SMILES: COc1cccc2c1nc(N)n1nc(C3CC3c3cccc(NC(C)=O)c3)nc21] | N-{3-[2-(5-amino-7- methoxy[1,2,4]triazolo [1,5-c]quinazolin-2-yl)cyclopropyl]phenyl} acetamide | 389 |
| Ex-6 | 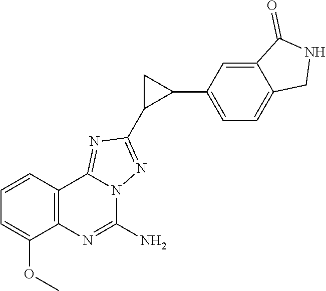 [SMILES: COc1cccc2c1nc(N)n1nc(C3CC3c3ccc4c(c3)C(=O)NC4)nc21] | 6-[2-(5-amino-7- methoxy[1,2,4]triazolo [1,5-c]quinazolin-2-yl)cyclopropyl]-2,3-dihydro-1H-isoindol- 1-one | 387 |
| Ex-7 | 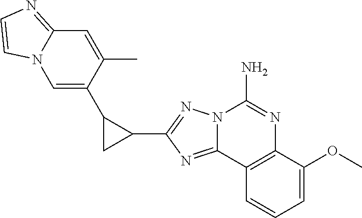 [SMILES: COc1cccc2c1nc(N)n1nc(C3CC3c3cn4ccnc4cc3C)nc21] | 7-methoxy-2-[2-(7- methylimidazo[1,2-alpyridin-6- yl)cyclopropyl][1,2,4] triazolo[1,5-c] quinazolin-5-amine | 386 |
| Ex-8 | 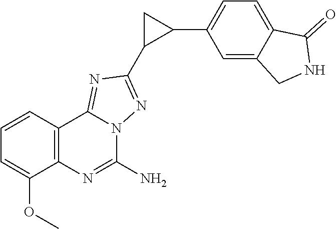 [SMILES: COc1cccc2c1nc(N)n1nc(C3CC3c3ccc4c(c3)CNC4=O)nc21] | 5-[2-(5-amino-7- methoxy[1,2,4]triazolo [1,5-c]quinazolin-2-yl)cyclopropyl]-2,3-dihydro-1H-isoindol- 1-one | 387 |
| Ex-9 | 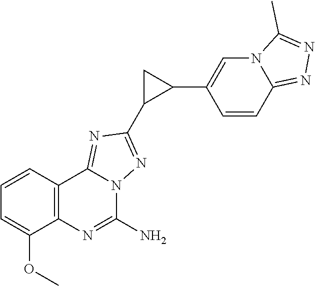 [SMILES: COc1cccc2c1nc(N)n1nc(C3CC3c3ccc4nnc(C)n4c3)nc21] | 7-methoxy-2-[2-(3- methyl[1,2,4]triazolo [4,3-a]pyridin-6-yl)cyclopropyl][1,2,4] triazolo[1,5-c] quinazolin-5-amine | 387 |
| Ex-10 | 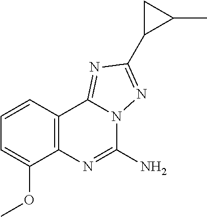 | 7-methoxy-2-[2- methylcyclopropyl] [1,2,4]triazolo[1,5-c] quinazolin-5-amine | 270 |
| Ex-11 | 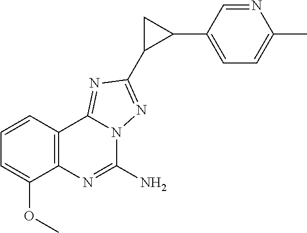 [SMILES: COc1cccc2c1nc(N)n1nc(C3CC3c3ccc(C)nc3)nc21] | 7-methoxy-2-[2-(6- methylpyridin-3- yl)cyclopropyl][1,2,4] triazolo[1,5-c] quinazolin-5-amine | 347 |
| Ex-12 | 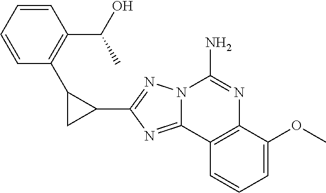 [SMILES: COc1cccc2c1nc(N)n1nc(C3CC3c3ccccc3[C@@H](C)O)nc21] | (1R)-1-{2-[2-(5- amino-7- methoxy[1,2,4]triazolo [1,5-c]quinazolin-2- yl)cyclopropyl]phenyl} ethan-1-ol | 376 |
| Ex-13 | 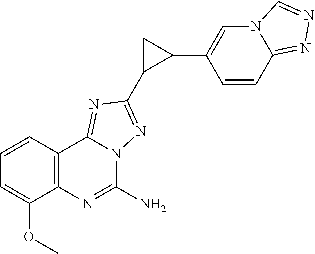 [SMILES: COc1cccc2c1nc(N)n1nc(C3CC3c3ccc4nncn4c3)nc21] | 7-methoxy-2-[2- ([1,2,4]triazolo[4,3-a] pyridin-6- yl)cyclopropyl][1,2,4] triazolo[1,5-c] quinazolin-5-amine | 373 |
| Ex-14 | 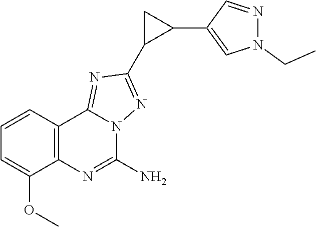 [SMILES: CCn1cc(C2CC2c2nc3c4cccc(OC)c4nc(N)n3n2)cn1] | 2-[2-(1-ethyl-1H- pyrazol-4- yl)cyclopropyl]-7- methoxy[1,2,4]triazolo [1,5-c]quinazolin-5- amine | 350 |
| Ex-15 | 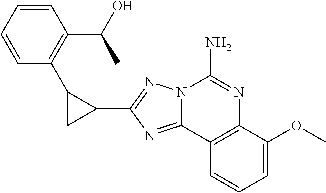 [SMILES: COc1cccc2c1nc(N)n1nc(C3CC3c3ccccc3[C@H](C)O)nc21] | (1S)-1-{2-[2-(5- amino-7- methoxy[1,2,4]triazolo [1,5-c]quinazolin-2- yl)cyclopropyl]phenyl} ethan-1-ol | 376 |
| Ex-16 | 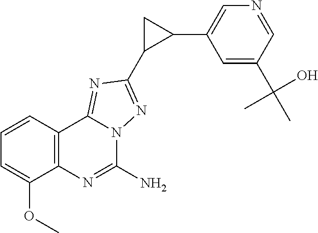 [SMILES: COc1cccc2c1nc(N)n1nc(C3CC3c3cncc(C(C)(C)O)c3)nc21] | 2-{5-[2-(5-amino-7- methoxy[1,2,4]triazolo [1,5-c]quinazolin-2- yl)cyclopropyl]pyridin- 3-yl} propan-2-ol | 391 |
| Ex-17 | 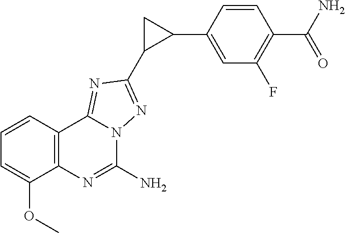 [SMILES: COc1cccc2c1nc(N)n1nc(C3CC3c3ccc(C(N)=O)c(F)c3)nc21] | 4-[2-(5-amino-7- methoxy[1,2,4]triazolo [1,5-c]quinazolin-2- yl)cyclopropyl]-2- fluorobenzamide | 393 |
| Ex-18 | 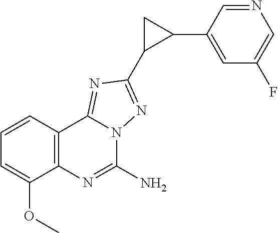 [SMILES: COc1cccc2c1nc(N)n1nc(C3CC3c3cncc(F)c3)nc21] | 2-[2-(5-fluoropyridin- 3-yl)cyclopropyl]-7- methoxy[1,2,4]triazolo [1,5-c]quinazolin-5- amine | 351 |
| Ex-19 | 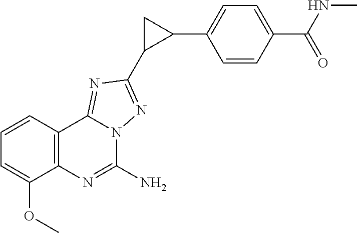 [SMILES: CNC(=O)c1ccc(C2CC2c2nc3c4cccc(OC)c4nc(N)n3n2)cc1] | 4-[2-(5-amino-7- methoxy[1,2,4]triazolo [1,5-c]quinazolin-2- yl)cyclopropyl]-N- methylbenzamide | 389 |
| Ex-20 | 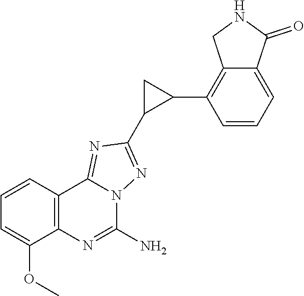 [SMILES: COc1cccc2c1nc(N)n1nc(C3CC3c3cccc4c3CNC4=O)nc21] | 4-[2-(5-amino-7- methoxy[1,2,4]triazolo [1,5-c]quinazolin-2- yl)cyclopropyl]-2,3- dihydro-1H-isoindol- 1-one | 387 |
| Ex-21 | 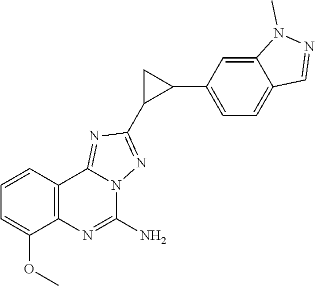 [SMILES: COc1cccc2c1nc(N)n1nc(C3CC3c3ccc4cnn(C)c4c3)nc21] | 7-methoxy-2-[2-(1- methyl-1H-indazol-6- yl)cyclopropyl][1,2,4] triazolo[1,5-c] quinazolin-5-amine | 386 |
| Ex-22 | 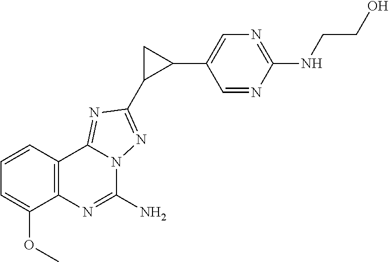 [SMILES: COc1cccc2c1nc(N)n1nc(C3CC3c3cnc(NCCO)nc3)nc21] | 2-({5-[2-(5-amino-7- methoxy[1,2,4]triazolo [1,5-c]quinazolin-2- yl)cyclopropyl]pyrimidin- 2-yl} amino)ethan-1-ol | 393 |
| Ex-23 | 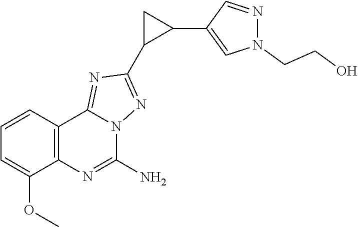 [SMILES: COc1cccc2c1nc(N)n1nc(C3CC3c3cnn(CCO)c3)nc21] | 2-{4-[2-(5-amino-7- methoxy[1,2,4]triazolo [1,5-c]quinazolin-2- yl)cyclopropyl]-1H- pyrazol-1-yl} ethan-1- ol | 366 |
| Ex-24 | 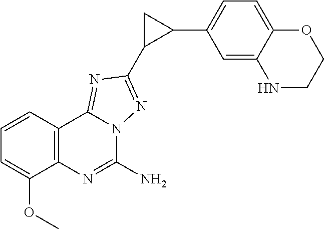 [SMILES: COc1cccc2c1nc(N)n1nc(C3CC3c3ccc4c(c3)NCCO4)nc21] | 2-[2-(3,4-dihydro- 2H-1,4-benzoxazin- 6-yl)cyclopropy1]-7- methoxy[1,2,4]triazolo [1,5-c]quinazolin-5- amine | 389 |
| Ex-25 | 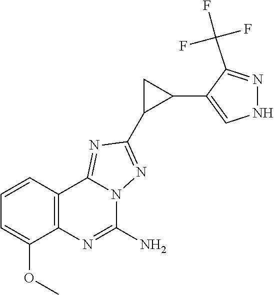 [SMILES: COc1cccc2c1nc(N)n1nc(C3CC3c3c[nH]nc3C(F)(F)F)nc21] | 7-methoxy-2-{2-[3- (trifluoromethyl)-1H- pyrazol-4- yl]cyclopropyl} [1,2,4] triazolo[1,5-c] quinazolin-5-amine | 390 |
| Ex-26 | 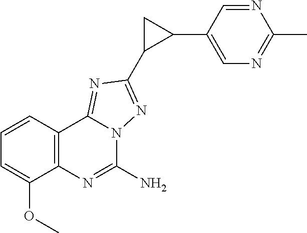 [SMILES: COc1cccc2c1nc(N)n1nc(C3CC3c3cnc(C)nc3)nc21] | 7-methoxy-2-[2-(2- methylpyrimidin-5- yl)cyclopropyl][1,2,4] triazolo[1,5-c] quinazolin-5-amine | 348 |
| Ex-27 | 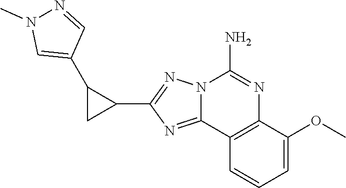 | 7-methoxy-2-[2-(1- methyl-1H-pyrazol-4- yl)cyclopropyl][1,2,4] triazolo[1,5-c] quinazolin-5-amine | 336 |

### Example 28: 2-(2-(5-amino-9-fluoro-7-methoxy-[1,2,4]triazolo[1,5-c]quinazolin-2-yl)cyclopropyl)propan-2-ol (Ex-28)

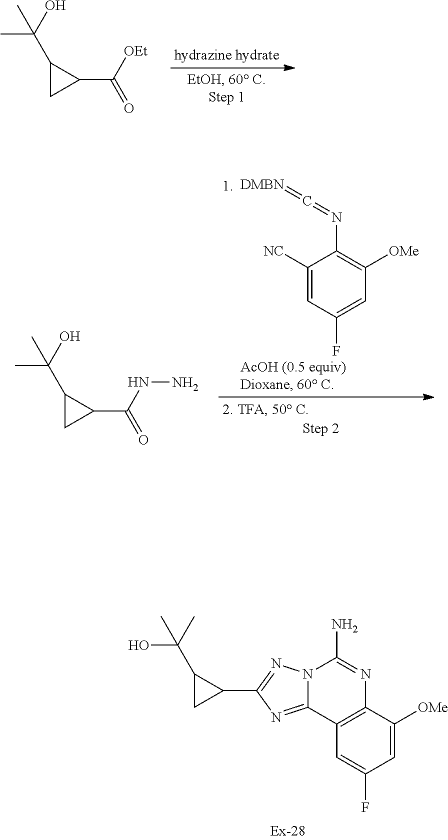

Step 1: Ethyl 2-(2-hydroxypropan-2-yl)cyclopropane-1-carboxylate (1.0 g, 5.81 mmol) was dissolved in EtOH (14.52 ml) in a 20 mL scintillation vial equipped with a stir bar. Hydrazine, H₂O (2.85 ml, 58.1 mmol) was added and the reaction mixture was heated to 60° C. for 48 hours. The reaction mixture was concentrated and then 2-(2-hydroxypropan-2-yl)cyclopropane-1-carbohydrazide was used in subsequent step without further purification. ESI MS m/z=159 [M+H]⁺.

Step 2: 2-(2-hydroxypropan-2-yl)cyclopropane-1-carbohydrazide (46.3 mg, 0.293 mmol) was dissolved in dioxane (977 μl) and acetic acid (8.39 μl, 0.146 mmol) was added. The reaction was stirred 2-((((2,4-dimethoxybenzyl)imino)methylene)amino)-5-fluoro-3-methoxybenzonitrile (100 mg, 0.293 mmol) was added. The reaction was then stirred at 60° C. for 16 hours. The crude mixture was subjected to silica gel column chromatography, eluting from 0-50% ethyl acetate in hexanes to the desired 2-(2-(5-((2,4-dimethoxybenzyl)amino)-9-fluoro-7-methoxy-[1,2,4]triazolo[1,5-c]quinazolin-2-yl)cyclopropyl)propan-2-ol. The intermediate was then exposed to TFA (677 μl, 8.79 mmol) and heated to 50° C. for 3 hours. The crude material was concentrated, re-dissolved in 3 mL DMSO, filtered, and submitted for mass-triggered RP-HPLC purification to yield 2-((2-(2-hydroxypropan-2-yl)cyclopropane-1-carbohydrazide)-2-(5-amino-9-fluoro-7-methoxy-[1,2,4]triazolo[1,5-c]quinazolin-2-yl)cyclopropyl)propan-2-ol, TFA Ex-28. ESI MS m/z=332 [M+H]I+. ¹H NMR (500 MHz, DMSO-d₆) δ 7.76 (s, 2H), 7.35 (dd, J=8.4, 2.7 Hz, 1H), 7.16 (dd, J=11.1, 2.7 Hz, 1H), 3.93 (s, 3H), 2.22 (dt, J=9.1, 4.7 Hz, 1H), 1.70-1.57 (m, 1H), 1.25-1.16 (m, 7H), 1.08 (dt, J=8.7, 4.1 Hz, 1H).

TABLE 3
Using the appropriate starting materials and a method similar to that described above,
the following examples were prepared:

| | | | Column/ | Exact |
|---|---|---|---|---|
| | | | Reaction | Mass |
| Example | Structure | Name | Conditions | [M + H]+ |
| Ex-29 | 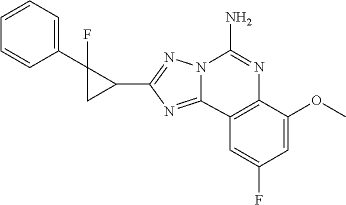 | 9-fluoro-2-[2-fluoro-2- phenylcyclopropyl]-7- methoxy[1,2,4]triazolo [1,5-c]quinazolin- 5-amine | Column: SJ, 21 × 250; Eluent: MeOH w/0.1% NH₄OH: 3:1:1 DMF/MeOH/ CH3CN Peak 4 | 368 |
| Ex-30 | 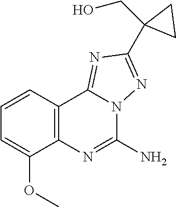 [SMILES: COc1cccc2c1nc(N)n1nc(C3(CO)CC3)nc21] | [1-(5-amino-7- methoxy[1,2,4]triazolo [1,5-c]quinazolin-2- yl)cyclopropyl]methanol | | 286 |
| Ex-31 | 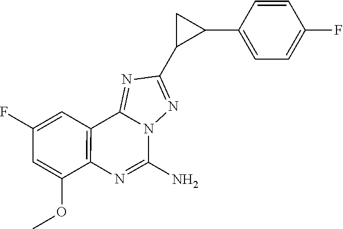 | 9-fluoro-2-[2-(4- fluorophenyl) cyclopropyl]-7- methoxy[1,2,4]triazolo [1,5-c]quinazolin- 5-amine | | 368 |
| Ex-32 | 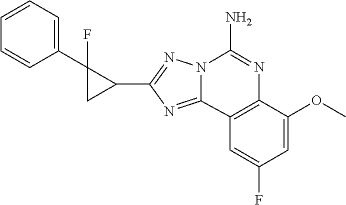 | 9-fluoro-2-[2-fluoro-2- phenylcyclopropyl]-7- methoxy[1,2,4]triazolo [1,5-c]quinazolin- 5-amine | Column: SJ, 21 × 250; Eluent: MeOH w/0.1% NH₄OH: 3:1:1 DMF/MeOH/ CH3CN Peak 2 | 368 |
| Ex-33 | 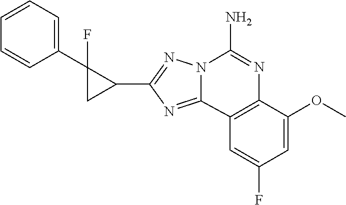 | 9-fluoro-2-[2-fluoro-2- phenylcyclopropyl]-7- methoxy[1,2,4]triazolo [1,5-c]quinazolin- 5-amine | Column: SJ, 21 × 250; Eluent: MeOH w/0.1% NH₄OH: 3:1:1 DMF/MeOH/ CH₃CN Peak 1 | 368 |
| Ex-34 | 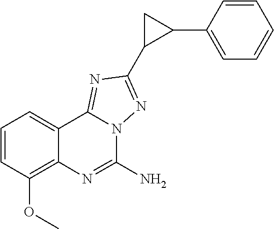 | 7-methoxy-2- [(1r,2r)-2- phenylcyclopropyl] [1,2,4]triazolo[1,5-c] quinazolin-5- amine | | 332 |
| Ex-35 | 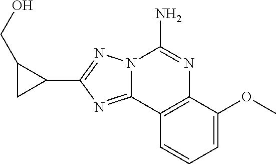 | [2-(5-amino-7- methoxy[1,2,4]triazolo [1,5-c]quinazolin-2- yl)cyclopropyl] methanol | Column: CC4, 21 × 250; Eluent: MeOH w/0.1% NH₄OH; 1:1 MeOH/CH₃CN Peak 1 | 286 |
| Ex-36 | 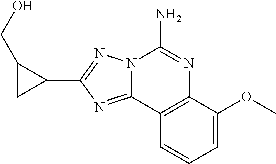 | [2-(5-amino-7- methoxy[1,2,4]triazolo [1,5-c]quinazolin-2- yl)cyclopropyl] methanol | Column: CC4, 21 × 250; Eluent: MeOH w/0.1% NH₄OH; 1:1 MeOH/CH₃CN Peak 2 | 286 |
| Ex-37 | 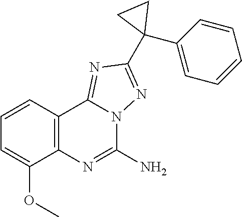 [SMILES: COc1cccc2c1nc(N)n1nc(C3(c4ccccc4)CC3)nc21] | 7-methoxy-2-(1- phenylcyclopropyl) [1,2,4]triazolo[1,5-c] quinazolin-5- amine | | 332 |
| Ex-38 | 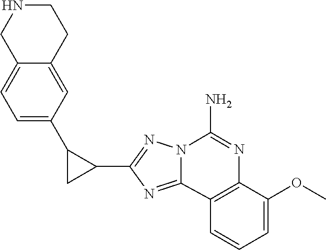 | 7-methoxy-2- [(1r,2r)-2-(1,2,3,4- tetrahydroisoquinolin- 6-yl)cyclopropyl]- 1,10b- dihydro[1,2,4]triazolo [1,5-c]quinazolin- 5-amine | | 387 |
| Ex-39 | 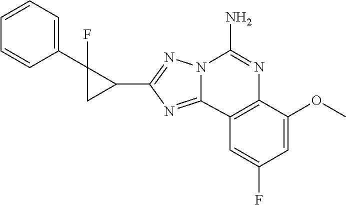 | 9-fluoro-2-[2-fluoro-2- phenylcyclopropyl]-7- methoxy[1,2,4]triazolo [1,5-c]quinazolin- 5-amine | Column: SJ, 21 × 250; Eluent: MeOH w/0.1% NH₄OH: 3:1:1 DMF/MeOH/ CH₃CN Peak 3 | 368 |

### Example 40: 2-(2-(4-fluorobenzyl)cyclopropyl)-7-methoxy-[1,2,4]triazolo[1,5-c]quinazolin-5-amine (Ex-40)

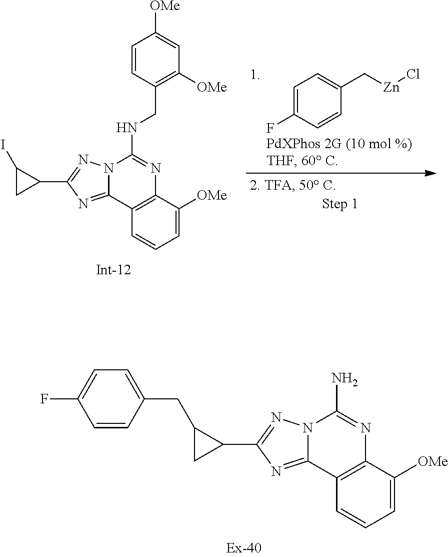

N-(2,4-dimethoxybenzyl)-2-(−iodocyclopropyl)-7-methoxy-[1,2,4]triazolo[1,5-c]quinazolin-5-amine (Int-12) (50 mg, 0.094 mmol), Xphos Pd G2 (7.40 mg, 9.41 μmol) and 4-fluorobenzyliczinc chloride (565 μl, 0.282 mmol) (0.5 M THF solution) were dissolved in THF (941 μl). The mixture was purged with N₂ for 5 min then heated at 60° C. for 3 hours. The mixture was quenched with a minimal amount of sat. aqueous NH₄Cl and Celite™ was added. The biphasic mixture was stirred for several minutes and then filtered through Celite™ with a top layer of anhydrous MgSO₄, followed by rinsing with DCM. The filtrate was concentrated and re-dissolved in TFA (580 μl, 7.53 mmol) and stirred at 50° C. for 2 hours. The reaction mixture was then removed from the heat, concentrated, quenched with sat. aq. NaHCO₃ and diluted with DCM. The DCM layer was collected and concentrated. The residual oil was re-dissolved in 1.5 mL DMSO, filtered, and submitted for mass-triggered RP-HPLC purification to yield 2-(2-(4-fluorobenzyl)cyclopropyl)-7-methoxy-[1,2,4]triazolo[1,5-c]quinazolin-5-amine, TFA (Ex-40). ESI MS m/z=364 [M+H]⁺. ¹H NMR (500 MHz, DMSO-d₆) δ 7.90 (s, 2H), 7.67 (dd, J=7.9, 1.2 Hz, 1H), 7.35 (dd, J=8.5, 5.7 Hz, 2H), 7.29 (t, J=7.9 Hz, 1H), 7.23 (d, J=7.1 Hz, 1H), 7.16-7.08 (m, 2H), 3.91 (s, 3H), 2.78 (m, 2H), 2.19 (dt, J=8.8, 4.6 Hz, 1H), 1.83-1.68 (m, 1H), 1.30 (dt, J=8.7, 4.4 Hz, 1H), 1.13 (ddd, J=9.9, 5.8, 4.2 Hz, 1H).

TABLE 4
Using the appropriate starting materials and a method similar to that described above, the following examples were prepared:

| Example | Structure | Name | Exact Mass [M + H]+ |
|---|---|---|---|
| Ex-41 | 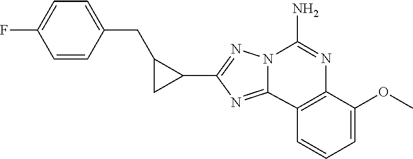 | 2-{2-[(4- fluorophenyl)methyl] cyclopropyl}-7- methoxy[1,2,4]triazolo [1,5-c]quinazolin-5-amine; isomer 2 | 364 |

### Example 42 and 43: 2-(4-((1s,3s)-3-(5-amino-7-methoxy-[1,2,4]triazolo[1,5-c]quinazolin-2-yl)cyclobutyl)phenyl)propan-2-ol (Ex-42) and 2-(4-((1r,3r)-3-(5-amino-7-methoxy-[1,2,4]triazolo[1,5-c]quinazolin-2-yl)cyclobutyl)phenyl)propan-2-ol (Ex-43)

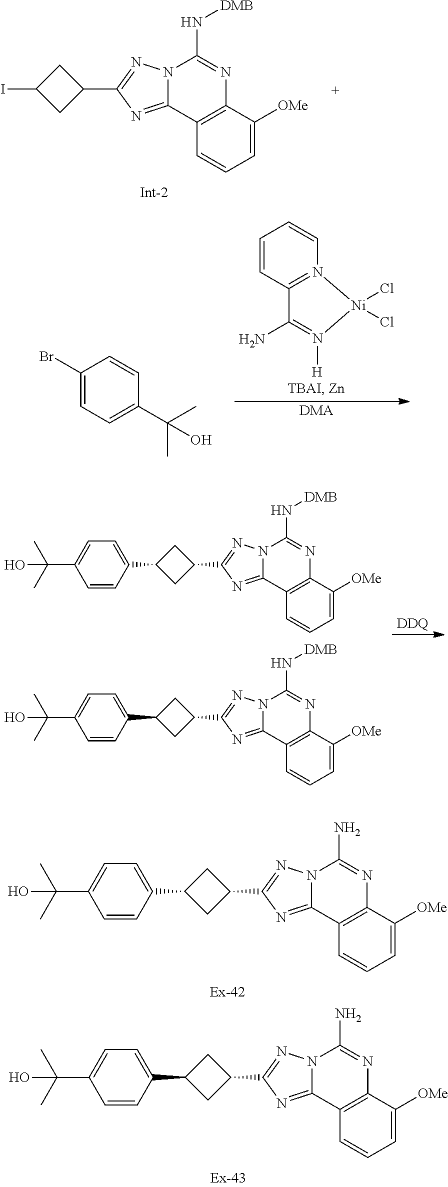

Step 1: A 5 mL Biotage® microwave vial equipped with a stir bar was charged with Ni(picolinimidamide)Cl₂·4H₂O (5.5 mg, 0.022 mmol), 2-(4-bromophenyl)propan-2-ol (79 mg, 0.367 mmol), N-(2,4-dimethoxy benzyl)-2-(3-iodocyclobutyl)-7-methoxy-[1,2,4]triazolo[1,5-c]quinazolin-5-amine (Int-2) (100 mg, 0.183 mmol), and tetrabutylammonium iodide (17 mg, 0.046 mmol). The vial was transferred into a glovebox, and zinc (36 mg, 0.550 mmol) and DMA (1.8 mL) were added. The reaction vial was sealed, removed from the glovebox, and heated at 60° C. and stirred for 16 h. After cooling, the reaction mixture was filtered through Celite™ and concentrated. The crude residue was purified by silica gel column chromatography (0-100% EtOAc in hex) to afford 2-(4-(3-(5-((2,4-dimethoxybenzyl)amino)-7-methoxy-[1,2,4]triazolo[1,5-c]quinazolin-2-yl)cyclobutyl)phenyl)propan-2-ol.

Step 2: A 40 mL scintillation vial equipped with a stir bar was charged with 2-(4-(3-(5-((2,4-dimethoxybenzyl)amino)-7-methoxy-[1,2,4]triazolo[1,5-c]quinazolin-2-yl)cyclobutyl)phenyl)propan-2-ol (42 mg, 0.076 mmol). DCM (900 L) and water (360 μL) were added, and the reaction mixture was cooled to 0° C. DDQ (26 mg, 0.114 mmol) was then added, and the resulting mixture was stirred at 0° C. for 1 h. The reaction was quenched with sat. aq. Na₂S₂O₃ (5 mL) and DCM (5 mL). After stirring at 25° C. for 20 min, the layers were separated, and the aq. layer was extracted with DCM (3×15 mL). The combined organic layers were dried over anhydrous MgSO₄, filtered and concentrated. The crude residue was taken up in DMSO (2 mL), filtered, and purified by reverse phase HPLC using the NH₄OH modifier. The diastereomers were then separated by achiral SFC purification (Lux-3 21×250 mm column with 25% MeOH (w/0.1% NH₄OH) as cosolvent) to provide the cis isomer as the first eluting peak, and the trans isomer as the second eluting peak. Each sample was then purified by silica gel chromatography (0-100% 3:1 EtOAc:EtOH in hex) to afford 2-(4-((1s,3s)-3-(5-amino-7-methoxy-[1,2,4]triazolo[1,5-c]quinazolin-2-yl)cyclobutyl)phenyl)propan-2-ol (Ex-42) and 2-(4-((1r,3r)-3-(5-amino-7-methoxy-[1,2,4]triazolo[1,5-c]quinazolin-2-yl)cyclobutyl)phenyl)propan-2-ol (Ex-43). Ex-42: ESI MS m/z=404 [M+H]⁺. ¹H NMR (600 MHz, MeOD-d₄) δ 7.89 (d, J=7.0 Hz, 1H), 7.44 (d, J=8.3 Hz, 2H), 7.38 (t, J=8.0 Hz, 1H), 7.31 (d, J=8.2 Hz, 2H), 7.27 (d, J=7.2 Hz, 1H), 3.90-3.81 (m, 1H), 3.69-3.60 (m, 1H), 2.91-2.83 (m, 2H), 2.70-2.62 (m, 2H), 1.53 (s, 6H). Ex-43: ESI MS m/z=404 [M+H]⁺. ¹H NMR (600 MHz, MeOD-d₄) δ 7.90 (d, J=8.1 Hz, 1H), 7.47 (d, J=8.3 Hz, 2H), 7.39 (t, J=8.0 Hz, 1H), 7.32 (d, J=8.1 Hz, 2H), 7.29 (d, J=7.1 Hz, 1H), 4.03 (s, 3H), 3.98-3.92 (m, 1H), 3.92-3.85 (m, 1H), 2.97-2.89 (m, 2H), 2.76-2.69 (m, 2H), 1.54 (s, 6H).

TABLE 5
Using the appropriate starting materials and a method similar to that described above,
the following examples were prepared:

| | | | Column/ | Exact |
|---|---|---|---|---|
| | | | Reaction | Mass |
| Example | Structure | Name | Conditions | [M + H]\|+ |
| Ex-44 | 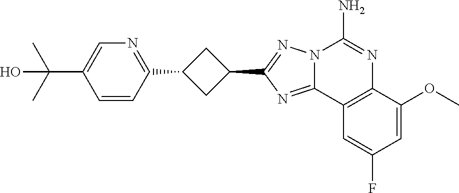 | 2-{6-[trans-3-(5- amino-9-fluoro-7- methoxy [1,2,4] triazolo[1,5-c]quinazolin- 2-yl)cyclobutyl]pyridin- 3-yl}propan-2-ol | | 423 |
| Ex-45 | 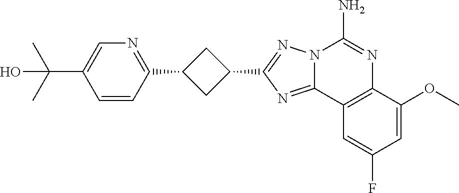 | 2-{6-[cis-3-(5-amino- 9-fluoro-7- methoxy [1,2,4]triazolo [1,5-c]quinazolin-2- yl)cyclobutyl]pyridin- 3-yl} propan-2-ol | Column: AD-H 21 × 250 mm Eluent: 30% MeOH (w/ 0.1% NH₄OH); 5.7 min elution | 423 |
| Ex-46 | 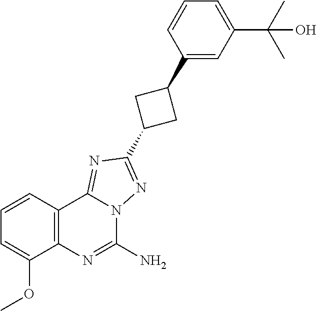 [SMILES: COc1cccc2c1nc(N)n1nc([C@H]3C[C@H](c4cccc(C(C)(C)O)c4)C3)nc21] | 2-{3-[trans-3-(5- amino-7- methoxy [1,2,4]triazolo [1,5-c]quinazolin-2- yl)cyclobutyl]phenyl} propan-2-ol | Column: Lux-3 21 × 250 mm Eluent: 40% MeOH (w/ 0.1% NH₄OH); 4.2 min elution | 404 |
| Ex-47 | 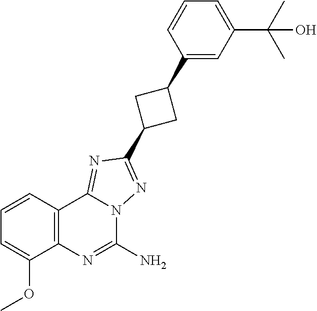 [SMILES: COc1cccc2c1nc(N)n1nc([C@H]3C[C@@H](c4cccc(C(C)(C)O)c4)C3)nc21] | 2-{3-[cis-3-(5-amino-7- methoxy [1,2,4]triazolo [1,5-c]quinazolin-2- yl)cyclobutyl]phenyl} propan-2-ol | Column: Lux-3 21 × 250 mm Eluent: 40% MeOH (w/ 0.1% NH₄OH); 3.8 min elution | 404 |
| Ex-48 | 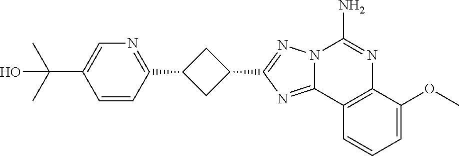 | 2-{6-[cis-3-(5-amino-7- methoxy [1,2,4]triazolo [1,5-c]quinazolin-2- yl)cyclobutyl]pyridin- 3-yl} propan-2-ol | Coupling done at 40° C. Column: SJ 21 × 250 mm Eluent: 40% MeOH (w/0.1% NH₄OH); 1.9 min elution | 405 |
| Ex-49 | 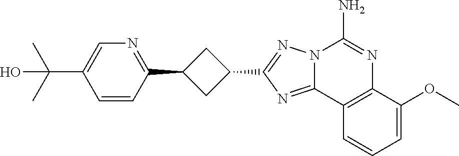 | 2-{6-[trans-3-(5- amino-7- methoxy [1,2,4]triazolo [1,5-c]quinazolin-2- yl)cyclobutyl]pyridin- 3-yl} propan-2-ol | Coupling done at 40° C.; Column: SJ 21 × 250 mm Eluent: 40% MeOH (w/0.1% NH₄OH); 2.7 min | 405 |
| | | | elution | |
| Ex-50 | 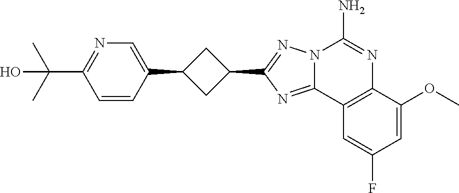 | 2-{4-[cis-3-(5-amino- 9-fluoro-7- methoxy [1,2,4]triazolo [1,5-c]quinazolin-2- yl)cyclobutyl]phenyl} propan-2-ol | Coupling done at 40° C.; Column: Lux-3 21 × 250 mm Eluent: 25% MeOH (w/0.1% NH₄OH); 3.8 min elution | 422 |
| Ex-51 | 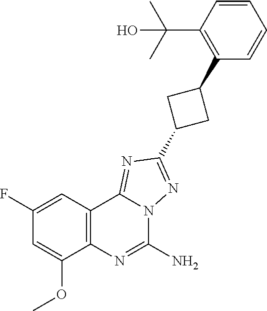 [SMILES: COc1cc(F)cc2c1nc(N)n1nc([C@H]3C[C@H](c4ccccc4C(C)(C)O)C3)nc21] | 2-{2-[trans-3-(5- amino-9-fluoro-7- methoxy [1,2,4]triazolo [1,5-c]quinazolin-2- yl)cyclobutyl phenyl} propan-2-ol | Coupling done at 40° C.; Column: Lux-3 21 × 250 mm Eluent: 25% MeOH (w/ 0.1% NH₄OH); Peak 1; 3.9 min elution | 422 |
| Ex-52 | 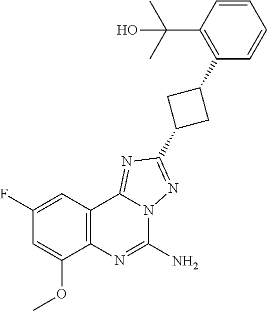 [SMILES: COc1cc(F)cc2c1nc(N)n1nc([C@H]3C[C@@H](c4ccccc4C(C)(C)O)C3)nc21] | 2-{2-[cis-3-(5-amino- 9-fluoro-7- methoxy [1,2,4]triazolo [1,5-c]quinazolin-2- yl)cyclobutyl phenyl} propan-2-ol | Coupling done at 40° C.; Column: Lux-3 21 × 250 mm Eluent: 25% MeOH (w/ 0.1% NH₄OH); Peak 2; 7.5 min elution | 422 |
| Ex-53 | 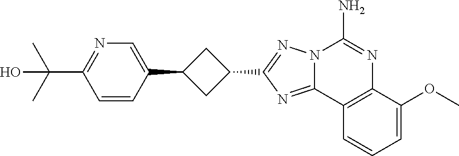 | 2-{5-[trans-3-(5- amino-7- methoxy [1,2,4]triazolo [1,5-c]quinazolin-2- yl)cyclobutyl]pyridin- 2-yl} propan-2-ol | Int-1 was used as cyclobutyl halide; Column: CCA 21 × 250 mm Eluent: 40% MeOH (w/ 0.1% NH₄OH); | 405 |
| | | | 4.5 min | |
| | | | elution | |
| Ex-54 | 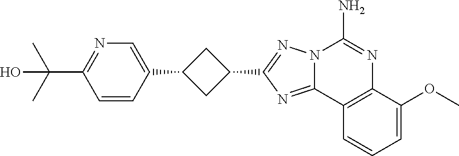 | 2-{5-[cis-3-(5-amino-7- methoxy [1,2,4]triazolo [1,5-c]quinazolin-2- yl)cyclobutyl]pyridin- 2-yl} propan-2-ol | Int-1 was used as cyclobutyl halide; Column: CCA 21 × 250 mm Eluent: 40% MeOH (w/ 0.1% | |
| | | | NH₄OH); | |
| | | | 3.5 min | |
| | | | elution | |
| Ex-55 | 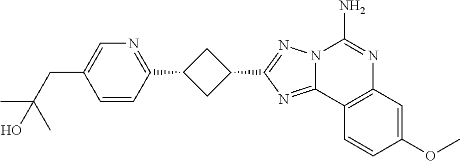 | 1-{6-[cis-3-(5-amino-8- methoxy [1,2,4]triazolo [1,5-c]quinazolin-2- yl)cyclobutyl]pyridin- 3-yl}-2- methylpropan-2-ol | Coupling done at 40° C.; Column: OJ-H 21 × 250 mm Eluent: 35% MeOH (w/ 0.1% NH₄OH); 3.1 min elution | 419 |
| Ex-56 | 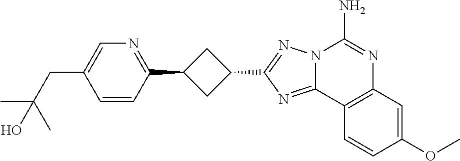 | 1-{6-[trans-3-(5- amino-8- methoxy [1,2,4]triazolo [1,5-c]quinazolin-2- yl)cyclobutyl]pyridin- 3-yl}-2- methylpropan-2-ol | Coupling done at 40° C.; Column: OJ-H 21 × 250 mm Eluent: 35% MeOH (w/ 0.1% NH₄OH); 4.6 min elution | 419 |
| Ex-57 | 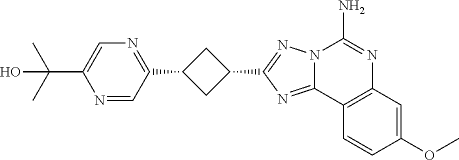 | 2-{5-[cis-3-(5-amino-8- methoxy [1,2,4]triazolo [1,5-c]quinazolin-2- yl)cyclobutyl]pyrazin- 2-yl} propan-2-ol | Coupling done at 40° C.; Column: OJ-H 21 × 250 mm Eluent: 35% MeOH (w/ 0.1% NH₄OH); 4.2 min elution | 406 |
| Ex-58 | 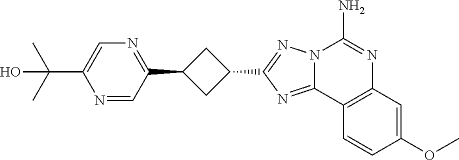 | 2-{5-[trans-3-(5- amino-8- methoxy [1,2,4]triazolo [1,5-c]quinazolin-2- yl)cyclobutyl]pyrazin- 2-yl} propan-2-ol | Coupling done at 40° C.; Column: OJ-H 21 × 250 mm Eluent: 35% MeOH (w/ 0.1% NH₄OH); 6.4 min elution | 406 |
| Ex-59 | 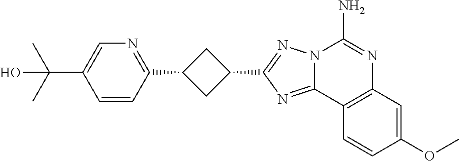 | 2-{6-[cis-3-(5-amino-8- methoxy[1,2,4]triazolo [1,5-c]quinazolin-2- yl)cyclobutyl]pyridin- 3-yl}propan-2-ol | Coupling done at 40° C.; Column: OJ-H 21 × 250 mm Eluent: 35% MeOH (w/ 0.1% NH₄OH); 3.5 min elution | 405 |
| Ex-60 | 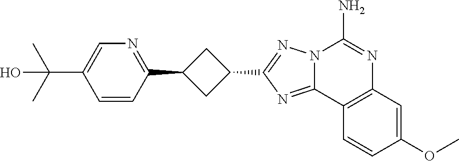 | 2-{6-[trans-3-(5- amino-8- methoxy [1,2,4]triazolo [1,5-c]quinazolin-2- yl)cyclobutyl]pyridin- 3-yl} propan-2-ol | Coupling done at 40° C.; Column: OJ-H 21 × 250 mm Eluent: 35% MeOH (w/ 0.1% NH₄OH); 5.5 min elution | 405 |
| Ex-61 | 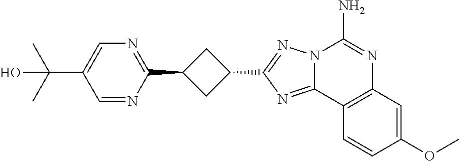 | 2-{2-[trans-3-(5- amino-8- methoxy [1,2,4]triazolo [1,5-c]quinazolin-2- yl)cyclobutyl]pyrimidin- 5-yl} propan-2-ol | Coupling done at 40° C. | 406 |
| Ex-62 | 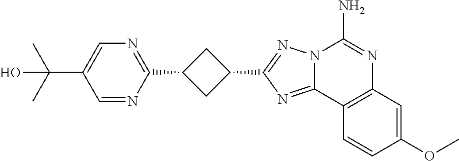 | 2-{2-[cis-3-(5-amino-8- methoxy [1,2,4]triazolo [1,5-c]quinazolin-2- yl)cyclobutyl]pyrimidin- 5-yl} propan-2-ol | Coupling done at 40° C. | 406 |
| Ex-63 | 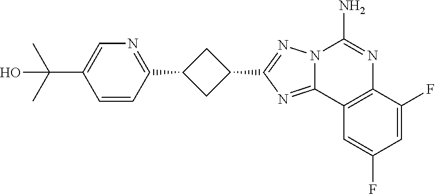 | 2-{6-[cis-3-(5-amino- 7,9- difluoro[1,2,4]triazolo [1,5-c]quinazolin-2- yl)cyclobutyl]pyridin- 3-yl} propan-2-ol | Coupling done at 40° C.; Column: SJ 21 × 250 mm Eluent: 40% MeOH (w/ 0.1% NH₄OH); 2.0 min elution | 411 |
| Ex-64 | 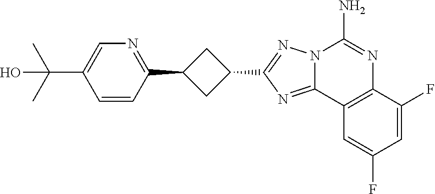 | 2-{6-[trans-3-(5- amino-7,9- difluoro[1,2,4]triazolo [1,5-c]quinazolin-2- yl)cyclobutyl]pyridin- 3-yl} propan-2-ol | Coupling done at 40° C.; Column: SJ 21 × 250 mm Eluent: 40% MeOH (w/ 0.1% NH₄OH); 2.9 min elution | 411 |
| Ex-65 | 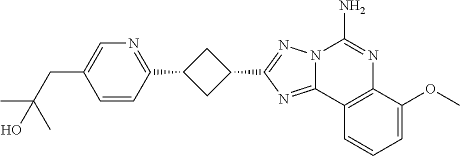 | 1-{6-[cis-3-(5-amino-7- methoxy [1,2,4]triazolo [1,5-c]quinazolin-2- yl)cyclobutyl]pyridin- 3-yl}-2- methylpropan-2-ol | Coupling done at 40° C. | 419 |
| Ex-66 | 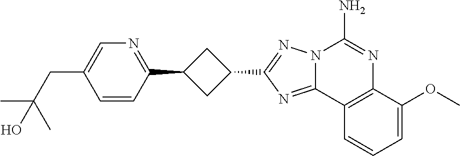 | 1-{6-[trans-3-(5- amino-7- methoxy [1,2,4]triazolo [1,5-c]quinazolin-2- yl)cyclobutyl]pyridin- 3-yl}-2- methylpropan-2-ol | Coupling done at 40° C. | 419 |

### Example 67 and 68: 2-((1s,3s)-3-(5-(2-aminopropan-2-yl)pyridin-2-yl)cyclobutyl)-7-methoxy-[1,2,4]triazolo[1,5-c]quinazolin-5-amine (Ex-67) and 2-((1r,3r)-3-(5-(2-aminopropan-2-yl)pyridin-2-yl)cyclobutyl)-7-methoxy-[1,2,4]triazolo[1,5-c]quinazolin-5-amine (Ex-68)

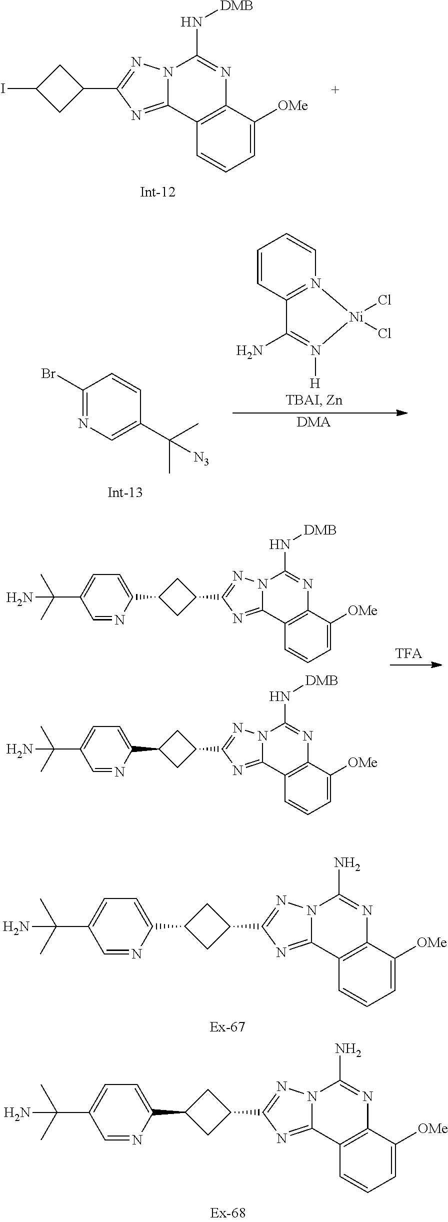

Two side-by-side 40 mL scintillation vials were each charged with Ni(picolinimidamide)Cl₂·4H₂O (129 mg, 0.513 mmol), 5-(2-azidopropan-2-yl)-2-bromopyridine (1.24 g, 5.13 mmol) (Int-13), N-(2,4-dimethoxybenzyl)-2-(3-iodocyclobutyl)-7-methoxy-[1,2,4]triazolo[1,5-c]quinazolin-5-amine (Int-_2) (1.40 g, 2.57 mmol), tetrabutylammonium iodide (237 mg, 0.642 mmol), and zinc (503 mg, 7.70 mmol). The reaction vials were evacuated and backfilled with nitrogen (3×). DMA (6.1 mL) was then added, and the reactions were degassed by bubbling argon for 15 min. The reaction mixture was then stirred at 50° C. for 16 h. After cooling, the reactions were combined, filtered through Celite™, and concentrated. The crude residue was purified by silica gel chromatography using 0-50% MeOH in DCM as eluent, followed by achiral SFC purification (BEH 21×250 mm column with 20% MeOH (w/0.1% NH₄OH) as cosolvent) to remove additional impurities, followed by chiral SFC purification (OJ-H 21×250 mm column with 30% MeOH (w/0.1% NH₄OH) as cosolvent) to provide 2-((1s,3s)-3-(5-(2-aminopropan-2-yl)pyridin-2-yl)cyclobutyl)-N-(2,4-dimethoxybenzyl)-7-methoxy-[1,2,4]triazolo[1,5-c]quinazolin-5-amine as the first eluting isomer, and 2-((1r,3r)-3-(5-(2-aminopropan-2-yl)pyridin-2-yl)cyclobutyl)-N-(2,4-dimethoxybenzyl)-7-methoxy-[1,2,4]triazolo[1,5-c]quinazolin-5-amine as the second eluting isomer. A 20 mL scintillation vial was charged with 2-((1s,3s)-3-(5-(2-aminopropan-2-yl)pyridin-2-yl)cyclobutyl)-N-(2,4-dimethoxybenzyl)-7-methoxy-[1,2,4]triazolo[1,5-c]quinazolin-5-amine. TFA (870 μL) was then added, and the reaction mixture was heated at 50° C. and stirred for 2 h. After cooling, the reaction was concentrated, and the residue was taken up in DMSO (2 mL), filtered, and purified by reverse phase HPLC using the TFA modifier to provide 2-((1s,3s)-3-(5-(2-aminopropan-2-yl)pyridin-2-yl)cyclobutyl)-7-methoxy-[1,2,4]triazolo[1,5-c]quinazolin-5-amine (Ex-67) ESI MS m/z=404 [M+H]⁺. ¹H NMR (600 MHz, MeOD-d₄) δ 8.63 (d, J=2.4 Hz, 1H), 7.95 (dd, J=8.3, 2.5 Hz, 1H), 7.87 (dd, J=8.0, 1.0 Hz, 1H), 7.45 (d, J=8.3 Hz, 1H), 7.37 (app t, J=8.0 Hz, 1H), 7.27 (d, J=7.3 Hz, 1H), 4.02 (s, 3H), 3.91 (ddd, J=18.0, 9.9, 8.0 Hz, 1H), 3.81 (ddd, J=18.3, 10.1, 8.1 Hz, 1H), 2.96-2.88 (m, 2H), 2.82-2.74 (m, 2H), 1.54 (s, 6H).

2-((1r,3r)-3-(5-(2-aminopropan-2-yl)pyridin-2-yl)cyclobutyl)-N-(2,4-dimethoxybenzyl)-7-methoxy-[1,2,4]triazolo[1,5-c]quinazolin-5-amine was subjected to similar reaction conditions to afford 2-((1r,3r)-3-(5-(2-aminopropan-2-yl)pyridin-2-yl)cyclobutyl)-7-methoxy-[1,2,4]triazolo[1,5-c]quinazolin-5-amine (Ex-M). ESI MS m/z=404 [M+H]⁺. ¹H NMR (600 MHz, MeOD-d₄) δ 8.69 (d, J=2.3 Hz, 1H), 7.95 (dd, J=8.2, 2.5 Hz, 1H), 7.90 (dd, J=8.0, 1.1 Hz, 1H), 7.42 (d, J=8.2 Hz, 1H), 7.38 (app t, J=8.0 Hz, 1H), 7.29 (d, J=7.2 Hz, 1H), 4.11-4.02 (m, 1H), 4.03 (s, 3H), 3.97-3.90 (m, 1H), 2.97-2.91 (m, 2H), 2.90-2.83 (m, 2H), 1.54 (s, 6H).

TABLE 6
Using the appropriate starting materials and a method similar to that described above, the following examples were prepared:
Catalyst system of NiI₂ and pyridine-2,6-bis(carboximidamide) dihydrochloride (45 mol %) will be referred to as “catalyst A”

| | | | Column/ | Exact |
|---|---|---|---|---|
| | | | Reaction | Mass |
| Example | Structure | Name | Conditions | [M + H]+ |
| Ex-69 | 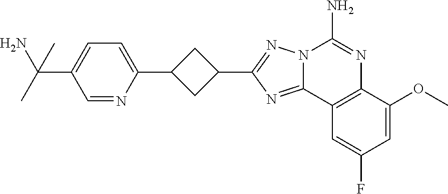 [SMILES: COc1cc(F)cc2c1nc(N)n1nc(C3CC(c4ccc(C(C)(C)N)cn4)C3)nc21] | 2-{3-[5-(2- aminopropan-2- yl)pyridin-2- yl]cyclobutyl}- 9-fluoro-7- methoxy [1,2,4] triazolo[1,5-c] quinazolin-5-amine | Coupling done at 40° C.; isomer was isolated via silica gel column chromatography directly (0-60% EtOAc in hex) | 422 |
| Ex-70 | 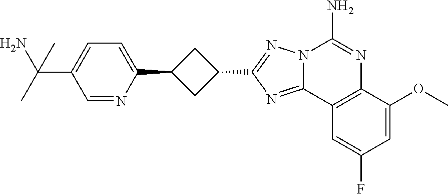 [SMILES: COc1cc(F)cc2c1nc(N)n1nc([C@H]3C[C@H](c4ccc(C(C)(C)N)cn4)C3)nc21] | 2-{trans-3-[6-(2- aminopropan-2- yl)pyridin-3- yl]cyclobutyl}- 9-fluoro-7- methoxy [1,2,4] triazolo[1,5-c] quinazolin-5- amine | Coupling done at 40 °C.; isomer was isolated via silica gel column chromatography directly (0- 60% EtOAc in hex) | 422 |
| Ex-71 | 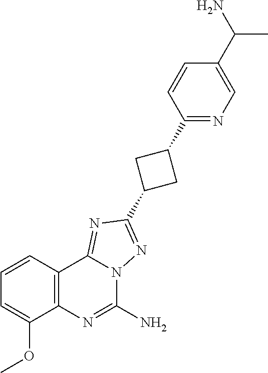 [SMILES: COc1cccc2c1nc(N)n1nc([C@H]3C[C@@H](c4ccc(C(C)N)cn4)C3)nc21] | 2-{cis-3-[5-(1- aminoethyl) pyridin-2- yl]cyclobutyl}-7- methoxy [1,2,4] triazolo[1,5-c] quinazolin-5-amine | Coupling done at 40° C.; catalyst A; Column: OJ-H 21 × 250 mm Eluent: 40% MeOH (w/ 0.1% NH₄OH); 1.9 min elution | 390 |
| Ex-72 | 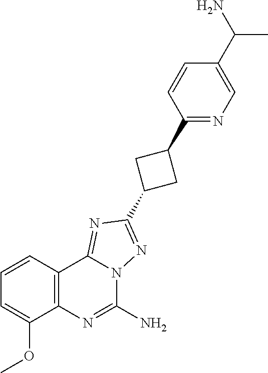 [SMILES: COc1cccc2c1nc(N)n1nc([C@H]3C[C@H](c4ccc(C(C)N)cn4)C3)nc21] | 2-{trans-3-[5-(1- aminoethyl) pyridin-2- yl]cyclobutyl}-7- methoxy [1,2,4] triazolo[1,5-c] quinazolin-5-amine | Coupling done at 40° C.; catalyst A; Column: OJ-H 21 × 250 mm Eluent: 40% MeOH (w/ 0.1% NH₄OH); 3.1 min elution | 390 |
| Ex-73 | 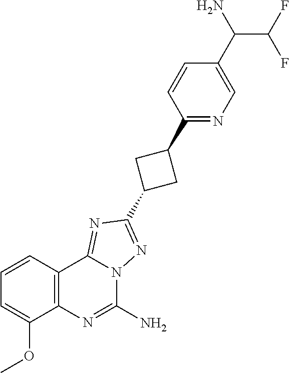 [SMILES: COc1cccc2c1nc(N)n1nc([C@H]3C[C@H](c4ccc(C(N)C(F)F)cn4)C3)nc21] | 2-{trans-3-[5-(1- amino-2,2- difluoroethyl) pyridin-2- yl]cyclobutyl}-7- methoxy[1,2,4] triazolo[1,5-c] quinazolin-5-amine | Coupling done at 70° C.; catalyst A; Column: BEH 21 × 250 mm Eluent: 10% MeOH (w/ 0.1% NH₄OH); 5.3 min elution | 426 |
| Ex-74 | 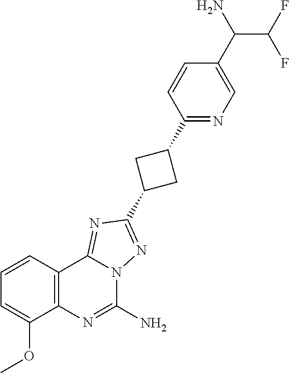 [SMILES: COc1cccc2c1nc(N)n1nc([C@H]3C[C@@H](c4ccc(C(N)C(F)F)cn4)C3)nc21] | 2-{cis-3-[5-(1- amino-2,2- difluoroethyl) pyridin-2- yl]cyclobutyl}-7- methoxy [1,2,4] triazolo[1,5-c] quinazolin-5-amine | Coupling done at 70° C.; catalyst A; Column: BEH 21 × 250 mm Eluent: 10% MeOH (w/ 0.1% NH₄OH); 6.1 min elution | 426 |
| Ex-75 | 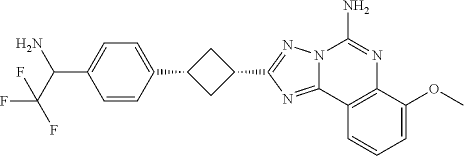 [SMILES: COc1cccc2c1nc(N)n1nc([C@H]3C[C@@H](c4ccc(C(N)C(F)(F)F)cc4)C3)nc21] | 2-{cis-3-[4-(1- amino-2,2,2- trifluoroethyl) phenyl] cyclobutyl}-7- methoxy[1,2,4] triazolo[1,5-c] quinazolin-5-amine | Coupling done at 70° C.; catalyst A; Column: AS-H 21 × 250 mm Eluent: MeOH (w/ 0.1% NH₄OH); 5.6 min elution; then Column: | 443 |
| | | | CCA 21 × 250 | |
| | | | mm Eluent: | |
| | | | MeOH (w/ | |
| | | | 0.1% NH₄OH); | |
| | | | 6.8 min elution | |
| Ex-76 | 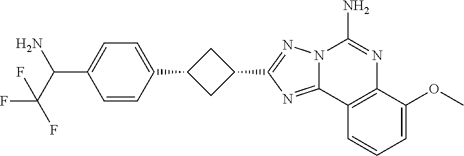 [SMILES: COc1cccc2c1nc(N)n1nc([C@H]3C[C@@H](c4ccc(C(N)C(F)(F)F)cc4)C3)nc21] | 2-{cis-3-[4-(1- amino-2,2,2- trifluoroethyl) phenyl] cyclobutyl}-7- methoxy [1,2,4] triazolo[1,5-c] quinazolin-5-amine | Coupling done at 70° C;. catalyst A; Column: AS-H 21 × 250 mm Eluent: MeOH (w/ 0.1% NH₄OH); 5.6 min elution; then Column: CCA | 443 |
| | | | 21 × 250 mm | |
| | | | Eluent: MeOH | |
| | | | (w/0.1% | |
| | | | NH₄OH); 5.5 | |
| | | | min elution | |
| Ex-77 | 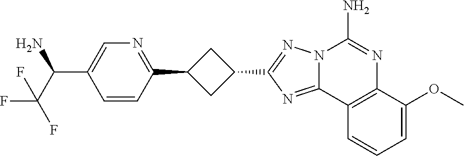 [SMILES: COc1cccc2c1nc(N)n1nc([C@H]3C[C@H](c4ccc([C@H](N)C(F)(F)F)cn4)C3)nc21] | 2-(trans-3-{5- [(1S)-1-amino- 2,2,2- trifluoroethyl] pyridin-2- yl}cyclobutyl)-7- methoxy[1,2,4] triazolo[1,5-c] quinazolin-5-amine | CCA 21 × 250 mm Eluent: MeOH (w/ 0.1% NH₄OH); 5.5 min elution (0-100% EtOAc in hex) | 444 |
| Ex-78 | 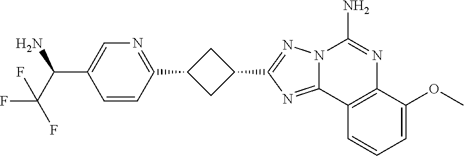 [SMILES: COc1cccc2c1nc(N)n1nc([C@H]3C[C@@H](c4ccc([C@H](N)C(F)(F)F)cn4)C3)nc21] | 2-(cis-3-{5- [(1S)-1-amino- 2,2,2- trifluoroethyl] pyridin-2-yl} cyclobutyl)-7- methoxy[1,2,4] triazolo[1,5-c] quinazolin-5-amine | Coupling done at 70° C.: catalyst A; isomer was isolated via silica gel column chromatography directly (0-100% EtOAc in hex) | 444 |
| Ex-79 | 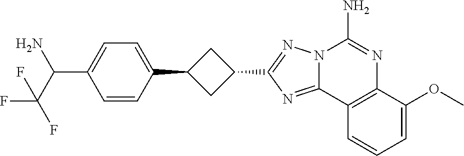 [SMILES: COc1cccc2c1nc(N)n1nc([C@H]3C[C@H](c4ccc(C(N)C(F)(F)F)cc4)C3)nc21] | 2-{trans-3-[4-(1- amino-2,2,2- trifluoroethyl) phenyl]cyclobutyl}- 7-methoxy [1,2,4] triazolo[1,5-c] quinazolin-5- amine | Coupling done at 70° C.; catalyst A; Column: AS-H 21 × 250 mm Eluent: MeOH (w/0.1% NH₄OH); 7.0 min elution | 443 |
| Ex-80 | 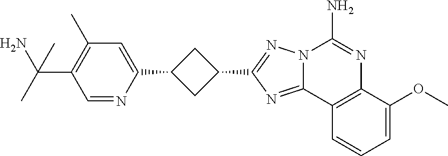 [SMILES: COc1cccc2c1nc(N)n1nc([C@H]3C[C@@H](c4cc(C)c(C(C)(C)N)cn4)C3)nc21] | 2-{cis-3-[5-(2- aminopropan-2-yl)-4- methylpyridin-2- yl]cyclobutyl}-7- methoxy [1,2,4] triazolo[1,5-c] quinazolin-5- amine | Coupling done at 20° C.; catalyst A; Column: AS 30 × 250 mm Eluent: EtOH (w/0.1% NH₄OH); 2.2 min elution | 418 |
| Ex-81 | 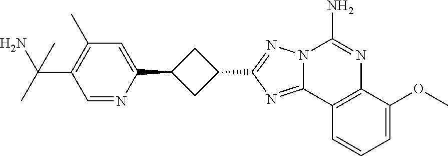 [SMILES: COc1cccc2c1nc(N)n1nc([C@H]3C[C@H](c4cc(C)c(C(C)(C)N)cn4)C3)nc21] | 2-{trans-3-[5-(2- aminopropan-2- yl)-4- methylpyridin-2- yl]cyclobutyl}-7- methoxy [1,2,4] triazolo[1,5-c] quinazolin-5-amine | Coupling done at 20° C.; catalyst A; Column: AS 30 × 250 mm Eluent: EtOH (w/0.1% NH₄OH); 2.6 min elution | 418 |

### Example 82 and 83: 2-((1s,3s)-3-(5-(2-aminopropan-2-yl)-3-methylpyridin-2-yl)cyclobutyl)-7-methoxy-[1.2.4]triazolo[1,5-c]quinazolin-5-amine (Ex-82) and 2-((1r,3r)-3-(5-(2-aminopropan-2-yl)-3-methylpyridin-2-yl)cyclobutyl)-7-methoxy-[1.2.4]triazolo[1,5-c]quinazolin-5-amine (Ex-83)

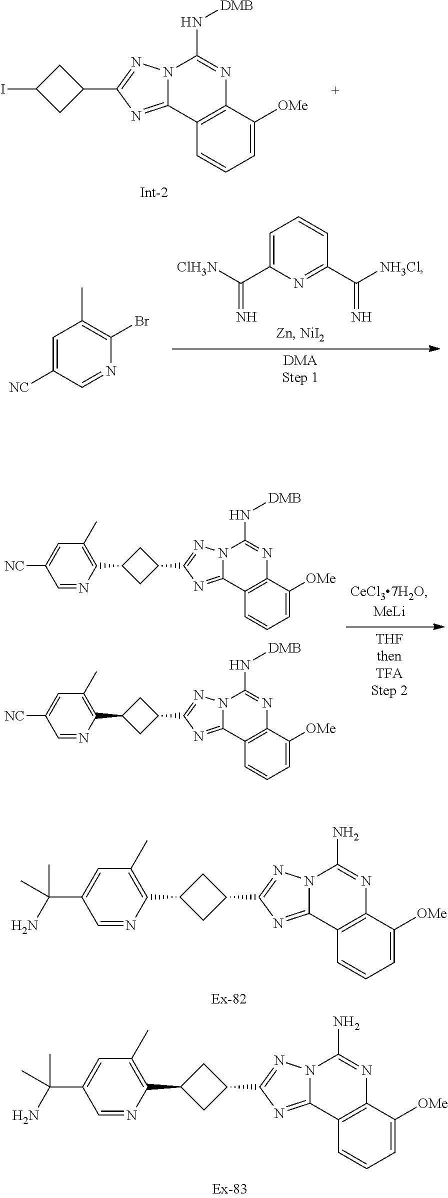

Step 1: A 30 mL vial was charged with 6-bromo-5-methylnicotinonitrile (158 mg, 0.804 mmol), N-(2,4-dimethoxybenzyl)-2-(3-iodocyclobutyl)-7-methoxy-[1,2,4]triazolo[1,5-c]quinazolin-5-amine (Int-2) (274 mg, 0.502 mmol), nickel(II) iodide (62.8 mg, 0.201 mmol), pyridine-2,6-bis(carboximidamide) dihydrochloride (47.0 mg, 0.201 mmol), and zinc (131 mg, 2.010 mmol), the vial was purged with Ar, followed by addition of DMA (5 mL), the reaction was placed in a pre-heated stirring plate at 70° C. The reaction was allowed to stir for 1 h. The sample was diluted with DCM filtered through a Celite™ pad, the pad was rinsed with DCM (20 mL), and the filtrates concentrated under reduced pressure. The crude was purified by silica gel column chromatography (0-50% EtOAc in hex) to afford two peaks of the desired coupled product corresponding to 6-((1s,3s)-3-(5-((3,4-dimethylbenzyl)amino)-7-methoxy-[1,2,4]triazolo[1,5-c]quinazolin-2-yl)cyclobutyl)-5-methylnicotinonitrile (peak 1) and 6-((1r,3r)-3-(5-((3,4-dimethylbenzyl)amino)-7-methoxy-[1,2,4]triazolo[1,5-c]quinazolin-2-yl)cyclobutyl)-5-methylnicotinonitrile (peak 2).

Step 2: A 20 mL vial containing a stir bar was charged with cerium(III) chloride heptahydrate (501 mg, 1.344 mmol, 12 equiv). The vial was placed on a pre-heated stir block and stirred at 150° C. to dry the solid for 16 hours under vacuum. The vial was cooled to room temperature under argon, at which point THF (1.1 mL) was added. The resulting suspension was stirred vigorously at room temperature under Ar for 1 hour. The mixture was cooled to −78° C. After stirring for 10 minutes, methyllithium in diethoxymethane (0.434 mL, 1.344 mmol, 12 equiv) was added dropwise over a period of ˜2 minutes. After an additional 1.5 h. of stirring, a solution of ((1s,3s)-3-(5-((3,4-dimethylbenzyl)amino)-7-methoxy-[1,2,4]triazolo[1,5-c]quinazolin-2-yl)cyclobutyl)-5-methylnicotinonitrile (60 mg, 0.112 mmol) in THF (1.12 mL) was added dropwise. The reaction continued to stir and was allowed to warm slowly for 1 h to ˜−50° C. The reaction was quenched with NH₄Cl (sat aq solution). DCM was added (10 mL) and the layers were separated. The aqueous layer was extracted with a mixture of IPA:CHCl₃ 1:3 (2×20 mL). The combined organic layers were concentrated, and the crude was taken up in TFA (1 mL) at room temperature. The reaction mixture was placed in a pre-heated stirring plate at 45° C., and the mixture was allowed to stir for 2 h. The reaction was concentrated under reduced pressure. The crude material was dissolved in 2 mL of DMSO, filtered and purified by reverse phase HPLC using the NH₄OH modifier to afford 2-((1s,3s)-3-(5-(2-aminopropan-2-yl)-3-methylpyridin-2-yl)cyclobutyl)-7-methoxy-[1,2,4]triazolo[1,5-c]quinazolin-5-amine (Ex-8). ESI MS m/z=418 [M+H]⁺. ¹H NMR (600 MHz, MeOD-d₄) δ 8.48 (d, J=2.2 Hz, 1H), 7.85 (dd, J=8.0, 1.1 Hz, 1H), 7.72 (d, J=1.9 Hz, 1H), 7.36 (t, J=8.0 Hz, 1H), 7.26 (d, J=7.3 Hz, 1H), 4.02 (s, 3H), 4.00-3.84 (m, 2H), 2.94-2.83 (m, 4H), 2.38 (s, 3H), 1.53 (s, 6H).

6-((1r,3r)-3-(5-((3,4-dimethylbenzyl)amino)-7-methoxy-[1,2,4]triazolo[1,5-c]quinazolin-2-yl)cyclobutyl)-5-methylnicotinonitrile was subjected to similar conditions as that for the synthesis of Ex-82 to afford 2-((1r,3r)-3-(5-(2-aminopropan-2-yl)-3-methylpyridin-2-yl)cyclobutyl)-7-methoxy-[1,2,4]triazolo[1,5-c]quinazolin-5-amine (Ex-83). ESI MS m/z=418 [M+H]⁺. ¹H NMR (600 MHz, MeOD-d₄) δ 8.56 (d, J=2.3 Hz, 1H), 7.92 (dd, J=8.0, 1.0 Hz, 1H), 7.73 (d, J=1.9 Hz, 1H), 7.40 (t, J=8.0 Hz, 1H), 7.30 (d, J=7.3 Hz, 1H), 4.23 (p, J=7.9 Hz, 1H), 4.04 (s, 3H), 3.98-3.89 (m, 1H), 2.98-2.90 (m, 4H), 2.34 (s, 3H), 1.56 (s, 6H).

TABLE 7
Using the appropriate starting materials and a method similar to that described above,
the following examples were prepared:

| | | | Column/ | Exact |
|---|---|---|---|---|
| | | | Reaction | Mass |
| Example | Structure | Name | Conditions | [M + H]+ |
| Ex-84 | 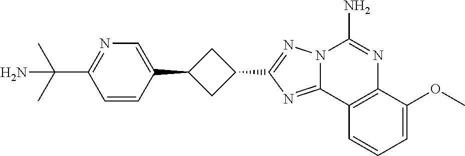 | 2-{trans-3-[6-(2- aminopropan-2- yl)pyridin-3- yl]cyclobutyl}-7- methoxy[1,2,4] triazolo[1,5- c]quinazolin-5- amine | Isomer was isolated via silica gel column chromatogra phy directly (0-100% EtOAc in hex) | 404 |
| Ex-85 | 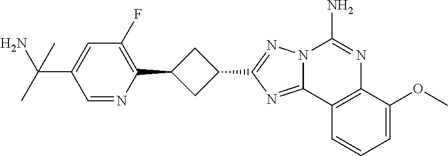 [SMILES: COc1cccc2c1nc(N)n1nc([C@H]3C[C@H](c4ncc(C(C)(C)N)cc4F)C3)nc21] | 2-{trans-3-[5-(2- aminopropan-2- yl)-3- fluoropyridin-2- yl]cyclobutyl}-7- methoxy [1,2,4] triazolo[1,5- c]quinazolin-5- amine | Column: Lux-3 21 × 250 mm Eluent: MeOH (w/ 0.1% NH₄OH); 3.5 min elution | 422 |
| Ex-86 | 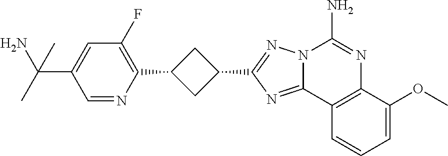 [SMILES: COc1cccc2c1nc(N)n1nc([C@H]3C[C@@H](c4ncc(C(C)(C)N)cc4F)C3)nc21] | 2-{cis-3-[5-(2- aminopropan-2- yl)-3- fluoropyridin-2- yl]cyclobutyl}-7- methoxy[1,2,4] triazolo[1,5- c]quinazolin-5- amine | Column: Lux-3 21 × 250 mm Eluent: MeOH (w/ 0.1% NH₄OH); 1.9 min elution | 422 |
| Ex-87 | 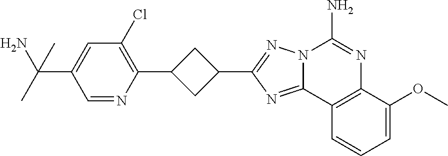 | 2-{3-[5-(2- aminopropan-2- yl)-3- chloropyridin-2- yl]cyclobutyl}-7- methoxy[1,2,4] triazolo[1,5- c]quinazolin-5- amine | Isomer was isolated via silica gel column chromatogra phy directly (0-100% EtOAc in hex); Peak 2 | 438 |
| Ex-88 | 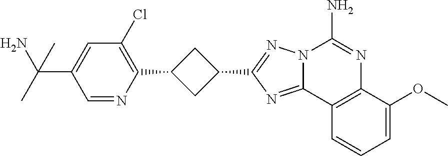 [SMILES: COc1cccc2c1nc(N)n1nc([C@H]3C[C@@H](c4ncc(C(C)(C)N)cc4Cl)C3)nc21] | 2-{cis-3-[5-(2- aminopropan-2- yl)-3- chloropyridin-2- yl]cyclobutyl}-7- methoxy[1,2,4] triazolo[1,5- c]quinazolin-5- amine | Isomer was isolated via silica gel column chromatogra phy directly (0-100% EtOAc in hex); Peak 1 | 438 |

### Example 89 and 90: 2-((1s,3s)-3-(5-(2-aminopropan-2-yl)-3-methylpyrazin-2-yl)cyclobutyl)-7-methoxy-[1,2,4]triazolo[1,5-c]quinazolin-5-amine (Ex-89) and 2-((1r,3r)-3-(5-(2-aminopropan-2-yl)-3-methylpyrazin-2-yl)cyclobutyl)-7-methoxy-[1,2,4]triazolo[1,5-c]quinazolin-5-amine (Ex-90)

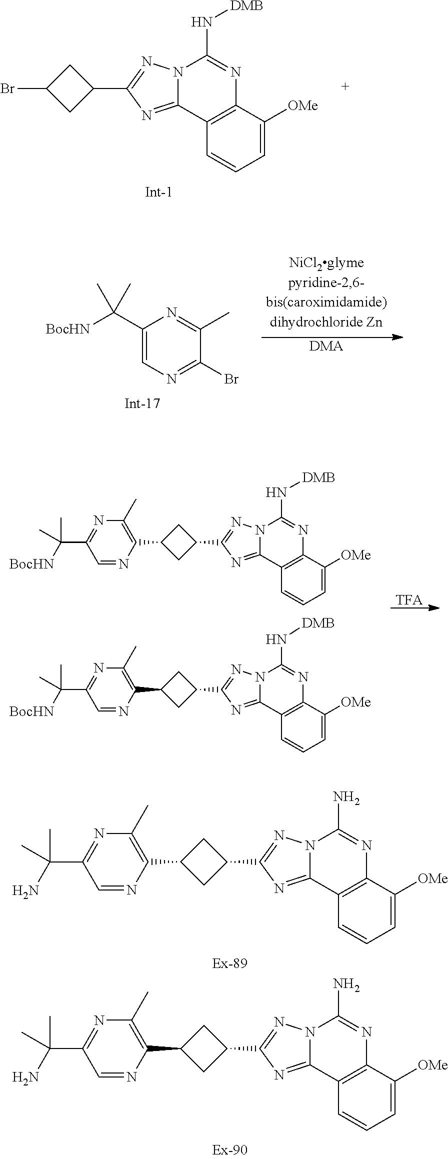

Pyridine-2,6-bis(carboximidamide) dihydrochloride (0.423 g, 1.806 mmol), nickel (II) chloride ethylene glycol dimethyl ether complex (0.397 g, 1.806 mmol), tert-butyl (2-(5-bromo-6-methylpyrazin-2-yl)propan-2-yl)carbamate (Int-17) (1.723 g, 5.22 mmol), 2-(3-bromocyclobutyl)-N-(2,4-dimethoxybenzyl)-7-methoxy-[1,2,4]triazolo[1,5-c]quinazolin-5-amine (Int-1) (2 g, 4.01 mmol), and zinc (1.05 g, 1.806 mmol) were added to a reaction vessel, followed by addition of DMA (20.01 ml) and then the vial was bubble degassed with argon for 5 minutes. The mixture was heated at 70° C. for 16 hours. The reaction was diluted with EtOAc (40 mL) and passed through a plug of Celite™. The filtrate was partitioned with sat. aq. NH₄Cl, separated and the aqueous layer extracted with EtOAc (100 mL×2). The organic layers were combined, washed with brine, dried over anhydrous Na₂SO₄, filtered, and concentrated under reduced pressure. The resulting residue was purified first by silica gel column chromatography (0-100% EtOAc in hex) and then by chiral SFC chromatography (AS-H, 21×250 mm; 20% MeOH with 0.1% NH₄OH modifier) to afford two diastereomers with peak 1 (cis) eluting at 3.4 minutes and peak 2 (trans) eluting at 4.2 minutes. The two diastereomers were then taken up in TFA (0.1 M) and heated at 50° C. for 2 hours. The reactions were concentrated and purified by reverse phase HPLC using the NH₄OH modifier to afford 2-((1s,3s)-3-(5-(2-aminopropan-2-yl)-3-methylpyrazin-2-yl)cyclobutyl)-7-methoxy-[1,2,4]triazolo[1,5-c]quinazolin-5-amine (Ex-89). ESI MS m/z=419 [M+H]⁺. ¹H NMR (400 MHz, MeOD-d₄) δ: 8.62 (s, 1H), 7.83-7.89 (m, 1H), 7.33-7.41 (m, 1H), 7.27 (d, J=7.2 Hz, 1H), 3.90-4.09 (m, 5H), 2.91 (t, J=9.2 Hz, 4H), 2.63 (s, 3H), 1.75 (s, 6H).

2-((1r,3r)-3-(5-(2-aminopropan-2-yl)-3-methylpyrazin-2-yl)cyclobutyl)-7-methoxy-[1,2,4]triazolo[1,5-c]quinazolin-5-amine (Ex-90). ESI MS m/z=419 [M+H]⁺. ¹H NMR (600 MHz, DMSO-d₆) δ 8.74 (s, 1H), 7.80-7.78 (m, 3H), 7.31 (t, J=7.9 Hz, 1H), 7.23 (d, J=7.7 Hz, 1H), 4.09 (p, J=7.8 Hz, 1H), 3.91 (s, 3H), 3.83 (t, J=6.7 Hz, 1H), 2.83 (dq, J=9.7, 5.8, 4.5 Hz, 4H), 2.45 (s, 3H), 2.07 (s, 2H), 1.40 (s, 6H).

TABLE 8
Using the appropriate starting materials and a method similar to that described above, the
following examples were prepared:

| | | | Column/ | Exact |
|---|---|---|---|---|
| | | | Reaction | Mass |
| Example | Structure | Name | Conditions | [M + H]+ |
| Ex-91 | 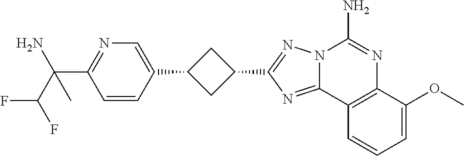 [SMILES: COc1cccc2c1nc(N)n1nc([C@H]3C[C@@H](c4ccc(C(C)(N)C(F)F)nc4)C3)nc21] | 2-{(1s,3s)-3-[6- (2-amino-1,1- difluoropropan- 2-yl)pyridin-3- yl]cyclobutyl}-7- methoxy[1,2,4] triazolo[1,5- c]quinazolin-5- amine | NiI₂ used as Ni source; Column: AS-H 21 × 250 mm Eluent: MeOH (w/ 0.1% NH₄OH); 2.1 min elution | 440 |
| Ex-92 | 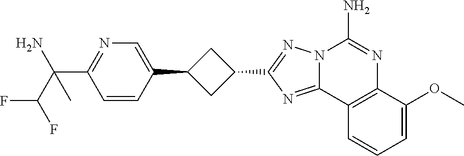 [SMILES: COc1cccc2c1nc(N)n1nc([C@H]3C[C@H](c4ccc(C(C)(N)C(F)F)nc4)C3)nc21] | 2-{(1r,3r)-3-[6- (2-amino-1,1- difluoropropan- 2-yl)pyridin-3- yl]cyclobutyl}-7- methoxy[1,2,4] triazolo[1,5- c]quinazolin-5- amine | NiI₂ used as Ni source; Column: AS-H 21 × 250 mm Eluent: MeOH (w/ 0.1% NH₄OH); 3.4 min elution | 440 |
| Ex-93 | 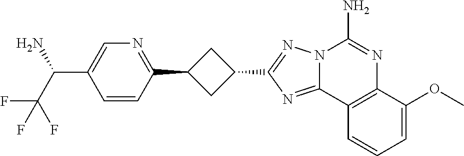 [SMILES: COc1cccc2c1nc(N)n1nc([C@H]3C[C@H](c4ccc([C@@H](N)C(F)(F)F)cn4)C3)nc21] | 2-(trans-3-{5- [(1R)-1-amino- 2,2,2- trifluoroethyl] pyridin-2- yl}cyclobutyl)-7- methoxy[1,2,4] triazolo[1,5- c]quinazolin-5- amine | NiI₂ used as Ni source; isomer was isolated via silica gel column chromatography directly (0- 100% EtOAc in hex); Peak 1 | 444 |
| Ex-94 | 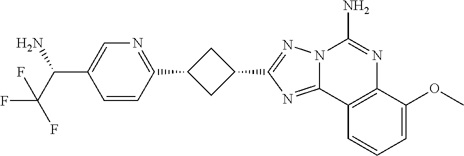 [SMILES: COc1cccc2c1nc(N)n1nc([C@H]3C[C@@H](c4ccc([C@@H](N)C(F)(F)F)cn4)C3)nc21] | 2-(cis-3-{5- [(1R)-1-amino- 2,2,2- trifluoroethyl] pyridin-2- yl}cyclobutyl)-7- methoxy[1,2,4] triazolo[1,5- c]quinazolin-5- amine | NiI₂ used as Ni source; isomer was isolated via silica gel column chromatography directly (0- 100% EtOAc in hex); Peak 2 | 444 |
| Ex-95 | 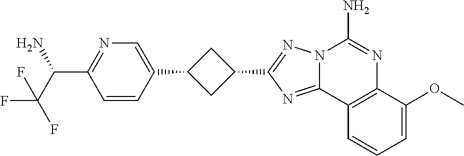 [SMILES: COc1cccc2c1nc(N)n1nc([C@H]3C[C@@H](c4ccc([C@@H](N)C(F)(F)F)nc4)C3)nc21] | 2-[(1s,3s)-3-{6- [(1R)-1-amino- 2,2,2- trifluoroethyl] pyridin-3- yl}cyclobutyl]-7- methoxy[1,2,4] triazolo[1,5- c]quinazolin-5- amine | NiI₂ used as N source; Column: OJ-H 21 × 250 mm Eluent: MeOH (w/ 0.1% NH₄OH); 5.1 min elution | 444 |
| Ex-96 | 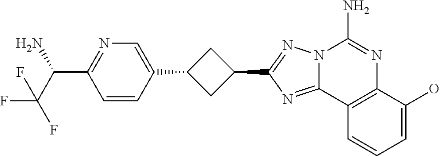 [SMILES: Nc1nc2c(O)cccc2c2nc([C@H]3C[C@H](c4ccc([C@@H](N)C(F)(F)F)nc4)C3)nn12] | 2-[(1r,3r)-3-{6- [(1R)-1-amino- 2,2,2- trifluoroethyl] pyridin-3- yl}cyclobutyl]-7- methoxy [1,2,4] triazolo[1,5- c]quinazolin-5- amine | NiI₂ used as Ni source; Column: OJ-H 21 × 250 mm Eluent: MeOH (w/ 0.1% NH₄OH); 5.9 min elution | 444 |
| Ex-97 | 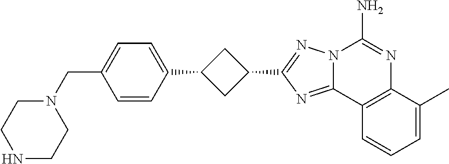 | 7-methoxy-2- (cis-3-{4- [(piperazin-1- yl)methyl]phenyl} cyclobutyl)[1,2, 4]triazolo[1,5- c]quinazolin-5- amine | NiI₂ used as Ni source; Column: Lux-3 21 × 250 mm Eluent: MeOH (w/ 0.1% NH₄OH); 4.3 min elution | 444 |
| Ex-98 | 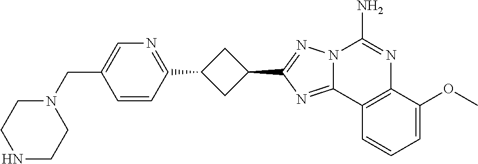 | 7-methoxy-2- (trans-3-{4- [(piperazin-1- yl)methyl]phenyl} cyclobutyl)[1,2, 4]triazolo[1,5- c]quinazolin-5- amine | NiI₂ used as Ni source; Column: Lux-3 21 × 250 mm Eluent: MeOH (w/ 0.1% NH₄OH); 3.0 min elution | 444 |
| Ex-99 | 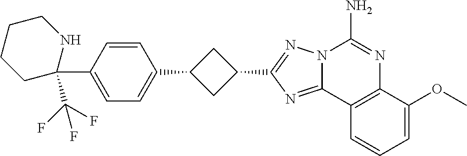 [SMILES: COc1cccc2c1nc(N)n1nc([C@H]3C[C@@H](c4ccc([C@@]5(C(F)(F)F)CCCCN5)cc4)C3)nc21] | 7-methoxy-2- (cis-3-{4-[(2R)- 2-(trifluoromethyl) piperidin-2- yl]phenyl} cyclobutyl)[1,2,4] triazolo[1,5- c]quinazolin-5- amine | NiI₂ used as Ni source; Column: CCA F4 21 × 250 mm Eluent: MeOH (w/ 0.1% NH₄OH); 5.1 min elution | 497 |
| Ex-100 | 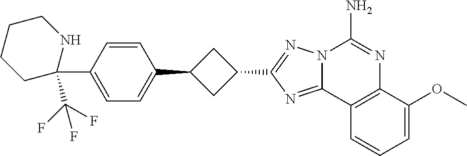 [SMILES: COc1cccc2c1nc(N)n1nc([C@H]3C[C@H](c4ccc([C@@]5(C(F)(F)F)CCCCN5)cc4)C3)nc21] | 7-methoxy-2- (trans-3-{4- [(2R)-2- (trifluoromethyl) piperidin-2- yl]phenyl} cyclobutyl)[1,2,4] triazolo[1,5- c]quinazolin-5- amine | Coupling done at 70° C.; catalyst A; Column: CCA F4 21 × 250 mm Eluent: MeOH (w/ 0.1% NH₄OH); 6.5 min elution | 497 |
| Ex-101 | 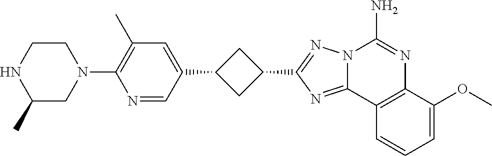 [SMILES: COc1cccc2c1nc(N)n1nc([C@H]3C[C@@H](c4cnc(N5CCN[C@H](C)C5)c(C)c4)C3)nc21] | 7-methoxy-2- (cis-3-{5- methyl-6-[(3R)- 3-methylpiperazin- 1-yl]pyridin-3- yl}cyclobutyl)[1, 2,4]triazolo[1,5- c]quinazolin-5- amine | NiI₂ used as Ni source; Column: Lux-3 21 × 250 mm Eluent: MeOH (w/ 0.1% NH₄OH); 4.6 min elution | 459 |
| Ex-102 | 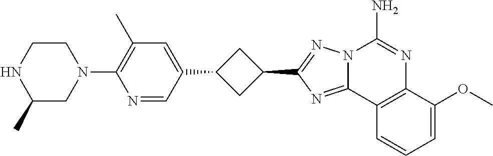 [SMILES: COc1cccc2c1nc(N)n1nc([C@H]3C[C@H](c4cnc(N5CCN[C@H](C)C5)c(C)c4)C3)nc21] | 7-methoxy-2- (trans-3-{5- methyl-6-[(3R)- 3-methylpiperazin- 1-yl]pyridin-3- yl}cyclobutyl)[1, 2,4]triazolo[1,5- c]quinazolin-5- amine | NiI₂ used as Ni source; Column: Lux-3 21 × 250 mm Eluent: MeOH (w/ 0.1% NH₄OH); 5.5 min elution | 459 |
| Ex-103 | 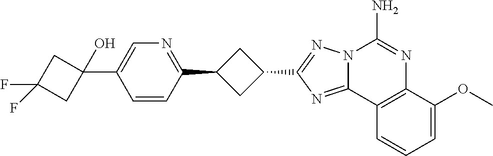 | 1-{6-[trans-3-(5- amino-7- methoxy[1,2,4] triazolo[1,5- c]quinazolin-2- yl)cyclobutyl] pyridin-3-yl}-3,3- difluorocyclobutan- 1-ol | NiI₂ used as Ni source; isomer was isolated via silica gel column chromatography directly (0- 100% EtOAc in hex); Peak 1 | 453 |
| Ex-104 | 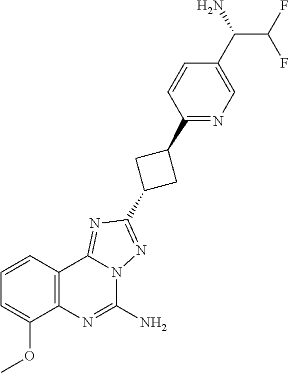 [SMILES: COc1cccc2c1nc(N)n1nc([C@H]3C[C@H](c4ccc([C@H](N)C(F)F)cn4)C3)nc21] | 2-(trans-3-{5- [(1S)-1-amino- 2,2-difluoroethyl] pyridin-2- yl}cyclobutyl)-7- methoxy[1,2,4] triazolo[1,5- c]quinazolin-5- amine | NiI₂ used as Ni source; TFA deprotection for 5 hours; isomer was isolated via silica gel column chromatography directly (0- 100% 3:1 EtOAc:EtOH in hex); Peak 1 | 426 |
| Ex-105 | 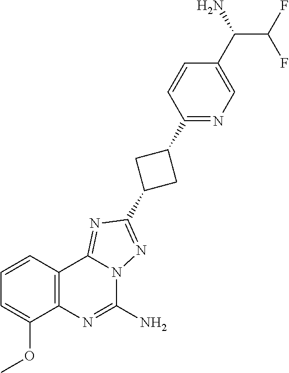 [SMILES: COc1cccc2c1nc(N)n1nc([C@H]3C[C@@H](c4ccc([C@H](N)C(F)F)cn4)C3)nc21] | 2-(cis-3-{5- [(1S)-1-amino- 2,2- difluoroethyl] pyridin-2- yl}cyclobutyl)-7- methoxy[1,2,4] triazolo[1,5- c]quinazolin-5- amine | NiI₂ used as Ni source; TFA deprotection for 5 hours; isomer was isolated via silica gel column chromatography directly (0- 100% 3:1 EtOAc:EtOH in hex); Peak 2 | 426 |
| Ex-106 | 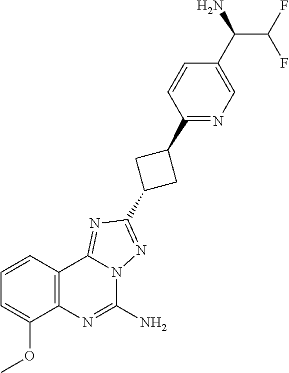 [SMILES: COc1cccc2c1nc(N)n1nc([C@H]3C[C@H](c4ccc([C@@H](N)C(F)F)cn4)C3)nc21] | 2-(trans-3-{5- [(1R)-1-amino- 2,2- difluoroethyl] pyridin-2- yl}cyclobutyl)-7- methoxy [1,2,4] triazolo[1,5- c]quinazolin-5- amine | NiI₂ used as Ni source; TFA deprotection for 5 hours; isomer was isolated via silica gel column chromatography directly (0- 100% 3:1 EtOAc:EtOH in hex); Peak 1 | 426 |
| Ex-107 | 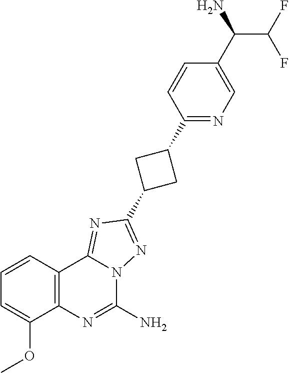 [SMILES: COc1cccc2c1nc(N)n1nc([C@H]3C[C@@H](c4ccc([C@@H](N)C(F)F)cn4)C3)nc21] | 2-(cis-3-{5- [(1R)-1-amino- 2,2- difluoroethyl] pyridin-2- yl}cyclobutyl)- 7- methoxy[1,2,4] triazolo[1,5- c]quinazolin-5- amine | NiI₂ used as Ni source; TFA deprotection ran for 5 hours; isomer was isolated via silica gel column chromatography directly (0- 100% 3:1 EtOAc:EtOH in hex); Peak 2 | 426 |
| Ex-108 | 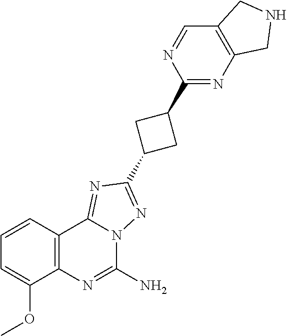 [SMILES: COc1cccc2c1nc(N)n1nc([C@H]3C[C@H](c4ncc5c(n4)CNC5)C3)nc21] | 2-((trans)-3-(6,7- dihydro-5H- pyrrolo[3,4- d]pyrimidin-2- yl)cyclobutyl)-7- methoxy- [1,2,4]triazolo[1, 5-c]quinazolin- 5-amine | NiI₂ used as Ni source; isomer was isolated via silica gel column chromatography directly (0- 100% 3:1 EtOAc:EtOH in hex); Peak 1 | 389 |
| Ex-109 | 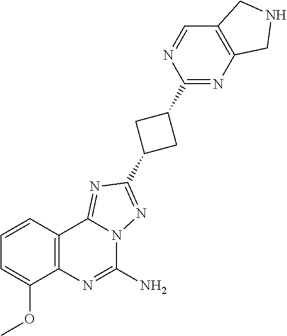 [SMILES: COc1cccc2c1nc(N)n1nc([C@H]3C[C@@H](c4ncc5c(n4)CNC5)C3)nc21] | 2-[(cis)-3-(6,7- dihydro-5H- pyrrolo[3,4- d]pyrimidin-2- yl)cyclobutyl]-7- methoxy [1,2,4] triazolo[1,5- c]quinazolin-5- amine | NiI₂ used as Ni source; isomer was isolated via silica gel column chromatography directly (0- 100% 3:1 EtOAc:EtOH in hex); Peak 2 | 389 |
| Ex-110 | 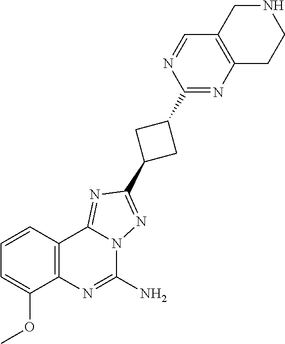 [SMILES: COc1cccc2c1nc(N)n1nc([C@H]3C[C@H](c4ncc5c(n4)CCNC5)C3)nc21] | 7-methoxy-2- [(trans)-3- (5,6,7,8- tetrahydropyrido [4,3- d]pyrimidin-2- yl)cyclobutyl][1, 2,4]triazolo[1,5- c]quinazolin-5- amine | NiI₂ used as Ni source; isomer was isolated via silica gel column chromatography directly (0- 100% 3:1 EtOAc: EtOH in hex); Peak 1; repurified using TFA method, then NH₄OH method to generate a purer sample. | 403 |
| Ex-111 | 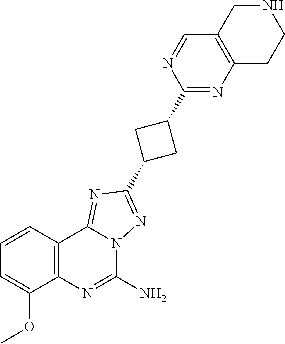 [SMILES: COc1cccc2c1nc(N)n1nc([C@H]3C[C@@H](c4ncc5c(n4)CCNC5)C3)nc21] | 7-methoxy-2- [(cis)-3-(5,6,7,8- tetrahydropyrido [4,3- d]pyrimidin-2- yl)cyclobutyl][1, 2,4]triazolo[1,5- c]quinazolin-5- amine | NiI₂ used as Ni source; isomer was isolated via silica gel column chromatography directly (0- 100% 3:1 EtOAc:EtOH in hex); Peak 2 | 403 |
| Ex-112 | 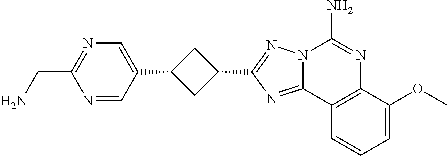 [SMILES: COc1cccc2c1nc(N)n1nc([C@H]3C[C@@H](c4cnc(CN)nc4)C3)nc21] | 2-{(cis)-3-[2- (aminomethyl) pyrimidin-5- yl]cyclobutyl}- 7- methoxy[1,2,4] triazolo[1,5- c]quinazolin-5- amine | NiI₂ used as Ni source; Column: AS-H 21 × 250 mm Eluent: 25% MeOH (w/ 0.1% NH₄OH); 3.5 min elution | 377 |
| Ex-113 | 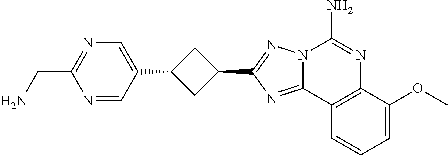 [SMILES: COc1cccc2c1nc(N)n1nc([C@H]3C[C@H](c4cnc(CN)nc4)C3)nc21] | 2-{trans-3-[2- (aminomethyl) pyrimidin-5- yl]cyclobutyl}- 7- methoxy[1,2,4] triazolo[1,5- c]quinazolin-5- amine | NiI₂ used as Ni source; Column: AS-H 21 × 250 mm Eluent: 25% MeOH (w/ 0.1% NH₄OH); 4.6 min elution; repurified | 377 |
| | | | using TFA | |
| | | | method, then | |
| | | | NH₄OH | |
| | | | method to | |
| | | | generate a | |
| | | | purer sample | |
| Ex-114 | 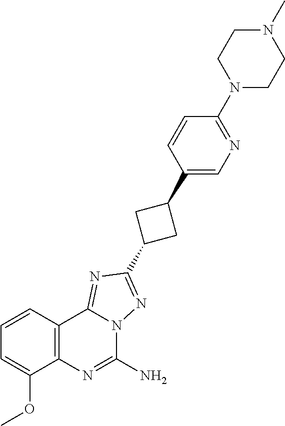 | 7-methoxy-2- {trans-3-[6-(4- methylpiperazin- 1-yl)pyridin-3- yl]cyclobutyl}[1, 2,4]triazolo[1,5- c]quinazolin-5- amine | NiI₂ used as Ni source; Column: Lux-3 21 × 250 mm Eluent: 30% MeOH (w/ 0.1% NH₄OH); 8.9 min elution | 445 |
| Ex-115 | 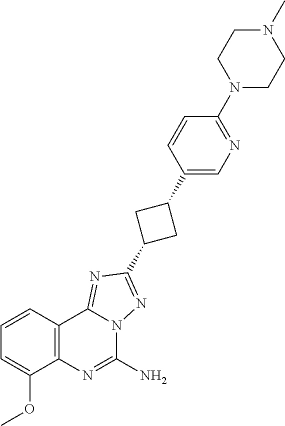 | 7-methoxy-2- {cis-3-[6-(4- methylpiperazin- 1-yl)pyridin-3- yl]cyclobutyl}[1, 2,4]triazolo[1,5- c]quinazolin-5- amine | NiI₂ used as Ni source; Column: Lux-3 21 × 250 mm Eluent: 30% MeOH (w/ 0.1% NH₄OH); 7.3 min elution | 445 |
| Ex-116 | 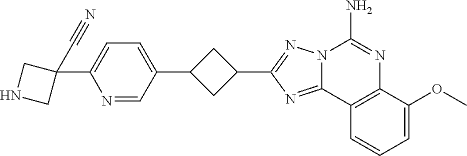 | 3-{5-[3-(5- amino-7- methoxy[1,2,4] triazolo[1,5- c]quinazolin-2- yl)cyclobutyl] pyridin-2- yl}azetidine-3- carbonitrile | NiI₂ used as Ni source; mixture of diastereomers; repurified using TFA method, then NH₄OH method to generate a | 427 |
| | | | purer sample | |
| Ex-117 | 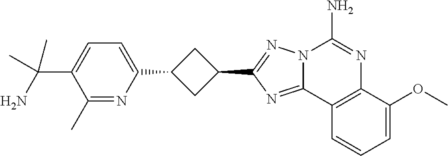 [SMILES: COc1cccc2c1nc(N)n1nc([C@H]3C[C@H](c4ccc(C(C)(C)N)c(C)n4)C3)nc21] | 2-{trans-3-[5-(2- aminopropan-2- yl)-6- methylpyridin-2- yl]cyclobutyl}- 7- methoxy[1,2,4] triazolo[1,5- c]quinazolin-5- amine | Isomer was isolated via silica gel column chromatography directly (0- 100% EtOAc in hex); Peak 1 | 418 |
| Ex-118 | 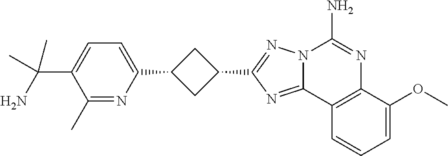 [SMILES: COc1cccc2c1nc(N)n1nc([C@H]3C[C@@H](c4ccc(C(C)(C)N)c(C)n4)C3)nc21] | 2-{cis-3-[5-(2- aminopropan-2- yl)-6- methylpyridin-2- yl]cyclobutyl}-7- methoxy[1,2,4] triazolo[1,5- c]quinazolin-5- amine | Isomer was isolated via silica gel column chromatography directly (0- 100% EtOAc in hex); Peak 2 | 418 |
| Ex-119 | 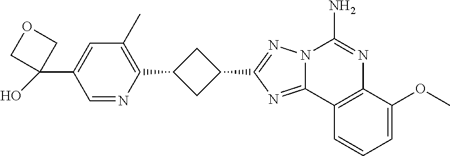 [SMILES: COc1cccc2c1nc(N)n1nc([C@H]3C[C@@H](c4ncc(C5(O)COC5)cc4C)C3)nc21] | 3-{6-[cis-3-(5- amino-7- methoxy[1,2,4] triazolo[1,5- c]quinazolin-2- yl)cyclobutyl]-5- methylpyridin-3- yl}oxetan-3-ol | Column: OJ-H 21 × 250 mm Eluent: 40% MeOH (w/ 0.1% NH₄OH); 3.4 min elution | 433 |
| Ex-120 | 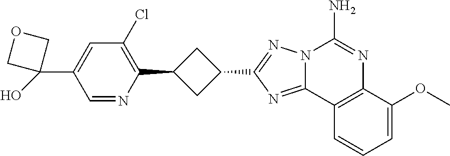 [SMILES: COc1cccc2c1nc(N)n1nc([C@H]3C[C@H](c4ncc(C5(O)COC5)cc4Cl)C3)nc21] | 3-{6-[trans-3-(5- amino-7- methoxy[1,2,4] triazolo[1,5- c]quinazolin-2- yl)cyclobutyl]-5- methylpyridin-3- yl}oxetan-3-ol | Column: OJ-H 21 × 250 mm Eluent: 40% MeOH (w/ 0.1% NH₄OH); 4.8 min elution | 433 |

### Example 121 and 122: 2-((1r,3r)-3-(5-(2-aminopropan-2-yl)-6-methylpyrazin-2-yl)cyclobutyl)-7-methoxy-[1,2,4]triazolo[1,5-c]quinazolin-5-amine (Ex-121) and 2-((1s,3s)-3-(5-(2-aminopropan-2-yl)-6-methylpyrazin-2-yl)cyclobutyl)-7-methoxy-[1,2,4]triazolo[1,5-c]quinazolin-5-amine (Ex-122)

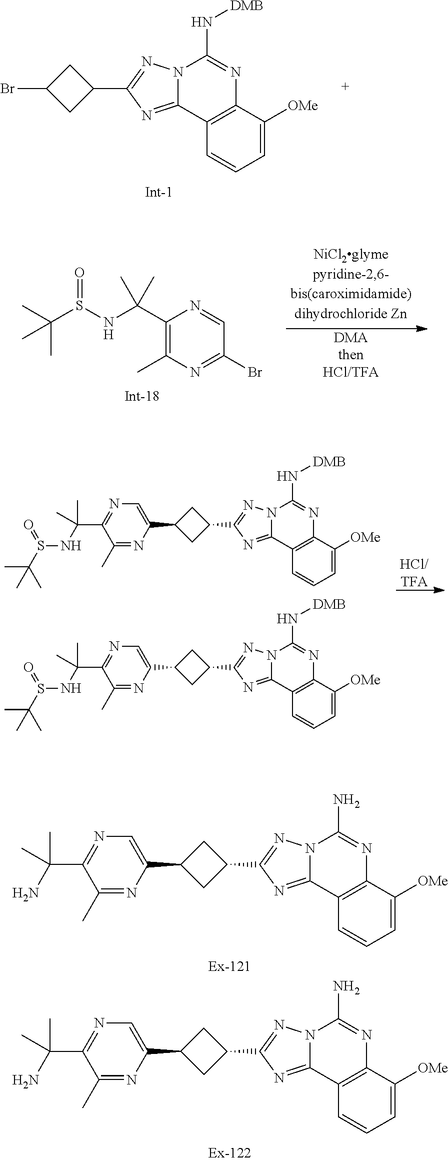

Pyridine-2,6-bis(carboximidamide) dihydrochloride (23.25 mg, 0.099 mmol), nickel (II) chloride ethylene glycol dimethyl ether complex (21.82 mg, 0.099 mmol), N-(2-(5-bromo-3-methylpyrazin-2-yl)propan-2-yl)-2-methylpropane-2-sulfinamide (Int-18) (96 mg, 0.287 mmol), 2-(3-bromocyclobutyl)-N-(2,4-dimethoxybenzyl)-7-methoxy-[1,2,4]triazolo[1,5-c]quinazolin-5-amine (Int-1) (110 mg, 0.221 mmol), and zinc (57.7 mg, 0.883 mmol) were added to a 40 mL vial, followed by addition of DMA (20.06 ml) and then the vial was bubble degassed with Ar for 5 min. The mixture was heated at 70° C. for 16 hours. The reaction was diluted with EtOAc (20 mL) and passed through a plug of Celite™. The filtrate was partitioned with sat. aq. NH₄Cl. The layers were separated and the aqueous layer was extracted with EtOAc (2×). The combined organic layers were washed with brine, dried over anhydrous Na₂SO₄, filtered, and concentrated under reduced pressure. The resulting crude residue was purified by silica gel column chromatography (0-100% 3:1 EtOAc:EtOH in hex) and further purified by SFC (BEH, 21×250 mm, 10% MeOH with 0.1% NH₄OH modifier) to afford N-(2-(5-((1r,3r)-3-(5-((2,4-dimethoxybenzyl)amino)-7-methoxy-[1,2,4]triazolo[1,5-c]quinazolin-2-yl)cyclobutyl)-3-methylpyrazin-2-yl)propan-2-yl)-2-methylpropane-2-sulfinamide (peak 1 eluting at 4.7 min) and N-(2-(5-((1s,3s)-3-(5-((2,4-dimethoxybenzyl)amino)-7-methoxy-[1,2,4]triazolo[1,5-c]quinazolin-2-yl)cyclobutyl)-3-methylpyrazin-2-yl)propan-2-yl)-2-methylpropane-2-sulfinamide (peak 2 eluting at 5.4 minutes). N-(2-(5-((1r,3r)-3-(5-((2,4-dimethoxybenzyl)amino)-7-methoxy-[1,2,4]triazolo[1,5-c]quinazolin-2-yl)cyclobutyl)-3-methylpyrazin-2-yl)propan-2-yl)-2-methylpropane-2-sulfinamide was dissolved in MeOH (32 μL) and HCl in dioxane (4M, 16 μL) was added. The reaction was stirred for 1 hour at room temperature. The reaction was then concentrated under reduced pressure and was taken up in TFA (122 μL) and stirred at 50° C. for 2 hours. The reaction was concentrated under reduced pressure and purified by reverse phase HPLC using the NH₄OH modifier to afford 2-((1r,3r)-3-(5-(2-aminopropan-2-yl)-6-methylpyrazin-2-yl)cyclobutyl)-7-methoxy-[1,2,4]triazolo[1,5-c]quinazolin-5-amine (Ex-121) ESI MS m/z=419 [M+H]⁺. ¹H NMR (600 MHz, DMSO-d₆) δ 8.32 (s, 1H), 7.79 (m, 3H), 7.31 (t, J=7.9 Hz, 1H), 7.23 (d, J=7.7 Hz, 1H), 3.96 (dt, J=13.0, 7.9 Hz, 2H), 3.91 (s, 3H), 2.87 (s, 3H), 2.83-2.74 (m, 4H), 1.49 (s, 6H).

The same procedure was repeated for N-(2-(5-((1s,3s)-3-(5-((2,4-dimethoxybenzyl)amino)-7-methoxy-[1,2,4]triazolo[1,5-c]quinazolin-2-yl)cyclobutyl)-3-methylpyrazin-2-yl)propan-2-yl)-2-methylpropane-2-sulfinamide to afford 2-((1s,3s)-3-(5-(2-aminopropan-2-yl)-6-methylpyrazin-2-yl)cyclobutyl)-N-(2,4-dimethoxybenzyl)-7-methoxy-[1,2,4]triazolo[1,5-c]quinazolin-5-amine (Ex-122). ESI MS m/z=419 [M+H]⁺. ¹H NMR (600 MHz, DMSO-d₆) δ 8.31 (s, 1H), 7.86-7.70 (m, 3H), 7.29 (t, J=7.9 Hz, 1H), 7.21 (d, J=7.3 Hz, 1H), 3.90 (s, 3H), 3.79 (dp, J=61.1, 9.2, 8.7 Hz, 2H), 2.82 (s, 3H), 2.78-2.69 (m, 4H), 1.47 (s, 6H).

TABLE 9
Using the appropriate starting materials and a method similar to that described above, the
following examples were prepared:

| | | | Column/ | Exact |
|---|---|---|---|---|
| | | | Reaction | Mass |
| Example | Structure | Name | Conditions | [M + H]+ |
| Ex-123 | 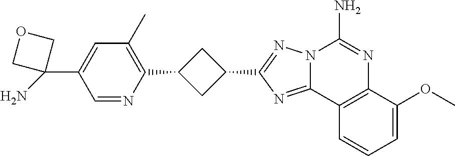 [SMILES: COc1cccc2c1nc(N)n1nc([C@H]3C[C@@H](c4ncc(C5(N)COC5)cc4C)C3)nc21] | 2-{cis-3-[5-(3- aminooxetan-3- yl)-3- methylpyridin-2- yl]cyclobutyl}-7- methoxy[1,2,4] triazolo[1,5- c]quinazolin-5- amine | Column: OJ-H 21 × 250 mm Eluent: 25% MeOH (w/ 0.1% NH₄OH); 4.8 + 5.6 min elution; Work up | 432 |
| | | | with | |
| | | | NaHCO₃ | |
| | | | done prior to | |
| | | | concentration/ | |
| | | | TFA | |
| | | | deprotection | |
| Ex-124 | 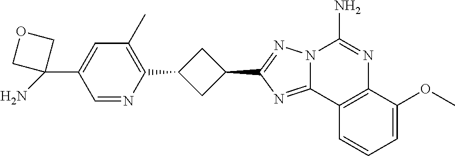 [SMILES: COc1cccc2c1nc(N)n1nc([C@H]3C[C@H](c4ncc(C5(N)COC5)cc4C)C3)nc21] | 2-{trans-3-[5-(3- aminooxetan-3- yl)-3- methylpyridin-2- yl]cyclobutyl}-7- methoxy[1,2,4] triazolo[1,5- c]quinazolin-5- amine | Column: OJ-H 21 × 250 mm Eluent: 25% MeOH (w/ 0.1% NH₄OH); 7.2 min elution; Work up | 432 |
| | | | with | |
| | | | NaHCO₃ | |
| | | | done prior | |
| | | | to | |
| | | | concentration/ | |
| | | | TFA | |
| | | | deprotection | |

### Example 125: 2-((1r,3r)-3-(5-(2-aminopropan-2-Yl)-3-cyclopropylpyrazin-2-yl)cyclobut)-7 methoxy-[1,2,4]triazolo[1,5-c]quinazolin-5-amine (Ex-125)

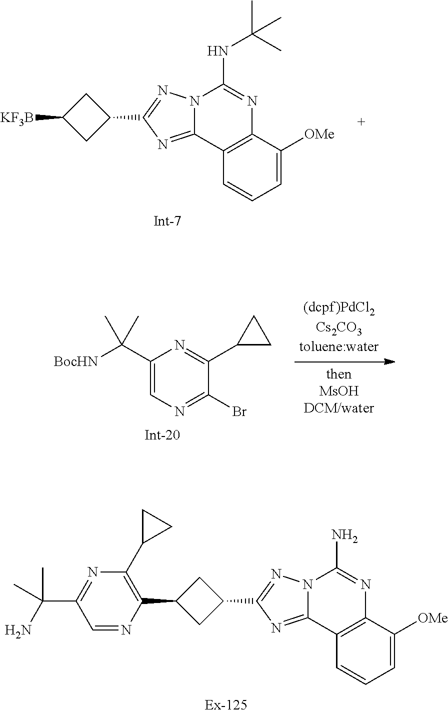

Tert-butyl (2-(5-bromo-6-cyclopropylpyrazin-2-yl)propan-2-yl)carbamate (Int-20) (121 mg, 0.340 mmol), N-(tert-butyl)-7-methoxy-2-((1r,3r)-3-(trifluoro-l4-boraneyl)cyclobutyl)-[1,2,4]triazolo[1,5-c]quinazolin-5-amine, potassium salt (Int-7) (176 mg, 0.408 mmol), cesium carbonate (443 mg, 1.359 mmol), and dichloro[1,1′-bis(dicyclohexylphosphino)ferrocene]palladium(II) (51.3 mg, 0.068 mmol) were placed in 40 mL vial. In a glove box, toluene (2177 μl) and degassed water (435 μl) were added. The reaction vessel was sealed and heated at 105° C. for 16 hours. The reaction was cooled to room temperature, diluted with water, and extracted with EtOAc (3×). The combined organic layers were washed with brine, dried over anhydrous Na₂SO₄, filtered, and concentrated under reduced pressure. The resulting residue was purified by silica gel column chromatography (0-70% EtOAc in hex) to afford the cross-coupled product. The cross-coupled product was then taken up in DCM (318 μl), followed by the dropwise addition of methanesulfonic acid (165 μl, 2.54 mmol) and water (17.16 μl, 0.953 mmol) at room temperature. The reaction was heated at 40° C. for 16 hours. After cooling to room temperature, the reaction was diluted with DCM and water. The DCM layer was removed, and the aqueous layer basified to ˜pH 10 with sat. aq. NaHCO₃. The aqueous layer was extracted with DCM (3×). The combined organic layers were dried over anhydrous Na₂SO₄, filtered, concentrated under reduced pressure, and purified by mass triggered reverse phase HPLC (MeCN/water with 0.1% NH₄OH modifier, linear gradient) to afford 2-((1r,3r)-3-(5-(2-aminopropan-2-yl)-3-cyclopropylpyrazin-2-yl)cyclobutyl)-7-methoxy-[1,2,4]triazolo[1,5-c]quinazolin-5-amine (Ex-125). ESI MS m/z=445 [M+H]⁺. ¹H NMR (600 MHz, DMSO-d₆) δ 8.64 (s, 1H), 7.79 (m, 3H), 7.31 (t, J=7.9 Hz, 1H), 7.23 (d, J=7.9 Hz, 1H), 4.38-4.25 (m, 1H), 3.91 (s, 3H), 3.83 (s, 1H), 2.88 (td, J=16.3, 14.2, 8.5 Hz, 4H), 2.17-2.10 (m, 1H), 1.36 (s, 6H), 0.98 (d, J=5.8 Hz, 4H).

TABLE 10
Using the appropriate starting materials and a method similar to that described above,
the following examples were prepared:

| | | | Exact |
|---|---|---|---|
| | | | Mass |
| Example | Structure | Name | [M + H]+ |
| Ex-126 | 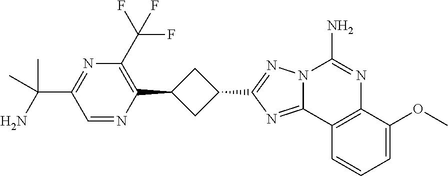 [SMILES: COc1cccc2c1nc(N)n1nc([C@H]3C[C@H](c4ncc(C(C)(C)N)nc4C(F)(F)F)C3)nc21] | 2-{trans-3-[5-(2- aminopropan-2-yl)-3- (trifluoromethyl)pyrazin- 2-yl]cyclobutyl}-7- methoxy[1,2,4]triazolo[1, 5-c]quinazolin-5-amine | 473 |

### Example 127: 2-((1r,3r)-3-(5-(2-aminopropan-2-yl)-3-methylpyrazin-2-yl)cyclobutyl)-7-chloro-[1,2,4]triazolo[1,5-c]quinazolin-5-amine (Ex-127)

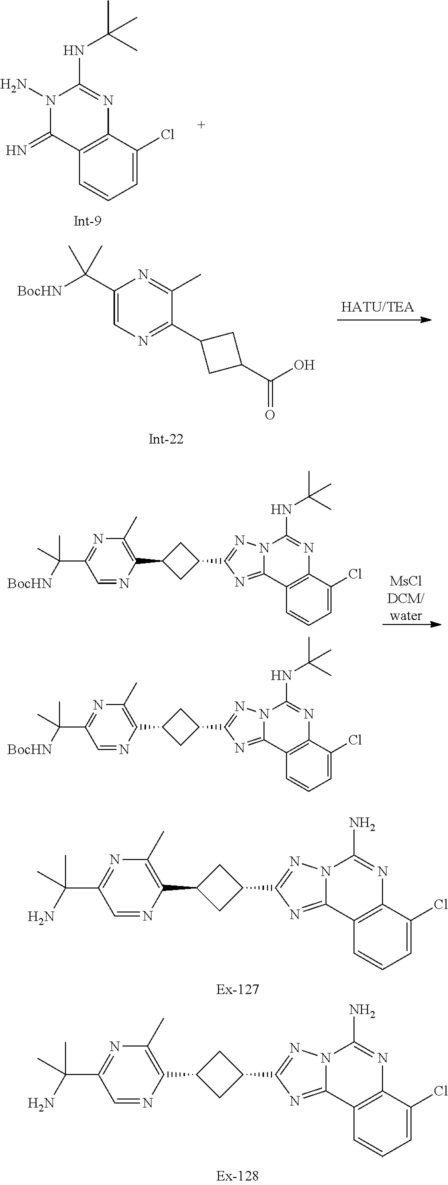

3-(5-(2-((tert-Butoxycarbonyl)amino)propan-2-yl)-3-methylpyrazin-2-yl)cyclobutane-1-carboxylic acid (Int-2) (67.1 mg, 0.192 mmol), N²-(tert-butyl)-8-chloro-4-iminoquinazoline-2,3(4H)-diamine (Int-9) (51.0 mg, 0.192 mmol) and HATU (73.0 mg, 0.192 mmol) were dissolved in THF (1920 μl). Triethylamine (29.4 μl, 0.211 mmol) was added and the mixture was stirred at 55° C. for 16 hours. The reaction was concentrated, diluted with water, and extracted with DCM (3×). The combined organic layers were dried over anhydrous Na₂SO₄, filtered, and concentrated under reduced pressure. The resulting residue was purified by silica gel column chromatography (0-50% EtOAc in hex) to afford 2-((1r,3r)-3-(5-(2-aminopropan-2-yl)-3-methylpyrazin-2-yl)cyclobutyl)-N-(tert-butyl)-7-chloro-[1,2,4]triazolo[1,5-c]quinazolin-5-amine (peak 1) and 2-((1s,3s)-3-(5-(2-aminopropan-2-yl)-3-methylpyrazin-2-yl)cyclobutyl)-N-(tert-butyl)-7-chloro-[1,2,4]triazolo[1,5-c]quinazolin-5-amine (peak 2). 2-((1r,3r)-3-(5-(2-aminopropan-2-yl)-3-methylpyrazin-2-yl)cyclobutyl)-N-(tert-butyl)-7-chloro-[1,2,4]triazolo[1,5-c]quinazolin-5-amine was taken up in DCM (305 l). Methanesulfonic acid (158 μl 2.438 mmol) was added dropwise followed by dropwise addition of water (16.47 μl, 0.914 mmol) at room temperature. The reaction was heated at 40° C. for 16 hours. The reaction was then diluted with DCM and water. The DCM layer was removed, and the aqueous layer basified to ˜pH 10 with sat. NaHCO₃. The aqueous layer was extracted with DCM (3×). The organic extracts were combined, dried over anhydrous Na₂SO₄, filtered, concentrated under reduced pressure, and purified by mass triggered reverse phase HPLC (MeCN/water with 0.1% NH₄OH modifier, linear gradient) to afford 2-((1r,3r)-3-(5-(2-aminopropan-2-yl)-3-methylpyrazin-2-yl)cyclobutyl)-7-chloro-[1,2,4]triazolo[1,5-c]quinazolin-5-amine (Ex-1271. ESI MS m/z=423 [M+H]⁺. ¹H NMR (500 MHz, DMSO-d₆) δ 8.74 (s, 1H), 8.20 (d, J=7.9 Hz, 1H), 8.10 (s, 2H), 7.84 (d, J=7.5 Hz, I1H), 7.34 (t, J=7.8 Hz, I1H), 4.10 (p, J=8.0 Hz, 1H), 3.84 (p, J=7.3 Hz, 1H), 2.91-2.75 (mi, 4H), 2.46 (s, 3H), 1.41 (s, 6H).

TABLE 11
Using the appropriate starting materials and a method similar to that described above,
the following examples were prepared:

| | | | Column/ | Exact |
|---|---|---|---|---|
| | | | Reaction | Mass |
| Example | Structure | Name | Conditions | [M + H]+ |
| Ex-128 | 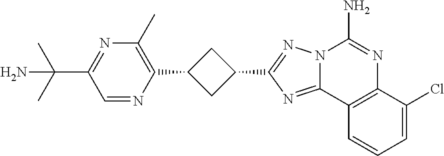 [SMILES: Cc1nc(C(C)(C)N)cnc1[C@H]1C[C@@H](c2nc3c4cccc(Cl)c4nc(N)n3n2)C1] | 2-{cis-3-(5-(2- aminopropan-2- yl)-3- methylpyrazin-2- yl)cyclobutyl}-7- chloro- [1,2,4]triazolo[1,5- c]quinazolin-5- amine | | 422 |
| Ex-129 | 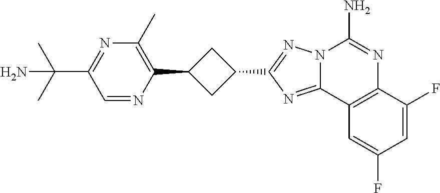 | 2-{trans-3-[5-(2- aminopropan-2- yl)-3- methylpyrazin-2- yl]cyclobutyl}- 7,9- difluoro[1,2,4] triazolo[1,5- c]quinazolin-5- amine | | 425 |
| Ex-130 | 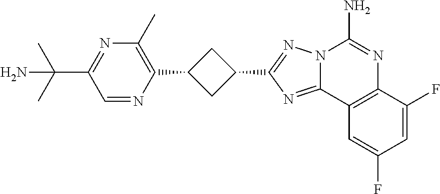 | 2-{cis-3-[5-(2- aminopropan-2- yl)-3- methylpyrazin-2- yl]cyclobutyl}- 7,9- difluoro[1,2,4] triazolo[1,5- c]quinazolin-5- amine | | 425 |
| Ex-131 | 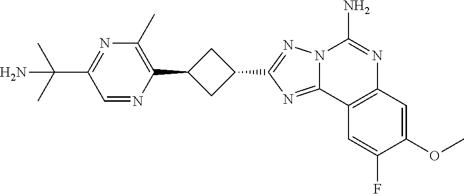 | 2-{trans-3-[5-(2- aminopropan-2- yl)-3- methylpyrazin-2- yl]cyclobutyl}-9- fluoro-8- methoxy[1,2,4] triazolo[1,5- c]quinazolin-5- amine | | 437 |
| Ex-132 | 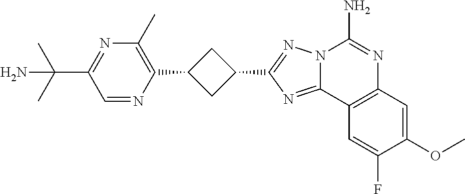 | 2-{cis-3-[5-(2- aminopropan-2- yl)-3- methylpyrazin-2- yl]cyclobutyl}-9- fluoro-8- methoxy[1,2,4] triazolo[1,5- c]quinazolin-5- amine | | 437 |
| Ex-133 | 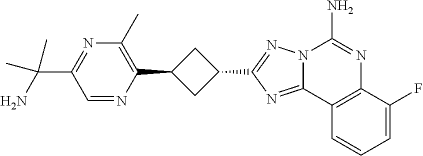 [SMILES: Cc1nc(C(C)(C)N)cnc1[C@H]1C[C@H](c2nc3c4cccc(F)c4nc(N)n3n2)C1] | 2-{trans-3-[5-(2- aminopropan-2- yl)-3- methylpyrazin-2- yl]cyclobutyl}-7- fluoro[1,2,4] triazolo[1,5- c]quinazolin-5- amine | Isolated by SFC after cross- coupling Column: CCO F4, 21 × 250 mm Eluent: 30% MeOH with 0.1% | 407 |
| | | | NH₄OH | |
| | | | modifier); | |
| | | | Peak at 2.9 | |
| | | | min; | |
| | | | Deprotection | |
| | | | done with | |
| | | | TFA | |
| Ex-134 | 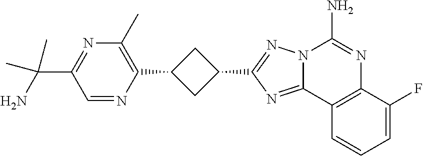 [SMILES: Cc1nc(C(C)(C)N)cnc1[C@H]1C[C@@H](c2nc3c4cccc(F)c4nc(N)n3n2)C1] | 2-{cis-3-[5-(2- aminopropan-2- yl)-3- methylpyrazin-2- yl]cyclobutyl}-7- fluoro[1,2,4] triazolo[1,5- c]quinazolin-5- amine | Isolated by SFC after cross- coupling Column: CCO F4, 21 × 250 mm Eluent: 30% MeOH with 0.1% | 407 |
| | | | NH₄OH | |
| | | | modifier); | |
| | | | Peak at 5.5 | |
| | | | min.; | |
| | | | Deprotection | |
| | | | done with | |
| | | | TFA | |

### Example 135 and 136: 7-methoxy-2-((1s,3s)-3-phenylcyclobutyl)-[1,2,4]triazolo[1,5-c]quinazolin-5-amine (Ex-135) and 7-methoxy-2-((1r,3r-3-phenylcyclobutyl-[1,2,4]triazolo[1,5-c]quinazolin-5-amine (Ex-136)

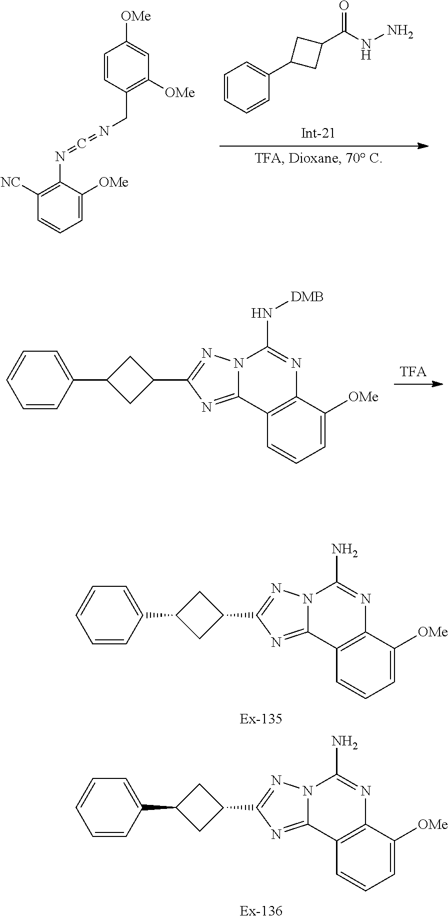

Step 1: 3-phenylcyclobutane-1-carbohydrazide (Int-2) (140 mg, 0.736 mmol, 1.2 equiv) and 2-((((2,4-dimethoxybenzyl)imino)methylene)amino)-3-methoxybenzonitrile (198 mg, 0.613 mmol, 1.0 equiv) were taken up in 1,4-dioxane (2 mL) and acetic acid (18 μL, 0.307 mmol, 0.5 equiv) and the mixture was heated and stirred at 70° C. for 16 hours. After cooling, the reaction mixture was directly purified by silica gel column chromatography (0-100% EtOAc in hex) to afford N-(2,4-dimethoxybenzyl)-7-methoxy-2-(3-phenylcyclobutyl)-[1,2,4]triazolo[1,5-c]quinazolin-5-amine. ESI MS m/z=496 [M+H]⁺.

Step 2: A 40 mL scintillation vial was charged with N-(2,4-dimethoxybenzyl)-7-methoxy-2-(3-phenylcyclobutyl)-[1,2,4]triazolo[1,5-c]quinazolin-5-amine (161 mg, 0.325 mmol, 1.0 equiv). TFA (3.2 mL) was then added, and the reaction mixture was heated at 50° C. and stirred for 2 hours. After cooling, the reaction was concentrated, and the residue was purified by reverse phase HPLC using the TFA modifier. The purified sample was taken up in 25% i-PrOH in CHCl₃ (10 mL) and sat. aq. NaHCO₃ (5 mL) was added. The resulting biphasic mixture was stirred at 25° C. for 1 hour. The layers were separated, and the aq. layer was extracted with 25% i-PrOH in CHCl₃ (2×10 mL). The combined organic layers were concentrated, and the residue was purified by achiral SFC (CCA 21×250 mm column with 25% MeOH (w/0.1% NH₄OH) as cosolvent), to afford 7-methoxy-2-((1s,3s)-3-phenylcyclobutyl)-[1,2,4]triazolo[1,5-c]quinazolin-5-amine (Ex-135) as the first eluting isomer and 7-methoxy-2-((1r,3r)-3-phenylcyclobutyl)-[1,2,4]triazolo[1,5-c]quinazolin-5-amine (Ex-136) as the second eluting isomer. (Ex-135): ESI MS m/z=346 [M+H]⁺. ¹H NMR (600 MHz, DMSO-d₆) δ 7.83-7.73 (m, 3H), 7.38-7.31 (m, 4H), 7.29 (app t, J=7.9 Hz, 1H), 7.24-7.19 (m, 2H), 3.90 (s, 3H), 3.80 (p, J=9.4 Hz, 1H), 3.63 (p, J=10.0 Hz, 1H), 2.85-2.76 (m, 2H), 2.60-2.52 (m, 2H). (Ex-136): ESI MS m/z=346 [M+H]⁺. ¹H NMR (600 MHz, DMSO-d₆) δ 7.85-7.75 (m, 3H), 7.40-7.33 (m, 4H), 7.31 (app t, J=7.9 Hz, 1H), 7.25-7.20 (m, 2H), 3.95-3.89 (m, 1H), 3.91 (s, 3H), 3.86-3.79 (m, 1H), 2.87-2.80 (m, 2H), 2.69-2.61 (m, 2H).

### Example 137: (1r,3r)-3-(5-amino-7-methoxy-[1,2,4]triazolo[1,5-c]quinazolin-2-yl)-1-(5-(2-hydroxypropan-2-yl)pyridin-2-yl)cyclobutan-1-ol (Ex-137)

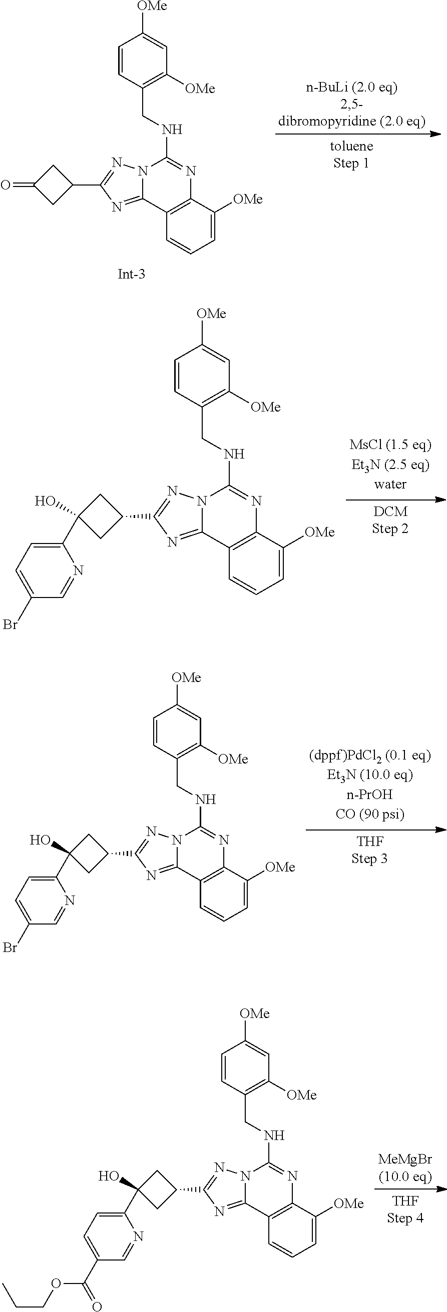 [SMILES: COc1ccc(CNc2nc3c(OC)cccc3c3nc(C4CC(=O)C4)nn23)c(OC)c1 ; COc1ccc(CNc2nc3c(OC)cccc3c3nc([C@H]4C[C@](O)(c5ccc(Br)cn5)C4)nn23)c(OC)c1 ; COc1ccc(CNc2nc3c(OC)cccc3c3nc([C@H]4C[C@@](O)(c5ccc(Br)cn5)C4)nn23)c(OC)c1 ; CCCOC(=O)c1ccc([C@]2(O)C[C@H](c3nc4c5cccc(OC)c5nc(NCc5ccc(OC)cc5OC)n4n3)C2)nc1]

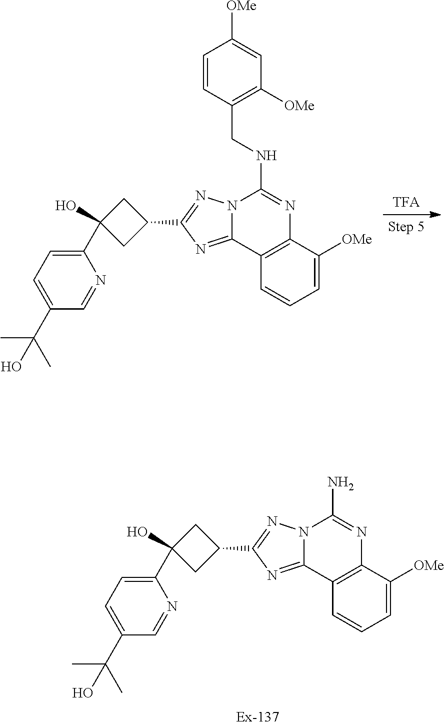 [SMILES: COc1ccc(CNc2nc3c(OC)cccc3c3nc([C@H]4C[C@@](O)(c5ccc(C(C)(C)O)cn5)C4)nn23)c(OC)c1 ; COc1cccc2c1nc(N)n1nc([C@H]3C[C@@](O)(c4ccc(C(C)(C)O)cn4)C3)nc21]

Step 1: A 25 mL round bottom flask was charged with 2,5-dibromopyridine (118 mg, 0.498 mmol, 2.0 equiv), then evacuated and backfilled with nitrogen. Toluene (2.5 mL) was added, and the reaction was cooled to −78° C. n-Butyllithium (2.5 M in hexanes, 0.199 mL, 0.498 mmol, 2.0 equiv) was added dropwise, and the resulting suspension was stirred at −78° C. for 30 min. A solution of 3-(5-((2,4-dimethoxybenzyl)amino)-7-methoxy-[1,2,4]triazolo[1,5-c]quinazolin-2-yl)cyclobutan-1-one (Int-3) (108 mg, 0.249 mmol, 1.0 equiv) in THF (2.5 mL) was added slowly over 10 min, and the resulting reaction mixture was stirred at −78° C. for 1 hour, then warmed to −40° C. over 30 min. The reaction was then quenched with water (10 mL) and DCM (10 mL) and stirred at 25° C. for 10 min. The layers were then separated, and the aq. layer was extracted with DCM (2×10 mL). The combined organic layers were dried over anhydrous MgSO₄, filtered and concentrated. The crude residue was purified by silica gel column chromatography (0-100% EtOAc in hex) to afford (1s,3s)-1-(5-bromopyridin-2-yl)-3-(5-((2,4-dimethoxybenzyl)amino)-7-methoxy-[1,2,4]triazolo[1,5-c]quinazolin-2-yl)cyclobutan-1-ol. ESI MS m/z=591/593 [M+H]⁺.

Step 2: A 20 mL scintillation vial was charged with (1s,3s)-1-(5-bromopyridin-2-yl)-3-(5-((2,4-dimethoxybenzyl)amino)-7-methoxy-[1,2,4]triazolo[1,5-c]quinazolin-2-yl)cyclobutan-1-ol (100 mg, 0.169 mmol) and THF (1.7 mL). After cooling to 0° C., Et₃N (59 μL, 0.423 mmol) and MsCl (20 μL, 0.254 mmol) were added. The resulting mixture was stirred at 0° C. for 10 min, then warmed to 25° C. and stirred for 1 h. Water (3 mL) was added, and the reaction was heated at 60° C. and stirred for 90 min. After cooling, DCM (5 mL) and water (5 mL) were added, and the layers were separated. The aq. layer was extracted with DCM (2×6 mL), and the combined organic layers were dried over anhydrous MgSO₄, filtered and concentrated. The crude residue was purified by silica gel column chromatography (0-100% EtOAc in hex) to provide (1r,3r)-1-(5-bromopyridin-2-yl)-3-(5-((2,4-dimethoxybenzyl)amino)-7-methoxy-[1,2,4]triazolo[1,5-c]quinazolin-2-yl)cyclobutan-1-ol. ESI MS m/z=591/593 [M+H]⁺.

Step 3: (1r,3r)-1-(5-bromopyridin-2-yl)-3-(5-((2,4-dimethoxybenzyl)amino)-7-methoxy-[1,2,4]triazolo[1,5-c]quinazolin-2-yl)cyclobutan-1-ol (60 mg, 0.101 mmol, 1.0 equiv), (dppf)PdCl₂ dichloromethane complex (83.0 mg, 0.102 mmol, 0.1 equiv), and dppf (7.5 mg, 0.014 mmol, 0.14 equiv) were added to 3 side-by-side vials and the vials were evacuated and backfilled with nitrogen (3×). A solution of Et₃N (1.42 mL, 10.2 mmol per reaction vial) in DMF (3.4 mL per reaction vial) and n-propanol (3.4 mL per reaction vial) was degassed by bubbling argon under sonication for 12 minutes, then added to each reaction vial. The reactions were then heated at 80° C. under —CO (90 psi) and stirred for 18 hours. After cooling, the reaction mixtures were combined and concentrated, and the crude residue was purified by silica gel column chromatography (0-100% EtOAc in hex) to afford propyl 6-((1r,3r)-3-(5-((2,4-dimethoxybenzyl)amino)-7-methoxy-[1,2,4]triazolo[1,5-c]quinazolin-2-yl)-1-hydroxycyclobutyl)nicotinate. ESI MS m/z=599 [M+H]⁺

Step 4: A 100 mL round bottom flask was charged with propyl 6-((l1,3r)-3-(5-((2,4-dimethoxybenzyl)amino)-7-methoxy-[1,2,4]triazolo[1,5-c]quinazolin-2-yl)-1-hydroxycyclobutyl)nicotinate (33 mg, 0.055 mmol, 1.0 equiv). THF (1.1 mL) was then added, and the reaction mixture was cooled to −30° C. MeMgBr (3 M in Et₂O, 0.18 mL, 0.55 mmol) was added dropwise, and the reaction was stirred at −30° C. for 10 min, then warmed to 25° C. over 30 min. The reaction was then quenched with sat. aq. NH₄Cl (2 mL), water (3 mL) and DCM (5 mL), and the resulting biphasic mixture was stirred at 25° C. for 10 min. The layers were then separated, and the aq. layer was extracted with DCM (2×6 mL). The combined organic layers were dried over anhydrous MgSO₄, filtered and concentrated under reduced pressure. The crude residue was purified by silica gel column chromatography (0-100% 3:1 EtOAc:EtOH in hex) to afford (1r,3r)-3-(5-((2,4-dimethoxybenzyl)amino)-7-methoxy-[1,2,4]triazolo[1,5-c]quinazolin-2-yl)-1-(5-(2-hydroxypropan-2-yl)pyridin-2-yl)cyclobutan-1-ol. ESI MS m/z=571 [M+H]⁺.

Step 5: (1r,3r)-3-(5-((2,4-dimethoxybenzyl)amino)-7-methoxy-[1,2,4]triazolo[1,5-c]quinazolin-2-yl)-1-(5-(2-hydroxypropan-2-yl)pyridin-2-yl)cyclobutan-1-ol (33 mg, 0.055 mmol) was taken up in TFA (550 μL) and the reaction was stirred at 50° C. for 2 hours. After cooling, the reaction mixture was concentrated and purified by reverse phase HPLC using the NH₄OH modifier to afford (1r,3r)-3-(5-amino-7-methoxy-[1,2,4]triazolo[1,5-c]quinazolin-2-yl)-1-(5-(2-hydroxypropan-2-yl)pyridin-2-yl)cyclobutan-1-ol (Ex-137). ESI MS m/z=421 [M+H]⁺. ¹H NMR (600 MHz, DMSO-d₆) δ 8.64 (d, J=2.3 Hz, 1H), 7.83 (dd, J=8.2, 2.4 Hz, 1H), 7.80-7.72 (m, 3H), 7.55 (d, J=8.3 Hz, 1H), 7.29 (app t, J=7.9 Hz, 1H), 7.23-7.18 (m, 1H), 5.87 (s, 1H), 5.16 (s, 1H), 4.10 (app p, J=9.1 Hz, 1H), 3.90 (s, 3H), 3.16-3.07 (m, 2H), 2.58-2.51 (m, 2H), 1.45 (s, 6H).

### Example 138: 2-(6-((l1,3r)-1-amino-3-(5-amino-7-methoxy-[1,2,4]triazolo[1,5-c]quinazolin-2-yl)cyclobutyl)pyridin-3-yl)propan-2-ol (Ex-138)

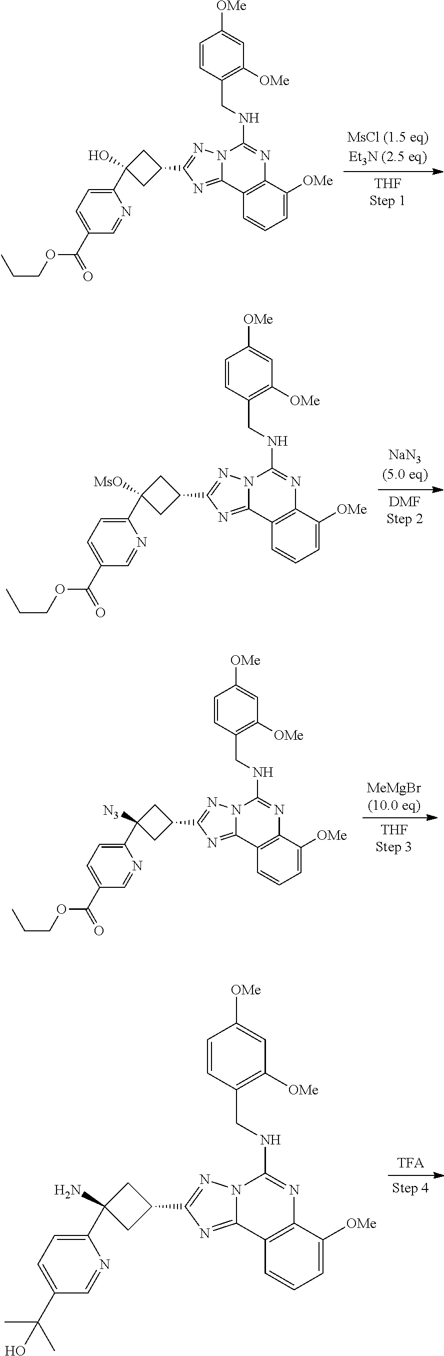 [SMILES: CCCOC(=O)c1ccc([C@]2(O)C[C@@H](c3nc4c5cccc(OC)c5nc(NCc5ccc(OC)cc5OC)n4n3)C2)nc1 ; CCCOC(=O)c1ccc([C@]2(OS(C)(=O)=O)C[C@@H](c3nc4c5cccc(OC)c5nc(NCc5ccc(OC)cc5OC)n4n3)C2)nc1 ; CCCOC(=O)c1ccc([C@]2(N=[N+]=[N-])C[C@H](c3nc4c5cccc(OC)c5nc(NCc5ccc(OC)cc5OC)n4n3)C2)nc1 ; COc1ccc(CNc2nc3c(OC)cccc3c3nc([C@H]4C[C@@](N)(c5ccc(C(C)(C)O)cn5)C4)nn23)c(OC)c1]

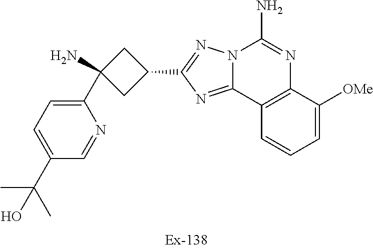 [SMILES: COc1cccc2c1nc(N)n1nc([C@H]3C[C@@](N)(c4ccc(C(C)(C)O)cn4)C3)nc21]

Step 1: A 30 mL scintillation vial was charged with propyl 6-((1s,3s)-3-(5-((2,4-dimethoxybenzyl)amino)-7-methoxy-[1,2,4]triazolo[1,5-c]quinazolin-2-yl)-1-hydroxycyclobutyl)nicotinate (70 mg, 0.117 mmol) and DCM (1.2 mL). The reaction mixture was cooled to 0° C., and Et₃N (41 μL, 0.292 mmol) and MsCl (14 μL, 0.175 mmol) were then added. The reaction was stirred for 10 min at 0° C., then warmed to 25° C. and stirred for 1 h. The reaction mixture was then quenched with water (5 mL) and DCM (5 mL), and the layers were separated. The aq. layer was extracted with DCM (2×6 mL), and the combined organic layers were dried over anhydrous MgSO₄, filtered, and concentrated to provide propyl 6-((1s,3s)-3-(5-((2,4-dimethoxybenzyl)amino)-7-methoxy-[1,2,4]triazolo[1,5-c]quinazolin-2-yl)-1-((methylsulfonyl)oxy)cyclobutyl)nicotinate, which was used without further purification. ESI MS m/z=677 [M+H]⁺.

Step 2: A 30 mL scintillation vial was charged with propyl 6-((1s,3s)-3-(5-((2,4-dimethoxybenzyl)amino)-7-methoxy-[1,2,4]triazolo[1,5-c]quinazolin-2-yl)-1-((methylsulfonyl)oxy)cyclobutyl)nicotinate (79 mg, 0.117 mmol) and DMF (1.2 mL). NaN₃ (38 mg, 0.584 mmol) was then added, and the reaction was heated to 60° C. and stirred for 16 h. After cooling, the reaction mixture was poured into Et₂O (50 mL), water (25 mL) and sat. aq. NaHCO₃ (10 mL). The layers were separated, and the aq. layer was extracted with Et₂O (2×40 mL). The combined organic layers were washed with water (2×20 mL) and brine (10 mL), dried over anhydrous MgSO₄, filtered and concentrated. The crude residue was purified by silica gel column chromatography (0-100% EtOAc in hex) to provide propyl 5-((1r,3r)-1-azido-3-(5-((2,4-dimethoxybenzyl)amino)-7-methoxy-[1,2,4]triazolo[1,5-c]quinazolin-2-yl)cyclobutyl)picolinate. ESI MS m/z=624 [M+H]⁺.

Step 3: Propyl 6-(1-azido-3-(5-((2,4-dimethoxybenzyl)amino)-7-methoxy-[1,2,4]triazolo[1,5-c]quinazolin-2-yl)cyclobutyl)nicotinate (100 mg, 0.160 mmol) in a 30 mL vial was dissolved in THF (3207 μl) and cooled to −30° C. Methylmagnesium bromide in THF (534 μl, 1.603 mmol) was then added dropwise, and the reaction was stirred at −30° C. for 10 minutes, then the cold bath was removed and the reaction was slowly warmed to room temperature over 30 min. The reaction was quenched with sat. aq. NH₄Cl (2 mL). DCM (5 mL) and water (3 mL) were added, and the layers were separated. The aqueous layer was extracted 2× with DCM (6 mL) using a phase separator cartridge. The combined organic layers were dried over anhydrous MgSO₄, filtered, and concentrated. The crude residue was purified by silica gel column chromatography (0-25% MeOH in DCM) to afford 2-(6-((1r,3r)-1-amino-3-(5-((2,4-dimethoxybenzyl)amino)-7-methoxy-[1,2,4]triazolo[1,5-c]quinazolin-2-yl)cyclobutyl)pyridin-3-yl)propan-2-ol. ESI MS m/z=596 [M+H]⁺.

Step 4: TFA (614 μL) was added to a 20 mL vial containing 2-(6-((l1,3r)-1-amino-3-(5-((2,4-dimethoxybenzyl)amino)-7-methoxy-[1,2,4]triazolo[1,5-c]quinazolin-2-yl)cyclobutyl)pyridin-3-yl)propan-2-ol (35 mg, 0.061 mmol) and the reaction was stirred at 50° C. for 2 hours. After cooling, the reaction mixture was concentrated and purified by reversed phase HPLC using the NH₄OH modifier to afford 2-(6-((l1,3r)-1-amino-3-(5-amino-7-methoxy-[1,2,4]triazolo[1,5-c]quinazolin-2-yl)cyclobutyl)pyridin-3-yl)propan-2-ol (Ex-138). ESI MS m/z=420 [M+H]^{+ 1}H NMR (600 MHz, DMSO-d₆) δ 8.62 (d, J=2.3 Hz, 1H), 7.82 (dd, J=8.2, 2.4 Hz, 1H), 7.78-7.70 (m, 3H), 7.51 (d, J=8.2 Hz, 1H), 7.27 (app t, J=7.9 Hz, 1H), 7.20 (d, J=7.0 Hz, 1H), 5.15 (s, 2H), 4.13 (p, J=9.1 Hz, 1H), 3.90 (s, 3H), 3.01-2.92 (m, 2H), 2.46-2.40 (m, 2H), 1.45 (s, 6H).

### Example 139: (1s,3s)-3-(5-amino-9-fluoro-7-methoxy-[1,2,4]triazolo[1,5-c]quinazolin-2-yl)-1-(6-(2-hydroxypropan-2-yl)pyridin-3-yl)cyclobutan-1-ol (Ex-139)

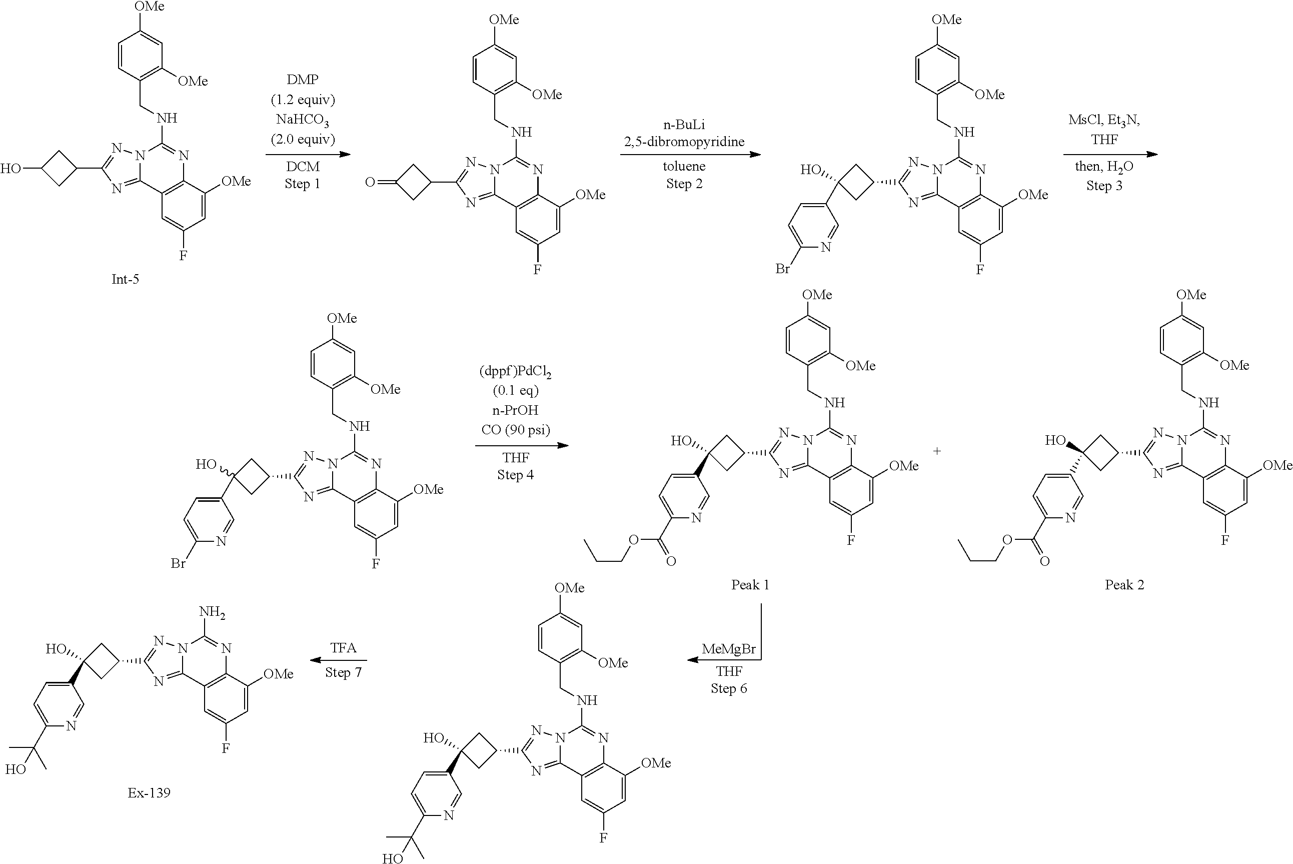

Step 1: A 250 mL round bottom flask was charged with 3-(5-((2,4-dimethoxybenzyl)amino)-9-fluoro-7-methoxy-[1,2,4]triazolo[1,5-c]quinazolin-2-yl)cyclobutan-1-ol (Int-5, (5 g, 11.03 mmol), Dess-Martin Periodinane (7.01 g, 16.54 mmol, 1.5 equiv) and NaHCO₃ (1.4 g, 16.54 mmol, 1.5 equiv), then DCM was added (110 mL). The resulting mixture was stirred for 2 hours at 25° C. The reaction was then quenched with sat. aq. Na₂S₂O₃ (70 mL), and the biphasic mixture was stirred for 10 min at 25° C. The layers were then separated, and the aq. layer was extracted with DCM (2×40 mL). The combined organic layers were dried over anhydrous Na₂SO₄, filtered and concentrated. The crude residue was purified by silica gel column chromatography (0-100% EtOAc in hex) to afford 3-(5-((2,4-dimethoxybenzyl)amino)-9-fluoro-7-methoxy-[1,2,4]triazolo[1,5-c]quinazolin-2-yl)cyclobutan-1-one. ESI MS m/z=452 [M+H]⁺.

Step 2: A 250 mL round bottom flask was charged with 2,5-dibromopyridine (2.9 g, 12.23 mmol, 2.4 equiv), then evacuated and backfilled with argon. Toluene (29 mL) was added, and the reaction was cooled to −78° C. n-Butyllithium (2.5 M in hexanes, 4.98 mL, 12.23 mmol, 2.4 equiv) was added dropwise, and the resulting suspension was stirred at −78° C. for 30 min. A suspension of 3-(5-((2,4-dimethoxybenzyl)amino)-7-methoxy-[1,2,4]triazolo[1,5-c]quinazolin-2-yl)cyclobutan-1-one (2.3 g, 5.09 mmol, 1.0 equiv) in THF (48 mL) was added slowly over 10 min, and the resulting reaction mixture was stirred at −78° C. for 1 hour. The reaction was then quenched with NH₄Cl (saturated aqueous solution) and DCM (10 mL) at −78° C. The resulting mixture was allowed to stir at room temperature. The layers were then separated, and the aq. layer was extracted with DCM (2×20 mL). The combined organic layers were dried over anhydrous Na₂SO₄, filtered and concentrated. The crude residue was purified by silica gel column chromatography (0-70% EtOAc in hex) to afford (1s,3s)-1-(6-bromopyridin-3-yl)-3-(5-((2,4-dimethoxybenzyl)amino)-9-fluoro-7-methoxy-[1,2,4]triazolo[1,5-c]quinazolin-2-yl)cyclobutan-1-ol. ESI MS m/z=609/611 [M+H]⁺.

Step 3: A 20 mL vial was charged with (1s,3s)-1-(6-bromopyridin-3-yl)-3-(5-((2,4-dimethoxybenzyl)amino)-9-fluoro-7-methoxy-[1,2,4]triazolo[1,5-c]quinazolin-2-yl)cyclobutan-1-ol (205 mg, 0.336 mmol) and THF (3.0 mL) at 0° C. Triethylamine (0.12 mL, 0.841 mmol) was added, followed by addition of methanesulfonyl chloride (0.03 mL, 0.437 mmol). After 10 min the ice bath was removed, and the reaction was allowed to stir at room temperature for 45 min. Water (3.0 mL) was added and the reaction mixture was allowed to stir at room temperature for 30 min. The reaction was then heated at 40° C. for 2 h. The mixture was then cooled to room temperature and diluted with DCM (10 mL). The layers were separated and the aq. layer was extracted with DCM (2×5 mL). The combined organic layers were dried over anhydrous Na₂SO₄, filtered and concentrated under reduced pressure. The resulting crude material (mixture of diastereomers) was taken to the next step without further purification. ESI MS m/z=609/611 [M+H]⁺.

Step 4: A vial was charged with (1-(6-bromopyridin-3-yl)-3-(5-((2,4-dimethoxybenzyl)amino)-9-fluoro-7-methoxy-[1,2,4]triazolo[1,5-c]quinazolin-2-yl)cyclobutan-1-ol (222 mg, 0.364 mmol), (dppf)PdCl₂ dichloromethane complex (29.7 mg, 0.036 mmol, 0.1 equiv), and dppf (40.4 mg, 0.073 mmol, 0.2 equiv), followed by addition of DMF (1.8 mL), Et₃N (0.51 mL, 3.64 mmol) and n-propanol (1.8 mL). The resulting mixture was purged with argon. The reaction was then heated at 80° C. under —CO (90 psi) and stirred for 18 hours. After cooling, the reaction mixture was concentrated, and the crude residue was purified by silica gel column chromatography (0-100% EtOAc in hex) to afford propyl 5-((1s,3s)-3-(5-((2,4-dimethoxybenzyl)amino)-9-fluoro-7-methoxy-[1,2,4]triazolo[1,5-c]quinazolin-2-yl)-1-hydroxycyclobutyl)picolinate (first eluting compound) and propyl 5-((l1,3r)-3-(5-((2,4-dimethoxybenzyl)amino)-9-fluoro-7-methoxy-[1,2,4]triazolo[1,5-c]quinazolin-2-yl)-1-hydroxycyclobutyl)picolinate (second eluting compound). ESI MS m/z=617 [M+H]⁺.

Step 5: A 5 mL round bottom flask was charged with propyl 5-((1s,3s)-3-(5-((2,4-dimethoxybenzyl)amino)-9-fluoro-7-methoxy-[1,2,4]triazolo[1,5-c]quinazolin-2-yl)-1-hydroxycyclobutyl)picolinate (95 mg, 0.154 mmol, 1.0 equiv), THF (1.5 mL) was then added, and the reaction mixture was cooled to −30° C. Methylmagnesium Bromide (3 M in Et₂O, 0.51 mL, 1.5 mmol) was added dropwise, and the reaction was stirred at −30° C. for 10 min, then warmed to 25° C. and stirred for 45 min. The reaction was then quenched with sat. aq. NH₄Cl and DCM. The resulting biphasic mixture was stirred at 25° C. for 10 min. The layers were then separated, and the aq. layer was extracted with DCM (2×6 mL). The combined organic layers were dried over anhydrous Na₂SO₄, filtered and concentrated under reduced pressure. The crude residue was taken to the next step without further purification. MS m/z=589 [M+H]⁺.

Step 6: (1s,3s)-3-(5-((2,4-Dimethoxybenzyl)amino)-9-fluoro-7-methoxy-[1,2,4]triazolo[1,5-c]quinazolin-2-yl)-1-(6-(2-hydroxypropan-2-yl)pyridin-3-yl)cyclobutan-1-ol (78 mg, 0.133 equiv) was taken up in TFA (1.3 mL) and was stirred at 50° C. for 1 hours. The reaction was concentrated under reduced pressure and purified by mass triggered reversed phase HPLC [basic method], to provide (1s,3s)-3-(5-amino-9-fluoro-7-methoxy-[1,2,4]triazolo[1,5-c]quinazolin-2-yl)-1-(6-(2-hydroxypropan-2-yl)pyridin-3-yl)cyclobutan-1-ol (Ex-139). MS m/z=439 [M+H]⁺. ¹H NMR (600 MHz, DMSO-d₆) δ 8.75 (d, J=1.9 Hz, 1H), 7.98 (dd, J=8.3, 2.4 Hz, 1H), 7.77 (s, 2H), 7.68 (d, J=8.3 Hz, 1H), 7.44 (dd, J=8.3, 2.7 Hz, 1H), 7.18 (dd, J=11.1, 2.7 Hz, 1H), 5.92 (s, 1H), 5.21 (s, 1H), 3.93 (s, 3H), 3.42 (p, J=9.3 Hz, 1H), 2.99-2.94 (m, 2H), 2.89-2.82 (m, 2H), 1.46 (s, 6H).

### Example 140: 2-((1s,3s)-3-(5-(1-amino-3,3-difluorocyclobutyl)pyridin-2-yl)cyclobutyl)-7-methoxy-[1,2,4]triazolo[1,5-c]quinazolin-5-amine. TFA salt (Ex-140)

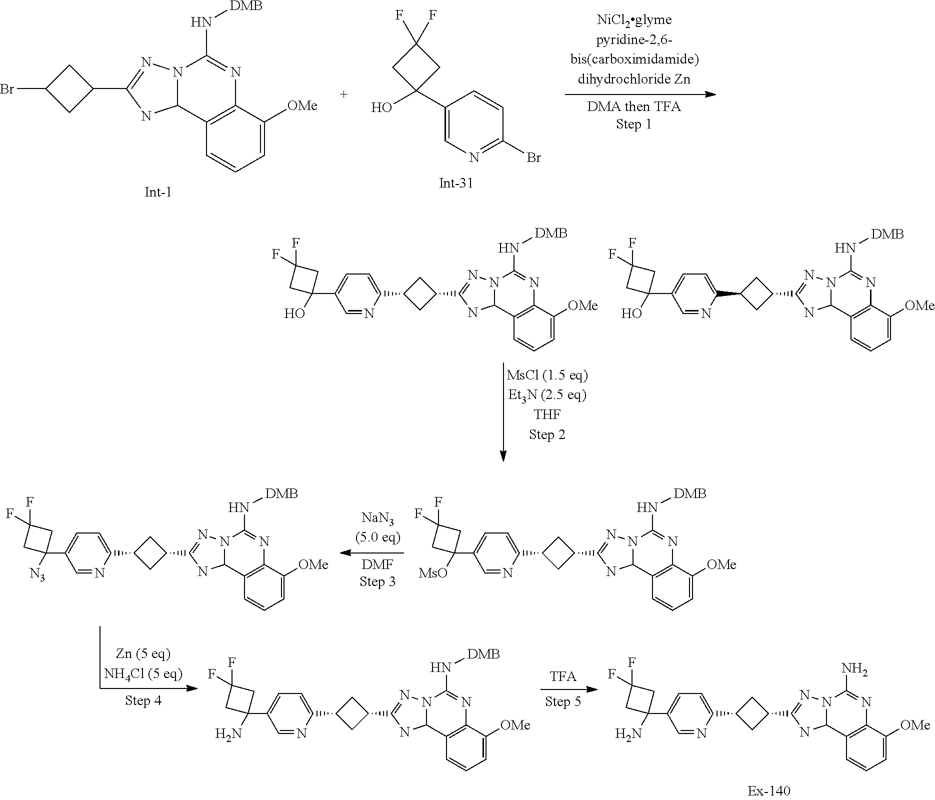

Step 1: Two side-by-side 40 mL scintillation vials equipped with stir bars were each charged with NiI₂ (238 mg, 0.762 mmol), 1-(6-bromopyridin-3-yl)-3,3-difluorocyclobutan-1-ol (Int-31) (302 mg, 1.14 mmol), 2-(3-bromocyclobutyl)-N-(2,4-dimethoxybenzyl)-7-methoxy-[1,2,4]triazolo[1,5-c]quinazolin-5-amine (Int-1) (950 mg, 1.91 mmol), pyridine-2,6-bis(carboximidamide) dihydrochloride (178 mg, 0.762 mmol), and zinc (499 mg, 7.62 mmol). the reaction vials were evacuated and backfilled with nitrogen (3×). DMA (19 mL) was added, and the vials were each heated at 70° C. and stirred for 4 h. After cooling, the reaction mixtures were combined, filtered through Celite™, and concentrated. The crude residue was purified by silica gel column chromatography (0-100% EtOAc in hex) to provide 1-(6-((l1,3r)-3-(5-((2,4-dimethoxybenzyl)amino)-7-methoxy-[1,2,4]triazolo[1,5-c]quinazolin-2-yl)cyclobutyl)pyridin-3-yl)-3,3-difluorocyclobutan-1-ol as the first eluting peak, and 1-(6-((1s,3s)-3-(5-((2,4-dimethoxybenzyl)amino)-7-methoxy-[1,2,4]triazolo[1,5-c]quinazolin-2-yl)cyclobutyl)pyridin-3-yl)-3,3-difluorocyclobutan-1-ol as the second eluting peak. MS m/z=603 [M+H]⁺.

Step 2: A sample of 1-(6-((1s,3s)-3-(5-((3,4-dimethylbenzyl)amino)-7-methoxy-[1,2,4]triazolo[1,5-c]quinazolin-2-yl)cyclobutyl)pyridin-3-yl)-3,3-difluorocyclobutan-1-ol (19 mg, 0.032 mmol) in DCM (315 μl) was cooled to 0° C. and triethylamine (13.18 μl, 0.095 mmol) followed by methanesulfonyl chloride (4.91 μl, 0.063 mmol) was then added. The resulting mixture was stirred for 10 minutes at 0° C. then warmed up to room temperature and stirred for 45 min. The reaction was then quenched with water (2 mL) and DCM (5 mL) was added. The layers were separated, and the aqueous layer was extracted 2× with DCM (5 mL). The combined organic layers were dried over anhydrous MgSO₄ and concentrated. The crude 1-(6-((1s,3s)-3-(5-((3,4-dimethylbenzyl)amino)-7-methoxy-[1,2,4]triazolo[1,5-c]quinazolin-2-yl)cyclobutyl)pyridin-3-yl)-3,3-difluorocyclobutyl methanesulfonate was used without further purification.

Step 3: A 20 mL vial containing 1-(6-((1s,3s)-3-(5-((3,4-dimethylbenzyl)amino)-7-methoxy-[1,2,4]triazolo[1,5-c]quinazolin-2-yl)cyclobutyl)pyridin-3-yl)-3,3-difluorocyclobutyl methanesulfonate (20 mg, 0.029 mmol) and a stir bar was dissolved in DMF (294 μl) and sodium azide (9.55 mg, 0.147 mmol) was added. The resulting mixture was stirred at 60° C. for 16 hours. The reaction was quenched with sat. aq. sodium bicarbonate (0.5 mL), water (1 mL) and diethyl ether (3 mL). The layers were separated, and the aqueous layer was extracted 2× with diethyl ether (3 mL). The combined organic layers were washed 2× with water (1 mL) and brine (1 mL), dried over anhydrous MgSO₄ and concentrated. The crude residue was purified by silica gel chromatography, eluting with 0-100% EtOAc in Hexanes, to provide 2-((1s,3s)-3-(5-(1-azido-3,3-difluorocyclobutyl)pyridin-2-yl)cyclobutyl)-N-(3,4-dimethylbenzyl)-7-methoxy-[1,2,4]triazolo[1,5-c]quinazolin-5-amine. MS m/z=628 [M+H]⁺.

Step 4: A 20 mL scintillation vial equipped with a stir bar was charged with 2-((1s,3s)-3-(5-(1-azido-3,3-difluorocyclobutyl)pyridin-2-yl)cyclobutyl)-N-(2,4-dimethoxybenzyl)-7-methoxy-[1,2,4]triazolo[1,5-c]quinazolin-5-amine (192 mg, 0.306 mmol), NH₄Cl (82 mg, 1.53 mmol), and zinc (100 mg, 1.53 mmol). The reaction vial was evacuated and backfilled with nitrogen (3×). EtOH (4.1 mL) and water (1.0 mL) were added, and the resulting mixture was heated at 80° C. and stirred for 1 h. After cooling, the reaction mixture was poured into 25% i-PrOH in CHCl₃ (30 mL), water (10 mL) and sat. aq. NaHCO₃ (10 mL), and the resulting biphasic mixture was stirred for 10 min. The layers were separated, and the aq. layer was extracted with 25% i-PrOH in CHCl₃ (3×30 mL). The combined organic layers were dried over anhydrous MgSO₄, filtered and concentrated. The crude residue was purified by silica gel column chromatography (0-50% MeOH in DCM) to afford 2-((1s,3s)-3-(5-(1-amino-3,3-difluorocyclobutyl)pyridin-2-yl)cyclobutyl)-N-(3,4-dimethylbenzyl)-7-methoxy-[1,2,4]triazolo[1,5-c]quinazolin-5-amine.

Step 5: 2-((1s,3s)-3-(5-(1-amino-3,3-difluorocyclobutyl)pyridin-2-yl)cyclobutyl)-N-(3,4-dimethylbenzyl)-7-methoxy-[1,2,4]triazolo[1,5-c]quinazolin-5-amine was then taken up in TFA (1.8 mL) and the reaction was heated at 50° C. and stirred for 2 h. After cooling, the reaction mixture was concentrated, and the residue was purified by reversed phase HPLC using the TFA modifier to afford 2-((1s,3s)-3-(5-(1-amino-3,3-difluorocyclobutyl)pyridin-2-yl)cyclobutyl)-7-methoxy-[1,2,4]triazolo[1,5-c]quinazolin-5-amine, TFA salt (Ex-140). MS m/z=452 [M+H]⁺. ¹H NMR (600 MHz, DMSO-d₆) δ 8.88 (br s, 2H), 8.82 (d, J=2.4 Hz, 1H), 8.08-7.99 (m, 3H), 7.78-7.74 (m, 1H), 7.52 (d, J=8.2 Hz, 1H), 7.33 (app t, J=7.9 Hz, 1H), 7.26 (d, J=7.5 Hz, 1H), 3.92 (s, 3H), 3.90-3.79 (m, 2H), 3.57-3.48 (m, 2H), 3.45-3.33 (m, 2H), 2.84-2.72 (m, 4H).

TABLE 12
Using the appropriate starting materials and a method similar to that described above,
the following examples were prepared:

| | | | Exact |
|---|---|---|---|
| | | | Mass |
| Example | Structure | Name | [M + H]+ |
| Ex-141 | 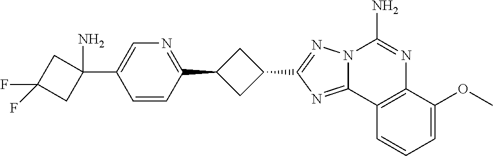 | 2-{trans-3-[5-(1-amino- 3,3- difluorocyclobutyl) pyridin-2- yl]cyclobutyl}-7- methoxy[1,2,4]triazolo [1,5-c]quinazolin-5- amine | 452 |

### Example 142, 143, 144, and 145: 2-(3-(5-(2-aminopropan-2-yl)pyridin-2-yl)cyclopentyl)-7-methoxy-[1,2,4]triazolo[1,5-c]quinazolin-5-amine (Ex-142, Ex-143, Ex-144, and Ex-145)

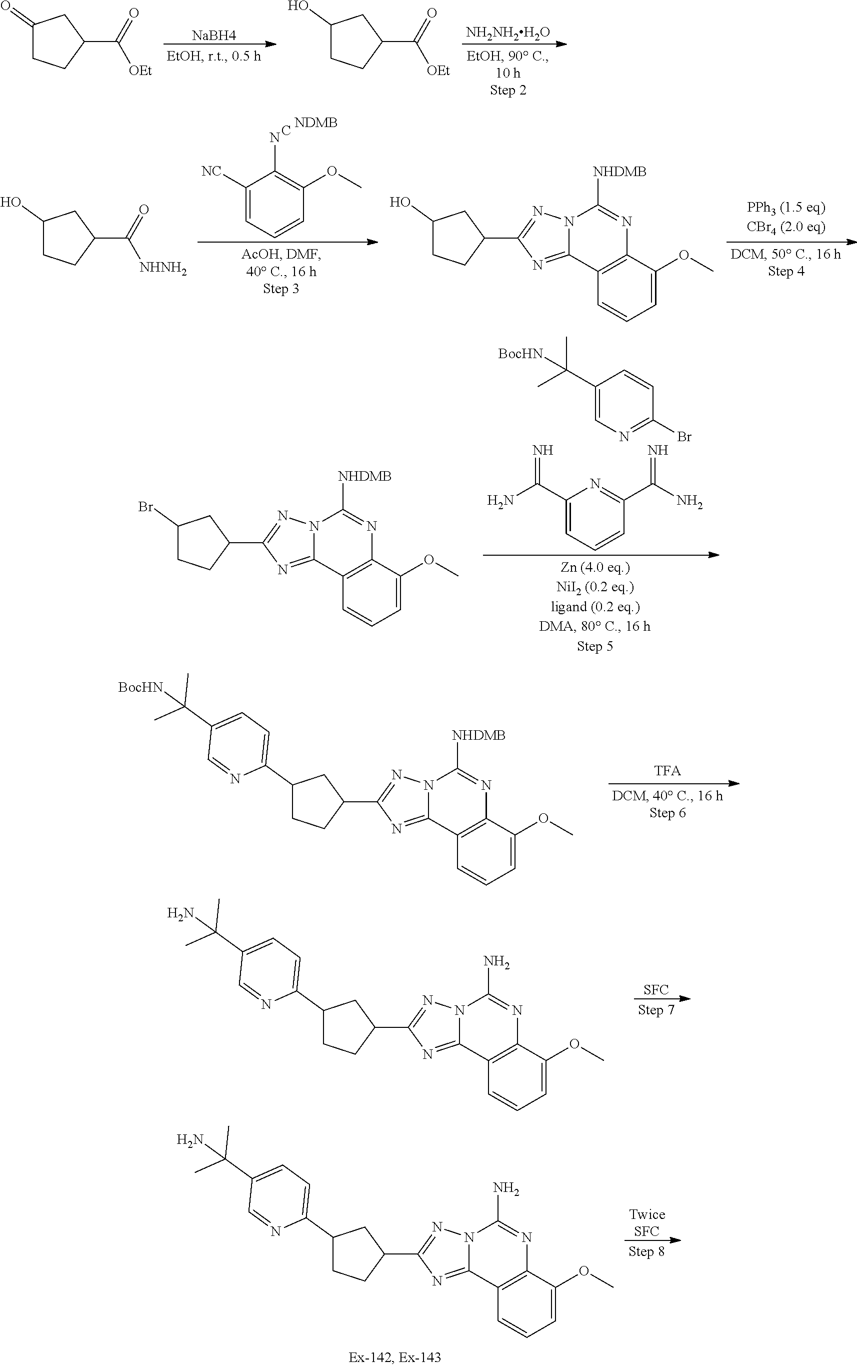

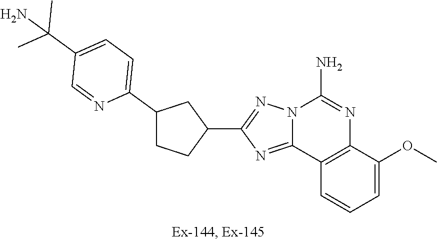 [SMILES: COc1cccc2c1nc(N)n1nc(C3CCC(c4ccc(C(C)(C)N)cn4)C3)nc21]

Step 1: NaBH₄ (0.484 g, 12.81 mmol) was added to a stirred mixture of ethyl 3-oxocyclopentanecarboxylate (1 g, 6.40 mmol) in EtOH (20 mL) at room temperature (20° C.), and the mixture was stirred at 20° C. for 0.5 h. The mixture was diluted with water (20 mL), filtered and extracted with EtOAc (20 mL×3). The combined organic layers were dried over anhydrous Na₂SO₄, filtered, and the filtrate was evaporated under reduced pressure to give ethyl 3-hydroxycyclopentanecarboxylate, which was used directly in the next step without further purification.

Step 2: To a solution of ethyl 3-hydroxycyclopentanecarboxylate (1 g, 6.32 mmol) in EtOH (20 mL) was added hydrazine hydrate (6.33 g, 126 mmol) at room temperature. The mixture was then stirred at 90° C. for 10 h. The mixture was evaporated under reduced pressure to give 3-hydroxycyclopentanecarbohydrazide, which was used directly in the next step without further purification.

Step 3: Acetic acid (0.05 mL, 0.873 mmol) and 2-((((2,4-dimethoxybenzyl)imino)methylene)amino)-3-methoxybenzonitrile (500 mg, 1.546 mmol) were added to a stirred solution of 3-hydroxycyclopentanecarbohydrazide (223 mg, 1.546 mmol) in DMF (5 mL) at 20° C. under N₂. After the addition was finished, the reaction was stirred at 40° C. under N₂. After the reaction was stirred at 40° C. for 16 h, the mixture was concentrated under reduced pressure. The resulting residue was purified by flash silica gel chromatography (ISCO®; Agela® Flash Column Silica-CS (4 g), Eluent of 0-55% ethyl acetate/petroleum ether gradient @30 mL/min) to give 3-(5-((2,4-dimethoxybenzyl)amino)-7-methoxy-[1,2,4]triazolo[1,5-c]quinazolin-2-yl)cyclopentanol.

Step 4: Ph₃P (193 mg, 0.734 mmol) and CBr₄ (325 mg, 0.979 mmol) at 20° C. under N₂ were added to a stirred solution of 3-(5-((2,4-dimethoxybenzyl)amino)-7-methoxy-[1,2,4]triazolo[1,5-c]quinazolin-2-yl)cyclopentanol (220 mg, 0.489 mmol) in DCM (6 mL). The reaction was stirred at 50° C. under N₂ for 16 h. The mixture was concentrated under reduced pressure and the resulting residue was purified by flash silica gel chromatography (ISCO®; Agela® Flash Column Silica-CS (2 g), Eluent of 0-25% ethyl acetate/petroleum ether gradient @30 mL/min) to give 2-(3-bromocyclopentyl)-N-(2,4-dimethoxybenzyl)-7-methoxy-[1,2,4]triazolo[1,5-c]quinazolin-5-amine.

Step 5: tert-butyl (2-(6-bromopyridin-3-yl)propan-2-yl)carbamate (185 mg, 0.585 mmol), pyridine-2,6-bis(carboximidamide) (20 mg, 0.123 mmol), zinc (77 mg, 1.171 mmol) and nickel(II) iodide (37 mg, 0.118 mmol) at 20° C. under N₂ were added to a stirred solution of 2-(3-bromocyclopentyl)-N-(2,4-dimethoxybenzyl)-7-methoxy-[1,2,4]triazolo[1,5-c]quinazolin-5-amine (150 mg, 0.293 mmol) in DMA (2 mL). The reaction was stirred at 80° C. under N₂ for 16 h. The mixture was cooled to room temperature, filtered, diluted with EtOAc (40 mL), and washed with water (40 mL) and brine (40 mL). The organic layer was concentrated under reduced pressure to afford a crude residue that was purified by flash silica gel chromatography (ISCO®; Agela® Flash Column Silica-CS (2 g), Eluent of 0-100% ethyl acetate/petroleum ether gradient @30 mL/min) to give tert-butyl(2-(6-(3-(5-((2,4-dimethoxybenzyl)amino)-7-methoxy-[1,2,4]triazolo[1,5-c]quinazolin-2-yl)cyclopentyl)pyridin-3-yl)propan-2-yl)carbamate, which was used in next step without further purification.

Step 6: To a stirred solution of tert-butyl (2-(6-(3-(5-((2,4-dimethoxybenzyl)amino)-7-methoxy-[1,2,4]triazolo[1,5-c]quinazolin-2-yl)cyclopentyl)pyridin-3-yl)propan-2-yl)carbamate (79 mg, 0.118 mmol) in DCM (2.000 mL) was added TFA (2 mL) at 20° C. under N₂. The reaction was stirred at 40° C. for 16 h then concentrated under reduced pressure. The resulting residue was purified by reversed phase HPLC fitted with Agela DuraShell C18 150*25 mm*5 um using water (0.1% TFA)—MeCN as eluents (Mobile phase A water (0.1% TFA), Mobile phase B acetonitrile, Detection wavelength 220 nm) and concentration to give 2-(3-(5-(2-aminopropan-2-yl)pyridin-2-yl)cyclopentyl)-7-methoxy-[1,2,4]triazolo[1,5-c]quinazolin-5-amine.

Step 7: 2-(3-(5-(2-aminopropan-2-yl)pyridin-2-yl)cyclopentyl)-7-methoxy-[1,2,4]triazolo[1,5-c]quinazolin-5-amine was purified by SFC (AD, 250×30 mm, EtOH with 0.1% NH₄OH mobile phase) to give 2-(3-(5-(2-aminopropan-2-yl)pyridin-2-yl)cyclopentyl)-7-methoxy-[1,2,4]triazolo[1,5-c]quinazolin-5-amine (Ex-142) (first eluting) and 2-(3-(5-(2-aminopropan-2-yl)pyridin-2-yl)cyclopentyl)-7-methoxy-[1,2,4]triazolo[1,5-c]quinazolin-5-amine (Ex-143) (second eluting) and 2-(3-(5-(2-aminopropan-2-yl)pyridin-2-yl)cyclopentyl)-7-methoxy-[1,2,4]triazolo[1,5-c]quinazolin-5-amine (Ex-144/Ex-145) (third eluting) as a mixture of two isomers. (Ex-142): ESI MS m/z=418 [M+H]⁺. ¹H NMR (400 MHz, CDCl₃) δ 8.65 (br s, 1H), 7.89 (br d, J=8.07 Hz, 1H), 7.65-7.75 (m, 1H), 7.25-7.35 (m, 1H), 7.20-7.22 (m, 1H), 7.08 (br d, J=7.83 Hz, 1H), 5.86 (br s, 2H), 3.99 (d, J=2.20 Hz, 3H), 3.55-3.65 (m, 1H), 3.34-3.45 (m, 1H), 2.53-2.64 (m, 1H), 2.09-2.39 (m, 5H), 1.45 (d, J=2.45 Hz, 6H), 1.24 (br s, 2H). (Ex-143): ESI MS m/z=418 [M+H]⁺. ¹H NMR (400 MHz, CDCl₃) δ 8.72 (s, 1H), 7.97 (d, J=8.07 Hz, 1H), 7.76-7.82 (m, 1H), 7.37 (t, J=7.95 Hz, 1H), 7.27-7.29 (m, 1H), 7.16 (br d, J=8.07 Hz, 1H), 5.93 (br s, 2H), 4.07 (s, 3H), 3.63-3.72 (m, 1H), 3.40-3.50 (m, 1H), 2.62-2.72 (m, 1H), 2.23-2.50 (m, 5H), 1.53 (s, 6H), 1.32 (br s, 2H).

Step 8: 2-(3-(5-(2-aminopropan-2-yl)pyridin-2-yl)cyclopentyl)-7-methoxy-[1,2,4]triazolo[1,5-c]quinazolin-5-amine (Ex-144/Ex-145) (20 mg, 0.048 mmol) was purified by SFC (OD-H, 250×30 mm, MeOH with 0.1% NH₄OH modifier) to give 2-(3-(5-(2-aminopropan-2-yl)pyridin-2-yl)cyclopentyl)-7-methoxy-[1,2,4]triazolo[1,5-c]quinazolin-5-amine (Ex-144) (first eluting) and 2-(3-(5-(2-aminopropan-2-yl)pyridin-2-yl)cyclopentyl)-7-methoxy-[1,2,4]triazolo[1,5-c]quinazolin-5-amine (Ex-145) (second eluting). (Ex-144): ESI MS m/z=418 [M+H]⁺. ¹H NMR (500 MHz, CDCl₃) δ 8.63 (br s, 1H), 7.90 (d, J=7.78 Hz, 1H), 7.75 (br d, J=6.26 Hz, 1H), 7.29-7.37 (m, 1H), 7.06-7.17 (m, 2H), 6.46 (br s, 2H), 4.07 (s, 3H), 3.64 (br d, J=15.72 Hz, 1H), 3.44 (s, 1H), 2.51 (br s, 1H), 2.35-2.45 (m, 1H), 2.16 (br d, J=6.71 Hz, 1H), 1.81 (br s, 3H), 1.25-1.28 (m, 6H). (Ex-145): ESI MS m/z=418 [M+H]⁺. ¹H NMR (500 MHz, CDCl₃) δ 8.73 (d, J=2.14 Hz, 1H), 7.94-7.99 (m, 1H), 7.77 (dd, J=2.52, 8.16 Hz, 1H), 7.37 (t, J=8.01 Hz, 1H), 7.20 (d, J=8.24 Hz, 1H), 7.15 (d, J=7.93 Hz, 1H), 6.12 (br s, 2H), 4.03-4.09 (m, 3H), 3.75-3.87 (m, 1H), 3.57-3.64 (m, 1H), 2.60 (ddd, J=5.87, 8.05, 13.39 Hz, 1H), 2.40-2.50 (m, 2H), 2.31-2.39 (m, 1H), 2.21-2.30 (m, 1H), 2.00-2.10 (m, 1H), 1.53 (s, 6H).

### REFERENCES

1. Huang. J.; Chan. J.; Chen. Y.; Berths. C. J.; Baucom. K. D.; Larsen, R. D.; Faul. M. M. J. Am. Chem. Soc. 2010, 132, 3674-3675
2. Ali, A.; Huang, X.; Kuang, R.; Lim, Y. H.; Wu, H.; Anand, R.; Yu, Y.; Metzger, E.; Lo, M. M. C.; Ying, P.; Stamford, A. W.; Tempest, P. 2017, Substituted Aminoquinazoline Compounds as A_{2A} Antagonist. WO2017008205 A1

### Biological Assays

The IC₅₀ values reported for each of the compounds shown above were measured in accordance with the methods described below. Method (A) describes the procedure used to measure A_{2A} binding affinity using radioligand binding. Method (B) describes the procedure used to measure A_{2A} binding affinity using SPA technology. The method used to measure A_{2B} binding affinity is also described below. The A_{2B} IC₅₀ value measured using the A_{2B} binding affinity assay is shown in the table next to the compound under the corresponding A_{2A} value. “XX” indicates that the IC₅₀ value was not available.

The A_{2A} receptor affinity binding assay measured the amount of binding of a tritiated ligand with high affinity for the A_{2A} adenosine receptor to membranes made from HEK293 or CHO cells recombinantly expressing the human A_{2A} adenosine receptor, in the presence of varying concentrations of a compound of the invention. The data were generated using either filtration binding or a homogenous scintillation proximity assay (SPA). In both assay formats, the tested compounds of the invention were solubilized in 100% DMSO and further diluted in 100% DMSO to generate, typically, a 10-point titration at half-log intervals such that the final assay concentrations did not exceed 10 μM of compound or 1% DMSO.

### Method (A): Measurement of A_{2A} Binding Affinity Using Radioligand Binding

148 μL (5 μg/mL) membranes (Perkin Elmer, Cat. No. RBHA2aM400UA) and 2 μL compounds of the invention to be tested (test compound) were transferred to individual wells of a 96-well polypropylene assay plate and incubated for 15 to 30 min at room temperature. [³H] SCH₅₈₂₆₁ ((7-(2-phenylethyl)-5-amino-2-(2-furyl)-pyrazolo-[4,3-e]-1,2,4-triazolo[1,5-c]pyrimidine)) was diluted in assay buffer (50 mM Tris pH 7.4, 10 mM MgCl₂, 0.005% Tween20) to a concentration of 4 nM and 50 μL transferred to each well of the assay plate. To define total and non-specific binding, wells containing 1% DMSO and 1 μM ZM241385 (Tocris Bioscience, Cat. No. 1036) respectively, were also included. The assay plate was incubated at room temperature for 60 min with agitation. Using a FilterMate Harvester® (Perkin Elmer), the contents of the assay plate were filtered through a UniFilter-96 PEI coated plate (Perkin Elmer Cat. No. 6005274 or 6005277). Filtering was achieved by aspirating the contents of the assay plate for 5 sec, then washing and aspirating the contents three times with ice-cooled wash buffer (50 mM Tris-HCl pH 7.4, 150 mM NaCl) and allowing the vacuum manifold to dry the plate for sec. The filter plate was incubated for at least 1 h at 55° C. and allowed to dry. The bottom of the filter plate was sealed with backing tape. 40 μL Ultima Gold™ (Perkin Elmer, Cat. No. 6013329) was added to each well of the filter plate and the top of the plate was sealed with TopSeal-A PLUS® clear plate seal (Perkin Elmer, Cat. No. 6050185). The plate was incubated for at least 20 min, and then the amount of radioactivity remaining in each well was determined using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

### Method (B): Measurement of A_{2A} Binding Affinity Using SPA

Binding affinity using SPA was conducted as follows. Test compounds (50 μL) were dispensed into individual wells of a 384-well OptiPlate™ well (Perkin Elmer) by Echo® acoustic liquid transfer (Labcyte). 20 μL of 1.25 nM [³H] SCH₅₈₂₆₁ ((7-(2-phenylethyl)-5-amino-2-(2-furyl)-pyrazolo-[4,3-e]-1,2,4-triazolo[1,5-c]pyrimidine)) in DPBS assay buffer (Dulbecco's phosphate buffered saline without calcium and magnesium, ThermoFisher Scientific, Cat. No. A1285601) supplemented with 10 mM MgCl₂ was added. A_{2A} receptor-expressing membranes were incubated with 20 μg/mL adenosine deaminase (Roche, Cat. No. 10 102 105 001) for 15 min at room temperature. The receptor-expressing membranes were then combined with wheat germ agglutinin-coated yttrium silicate SPA beads (GE Healthcare, Cat. No. RPNQ0023) in a ratio of 1:1000 (w/w) and incubated for 30 min at room temperature. 30 μL of the membrane/bead mixture (0.25 μg and 25 μg per well respectively) were added to the 384-well OptiPlate™ well. To define total and non-specific binding, wells containing 1% DMSO or 1 μM CGS15943 (Tocris Bioscience, Cat. No. 1699) respectively were also included in the experiment. The plate was incubated for 1 h at room temperature with agitation. The assay plate was then incubated for an h to allow the beads to settle before data were collected using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

### Measurement of A_{2B} Binding Affinity

The reported affinity of the compounds of the invention for the human A_{2B} adenosine receptor was determined experimentally using a radioligand filtration binding assay. This assay measures the amount of binding of a tritiated proprietary A_{2B} receptor antagonist, in the presence and absence of a compound of the invention, to membranes made from HEK293 cells recombinantly expressing the human A_{2B} adenosine receptor (Perkin Elmer, Cat. No. ES-013-C). To perform the assay, compounds of the invention to be tested were first solubilized in 100% DMSO and further diluted in 100% DMSO to generate, typically, a 10-point titration at half-log intervals such that the final assay concentrations did not exceed 10 μM of compound or 1% DMSO. 148 μL (135 μg/mL) membranes and 2 μL test compounds were transferred to individual wells of a 96-well polypropylene assay plate and incubated for 15 to 30 min at room temperature with agitation. Tritiated radioligand was diluted to a concentration of 14 nM in assay buffer (phosphate buffered saline without Magnesium and Calcium, pH 7.4; GE Healthcare Life Sciences, Cat. No. SH30256.01) and then 50 μL of the solution were transferred to each well of the assay plate. To define total and non-specific binding, wells containing 1% DMSO and 20 μM N-ethylcarboxamidoadenosine (Tocris Bioscience, Cat. No. 1691) respectively, were also included. The wells of the assay plate were incubated at room temperature for 60 min with agitation, then filtered using a FilterMate Harvester® (Perkin Elmer) or similar equipment through a UniFilter-96® PEI coated plate (Perkin Elmer Cat. No. 6005274 or 6005277). Filtering was achieved by aspirating the contents of the assay plate for 5 sec, then washing and aspirating the contents three times with ice-cooled wash buffer (assay buffer supplemented with 0.0025% Brij58) and allowing the vacuum manifold to dry the plate for 30 sec. The filter plate was incubated for at least 1 h at 55° C. and allowed to dry. The bottom of the filter plate was then sealed with backing tape. 40 μL Ultima Gold™ (Perkin Elmer, Cat. No. 6013329) was added to each well of the filter plate and the top of the plate was sealed with TopSeal-A PLUS® clear plate seal (Perkin Elmer, Cat. No. 6050185). The plates were then incubated for at least 20 min, and then the amount of radioactivity remaining in each well was determined using a TopCount® (Perkin Elmer) scintillation counter. After normalization to total and non-specific binding, the percent effect at each compound concentration was calculated. The plot of percent effect versus the log of compound concentration was analyzed electronically using a 4-parameter logistic fit based on the Levenberg-Marquardt algorithm to generate IC₅₀ values.

Data Table

| | A_{2A} binding IC₅₀ | A_{2B} binding |
|---|---|---|
| Example | (nM) | IC₅₀ (nM) |
| Ex-1 | 17.4 | 26.3 |
| Ex-2 | 2.6 | 50.4 |
| Ex-3 | 0.3 | 2.9 |
| Ex-4 | 0.4 | 25.0 |
| Ex-5 | 0.5 | 15.4 |
| Ex-6 | 0.9 | 13.2 |
| Ex-7 | 1.3 | 66.1 |
| Ex-8 | 1.5 | 31.0 |
| Ex-9 | 2.0 | 337.3 |
| Ex-10 | 2.6 | 45.4 |
| Ex-11 | 3.1 | 78.4 |
| Ex-12 | 3.2 | 281.3 |
| Ex-13 | 3.3 | 151.5 |
| Ex-14 | 3.3 | 56.1 |
| Ex-15 | 3.4 | 1840.0 |
| Ex-16 | 4.0 | 15.5 |
| Ex-17 | 4.1 | 40.7 |
| Ex-18 | 5.5 | 252.3 |
| Ex-19 | 5.8 | 32.9 |
| Ex-20 | 6.5 | 394.8 |
| Ex-21 | 6.9 | 103.9 |
| Ex-22 | 7.7 | 132.7 |
| Ex-23 | 8.3 | 118.0 |
| Ex-24 | 9.0 | 42.6 |
| Ex-25 | 9.9 | 96.3 |
| Ex-26 | 12.3 | 718.1 |
| Ex-27 | 97.4 | 864.5 |
| Ex-28 | 1.5 | XX |
| Ex-29 | 3.5 | 45.0 |
| Ex-30 | 43.0 | 905.6 |
| Ex-31 | 44.2 | 27.3 |
| Ex-32 | 377.4 | 600.6 |
| Ex-33 | 471.7 | 62.4 |
| Ex-34 | 0.1 | 18.2 |
| Ex-35 | 7.3 | 104.7 |
| Ex-36 | 20.5 | 805.2 |
| Ex-37 | 17.7 | 1980.0 |
| Ex-38 | 1.1 | 3.1 |
| Ex-39 | 0.6 | 71.9 |
| Ex-40 | 1.4 | 7.6 |
| Ex-41 | 7.0 | 127.1 |
| Ex-42 | 0.6 | XX |
| Ex-43 | 10.9 | 10.2 |
| Ex-44 | 2.4 | XX |
| Ex-45 | 1.6 | 6.2 |
| Ex-46 | 18.3 | 69.0 |
| Ex-47 | 9.8 | 10.6 |
| Ex-48 | 1.2 | XX |
| Ex-49 | 1.3 | 3.3 |
| Ex-50 | 1.0 | XX |
| Ex-51 | XX | XX |
| Ex-52 | 3.0 | XX |
| Ex-53 | 8.8 | XX |
| Ex-54 | 1.1 | 6.9 |
| Ex-55 | 15.5 | XX |
| Ex-56 | 17.8 | XX |
| Ex-57 | 47.4 | XX |
| Ex-58 | 136.4 | 390.7 |
| Ex-59 | 5.7 | XX |
| Ex-60 | 7.9 | XX |
| Ex-61 | 42.6 | XX |
| Ex-62 | 107.3 | XX |
| Ex-63 | 7.0 | 0.4 |
| Ex-64 | 16.0 | 1.7 |
| Ex-65 | 2.1 | XX |
| Ex-66 | 2.0 | XX |
| Ex-67 | 4.3 | 1.8 |
| Ex-68 | 2.0 | 2.2 |
| Ex-69 | 6.2 | 0.3 |
| Ex-70 | 8.0 | XX |
| Ex-71 | 18.4 | XX |
| Ex-72 | 7.9 | XX |
| Ex-73 | 4.8 | XX |
| Ex-74 | 4.0 | XX |
| Ex-75 | 0.3 | XX |
| Ex-76 | 0.5 | XX |
| Ex-77 | 1.3 | XX |
| Ex-78 | 1.9 | XX |
| Ex-79 | 2.7 | XX |
| Ex-80 | 4.5 | XX |
| Ex-81 | 4.8 | XX |
| Ex-82 | 5.3 | XX |
| Ex-83 | 0.6 | 1.6 |
| Ex-84 | 8.2 | XX |
| Ex-85 | 2.7 | 1.3 |
| Ex-86 | 12.7 | XX |
| Ex-87 | 0.8 | 2.7 |
| Ex-88 | 6.6 | XX |
| Ex-89 | 27.7 | XX |
| Ex-90 | 0.6 | 11.2 |
| Ex-91 | 1.1 | XX |
| Ex-92 | 14.1 | XX |
| Ex-93 | 2.3 | XX |
| Ex-94 | 3.8 | XX |
| Ex-95 | 2.9 | XX |
| Ex-96 | 14.0 | XX |
| Ex-97 | 0.3 | XX |
| Ex-98 | 1.6 | XX |
| Ex-99 | 0.6 | XX |
| Ex-100 | 3.2 | XX |
| Ex-101 | 0.7 | XX |
| Ex-102 | 1.5 | XX |
| Ex-103 | 1.1 | XX |
| Ex-104 | 2.6 | XX |
| Ex-105 | 2.7 | XX |
| Ex-106 | 4.1 | XX |
| Ex-107 | 8.2 | XX |
| Ex-108 | 10.7 | XX |
| Ex-109 | 86.1 | XX |
| Ex-110 | 16.7 | XX |
| Ex-111 | 117.2 | XX |
| Ex-112 | 4.2 | XX |
| Ex-113 | 2.2 | XX |
| Ex-114 | 1.3 | XX |
| Ex-115 | 1.7 | XX |
| Ex-116 | 3.1 | XX |
| Ex-117 | 8.1 | XX |
| Ex-118 | 4.8 | XX |
| Ex-119 | 1.8 | XX |
| Ex-120 | 0.5 | XX |
| Ex-121 | 3.7 | XX |
| Ex-122 | 15.9 | XX |
| Ex-123 | 1.8 | XX |
| Ex-124 | 0.4 | XX |
| Ex-125 | 2.6 | XX |
| Ex-126 | 4.6 | XX |
| Ex-127 | 2.9 | XX |
| Ex-128 | 52.0 | XX |
| Ex-129 | 4.5 | XX |
| Ex-130 | 89.2 | XX |
| Ex-131 | 2.5 | XX |
| Ex-132 | 37.2 | XX |
| Ex-133 | 5.7 | XX |
| Ex-134 | 148.4 | XX |
| Ex-135 | 7.3 | 35.3 |
| Ex-136 | 2.2 | 116.1 |
| Ex-137 | 4.0 | 2.1 |
| Ex-138 | 9.8 | 12.5 |
| Ex-139 | 35.9 | 2491.0 |
| Ex-140 | 5.4 | XX |
| Ex-141 | 3.1 | XX |
| Ex-142 | 43.0 | XX |
| Ex-143 | 3.8 | XX |
| Ex-144 | 1.4 | 0.8 |
| Ex-145 | 11.9 | XX |

XX: not determined

1. A compound having a structural Formula (I): 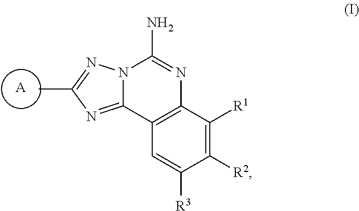
or a pharmaceutically acceptable salt thereof, wherein
R¹, R² and R³ are independently selected from the group consisting of hydrogen, halogen, —CN, —OH, C₁-C₆alkyl, —OC₁-C₆alkyl, and —OC₁-C₆haloalkyl, wherein R¹, R² and R³ are not simultaneously hydrogen;
ring A is a moiety selected from 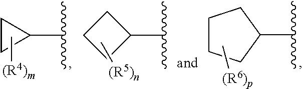
wherein
R⁴, R⁵ and R⁶ are independently selected from the group consisting of:
halogen,
—OH,
—CN,
C₁-C₆alkyl,
C₁-C₆alkyl-OH,
C₁-C₆haloalkyl,
C₃-C₆cycloalkyl,
C₁-C₆alkylC₃-C₆cycloalkyl,
aryl,
C₁-C₆alkylaryl,
heteroaryl,
C₁-C₆alkylheteroaryl,
heterocycloalkyl,
C₁-C₆alkylheterocycloalkyl,
—SO₂C₁-C₆alkyl, and
—N(R⁷)₂; and
m, n, and p are independently selected from the group consisting of 0, 1, 2 and 3; or
m, n or p is 2, and the two R⁴, R or R⁶, respectively, together with the carbon to which they are attached, form a C₃-C₆cycloalkyl or form a nitrogen containing ring, wherein the C₃-C₆cycloalkyl or nitrogen containing ring is unsubstituted or substituted with —COphenylC₁-C₆alkyl-OH or —COOC₁-C₆alkylphenyl;
wherein any of the above C₃-C₆cycloalkyl, aryl, C₁-C₆alkylaryl, heteroaryl, C₁-C₆alkylheteroaryl, or heterocycloalkyl is unsubstituted or substituted with one to three substituents independently selected from the group consisting of halogen, —CN, —OH, C₁-C₆alkyl, oxo, —CON(R⁷)₂, C₁-C₆alkyl-OH, C₁-C₆haloalkyl, NHC₁-C₆alkyl-OH, NHCO(C₁-C₆alkyl), —SO₂NH₂, C₁-C₆alkenyl, —OC₁-C₆alkyl, —OC₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl, wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH; and
R⁷ is hydrogen, —CN, C₁-C₆alkyl, C₁-C₆alkylOH, C₁-C₆alkylCN, C₁-C₆alkylheterocycloalkyl, C₁-C₆alkylOC₁-C₆alkyl, —OC₁-C₆alkyl, C₁-C₆alkenyl, heterocycloalkyl, heteroaryl, aryl or C₃-C₆cycloalkyl, wherein the C₃-C₆cycloalkyl, aryl or heteroaryl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran.

2. A compound having a structural Formula (I): 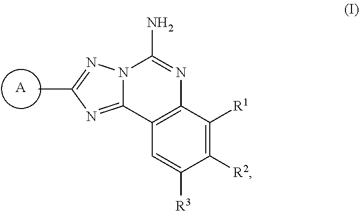
or a pharmaceutically acceptable salt thereof, wherein
R¹, R² and R³ are independently selected from the group consisting of hydrogen, halogen, —CN, —OH, C₁-C₆alkyl, —OC₁-C₆alkyl, and —OC₁-C₆haloalkyl, wherein R¹, R² and R³ are not simultaneously hydrogen;
ring A is a moiety selected from 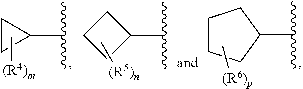
wherein
R⁵ is selected from the group consisting of:
halogen,
C₁-C₆alkyl,
C₁-C₆alkyl-OH,
C₁-C₆haloalkyl,
C₃-C₆cycloalkyl,
aryl,
C₁-C₆alkylaryl,
heteroaryl, and
heterocycloalkyl; and
m is 0, 1,2 or 3; or
m is 2 and the two R⁴, together with the carbon to which they are attached, form a C₃-C₆cycloalkyl or form a nitrogen containing ring, wherein the nitrogen containing ring is unsubstituted or substituted with —COphenylC₁-C₆alkyl-OH or —COOC₁-C₆alkylphenyl;
wherein any of the above C₃-C₆cycloalkyl, aryl, C₁-C₆alkylaryl, heteroaryl, or heterocycloalkyl is unsubstituted or substituted with one to three substituents independently selected from the group consisting of halogen, C₁-C₆alkyl, —CONH₂, oxo, —CONH(C₁-C₆alkyl), C₁-C₆alkyl-OH, NHCO(C₁-C₆alkyl), C₁-C₆haloalkyl, NHC₁-C₆alkyl-OH and —SO₂NH₂;
R⁵ is selected from the group consisting of:
halogen,
—OH,
CN,
C₁-C₆alkyl,
C₁-C₆alkylOH,
aryl,
C₁-C₆alkylheteroaryl;
heteroaryl,
heterocycloalkyl,
SO₂C₁-C₆alkyl, and
—N(R⁷)₂,
wherein any of the above aryl, C₁-C₆alkylheteroaryl, heteroaryl, or heterocycloalkyl is unsubstituted or substituted with one to three substituents independently selected from the group consisting of halogen, —CN, —OH, C₁-C₆alkylOH, C₁-C₆alkyl, oxo, C₁-C₆alkenyl, —OC₁-C₆alkyl, —OC₁-C₆haloalkyl, C₁-C₆haloalkyl, —N(R⁷)₂, C₁-C₆alkylN(R⁷)₂, C₁-C₆alkylheterocycloalkyl, heterocycloalkyl, heteroaryl, C₃-C₆cycloalkyl, C₁-C₆alkylheteroaryl, —COOC₁-C₆alkyl and —COC₁-C₆alkylaryl; wherein the C₁-C₆alkylheterocycloalkyl, heterocycloalkyl, heteroaryl, C₃-C₆cycloalkyl, C₁-C₆alkylheteroaryl, C₁-C₆alkylOH, C₁-C₆alkylN(R⁷)₂ is unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH;
R⁷ is hydrogen, —CN, C₁-C₆alkyl, C₁-C₆alkylOH, C₁-C₆alkylCN, C₁-C₆alkylheterocycloalkyl, C₁-C₆alkylOC₁-C₆alkyl, —OC₁-C₆alkyl, C₁-C₆alkenyl, heterocycloalkyl, heteroaryl, aryl or C₃-C₆cycloalkyl, wherein the C₃-C₆cycloalkyl, aryl or heteroaryl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran; and
n is 0, 1,2 or 3; or
n is 2 and the two R⁵, together with the carbon to which they are attached, form a nitrogen containing ring, wherein the nitrogen containing ring can be unsubstituted or substituted with —COphenylC₁-C₆alkyl-OH, —COOC₁-C₆alkylphenyl; and
R⁶ is selected from the group consisting of:
halogen,
—OH, and
heteroaryl, wherein the heteroaryl is unsubstituted or substituted with C₁-C₆alkylNH₂; and p is 0, 1,2 or 3.

3. The compound of claim 1, or a pharmaceutically acceptable salt thereof, wherein ring A is 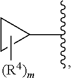
wherein:
R⁴ is selected from the group consisting of:
halogen,
C₁-C₆alkyl,
C₁-C₆alkyl-OH,
aryl,
C₁-C₆alkylaryl,
heteroaryl, and
heterocycloalkyl,
wherein any of the above aryl, C₁-C₆alkylaryl, heteroaryl, or heterocycloalkyl is unsubstituted or substituted with one to three substituents independently selected from the group consisting of halogen, C₁-C₆alkyl, oxo, —CONH₂, —CONH(C₁-C₆alkyl), C₁-C₆alkyl-OH, C₁-C₆haloalkyl, NHC₁-C₆alkyl-OH, NHCO(C₁-C₆alkyl) and —SO₂NH₂; and
m is 1,2 or 3.

4. The compound of claim 1, or a pharmaceutically acceptable salt thereof, wherein A is 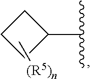
wherein R⁵ is selected from the group consisting of:
halogen,
—OH,
aryl,
heteroaryl,
heterocycloalkyl, and
—N(R⁷)₂;
wherein any of the above aryl, heteroaryl, or heterocycloalkyl is unsubstituted or substituted with one to three substituents independently selected from the group consisting of halogen, —CN, —OH, C₁-C₆alkylOH, C₁-C₆alkyl, C₁-C₆alkenyl, —OC₁-C₆alkyl, —OC₁-C₆haloalkyl, C₁-C₆haloalkyl, —N(R⁷)₂, C₁-C₆alkylN(R⁷)₂, C₁-C₆alkylpiperazinyl, oxetane, pyrrolidinyl, oxa-azabicycloheptane, C₁-C₆alkyloxa-azabicycloheptane, C₁-C₆alkylthiomorpholineoxide, thiomorpholineoxide, piperidinyl, C₃-C₆cycloalkyl, C₁-C₆alkylpyrrolidinyl, azetidine, C₁-C₆alkylazetidine, —COOC₁-C₆alkyl, morpholine, C₁-C₆alkylmorpholine, —CON(R⁷)₂, azabicyclooctane, C₁-C₆alkylazabicyclooctane, azabicycloheptane, C₁-C₆alkylazabicycloheptane, and —COC₁-C₆alkylphenyl; wherein the oxetane, C₃-C₆cycloalkyl, pyrrolidinyl, piperazinyl, C₁-C₆alkylpyrrolidinyl, azetidine, C₁-C₆alkylazetidine, piperidinyl, C₁-C₆alkylOH, C₁-C₆alkylN(R⁷)₂ and —COC₁-C₆alkylphenyl are unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH;
R⁷ is hydrogen, —CN, C₁-C₆alkylCN, C₁-C₆alkyl, tetrahydronaphthyridine, pyridinyl, phenyl, imidazolyl, pyrazinyl, bicyclopentane, C₁-C₆alkylOH, C₃-C₆cycloalkyl, oxetanyl, C₁-C₆alkenyl, C₁-C₆alkyltetrahydrofuran, —OC₁-C₆alkyl, or C₁-C₆alkylOC₁-C₆alkyl, wherein the C₃-C₆cycloalkyl, phenyl, pyrazinyl, or pyridinyl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran; and
n is 1,2 or 3.

5. The compound of claim 1, or a pharmaceutically acceptable salt thereof, wherein ring A is 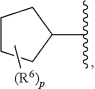
wherein R⁶ is selected from the group consisting of:
halogen,
—OH, and
pyridinyl, wherein the pyridinyl is substituted with C₁-C₆alkylNH₂; and
p is 1, 2, or 3.

6. The compound of claim 1, or a pharmaceutically acceptable salt thereof, wherein A is 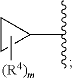
R⁴ is selected from the group consisting of halogen, C₁-C₆alkyl, C₁-C₆alkyl-OH, C₁-C₆haloalkyl, C₃-C₆cycloalkyl, C₁-C₆alkylphenyl, pyrazolyl, pyridinyl, pyrazinyl, phenyl, isoindolinone, oxadiazolyl, triazolyl, pyrimidinyl, pyridazinyl, benzimidazolyl, triazolopyridinyl, dihydrobenzooxazine, tetrahydroquinoline and imidazolyl, wherein the C₃-C₆cycloalkyl, C₁-C₆alkylphenyl, pyrazolyl, pyridinyl, pyrazinyl, phenyl, isoindolinone, oxadiazolyl, thiazolyl, triazolyl, pyrimidinyl, pyridazinyl, benzimidazolyl, triazolopyridinyl, dihydrobenzooxazine, tetrahydroquinoline and imidazolyl is unsubstituted or substituted with one to three substituents independently selected from the group consisting of halogen, C₁-C₆alkyl, —CONH₂, —CONH(C₁-C₆alkyl), C₁-C₆alkyl-OH, C₁-C₆haloalkyl, NHC₁-C₆alkyl-OH and —SO₂NH₂; and
m is 0, 1,2 or 3.

7. The compound of claim 1, or a pharmaceutically acceptable salt thereof, wherein A is 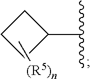
R⁵ is selected from the group consisting of halogen, —OH, —CN, C₁-C₆alkyl, C₁-C₆alkyl-OH, phenyl, C₁-C₆alkylpyridinyl, pyridinyl, pyrimidinyl, triazolyl, pyrazinyl, imidazolyl, oxadiazolyl, dihydrocyclopentapyridinyl, dihydroimidazopyrazinyl, dihydrotriazolopyridinyl, dihydropyrrolopyrimidinyl, tetrahydroimidazopyrazinyl, tetrahydrotriazolopyridinyl, tetrahydropyridopyrimidinyl, oxidaneylpyridinyl, tetrahydronaphthyridinyl, pyridinone, —SO₂C₁-C₆alkyl, and NHR⁷, wherein the phenyl, C₁-C₆alkylpyridinyl, pyridinyl, pyrimidinyl, triazolyl, pyrazinyl, imidazolyl, oxadiazolyl, dihydrocyclopentapyridinyl, dihydroimidazopyrazinyl, dihydrotriazolopyridinyl, dihydropyrrolopyrimidinyl, tetrahydroimidazopyrazinyl, tetrahydrotriazolopyridinyl, tetrahydropyridopyrimidinyl, oxidaneylpyridinyl, tetrahydronaphthyridinyl and pyridinone are unsubstituted or substituted with one to three substituents independently selected from the group consisting of halogen, —CN, —OH, C₁-C₆alkylOH, C₁-C₆alkyl, C₁-C₆alkenyl, —OC₁-C₆alkyl, —OC₁-C₆haloalkyl, C₁-C₆haloalkyl, —N(R⁷)₂, C₁-C₆alkylN(R⁷)₂, C₁-C₆alkylpiperazinyl, oxetane, pyrrolidinyl, oxa-azabicycloheptane, C₁-C₆alkyloxa-azabicycloheptane, C₁-C₆alkylthiomorpholineoxide, thiomorpholineoxide, piperidinyl, C₃-C₆cycloalkyl, C₁-C₆alkylpyrrolidinyl, azetidine, C₁-C₆alkylazetidine, —COOC₁-C₆alkyl, morpholine, C₁-C₆alkylmorpholine, —CON(R⁷)₂, azabicyclooctane, C₁-C₆alkylazabicyclooctane, azabicycloheptane and C₁-C₆alkylazabicycloheptane, and —COC₁-C₆alkylphenyl, wherein the oxetane, C₃-C₆cycloalkyl, pyrrolidinyl, piperazinyl, C₁-C₆alkylpyrrolidinyl, azetidine, C₁-C₆alkylazetidine, piperidinyl, C₁-C₆alkylOH, C₁-C₆alkylN(R⁷)₂ and —COC₁-C₆alkylphenyl are unsubstituted or substituted with one to three substituents independently selected from the group consisting of —CN, C₁-C₆alkyl, halogen, —OH, C₁-C₆haloalkyl, —N(R⁷)₂, —OC₁-C₆alkyl and C₁-C₆alkylOH;
R⁷ is hydrogen, —CN, C₁-C₆alkylCN, C₁-C₆alkyl, tetrahydronaphthyridine, pyridinyl, phenyl, imidazolyl, pyrazinyl, bicyclopentane, C₁-C₆alkylOH, C₃-C₆cycloalkyl, oxetanyl, C₁-C₆alkenyl, C₁-C₆alkyltetrahydrofuran, —OC₁-C₆alkyl, or C₁-C₆alkylOC₁-C₆alkyl, wherein the C₃-C₆cycloalkyl, phenyl, pyrazinyl, or pyridinyl is unsubstituted or substituted with —CN, C₁-C₆alkyl, —OC₁-C₆alkyl, C₁-C₆alkylOH, —COOC₁-C₆alkyl or C₁-C₆alkyltetrahydrofuran; and
n is 0, 1,2 or 3.

8. The compound of claim 1, or a pharmaceutically acceptable salt thereof, wherein A is 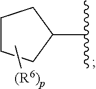
R⁶ is pyridinyl, wherein the pyridinyl, is unsubstituted or substituted with C₁-C₆alkylNH₂; and
p is 1.

9. The compound of claim 1, or a pharmaceutically acceptable salt thereof, wherein R¹, R² and R³ are independently selected from the group consisting of hydrogen, halogen, and —OC₁-C₆alkyl, wherein R¹, R² and R³ are not simultaneously hydrogen.

10. The compound of claim 1, or a pharmaceutically acceptable salt thereof, wherein R¹ is hydrogen, R² is methoxy and R³ is fluorine or hydrogen.

11. The compound of claim 1, or a pharmaceutically acceptable salt thereof, wherein R¹ is fluorine or chlorine, R² is hydrogen and R³ is fluorine or hydrogen.

12. The compound of claim 1, or a pharmaceutically acceptable salt thereof, wherein R¹ is methoxy, R² is hydrogen and R³ is fluorine or hydrogen.

13. The compound of claim 1, or a pharmaceutically acceptable salt thereof, wherein ring A is 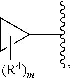
wherein:
m is 2 and the two R⁴ form a C₃-C₆cycloalkyl.

14. The compound of claim 1, or a pharmaceutically acceptable salt thereof, wherein ring A is 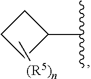
wherein n is 2 and the two R⁵ form a nitrogen containing ring, wherein the nitrogen containing ring can be unsubstituted or substituted with —COphenylC₁-C₆alkyl-OH, or —COOC₁-C₆alkylphenyl.

15. The compound of claim 1, or a pharmaceutically acceptable salt thereof, wherein said compound is selected from:

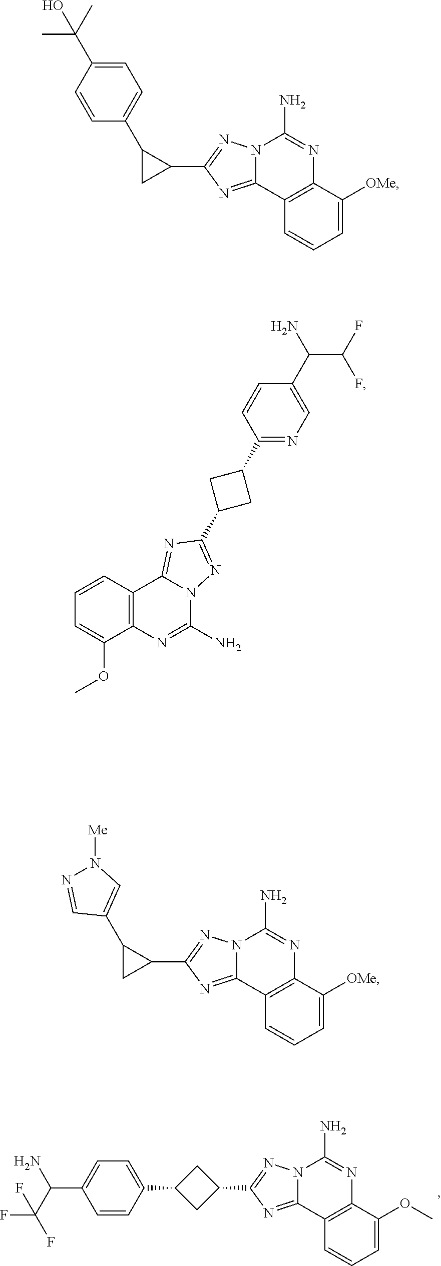 [SMILES: COc1cccc2c1nc(N)n1nc(C3CC3c3ccc(C(C)(C)O)cc3)nc21 ; COc1cccc2c1nc(N)n1nc([C@H]3C[C@@H](c4ccc(C(N)C(F)F)cn4)C3)nc21 ; COc1cccc2c1nc(N)n1nc(C3CC3c3cnn(C)c3)nc21 ; COc1cccc2c1nc(N)n1nc([C@H]3C[C@@H](c4ccc(C(N)C(F)(F)F)cc4)C3)nc21]

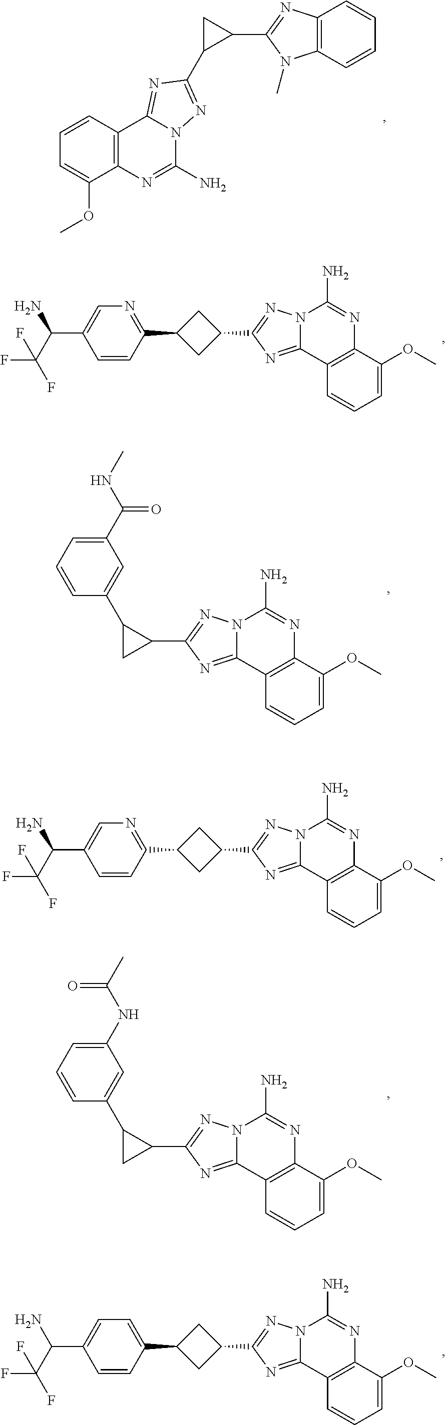 [SMILES: COc1cccc2c1nc(N)n1nc(C3CC3c3nc4ccccc4n3C)nc21 ; COc1cccc2c1nc(N)n1nc([C@H]3C[C@H](c4ccc([C@H](N)C(F)(F)F)cn4)C3)nc21 ; CNC(=O)c1cccc(C2CC2c2nc3c4cccc(OC)c4nc(N)n3n2)c1 ; COc1cccc2c1nc(N)n1nc([C@H]3C[C@@H](c4ccc([C@H](N)C(F)(F)F)cn4)C3)nc21 ; COc1cccc2c1nc(N)n1nc(C3CC3c3cccc(NC(C)=O)c3)nc21 ; COc1cccc2c1nc(N)n1nc([C@H]3C[C@H](c4ccc(C(N)C(F)(F)F)cc4)C3)nc21]

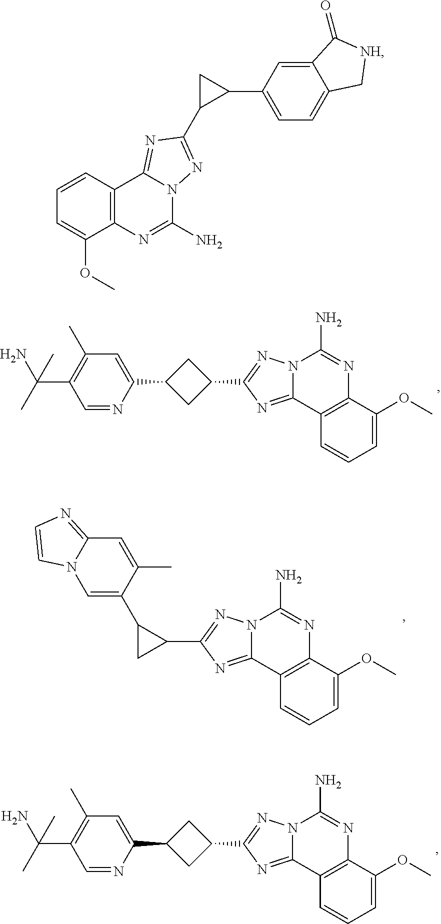

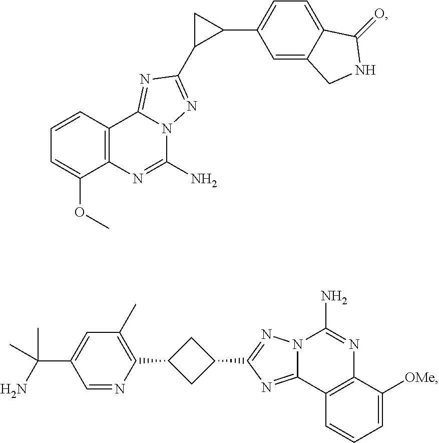 [SMILES: COc1cccc2c1nc(N)n1nc(C3CC3c3ccc4c(c3)CNC4=O)nc21 ; COc1cccc2c1nc(N)n1nc([C@H]3C[C@@H](c4ncc(C(C)(C)N)cc4C)C3)nc21]

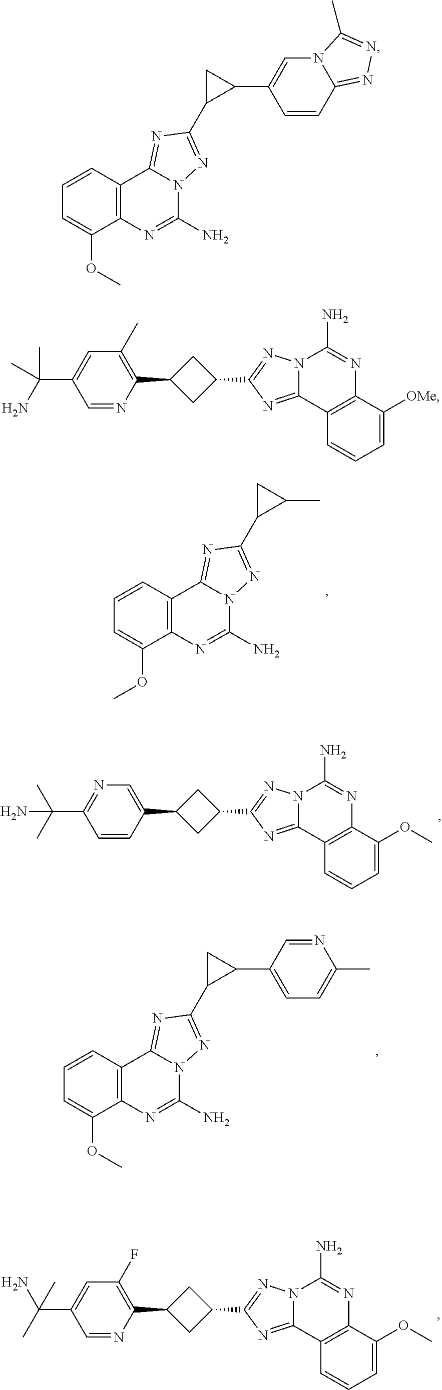

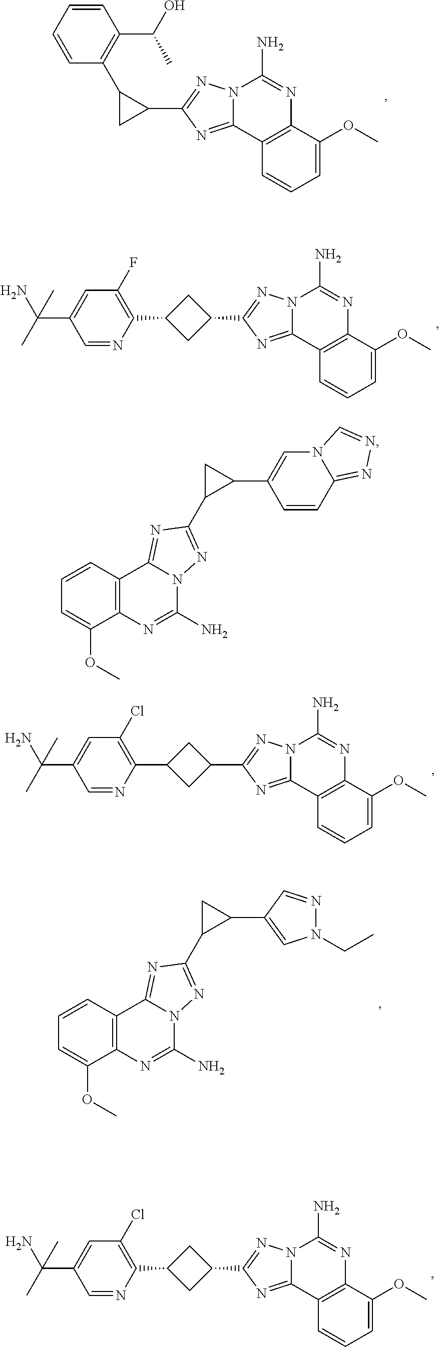

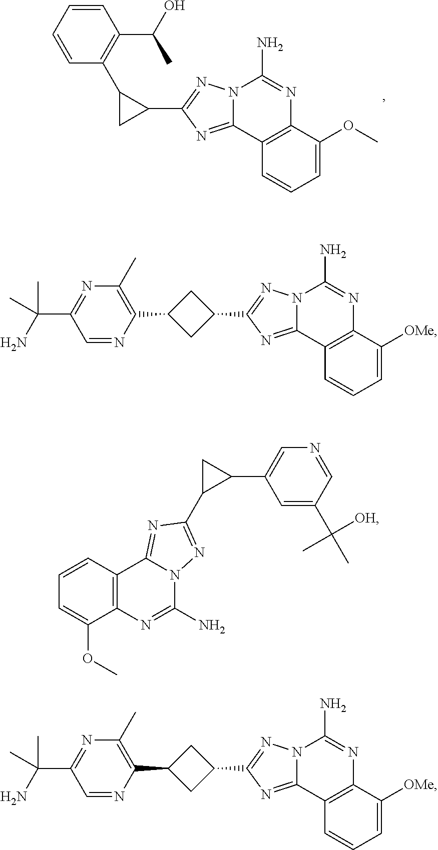

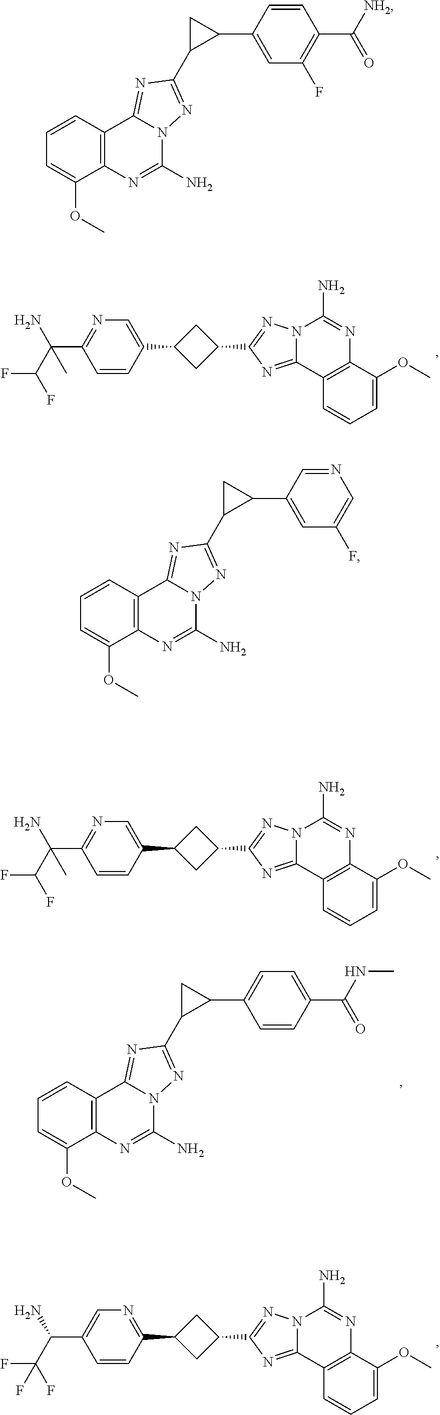 [SMILES: COc1cccc2c1nc(N)n1nc(C3CC3c3ccc(C(N)=O)c(F)c3)nc21 ; COc1cccc2c1nc(N)n1nc([C@H]3C[C@@H](c4ccc(C(C)(N)C(F)F)nc4)C3)nc21 ; COc1cccc2c1nc(N)n1nc(C3CC3c3cncc(F)c3)nc21 ; COc1cccc2c1nc(N)n1nc([C@H]3C[C@H](c4ccc(C(C)(N)C(F)F)nc4)C3)nc21 ; CNC(=O)c1ccc(C2CC2c2nc3c4cccc(OC)c4nc(N)n3n2)cc1 ; COc1cccc2c1nc(N)n1nc([C@H]3C[C@H](c4ccc([C@@H](N)C(F)(F)F)cn4)C3)nc21]

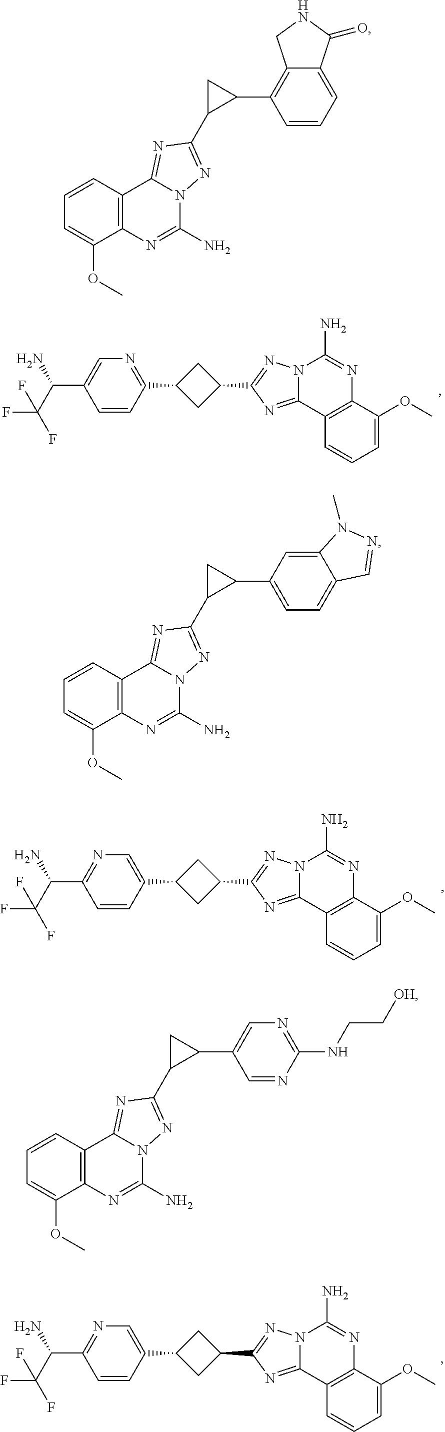

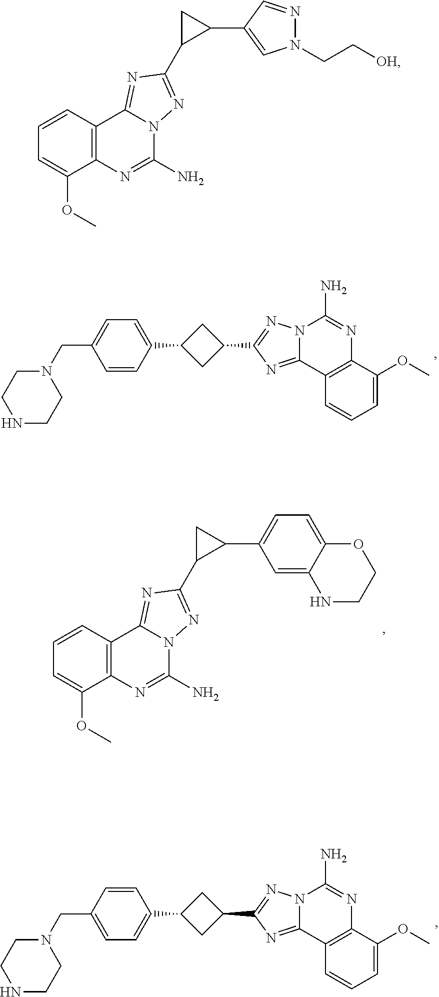 [SMILES: COc1cccc2c1nc(N)n1nc(C3CC3c3cnn(CCO)c3)nc21 ; COc1cccc2c1nc(N)n1nc([C@H]3C[C@@H](c4ccc(CN5CCNCC5)cc4)C3)nc21 ; COc1cccc2c1nc(N)n1nc(C3CC3c3ccc4c(c3)NCCO4)nc21 ; COc1cccc2c1nc(N)n1nc([C@H]3C[C@H](c4ccc(CN5CCNCC5)cc4)C3)nc21]

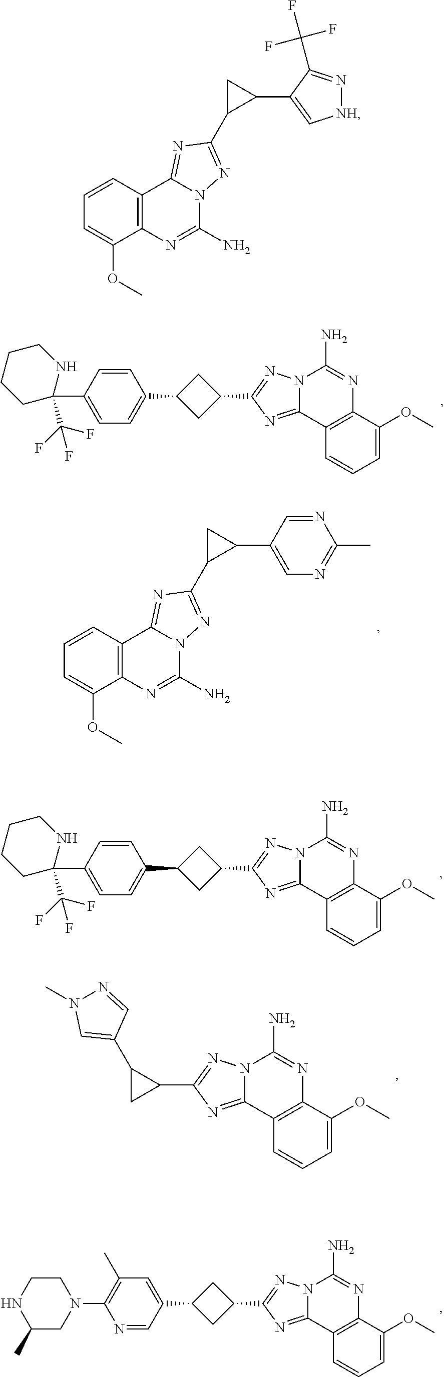

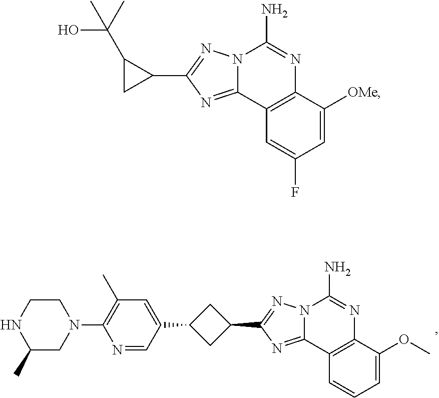 [SMILES: COc1cc(F)cc2c1nc(N)n1nc(C3CC3C(C)(C)O)nc21 ; COc1cccc2c1nc(N)n1nc([C@H]3C[C@H](c4cnc(N5CCN[C@H](C)C5)c(C)c4)C3)nc21]

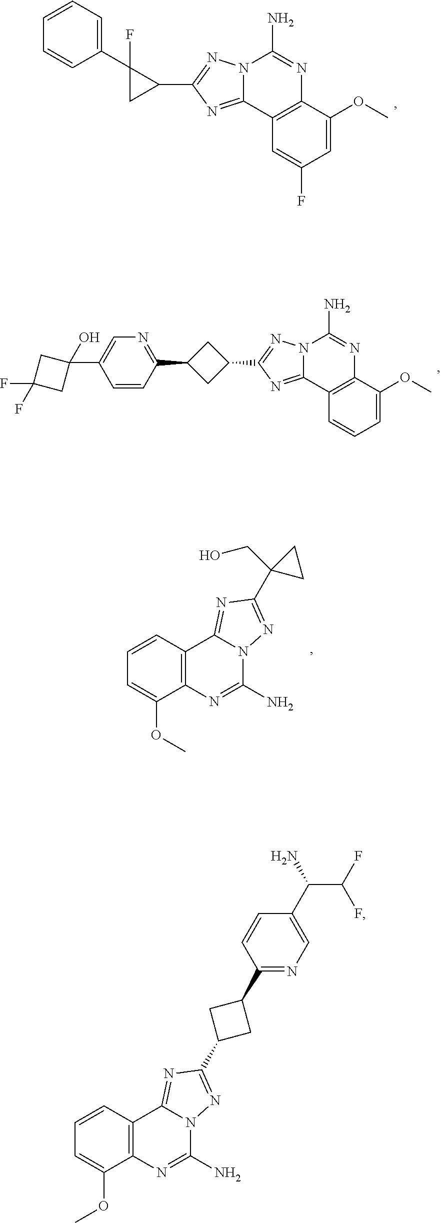 [SMILES: COc1cc(F)cc2c1nc(N)n1nc(C3CC3(F)c3ccccc3)nc21 ; COc1cccc2c1nc(N)n1nc([C@H]3C[C@H](c4ccc(C5(O)CC(F)(F)C5)cn4)C3)nc21 ; COc1cccc2c1nc(N)n1nc(C3(CO)CC3)nc21 ; COc1cccc2c1nc(N)n1nc([C@H]3C[C@H](c4ccc([C@H](N)C(F)F)cn4)C3)nc21]

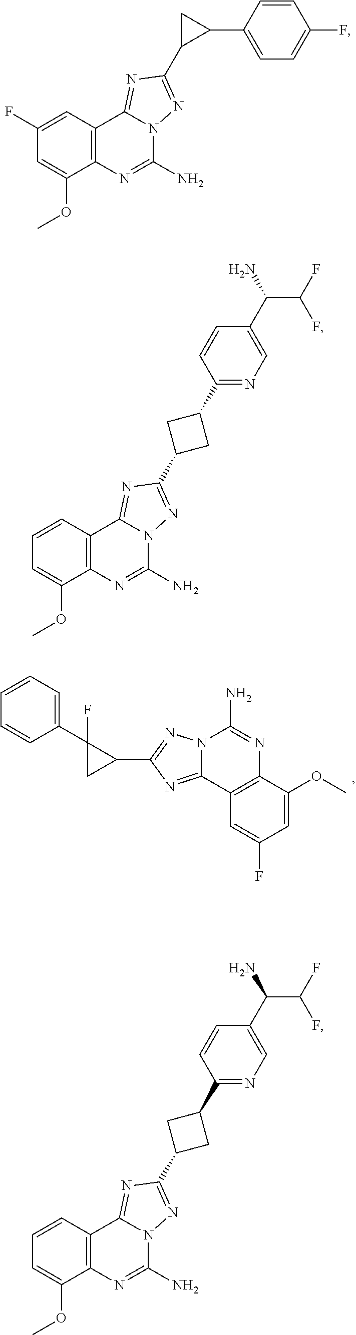

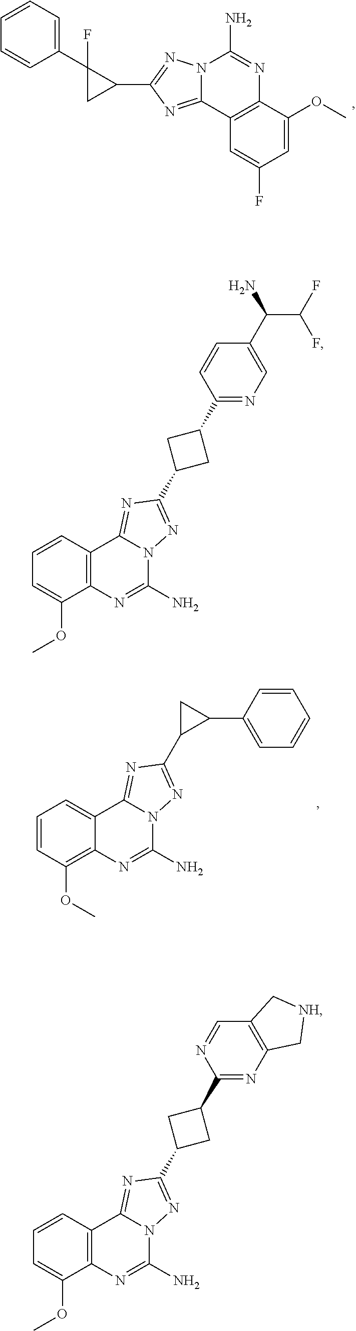 [SMILES: COc1cc(F)cc2c1nc(N)n1nc(C3CC3(F)c3ccccc3)nc21 ; COc1cccc2c1nc(N)n1nc([C@H]3C[C@@H](c4ccc([C@@H](N)C(F)F)cn4)C3)nc21 ; COc1cccc2c1nc(N)n1nc(C3CC3c3ccccc3)nc21 ; COc1cccc2c1nc(N)n1nc([C@H]3C[C@H](c4ncc5c(n4)CNC5)C3)nc21]

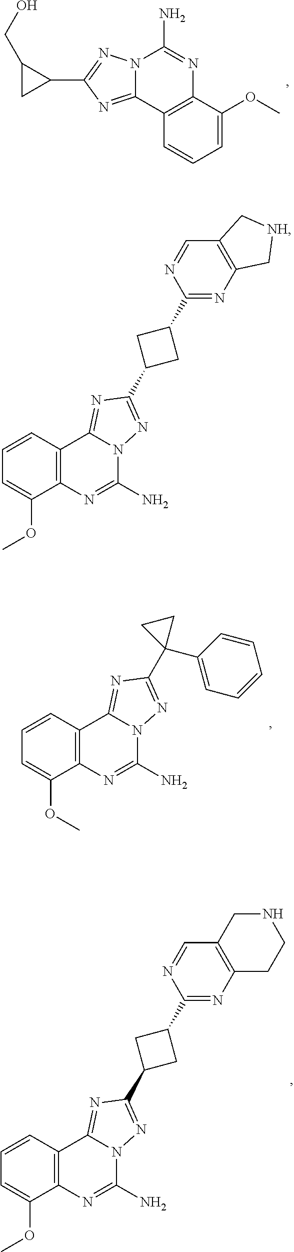 [SMILES: COc1cccc2c1nc(N)n1nc(C3CC3CO)nc21 ; COc1cccc2c1nc(N)n1nc([C@H]3C[C@@H](c4ncc5c(n4)CNC5)C3)nc21 ; COc1cccc2c1nc(N)n1nc(C3(c4ccccc4)CC3)nc21 ; COc1cccc2c1nc(N)n1nc([C@H]3C[C@H](c4ncc5c(n4)CCNC5)C3)nc21]

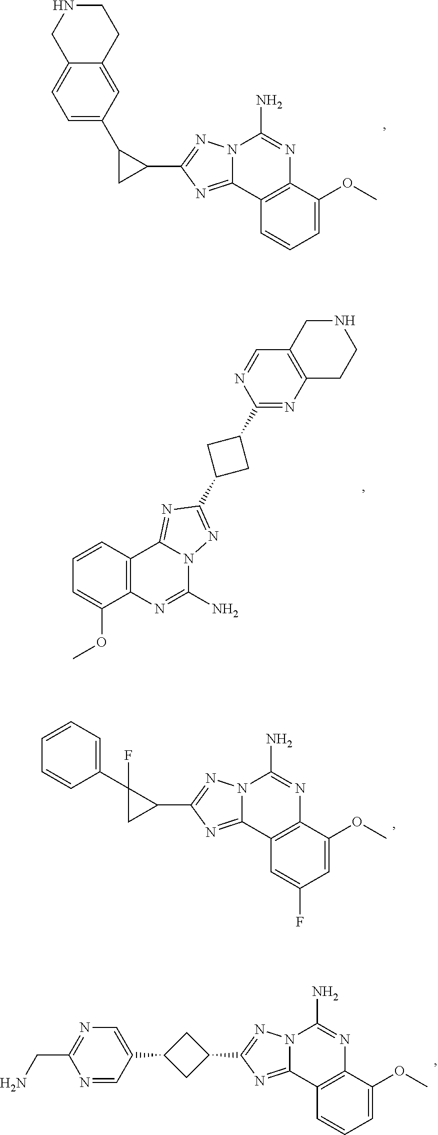 [SMILES: COc1cccc2c1nc(N)n1nc(C3CC3c3ccc4c(c3)CCNC4)nc21 ; COc1cccc2c1nc(N)n1nc([C@H]3C[C@@H](c4ncc5c(n4)CCNC5)C3)nc21 ; COc1cc(F)cc2c1nc(N)n1nc(C3CC3(F)c3ccccc3)nc21 ; COc1cccc2c1nc(N)n1nc([C@H]3C[C@@H](c4cnc(CN)nc4)C3)nc21]

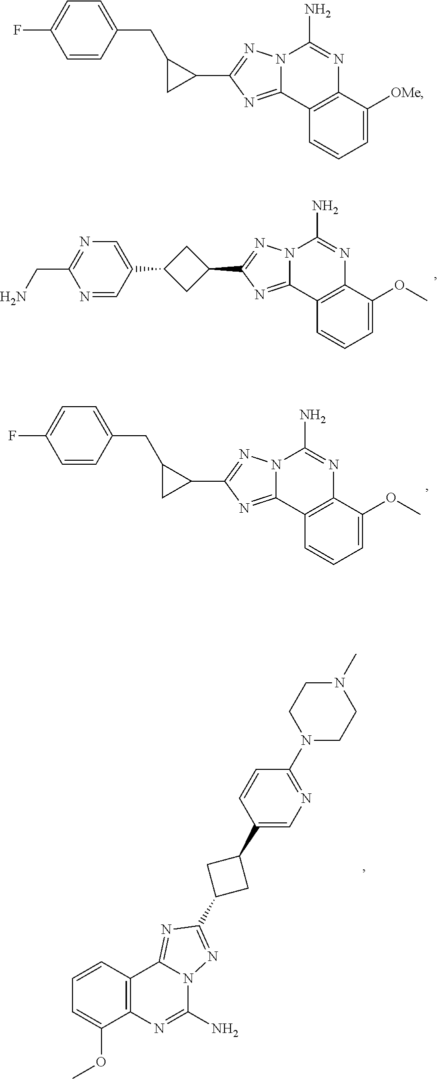 [SMILES: COc1cccc2c1nc(N)n1nc(C3CC3Cc3ccc(F)cc3)nc21 ; COc1cccc2c1nc(N)n1nc([C@H]3C[C@H](c4cnc(CN)nc4)C3)nc21 ; COc1cccc2c1nc(N)n1nc(C3CC3Cc3ccc(F)cc3)nc21 ; COc1cccc2c1nc(N)n1nc([C@H]3C[C@H](c4ccc(N5CCN(C)CC5)nc4)C3)nc21]

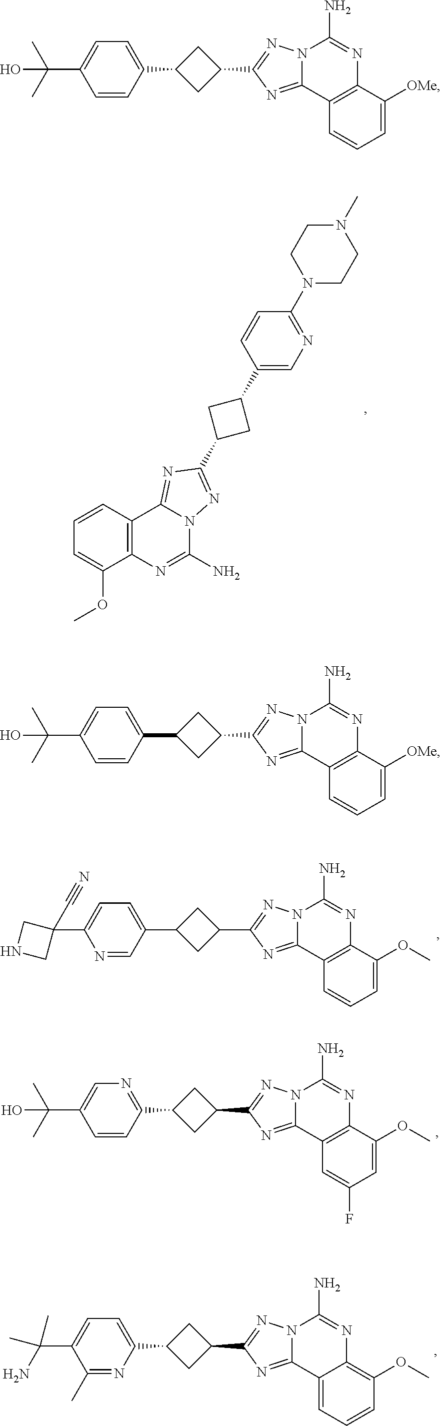

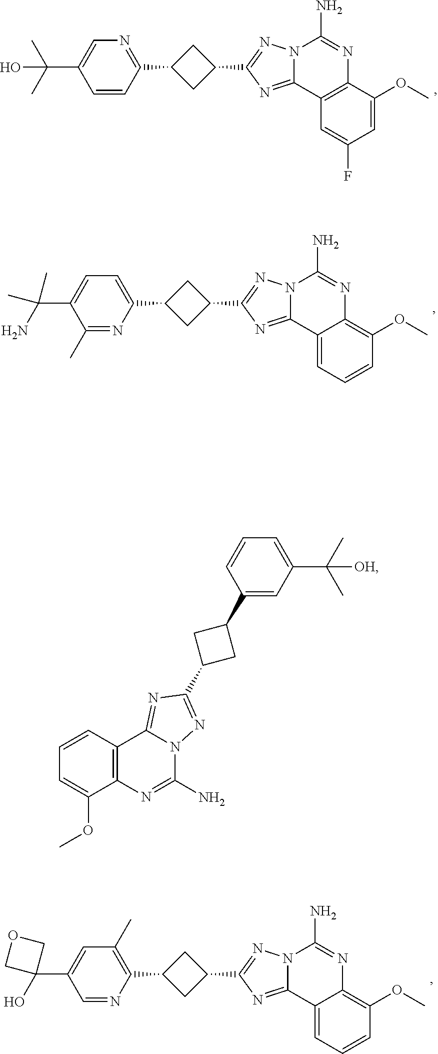

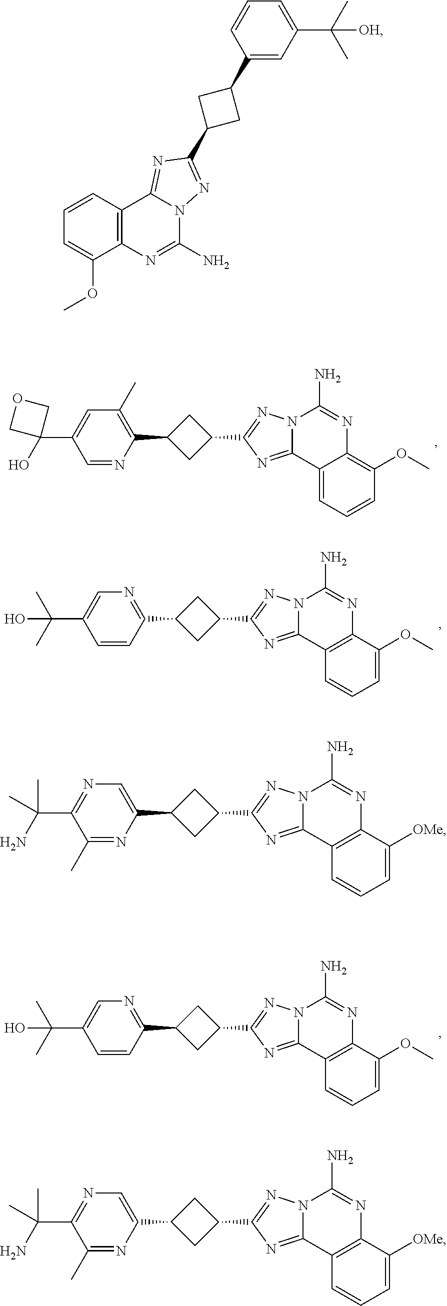

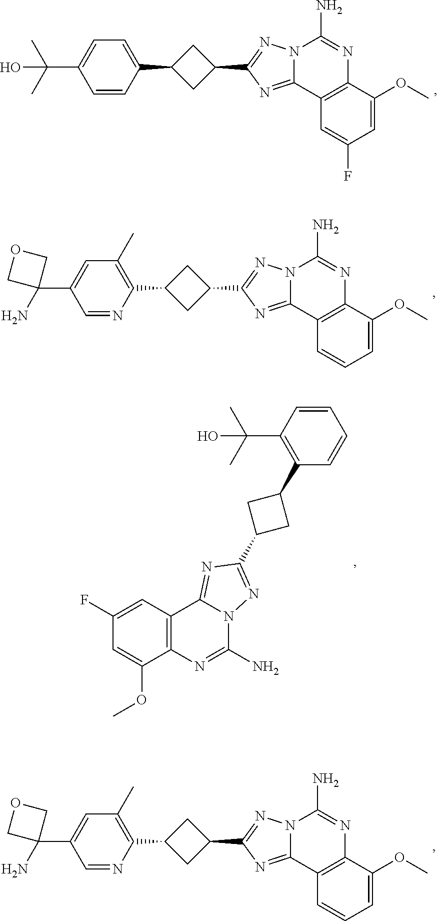

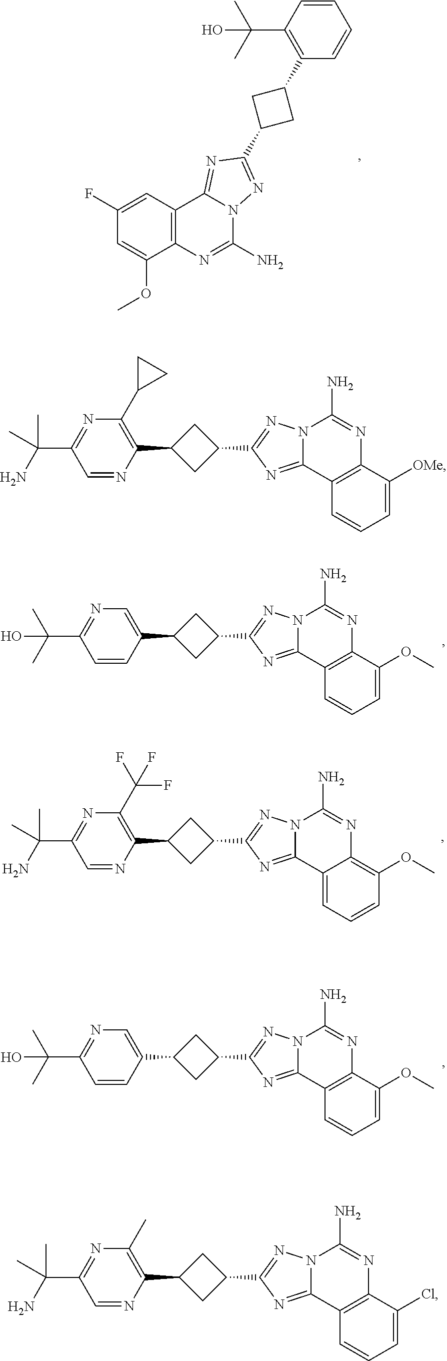

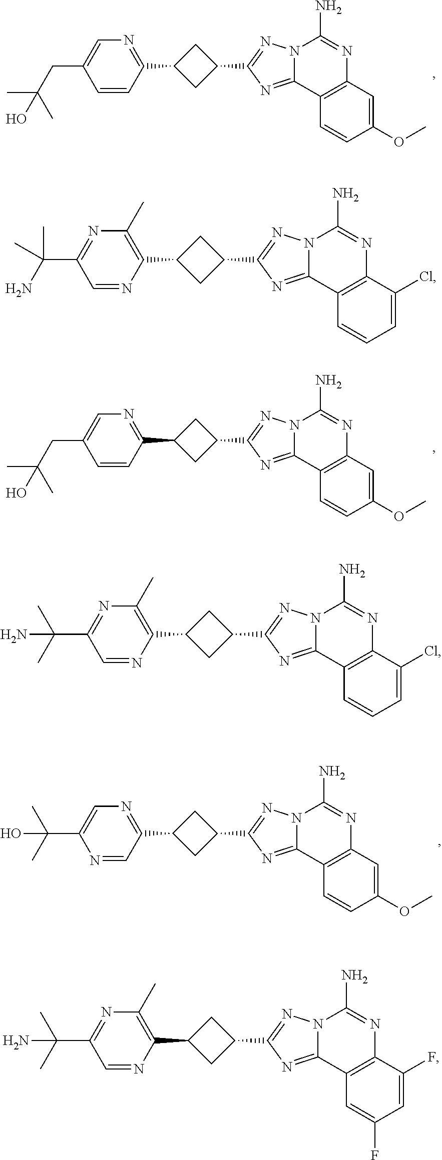

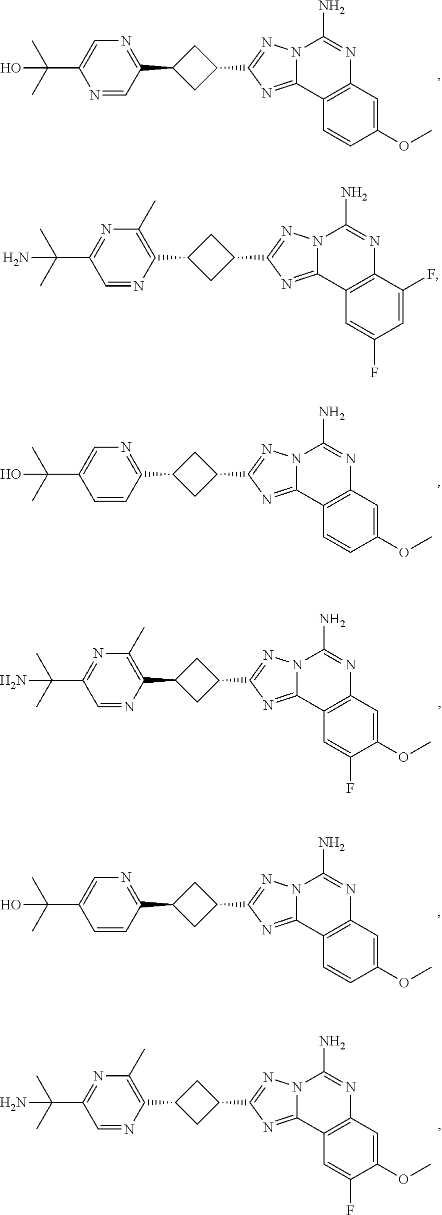 [SMILES: COc1ccc2c(c1)nc(N)n1nc([C@H]3C[C@H](c4cnc(C(C)(C)O)cn4)C3)nc21 ; Cc1nc(C(C)(C)N)cnc1[C@H]1C[C@@H](c2nc3c4cc(F)cc(F)c4nc(N)n3n2)C1 ; COc1ccc2c(c1)nc(N)n1nc([C@H]3C[C@@H](c4ccc(C(C)(C)O)cn4)C3)nc21 ; COc1cc2nc(N)n3nc([C@H]4C[C@H](c5ncc(C(C)(C)N)nc5C)C4)nc3c2cc1F ; COc1ccc2c(c1)nc(N)n1nc([C@H]3C[C@H](c4ccc(C(C)(C)O)cn4)C3)nc21 ; COc1cc2nc(N)n3nc([C@H]4C[C@@H](c5ncc(C(C)(C)N)nc5C)C4)nc3c2cc1F]

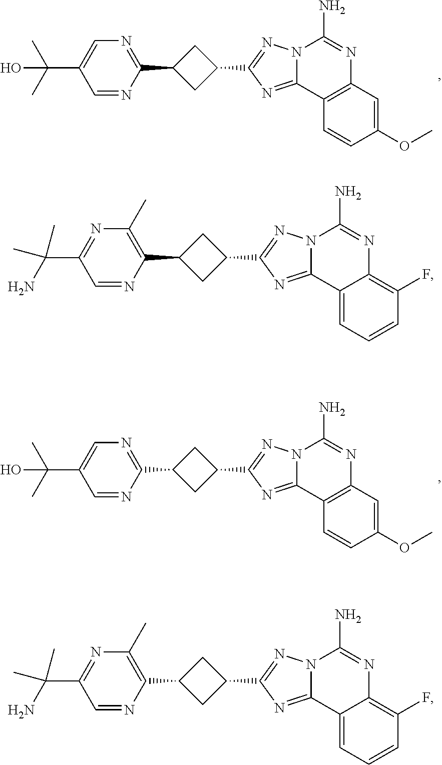

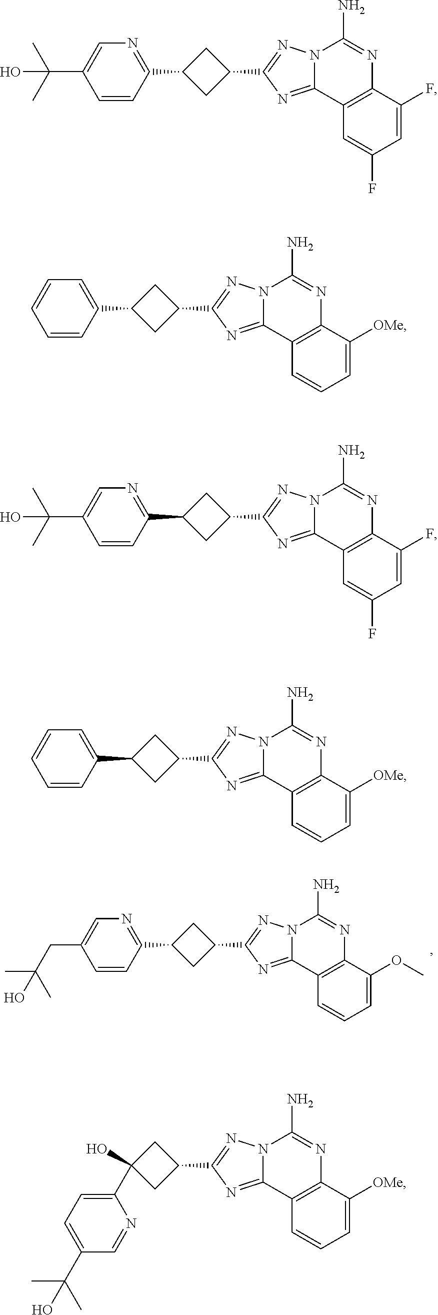

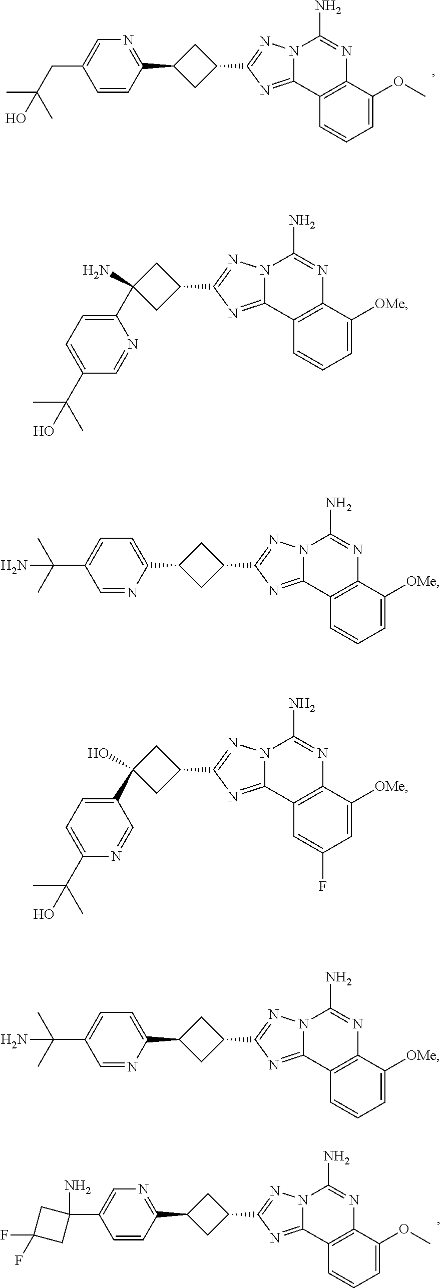

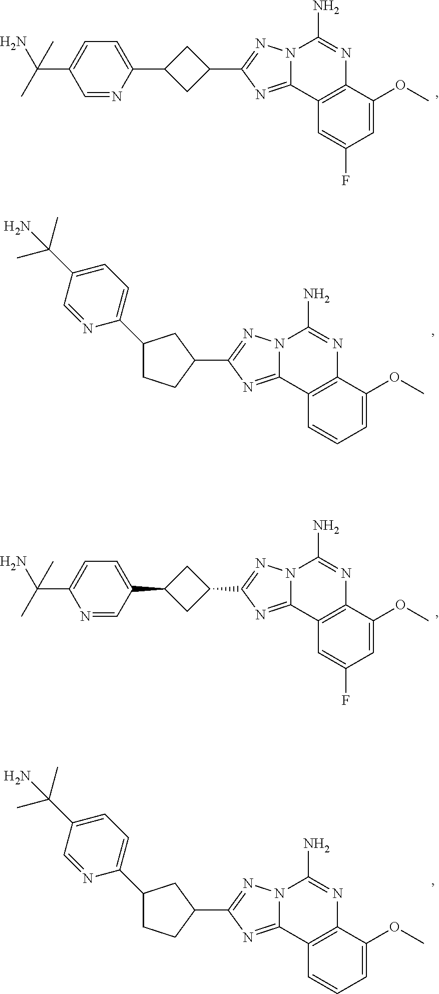

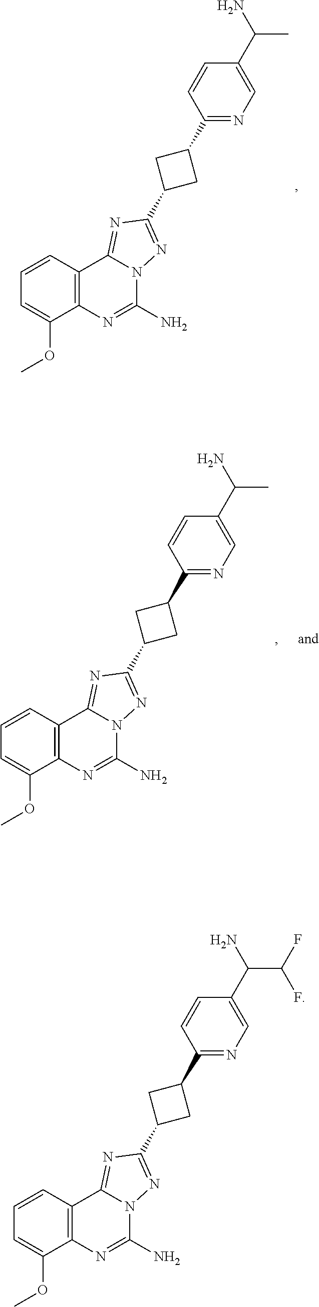 [SMILES: COc1cccc2c1nc(N)n1nc([C@H]3C[C@@H](c4ccc(C(C)N)cn4)C3)nc21 ; COc1cccc2c1nc(N)n1nc([C@H]3C[C@H](c4ccc(C(C)N)cn4)C3)nc21 ; COc1cccc2c1nc(N)n1nc([C@H]3C[C@H](c4ccc(C(N)C(F)F)cn4)C3)nc21]

16. A pharmaceutical composition comprising a compound of claim 1, or a pharmaceutically acceptable salt thereof, and a pharmaceutically acceptable carrier.

17. A method of treating cancer comprising administering an effective amount of a compound of claim 1, or a pharmaceutically acceptable salt thereof, to a person in need thereof.

18. The method of claim 17, wherein said cancer is selected from melanoma, head & neck cancer, classical Hodgkin lymphoma, urothelial carcinoma, gastric cancer, cervical cancer, primary mediastinal large-B-cell lymphoma, microsatellite instability-high cancer, non-small cell lung cancer, hepatocellular carcinoma, clear cell kidney cancer, colorectal cancer, breast cancer, squamous cell lung cancer, basal carcinoma, sarcoma, bladder cancer, endometrial cancer, pancreatic cancer, liver cancer, gastrointestinal cancer, multiple myeloma, renal cancer, mesothelioma, ovarian cancer, anal cancer, biliary tract cancer, esophageal cancer, salivary cancer, prostate cancer, and metastatic castration resistant prostate cancer.

19. The method of claim 17, wherein said compound, or a pharmaceutically acceptable salt thereof, is administered in combination with an additional therapeutic agent.

20. The method of claim 19, wherein said additional therapeutic agent is a PD-1 antagonist.

21. The method of claim 20, wherein said PD-1 antagonist is selected from pembrolizumab, nivolumab, atezolizumab, durvalumab, avelumab, cemiplimab, and dostarlimab.

22. The method of claim 20, wherein said PD-1 antagonist is pembrolizumab.

23. The method of claim 17, wherein said cancer is lung cancer, colorectal cancer, head and neck cancer or cervical cancer.
